(19) United States
(12) Patent Application Publication
(10) Pub. No.: US 2024/0287518 A1
(43) Pub. Date: Aug. 29, 2024

(54) TREATMENT OF MTRES1 RELATED DISEASES AND DISORDERS

(71) Applicant: Empirico Inc., San Diego, CA (US)

(72) Inventors: Omri GOTTESMAN, San Diego, CA (US); Shannon BRUSE, San Diego, CA (US); Paul BUSKE, Madison, WI (US); Brian CAJES, San Diego, CA (US); David JAKUBOSKY, San Diego, CA (US); Sarah KLEINSTEIN, San Diego, CA (US); David LEWIS, Madison, WI (US); David ROZEMA, Cross Plains, WI (US); John VEKICH, San Diego, CA (US)

(21) Appl. No.: 18/568,758

(22) PCT Filed: Jun. 14, 2022
(86) PCT No.: PCT/US2022/033356

§ 371 Date: Dec. 8, 2023

Related U.S. Application Data
(60) Provisional application No. 63/211,379, filed on Jun. 16, 2021.

(51) Int. Cl. C12N 15/113; A61P 25/28

(52) U.S. Cl. CPC C12N 15/113; A61P 25/28; C12N 2310/14; C12N 2310/315; C12N 2310/321; C12N 2310/3231; C12N 2310/351

(57) ABSTRACT

Disclosed herein are compositions comprising an oligonucleotide that targets MTRES1. The oligonucleotide may include a small interfering RNA (siRNA) or an antisense oligonucleotide (ASO). Also provided herein are methods of treating conditions associated with MTRES1 gene mutations that include providing an oligonucleotide that targets MTRES1 in a subject.

### CROSS-REFERENCE

This application claims the benefit of U.S. Provisional Application No. 63/211,379, filed Jun. 16, 2021, which application is incorporated herein by reference in its entirety.

### BACKGROUND

Neurological disorders are a common problem, particularly in the older population. Improved therapeutics are needed for treating these disorders.

### SUMMARY

Described herein are compositions comprising an oligonucleotide that targets MTRES1. Described herein are compositions comprising an oligonucleotide that targets MTRES1 and when administered to a subject in an effective amount reduces a MTRES1 mRNA or protein level. Described herein are compositions comprising an oligonucleotide that targets MTRES1 and when administered to a subject in an effective amount decreases central nervous system (CNS) MTRES1. In some embodiments, the CNS MTRES1 decreased by about 10% or more, as compared to prior to administration. Disclosed herein, in some embodiments, are compositions comprising an oligonucleotide that targets MTRES1 and when administered to a subject in an effective amount increases cognitive function or slows cognitive decline. In some embodiments, the cognitive function is increased by about 10% or more, as compared to prior to administration. In some embodiments, the cognitive decline is slowed by about 10% or more, as compared to prior to administration. Disclosed herein, in some embodiments, are compositions comprising an oligonucleotide that targets MTRES1 and when administered to a subject in an effective amount decreases a marker of neurodegeneration. In some embodiments, the marker of neurodegeneration comprises a central nervous system (CNS) or cerebrospinal fluid (CSF) marker of neurodegeneration. In some embodiments, the marker of neurodegeneration comprises a measurement of central nervous system (CNS) amyloid plaques, CNS tau accumulation, cerebrospinal fluid (CSF) beta-amyloid 42, CSF tau, CSF phospho-tau, CSF or plasma neurofilament light chain (NfL), Lewy bodies, or CSF alpha-synuclein. In some embodiments, the marker of neurodegeneration is decreased by about 10% or more, as compared to prior to administration. Disclosed herein, in some embodiments, are compositions comprising an oligonucleotide that targets MTRES1 and when administered to a subject in an effective amount increases cognitive function. In some embodiments, the cognitive function is increased by about 10% or more, as compared to prior to administration. Disclosed herein, in some embodiments, are compositions comprising an oligonucleotide that targets MTRES1 and when administered to a subject in an effective amount decreases central nervous system (CNS) amyloid plaques, CNS tau accumulation, cerebrospinal fluid (CSF) beta-amyloid 42, CSF tau, CSF phospho-tau, Lewy bodies, or CSF alpha-synuclein. In some embodiments, the CNS amyloid plaques, CNS tau accumulation, CSF beta-amyloid 42, CSF tau, CSF phospho-tau, Lewy bodies, or CSF alpha-synuclein, is decreased by about 10% or more, as compared to prior to administration. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the oligonucleotide comprises a modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP)nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the oligonucleotide comprises 1, 2, 3, 4, 5, 6, 7, 8, 9, 10, 11, 12, 13, 14, 15, 16, 17, 18, 19, 20, or 21 modified nucleosides. In some embodiments, the oligonucleotide comprises a lipophilic moiety attached at a 3′ or 5′ terminus of the oligonucleotide. In some embodiments, the lipophilic moiety comprises cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl, palmitic acid, myristic acid, 03-(oleoyl)lithocholic acid, 03-(oleoyl)cholenic acid, ibuprofen, naproxen, dimethoxytrityl, or phenoxazine. In some embodiments, the lipophilic moiety comprises a C4-C30 hydrocarbon chain. In some embodiments, the lipophilic moiety comprises a lipid. In some embodiments, the lipid comprises myristoyl, palmitoyl, stearoyl, lithocholoyl, docosanoyl, docosahexaenoyl, myristyl, palmityl stearyl, or α-tocopherol, or a combination thereof. In some embodiments, the oligonucleolide comprises a small interfering RNA (siRNA) comprising a sense strand and an antisense strand. In some embodiments, the sense strand is 12-30 nucleosides in length. In some embodiments, the antisense strand is 12-30 nucleosides in length. Disclosed herein, in some embodiments, are compositions comprising an oligonucleotide that inhibits the expression of MTRES1, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 2443. In some embodiments, any one of the following is true with regard to the sense strand: all purines comprise 2′ fluoro modified purines, and all pyrimidines comprise a mixture of 2′ fluoro and 2′ methyl modified pyrimidines; all purines comprise 2′ methyl modified purines, and all pyrimidines comprise a mixture of 2′ fluoro and 2′ methyl modified pyrimidines; all purines comprise 2′ fluoro modified purines, and all pyrimidines comprise 2′ methyl modified pyrimidines; all pyrimidines comprise 2′ fluoro modified pyrimidines, and all purines comprise a mixture of 2′ fluoro and 2′ methyl modified purines; all pyrimidines comprise 2′ methyl modified pyrimidines, and all purines comprise a mixture of 2′ fluoro and 2′ methyl modified purines; or all pyrimidines comprise 2′ fluoro modified pyrimidines, and all purines comprise 2′ methyl modified purines. In some embodiments, the sense strand comprises any one of modification patterns 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, or 32S. In some embodiments, any one of the following is true with regard to the antisense strand: all purines comprise 2′ fluoro modified purines, and all pyrimidines comprise a mixture of 2′ fluoro and 2′ methyl modified pyrimidines; all purines comprise 2′ methyl modified purines, and all pyrimidines comprise a mixture of 2′ fluoro and 2′ methyl modified pyrimidines; all purines comprise 2′ methyl modified purines, and all pyrimidines comprise 2′ fluoro modified pyrimidines; all pyrimidines comprise 2′ fluoro modified pyrimidines, and all purines comprise a mixture of 2′ fluoro and 2′ methyl modified purines; all pyrimidines comprise 2′ methyl modified pyrimidines, and all purines comprise a mixture of 2′ fluoro and 2′ methyl modified purines; or all pyrimidines comprise 2′ methyl modified pyrimidines, and all purines comprise 2′ fluoro modified purines. In some embodiments, the antisense strand comprises any one of modification patterns 1AS, 2AS, 3AS, 4AS, 5AS, 6AS, 7AS, 8AS, 9AS or 10AS. In some embodiments, the oligonucleotide comprises a phosphate at the 5′ end of the antisense strand. In some embodiments, the oligonucleotide comprises a phosphate mimic at the 5′ end of the antisense strand. In some embodiments, the phosphate mimic comprises a 5′-vinyl phosphonate (VP). In some embodiments, the sense strand comprises the nucleic acid sequence of any one of SEQ ID NOs: 1-1140, and the antisense strand comprises the nucleic acid sequence of any one of SEQ ID NOs: 1141-2280. In some embodiments, the oligonucleotide comprises an antisense oligonucleotide (ASO). In some embodiments, the ASO is 12-30 nucleosides in length. Disclosed herein, in some embodiments, are compositions comprising an oligonucleotide that inhibits the expression of MTRES1, wherein the oligonucleotide comprises an ASO about 12-30 nucleosides in length and a nucleoside sequence complementary to about 12-30 contiguous nucleosides of SEQ ID NO: 2443. Some embodiments include a pharmaceutically acceptable carrier. Disclosed herein, in some embodiments, are methods of treating a subject having a neurological disorder, comprising administering an effective amount of the composition to the subject. In some embodiments, the neurological disorder comprises dementia, Alzheimer's disease, delirium, cognitive decline, vascular dementia, or Parkinson's disease.

### BRIEF DESCRIPTION OF THE DRAWINGS

FIG. 1 is an image of a western blot of MTRES1 protein.

FIG. 2 is a plot quantifying MTRES1 western blot data.

FIG. 3 is a plot of MTRES1 mRNA blot data.

### DETAILED DESCRIPTION

Large-scale human genetic data can improve the success rate of pharmaceutical discovery and development. A Genome Wide Association Study (GWAS) may detect associations between genetic variants and traits in a population sample. A GWAS may enable better understanding of the biology of disease, and provide applicable treatments. A GWAS can utilize genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. Association statistics that may be used in a GWAS are p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation can identify the causal genetic variant identified via GWAS, and in many cases may lead to the identification of disease-causing genes. In particular, understanding the functional effect of a causal genetic variant (for example, loss of protein function, gain of protein function, increase in gene expression, or decrease in gene expression) may allow that variant to be used as a proxy for therapeutic modulation of the target gene, or to gain insight into potential therapeutic efficacy and safety of a therapeutic that modulates that target.

Identification of such gene-disease associations has provided insights into disease biology and may be used to identify novel therapeutic targets for the pharmaceutical industry. In order to translate the therapeutic insights derived from human genetics, disease biology in patients may be exogenously ‘programmed’ into replicating the observation from human genetics. There are several potential options for therapeutic modalities that may be brought to bear in translating therapeutic targets identified via human genetics into novel medicines. These may include well established therapeutic modalities such as small molecules and monoclonal antibodies, maturing modalities such as oligonucleotides, and emerging modalities such as gene therapy and gene editing. The choice of therapeutic modality can depend on several factors including the location of a target (for example, intracellular, extracellular, or secreted), a relevant tissue (for example, brain) and a relevant indication.

The MTRES1 gene is located on chromosome 6, and encodes mitochondrial transcription rescue factor 1 (MTRES1), also known as chromosome 6 open reading frame 203 (C6orf203). The MTRES1 gene may also be referred to as the C6orf203 gene. MTRES1 may include 240 amino acids and have a mass of about 28 kDa. MTRES1 may be expressed in neural cells. MTRES1 may be cytoplasmic or intracellular. MTRES1 may be localized in mitochondria within the cell. MTRES1 may be involved in mitochondrial transcription regulation. An example of a MTRES1 amino acid sequence, and further description of MTRES1 is included at uniprot.org under accession no. Q9P0P8 (last modified Oct. 1, 2000).

Here it is shown that loss of function MTRES1 variants may protect against neurological diseases. For example, a loss of function MTRES1 variant was associated with protective associations against Alzheimer's disease, family history of Alzheimer's disease, dementia, vascular dementia, anticholinesterase medication use, and delirium. Therefore, inhibition of MTRES1 may serve as a therapeutic for treatment of a neurological disorder such as dementia, Alzheimer's disease, delirium, cognitive decline, vascular dementia, or Parkinson's disease.

Disclosed herein are compositions comprising an oligonucleotide that targets MTRES1. Where inhibition or targeting of MTRES1 is disclosed, it is contemplated that some embodiments may include inhibiting or targeting a MTRES1 protein or MTRES1 RNA. For example, by inhibiting or targeting an RNA (e.g. mRNA) encoded by the MTRES1 gene using an oligonucleotide described herein, the MTRES1 protein may be inhibited or targeted as a result of there being less production of the MTRES1 protein by translation of the MTRES1 RNA; or a MTRES1 protein may be targeted or inhibited by an oligonucleotide that binds or interacts with a MTRES1 RNA and reduces production of the MTRES1 protein from the MTRES1 RNA. Thus, targeting MTRES1 may refer to binding a MTRES1 RNA and reducing MTRES1 RNA or protein levels. The oligonucleotide may include a small interfering RNA (siRNA) or an antisense oligonucleotide (ASO). Also provided herein are methods of treating a neurological disorder by providing an oligonucleotide that targets MTRES1 to a subject in need thereof.

### I. Compositions

Disclosed herein, in some embodiments, are compositions comprising an oligonucleotide. In some embodiments, the composition comprises an oligonucleotide that targets MTRES1. In some embodiments, the composition consists of an oligonucleotide that targets MTRES1. In some embodiments, the oligonucleotide reduces MTRES1 mRNA expression in the subject. In some embodiments, the oligonucleotide reduces MTRES1 protein expression in the subject. The oligonucleotide may include a small interfering RNA (siRNA) described herein. The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder as described herein. Some embodiments relate to use of a composition comprising an oligonucleotide, in a method of treating a disorder as described herein.

Some embodiments include a composition comprising an oligonucleotide that targets MTRES1 and when administered to a subject in an effective amount decreases MTRES1 mRNA or protein levels in a cell, fluid or tissue. In some embodiments, the composition comprises an oligonucleotide that targets MTRES1 and when administered to a subject in an effective amount decreases MTRES1 mRNA levels in a cell or tissue. In some embodiments, the cell is a neural cell such as a central nervous system (CNS) cell. Some examples of CNS cells include neurons, glia, microglia, astrocytes, or oligodendrocytes. In some embodiments, the tissue is CNS or brain tissue. In some embodiments, the MTRES1 mRNA levels are decreased by about 2.5% or more, about 5% or more, or about 7.5% or more, as compared to prior to administration. In some embodiments, the MTRES1 mRNA levels are decreased by about 10% or more, as compared to prior to administration. In some embodiments, the MTRES1 mRNA levels are decreased by about 20% or more, about 30% or more, about 40% or more, about 50% or more, about 60% or more, about 70% or more, about 80% or more, about 90% or more, or about 100%, as compared to prior to administration. In some embodiments, the MTRES1 mRNA levels are decreased by no more than about 2.5%, no more than about 5%, or no more than about 7.5%, as compared to prior to administration. In some embodiments, the MTRES1 mRNA levels are decreased by no more than about 10%, as compared to prior to administration. In some embodiments, the MTRES1 mRNA levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the MTRES1 mRNA levels are decreased by 2.5%, 5%, 7.5%, 10%, 1%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount decreases MTRES1 protein levels in a cell, fluid or tissue. In some embodiments, the cell is a neural cell such as a central nervous system (CNS) cell. Some examples of CNS cells include neurons, glia, microglia, astrocytes, or oligodendrocytes. In some embodiments, the tissue is CNS or brain tissue. In some embodiments, the MTRES1 protein levels are decreased by about 2.5% or more, about 5% or more, or about 7.5% or more, as compared to prior to administration. In some embodiments, the MTRES1 protein levels are decreased by about 10% or more, as compared to prior to administration. In some embodiments, the MTRES1 protein levels are decreased by about 20% or more, about 30% or more, about 40% or more, about 50% or more, about 60% or more, about 70% or more, about 80% or more, about 90% or more, or about 100%, as compared to prior to administration. In some embodiments, the MTRES1 protein levels are decreased by no more than about 2.5%, no more than about 5%, or no more than about 7.5%, as compared to prior to administration. In some embodiments, the MTRES1 protein levels are decreased by no more than about 10%, as compared to prior to administration. In some embodiments, the MTRES1 protein levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the MTRES1 protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount diminishes a neurological disorder phenotype. The neurological disorder disease may include dementia, Alzheimer's disease, delirium, cognitive decline, vascular dementia, or Parkinson's disease. In some embodiments, the neurological disorder phenotype is decreased by about 2.5% or more, about 5% or more, or about 7.5% or more, as compared to prior to administration. In some embodiments, the neurological disorder phenotype is decreased by about 10% or more, as compared to prior to administration. In some embodiments, the neurological disorder phenotype is decreased by about 20% or more, about 30% or more, about 40% or more, about 50% or more, about 60% or more, about 70% or more, about 80% or more, about 90% or more, or about 100%, as compared to prior to administration. In some embodiments, the neurological disorder phenotype is decreased by no more than about 2.5%, no more than about 5%, or no more than about 7.5%, as compared to prior to administration. In some embodiments, the neurological disorder phenotype is decreased by no more than about 10%, as compared to prior to administration. In some embodiments, the neurological disorder phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the neurological disorder phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount enhances a protective phenotype against a neurological disorder in the subject. The neurological disorder may include dementia, Alzheimer's disease, delirium, cognitive decline, vascular dementia, or Parkinson's disease. In some embodiments, the protective phenotype is increased by about 2.5% or more, about 5% or more, or about 7.5% or more, as compared to prior to administration. In some embodiments, the protective phenotype is increased by about 10% or more, as compared to prior to administration. In some embodiments, the protective phenotype is increased by about 20% or more, about 30% or more, about 40% or more, about 50% or more, about 60% or more, about 70% or more, about 80% or more, about 90% or more, or about 100% or more, as compared to prior to administration. In some embodiments, the protective phenotype is increased by about 200% or more, about 300% or more, about 400% or more, about 500% or more, about 600% or more, about 700% or more, about 800% or more, about 900% or more, or about 1000% or more, as compared to prior to administration. In some embodiments, the protective phenotype is increased by no more than about 2.5%, no more than about 5%, or no more than about 7.5%, as compared to prior to administration. In some embodiments, the protective phenotype is increased by no more than about 10%, as compared to prior to administration. In some embodiments, the protective phenotype is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the protective phenotype is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the protective phenotype is increased by 2.5%, 5%, 7.5%, 1, 1%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets MTRES1 and when administered to a subject in an effective amount decreases a marker of neurodegeneration in the subject. Some example markers of neurodegeneration may include central nervous system (CNS) amyloid plaques, CNS tau accumulation, cerebrospinal fluid (CSF) beta-amyloid 42, CSF tau, CSF phospho-tau, CSF or plasma neurofilament light chain (NfL), Lewy bodies, or CSF alpha-synuclein. In some embodiments, the marker of neurodegeneration is decreased by about 2.5% or more, about 5% or more, or about 7.5% or more, as compared to prior to administration. In some embodiments, the marker of neurodegeneration is decreased by about 10% or more, as compared to prior to administration. In some embodiments, the marker of neurodegeneration is decreased by about 20% or more, about 30% or more, about 40% or more, about 50% or more, about 60% or more, about 70% or more, about 80% or more, about 90% or more, or about 100%, as compared to prior to administration. In some embodiments, the marker of neurodegeneration is decreased by no more than about 2.5%, no more than about 5%, or no more than about 7.5%, as compared to prior to administration. In some embodiments, the marker of neurodegeneration is decreased by no more than about 10%, as compared to prior to administration. In some embodiments, the marker of neurodegeneration is decreased by no more than about 20%, no more than about 30%, no more than about 40%, no more than about 50%, no more than about 60%, no more than about 70%, no more than about 80%, or no more than about 90%, as compared to prior to administration. In some embodiments, the marker of neurodegeneration is decreased by 2.5%, 5%, 7.5%, 10%, 15%0, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount decreases central nervous system (CNS) amyloid plaques in the subject. In some embodiments, the CNS amyloid plaques are decreased by about 2.5% or more, about 5% or more, or about 7.5% or more, as compared to prior to administration. In some embodiments, the CNS amyloid plaques are decreased by about 10% or more, as compared to prior to administration. In some embodiments, the CNS amyloid plaques are decreased by about 20% or more, about 30% or more, about 40% or more, about 50% or more, about 60% or more, about 70% or more, about 80% or more, about 90% or more, or about 100%, as compared to prior to administration. In some embodiments, the CNS amyloid plaques are decreased by no more than about 2.5%, no more than about 5%, or no more than about 7.5%, as compared to prior to administration. In some embodiments, the CNS amyloid plaques are decreased by no more than about 10%, as compared to prior to administration. In some embodiments, the CNS amyloid plaques are decreased by no more than about 20%, no more than about 30%, no more than about 40%, no more than about 50%, no more than about 60%, no more than about 70%, no more than about 80%, or no more than about 90%, as compared to prior to administration. In some embodiments, the CNS amyloid plaques are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount decreases central nervous system (CNS) tau accumulation in the subject. In some embodiments, the CNS tau accumulation is decreased by about 2.5% or more, about 5% or more, or about 7.5% or more, as compared to prior to administration. In some embodiments, the CNS tau accumulation is decreased by about 10% or more, as compared to prior to administration. In some embodiments, the CNS tau accumulation is decreased by about 20% or more, about 30% or more, about 40% or more, about 50% or more, about 60% or more, about 70% or more, about 80% or more, about 90% or more, or about 100%, as compared to prior to administration. In some embodiments, the CNS tau accumulation is decreased by no more than about 2.5%, no more than about 5%, or no more than about 7.5%, as compared to prior to administration. In some embodiments, the CNS tau accumulation is decreased by no more than about 10%, as compared to prior to administration. In some embodiments, the CNS tau accumulation is decreased by no more than about 20%, no more than about 30%, no more than about 40%, no more than about 50%, no more than about 60%, no more than about 70%, no more than about 80%, or no more than about 90%, as compared to prior to administration. In some embodiments, the CNS tau accumulation is decreased by 2.5%, 5%, 7.5%, 10%, 15%, 20%, 25%, 30%, 35%, 40%, 45%, 50%, 55%, 60%, 6%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount decreases cerebrospinal fluid (CSF) beta-amyloid 42 in the subject. In some embodiments, the CSF beta-amyloid 42 is decreased by about 2.5% or more, about 5% or more, or about 7.5% or more, as compared to prior to administration. In some embodiments, the CSF beta-amyloid 42 is decreased by about 10% or more, as compared to prior to administration. In some embodiments, the CSF beta-amyloid 42 is decreased by about 20% or more, about 30% or more, about 40% or more, about 50% or more, about 60% or more, about 70% or more, about 80% or more, about 90% or more, or about 100%, as compared to prior to administration. In some embodiments, the CSF beta-amyloid 42 is decreased by no more than about 2.5%, no more than about 5%, or no more than about 7.5%, as compared to prior to administration. In some embodiments, the CSF beta-amyloid 42 is decreased by no more than about 10%, as compared to prior to administration. In some embodiments, the CSF beta-amyloid 42 is decreased by no more than about 20%, no more than about 30%, no more than about 40%, no more than about 50%, no more than about 60%, no more than about 70%, no more than about 80%, or no more than about 90%, as compared to prior to administration. In some embodiments, the CSF beta-amyloid 42 is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount decreases cerebrospinal fluid (CSF) tau in the subject. In some embodiments, the CSF tau is decreased by about 2.5% or more, about 5% or more, or about 7.5% or more, as compared to prior to administration. In some embodiments, the CSF tau is decreased by about 10% or more, as compared to prior to administration. In some embodiments, the CSF tau is decreased by about 20% or more, about 30% or more, about 40% or more, about 50% or more, about 60% or more, about 70% or more, about 80% or more, about 90% or more, or about 100%, as compared to prior to administration. In some embodiments, the CSF tau is decreased by no more than about 2.5%, no more than about 5%, or no more than about 7.5%, as compared to prior to administration. In some embodiments, the CSF tau is decreased by no more than about 10%, as compared to prior to administration. In some embodiments, the CSF tau is decreased by no more than about 20%, no more than about 30%, no more than about 40%, no more than about 50%, no more than about 60%, no more than about 70%, no more than about 80%, or no more than about 90%, as compared to prior to administration. In some embodiments, the CSF tau is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount decreases cerebrospinal fluid (CSF) tau in the subject. In some embodiments, the CSF phospho-tau is decreased by about 2.5% or more, about 5% or more, or about 7.5% or more, as compared to prior to administration. In some embodiments, the CSF phospho-tau is decreased by about 10% or more, as compared to prior to administration. In some embodiments, the CSF phospho-tau is decreased by about 20% or more, about 30% or more, about 40% or more, about 50% or more, about 60% or more, about 70% or more, about 80% or more, about 90% or more, or about 100%, as compared to prior to administration. In some embodiments, the CSF phospho-tau is decreased by no more than about 2.5%, no more than about 5%, or no more than about 7.5%, as compared to prior to administration. In some embodiments, the CSF phospho-tau is decreased by no more than about 10%, as compared to prior to administration. In some embodiments, the CSF phospho-tau is decreased by no more than about 20%, no more than about 30%, no more than about 40%, no more than about 50%, no more than about 60%, no more than about 70%, no more than about 80%, or no more than about 90%, as compared to prior to administration. In some embodiments, the CSF phospho-tau is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount decreases cerebrospinal fluid (CSF) alpha-synuclein in the subject. In some embodiments, the CSF alpha-synuclein is decreased by about 2.5% or more, about 5% or more, or about 7.5% or more, as compared to prior to administration. In some embodiments, the CSF alpha-synuclein is decreased by about 10% or more, as compared to prior to administration. In some embodiments, the CSF alpha-synuclein is decreased by about 20% or more, about 30% or more, about 40% or more, about 50% or more, about 60% or more, about 70% or more, about 80% or more, about 90% or more, or about 100%, as compared to prior to administration. In some embodiments, the CSF alpha-synuclein is decreased by no more than about 2.5%, no more than about 5%, or no more than about 7.5%, as compared to prior to administration. In some embodiments, the CSF alpha-synuclein is decreased by no more than about 10%, as compared to prior to administration. In some embodiments, the CSF alpha-synuclein is decreased by no more than about 20%, no more than about 30%, no more than about 40%, no more than about 50%, no more than about 60%, no more than about 70%, no more than about 80%, or no more than about 90%, as compared to prior to administration. In some embodiments, the CSF alpha-synuclein is decreased by 2.5%, 5%, 7.5%, 1%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MTRES1 and when administered to a subject in an effective amount decreases Lewy bodies in the subject. In some embodiments, the Lewy bodies are decreased by about 2.5% or more, about 5% or more, or about 7.5% or more, as compared to prior to administration. In some embodiments, the Lewy bodies are decreased by about 10% or more, as compared to prior to administration. In some embodiments, the Lewy bodies are decreased by about 20% or more, about 30% or more, about 40% or more, about 50% or more, about 60⁰% or more, about 70% or more, about 80% or more, about 90% or more, or about 100%, as compared to prior to administration. In some embodiments, the Lewy bodies are decreased by no more than about 2.5%, no more than about 5%, or no more than about 7.5%, as compared to prior to administration. In some embodiments, the Lewy bodies are decreased by no more than about 10%, as compared to prior to administration. In some embodiments, the Lewy bodies are decreased by no more than about 20%, no more than about 30%, no more than about 40%, no more than about 50%, no more than about 60%, no more than about 70%, no more than about 80%, or no more than about 90%, as compared to prior to administration. In some embodiments, the Lewy bodies are decreased by 2.5%, 5%, 7.5%, 10%, 15%, 20%, 25%, 30%, 35%, 40%, 45%, 50%, 55%, 60%, 6%, 70%, 7%, 80%, 85%, 90%, 95%, or 100%, or by a range defined by any of the two aforementioned percentages.

In some embodiments, the composition comprises an oligonucleotide that targets MTRES1 and when administered to a subject in an effective amount increases cognitive function. In some embodiments, the cognitive function is increased by about 2.5% or more, about 5% or more, or about 7.5% or more, as compared to prior to administration. In some embodiments, the cognitive function is increased by about 10% or more, as compared to prior to administration. In some embodiments, the cognitive function is increased by about 20% or more, about 30% or more, about 40% or more, about 50% or more, about 60% or more, about 70% or more, about 80% or more, about 90% or more, or about 100% or more, as compared to prior to administration. In some embodiments, the cognitive function is increased by about 200% or more, about 300% or more, about 400% or more, about 500% or more, about 600% or more, about 700% or more, about 800% or more, about 900% or more, or about 1000% or more, as compared to prior to administration. In some embodiments, the cognitive function is increased by no more than about 2.5%, no more than about 5%, or no more than about 7.5%, as compared to prior to administration. In some embodiments, the cognitive function is increased by no more than about 10%, as compared to prior to administration. In some embodiments, the cognitive function is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the cognitive function is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the cognitive function is increased by 2.5%, 5%, 7.5%, 10, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

A. siRNAs

In some embodiments, the composition comprises an oligonucleotide that targets MTRES1, wherein the oligonucleotide comprises a small interfering RNA (siRNA). In some embodiments, the composition comprises an oligonucleotide that targets MTRES1, wherein the oligonucleotide comprises a small interfering RNA (siRNA) comprising a sense strand and an antisense strand.

In some embodiments, the composition comprises an oligonucleotide that inhibits the expression of MTRES1, wherein the oligonucleotide comprises an siRNA comprising a sense strand and an antisense strand, wherein the sense strand is 12-30 nucleosides in length. In some embodiments, the composition comprises a sense strange that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The sense strand may be 14-30 nucleosides in length. In some embodiments, the composition comprises an antisense strand is 12-30 nucleosides in length. In some embodiments, the composition comprises an antisense strand that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The antisense strand may be 14-30 nucleosides in length.

In some embodiments, the composition comprises an oligonucleotide that inhibits the expression of MTRES1, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of a full-length human MTRES1 mRNA sequence such as SEQ ID NO: 2443. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 2443.

In some embodiments, the composition comprises an oligonucleotide that inhibits the expression of MTRES1, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of a full-length human MTRES1 mRNA sequence such as SEQ ID NO: 2462. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 2462.

In some embodiments, the composition comprises an oligonucleotide that inhibits the expression of MTRES1, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of MTRES1, wherein the oligonucleotide comprises an siRNA comprising a sense strand and an antisense strand, wherein the siRNA binds with a 19mer in a human MTRES1 mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a human MTRES1 mRNA.

In some embodiments, the composition comprises an oligonucleotide that inhibits the expression of MTRES1, wherein the oligonucleotide comprises an siRNA comprising a sense strand and an antisense strand, wherein the siRNA binds with a 17mer in a non-human primate MTRES1 mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate MTRES1 mRNA.

In some embodiments, the composition comprises an oligonucleotide that inhibits the expression of MTRES1, wherein the oligonucleotide comprises an siRNA comprising a sense strand and an antisense strand, wherein the siRNA binds with a human MTRES1 mRNA and less than or equal to 20 human off-targets, with no more than 2 mismatches in the antisense strand. In some embodiments, the siRNA binds with a human MTRES1 mRNA and less than or equal to 10 human off-targets, with no more than 2 mismatches in the antisense strand. In some embodiments, the siRNA binds with a human MTRES1 mRNA and less than or equal to 30 human off-targets, with no more than 2 mismatches in the antisense strand. In some embodiments, the siRNA binds with a human MTRES1 mRNA and less than or equal to 40 human off-targets, with no more than 2 mismatches in the antisense strand. In some embodiments, the siRNA binds with a human MTRES1 mRNA and less than or equal to 50 human off-targets, with no more than 2 mismatches in the antisense strand. In some embodiments, the siRNA binds with a human MTRES1 mRNA and less than or equal to 10 human off-targets, with no more than 3 mismatches in the antisense strand. In some embodiments, the siRNA binds with a human MTRES1 mRNA and less than or equal to 20 human off-targets, with no more than 3 mismatches in the antisense strand. In some embodiments, the siRNA binds with a human MTRES1 mRNA and less than or equal to 30 human off-targets, with no more than 3 mismatches in the antisense strand. In some embodiments, the siRNA binds with a human MTRES1 mRNA and less than or equal to 40 human off-targets, with no more than 3 mismatches in the antisense strand. In some embodiments, the siRNA binds with a human MTRES1 mRNA and less than or equal to 50 human off-targets, with no more than 3 mismatches in the antisense strand.

In some embodiments, the composition comprises an oligonucleotide that inhibits the expression of MTRES1, wherein the oligonucleotide comprises an siRNA comprising a sense strand and an antisense strand, siRNA binds with a human MTRES1 mRNA target site that does not harbor an SNP, with a minor allele frequency (MAF) greater or equal to 1% (pos. 2-18). In some embodiments, the MAF is greater or equal to about 2%, about 3%, about 4%, about 5%, about 6%, about 7%, about 8%, about 9%, about 10%, about 110, about 12%, about 13%, about 14%, about 15%, about 16%, about 17%, about 18%, about 19%, or about 20%.

In some embodiments, the composition comprises an oligonucleotide that inhibits the expression of MTRES1, wherein the oligonucleotide comprises an siRNA comprising a sense strand and an antisense strand, wherein the sense strand comprises a nucleoside sequence comprising or consisting of the sequence of any one of SEQ ID NOs: 1-1140, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 1-1140, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 1-1140, or a nucleic acid sequence thereof having 1 or 2 nucleoside additions at the 3′ end. In some embodiments, the composition comprises an oligonucleotide that inhibits the expression of MTRES1, wherein the oligonucleotide comprises an siRNA comprising a sense strand and an antisense strand, wherein the sense strand comprises a nucleoside sequence comprising or consisting of the sequence of any one of SEQ ID NOs: 1-1140.

In some embodiments, the composition comprises an oligonucleotide that inhibits the expression of MTRES1, wherein the oligonucleotide comprises an siRNA comprising a sense strand and an antisense strand, wherein the antisense strand comprises a nucleoside sequence comprising or consisting of the sequence of any one of SEQ ID NOs: 1141-2280, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 1141-2280, or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 1141-2280, or a nucleic acid sequence thereof having 1 or 2 nucleoside additions at the 3′ end. In some embodiments, the composition comprises an oligonucleotide that inhibits the expression of MTRES1, wherein the oligonucleotide comprises an siRNA comprising a sense strand and an antisense strand, wherein the antisense strand comprises a nucleoside sequence comprising or consisting of the sequence of any one of SEQ ID NOs: 1141-2280.

In some embodiments, the siRNA comprises the sense strand and/or the antisense strand sequence of an siRNA in any one of Tables 2-7, or a nucleic acid sequence thereof having 3 or 4 nucleoside substitutions, additions, or deletions. In some embodiments, the siRNA comprises the sense strand and/or the antisense strand sequence of an siRNA in any one of Tables 2-7, or a nucleic acid sequence thereof having 1 or 2 nucleoside substitutions, additions, or deletions. In some embodiments, the siRNA comprises the sense strand and/or the antisense strand sequence of an siRNA in any one of Tables 2-7. In some embodiments, the siRNA is cross-reactive with a non-human primate (NHP) MTRES1 mRNA. The siRNA may include one or more internucleoside linkages and/or one or more nucleoside modifications.

In some embodiments, the siRNA comprises the sense strand and/or the antisense strand sequence of an siRNA in Table 11B, or a nucleic acid sequence thereof having 3 or 4 nucleoside substitutions, additions, or deletions. In some embodiments, the siRNA comprises the sense strand and/or the antisense strand sequence of an siRNA in Table 11B, or a nucleic acid sequence thereof having 1 or 2 nucleoside substitutions, additions, or deletions. In some embodiments, the siRNA comprises the sense strand and/or the antisense strand sequence of an siRNA in Table 11B. In some embodiments, the siRNA is cross-reactive with a non-human primate (NHP) MTRES1 mRNA. The siRNA may include one or more internucleoside linkages and/or one or more nucleoside modifications. The siRNA may include a moiety such as a lipid moiety or a GalNAc moiety.

In some embodiments, the siRNA comprises the sense strand and/or the antisense strand sequence of an siRNA in Table 13B, or a nucleic acid sequence thereof having 3 or 4 nucleoside substitutions, additions, or deletions. In some embodiments, the siRNA comprises the sense strand and/or the antisense strand sequence of an siRNA in Table 13B, or a nucleic acid sequence thereof having 1 or 2 nucleoside substitutions, additions, or deletions. In some embodiments, the siRNA comprises the sense strand and/or the antisense strand sequence of an siRNA in Table 13B. In some embodiments, the siRNA is cross-reactive with a non-human primate (NHP) MTRES1 mRNA. The siRNA may include one or more internucleoside linkages and/or one or more nucleoside modifications. The siRNA may include a moiety such as a lipid moiety or a GalNAc moiety.

In some embodiments, the siRNA comprises the sense strand and/or the antisense strand sequence of an siRNA in Table 15B, or a nucleic acid sequence thereof having 3 or 4 nucleoside substitutions, additions, or deletions. In some embodiments, the siRNA comprises the sense strand and/or the antisense strand sequence of an siRNA in Table 15B, or a nucleic acid sequence thereof having 1 or 2 nucleoside substitutions, additions, or deletions. In some embodiments, the siRNA comprises the sense strand and/or the antisense strand sequence of an siRNA in Table 15B. In some embodiments, the siRNA is cross-reactive with a non-human primate (NHP) MTRES1 mRNA. The siRNA may include one or more internucleoside linkages and/or one or more nucleoside modifications. The siRNA may include a moiety such as a lipid moiety or a GalNAc moiety.

In some embodiments, the siRNA comprises the sense strand and/or the antisense strand sequence of an siRNA of subset A, or a nucleic acid sequence thereof having 3 or 4 nucleoside substitutions, additions, or deletions. In some embodiments, the siRNA comprises the sense strand and/or the antisense strand sequence of an siRNA of subset A, or a nucleic acid sequence thereof having 1 or 2 nucleoside substitutions, additions, or deletions. In some embodiments, the siRNA comprises the sense strand and/or the antisense strand sequence of an siRNA of subset A. In some embodiments, the siRNA is cross-reactive with a non-human primate (NHP) MTRES1 mRNA. The siRNA may include one or more internucleoside linkages and/or one or more nucleoside modifications.

In some embodiments, the siRNA comprises the sense strand and/or the antisense strand sequence of an siRNA of subset B, or a nucleic acid sequence thereof having 3 or 4 nucleoside substitutions, additions, or deletions. In some embodiments, the siRNA comprises the sense strand and/or the antisense strand sequence of an siRNA of subset B, or a nucleic acid sequence thereof having 1 or 2 nucleoside substitutions, additions, or deletions. In some embodiments, the siRNA comprises the sense strand and/or the antisense strand sequence of an siRNA of subset B. In some embodiments, the siRNA is cross-reactive with a non-human primate (NHP) MTRES1 mRNA. The siRNA may include one or more internucleoside linkages and/or one or more nucleoside modifications.

In some embodiments, the siRNA comprises the sense strand and/or the antisense strand sequence of an siRNA of subset C, or a nucleic acid sequence thereof having 3 or 4 nucleoside substitutions, additions, or deletions. In some embodiments, the siRNA comprises the sense strand and/or the antisense strand sequence of an siRNA of subset C, or a nucleic acid sequence thereof having 1 or 2 nucleoside substitutions, additions, or deletions. In some embodiments, the siRNA comprises the sense strand and/or the antisense strand sequence of an siRNA of subset C. In some embodiments, the siRNA is cross-reactive with a non-human primate (NHP) MTRES1 mRNA. The siRNA may include one or more internucleoside linkages and/or one or more nucleoside modifications.

In some embodiments, the siRNA comprises the sense strand and/or the antisense strand sequence of an siRNA of subset D, or a nucleic acid sequence thereof having 3 or 4 nucleoside substitutions, additions, or deletions. In some embodiments, the siRNA comprises the sense strand and/or the antisense strand sequence of an siRNA of subset D, or a nucleic acid sequence thereof having 1 or 2 nucleoside substitutions, additions, or deletions. In some embodiments, the siRNA comprises the sense strand and/or the antisense strand sequence of an siRNA of subset D. In some embodiments, the siRNA is cross-reactive with a non-human primate (NHP) MTRES1 mRNA. The siRNA may include one or more internucleoside linkages and/or one or more nucleoside modifications.

In some embodiments, the siRNA comprises the sense strand and/or the antisense strand sequence of an siRNA of subset E, or a nucleic acid sequence thereof having 3 or 4 nucleoside substitutions, additions, or deletions. In some embodiments, the siRNA comprises the sense strand and/or the antisense strand sequence of an siRNA of subset E, or a nucleic acid sequence thereof having 1 or 2 nucleoside substitutions, additions, or deletions. In some embodiments, the siRNA comprises the sense strand and/or the antisense strand sequence of an siRNA of subset E. In some embodiments, the siRNA is cross-reactive with a non-human primate (NHP) MTRES1 mRNA. The siRNA may include one or more internucleoside linkages and/or one or more nucleoside modifications.

In some embodiments, the siRNA comprises the sense strand and/or the antisense strand sequence of an siRNA of subset F, or a nucleic acid sequence thereof having 3 or 4 nucleoside substitutions, additions, or deletions. In some embodiments, the siRNA comprises the sense strand and/or the antisense strand sequence of an siRNA of subset F, or a nucleic acid sequence thereof having 1 or 2 nucleoside substitutions, additions, or deletions. In some embodiments, the siRNA comprises the sense strand and/or the antisense strand sequence of an siRNA of subset F. In some embodiments, the siRNA is cross-reactive with a non-human primate (NHP) MTRES1 mRNA. The siRNA may include one or more internucleoside linkages and/or one or more nucleoside modifications.

In some embodiments, the siRNA comprises a sense strand having a sequence in accordance with SEQ ID NO: 2576. In some embodiments, the sense strand sequence comprises or consists of sequence at least 75% identical to SEQ ID NO: 2576, at least 80% identical to SEQ ID NO: 2576, at least 85% identical to SEQ ID NO: 2576, at least 90% identical to SEQ ID NO: 2576, or at least 95% identical to SEQ ID NO: 2576. In some embodiments, the sense strand sequence comprises or consists of the sequence of SEQ ID NO: 2576, or a sense strand sequence thereof having 1, 2, 3, or 4 nucleoside substitutions, additions, or deletions. In some embodiments, the sense strand sequence comprises or consists of the sequence of SEQ ID NO: 2576, or a sense strand sequence thereof having 1 or 2 nucleoside substitutions, additions, or deletions. In some embodiments, the sense strand sequence comprises or consists of a sequence 100% identical to SEQ ID NO: 2576. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 2638. In some embodiments, the antisense strand sequence comprises or consists of sequence at least 75% identical to SEQ ID NO: 2638, at least 80% identical to SEQ ID NO: 2638, at least 85% identical to SEQ ID NO: 2638, at least 90% identical to SEQ ID NO: 2638, or at least 95% identical to SEQ ID NO: 2638. In some embodiments, the antisense strand sequence comprises or consists of the sequence of SEQ ID NO 2638, or an antisense strand sequence thereof having 1, 2, 3, or 4 nucleoside substitutions, additions, or deletions. In some embodiments, the antisense strand sequence comprises or consists of the sequence of SEQ ID NO: 2638, or an antisense strand sequence thereof having 1 or 2 nucleoside substitutions, additions, or deletions. In some embodiments, the antisense strand sequence comprises or consists of a sequence 100% identical to SEQ ID NO: 2638. The antisense strand may comprise any modifications or modification pattern described herein. The antisense strand may comprise a moiety such as a GalNAc moiety or a lipid moiety.

In some embodiments, the siRNA comprises a sense strand having a sequence in accordance with SEQ ID NO: 2582. In some embodiments, the sense strand sequence comprises or consists of sequence at least 75% identical to SEQ ID NO: 2582, at least 80% identical to SEQ ID NO: 2582, at least 85% identical to SEQ ID NO: 2582, at least 90% identical to SEQ ID NO: 2582, or at least 95% identical to SEQ ID NO: 2582. In some embodiments, the sense strand sequence comprises or consists of the sequence of SEQ ID NO 2582, or a sense strand sequence thereof having 1, 2, 3, or 4 nucleoside substitutions, additions, or deletions. In some embodiments, the sense strand sequence comprises or consists of the sequence of SEQ ID NO: 2582, or a sense strand sequence thereof having 1 or 2 nucleoside substitutions, additions, or deletions. In some embodiments, the sense strand sequence comprises or consists of a sequence 100% identical to SEQ ID NO: 2582. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 2644. In some embodiments, the antisense strand sequence comprises or consists of sequence at least 75% identical to SEQ ID NO: 2644, at least 80% identical to SEQ ID NO: 2644, at least 85% identical to SEQ ID NO: 2644, at least 90% identical to SEQ ID NO: 2644, or at least 95% identical to SEQ ID NO: 2644. In some embodiments, the antisense strand sequence comprises or consists of the sequence of SEQ ID NO 2644, or an antisense strand sequence thereof having 1, 2, 3, or 4 nucleoside substitutions, additions, or deletions. In some embodiments, the antisense strand sequence comprises or consists of the sequence of SEQ ID NO: 2644, or an antisense strand sequence thereof having 1 or 2 nucleoside substitutions, additions, or deletions. In some embodiments, the antisense strand sequence comprises or consists of a sequence 100% identical to SEQ ID NO: 2644. The antisense strand may comprise any modifications or modification pattern described herein. The antisense strand may comprise a moiety such as a GalNAc moiety or a lipid moiety.

In some embodiments, the siRNA comprises a sense strand having a sequence in accordance with SEQ ID NO: 2583. In some embodiments, the sense strand sequence comprises or consists of sequence at least 75% identical to SEQ ID NO: 2583, at least 80% identical to SEQ ID NO: 2583, at least 85% identical to SEQ ID NO: 2583, at least 90% identical to SEQ ID NO: 2583, or at least 95% identical to SEQ ID NO: 2583. In some embodiments, the sense strand sequence comprises or consists of the sequence of SEQ ID NO 2583, or a sense strand sequence thereof having 1, 2, 3, or 4 nucleoside substitutions, additions, or deletions. In some embodiments, the sense strand sequence comprises or consists of the sequence of SEQ ID NO: 2583, or a sense strand sequence thereof having 1 or 2 nucleoside substitutions, additions, or deletions. In some embodiments, the sense strand sequence comprises or consists of a sequence 100% identical to SEQ ID NO: 2583. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 2645. In some embodiments, the antisense strand sequence comprises or consists of sequence at least 75% identical to SEQ ID NO: 2645, at least 80% identical to SEQ ID NO: 2645, at least 85% identical to SEQ ID NO: 2645, at least 90% identical to SEQ ID NO: 2645, or at least 95% identical to SEQ ID NO: 2645. In some embodiments, the antisense strand sequence comprises or consists of the sequence of SEQ ID NO 2645, or an antisense strand sequence thereof having 1, 2, 3, or 4 nucleoside substitutions, additions, or deletions. In some embodiments, the antisense strand sequence comprises or consists of the sequence of SEQ ID NO: 2645, or an antisense strand sequence thereof having 1 or 2 nucleoside substitutions, additions, or deletions. In some embodiments, the antisense strand sequence comprises or consists of a sequence 100% identical to SEQ ID NO: 2645. The antisense strand may comprise any modifications or modification pattern described herein. The antisense strand may comprise a moiety such as a GalNAc moiety or a lipid moiety.

In some embodiments, the siRNA comprises a sense strand having a sequence in accordance with SEQ ID NO: 2584. In some embodiments, the sense strand sequence comprises or consists of sequence at least 75% identical to SEQ ID NO: 2584, at least 80% identical to SEQ ID NO: 2584, at least 85% identical to SEQ ID NO: 2584, at least 90% identical to SEQ ID NO: 2584, or at least 95% identical to SEQ ID NO: 2584. In some embodiments, the sense strand sequence comprises or consists of the sequence of SEQ ID NO 2584, or a sense strand sequence thereof having 1, 2, 3, or 4 nucleoside substitutions, additions, or deletions. In some embodiments, the sense strand sequence comprises or consists of the sequence of SEQ ID NO: 2584, or a sense strand sequence thereof having 1 or 2 nucleoside substitutions, additions, or deletions. In some embodiments, the sense strand sequence comprises or consists of a sequence 100% identical to SEQ ID NO: 2584. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 2646. In some embodiments, the antisense strand sequence comprises or consists of sequence at least 75% identical to SEQ ID NO: 2646, at least 80% identical to SEQ ID NO: 2646, at least 85% identical to SEQ ID NO: 2646, at least 90% identical to SEQ ID NO: 2646, or at least 95% identical to SEQ ID NO: 2646. In some embodiments, the antisense strand sequence comprises or consists of the sequence of SEQ ID NO 2646, or an antisense strand sequence thereof having 1, 2, 3, or 4 nucleoside substitutions, additions, or deletions. In some embodiments, the antisense strand sequence comprises or consists of the sequence of SEQ ID NO: 2646, or an antisense strand sequence thereof having 1 or 2 nucleoside substitutions, additions, or deletions. In some embodiments, the antisense strand sequence comprises or consists of a sequence 100% identical to SEQ ID NO: 2646. The antisense strand may comprise any modifications or modification pattern described herein. The antisense strand may comprise a moiety such as a GalNAc moiety or a lipid moiety.

In some embodiments, the siRNA comprises a sense strand having a sequence in accordance with SEQ ID NO: 2604. In some embodiments, the sense strand sequence comprises or consists of sequence at least 75% identical to SEQ ID NO: 2604, at least 80% identical to SEQ ID NO: 2604, at least 85% identical to SEQ ID NO: 2604, at least 90% identical to SEQ ID NO: 2604, or at least 95% identical to SEQ ID NO: 2604. In some embodiments, the sense strand sequence comprises or consists of the sequence of SEQ ID NO 2604, or a sense strand sequence thereof having 1, 2, 3, or 4 nucleoside substitutions, additions, or deletions. In some embodiments, the sense strand sequence comprises or consists of the sequence of SEQ ID NO: 2604, or a sense strand sequence thereof having 1 or 2 nucleoside substitutions, additions, or deletions. In some embodiments, the sense strand sequence comprises or consists of a sequence 100% identical to SEQ ID NO: 2604. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 2666. In some embodiments, the antisense strand sequence comprises or consists of sequence at least 75% identical to SEQ ID NO: 2666, at least 80% identical to SEQ ID NO: 2666, at least 85% identical to SEQ ID NO: 2666, at least 90% identical to SEQ ID NO: 2666, or at least 95% identical to SEQ ID NO: 2666. In some embodiments, the antisense strand sequence comprises or consists of the sequence of SEQ ID NO 2666, or an antisense strand sequence thereof having 1, 2, 3, or 4 nucleoside substitutions, additions, or deletions. In some embodiments, the antisense strand sequence comprises or consists of the sequence of SEQ ID NO: 2666, or an antisense strand sequence thereof having 1 or 2 nucleoside substitutions, additions, or deletions. In some embodiments, the antisense strand sequence comprises or consists of a sequence 100% identical to SEQ ID NO: 2666. The antisense strand may comprise any modifications or modification pattern described herein. The antisense strand may comprise a moiety such as a GalNAc moiety or a lipid moiety.

### B. ASOs

In some embodiments, the composition comprises an oligonucleotide that inhibits the expression of MTRES1, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

In some embodiments, the composition comprises an oligonucleotide that inhibits the expression of MTRES1, wherein the oligonucleotide comprises an ASO about 12-30 nucleosides in length and comprising a nucleoside sequence complementary to about 12-30 contiguous nucleosides of a full-length human MTRES1 mRNA sequence such as SEQ ID NO: 2443; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 2443.

In some embodiments, the composition comprises an oligonucleotide that inhibits the expression of MTRES1, wherein the oligonucleotide comprises an ASO about 12-30 nucleosides in length and comprising a nucleoside sequence complementary to about 12-30 contiguous nucleosides of a full-length human MTRES1 mRNA sequence such as SEQ ID NO: 2462; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 2462.

### C. Modification Patterns

In some embodiments, the composition comprises an oligonucleotide that inhibits the expression of MTRES1, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. A phosphorothioate may include a nonbridging oxygen atom in a phosphate backbone of the oligonucleotide that is replaced by sulfur. Modified internucleoside linkages may be included in siRNAs or ASOs. Benefits of the modified internucleoside linkage may include decreased toxicity or improved pharmacokinetics.

In some embodiments, the composition comprises an oligonucleotide that inhibits the expression of MTRES1, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that inhibits the expression of MTRES1, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

Some embodiments include an oligonucleotide comprising: a sense strand having a 5′ end, a 3′ end and a region of complementarity with an antisense strand; an antisense strand having a 5′end, a 3′end and a region of complementarity with the sense strand and a region of complementarity to an mRNA target; an overhang region at the 3′ end of the sense strand having at least 3 contiguous phosphorothioated nucleotides; and an overhang region at the 3′ end of the antisense strand having at least 3 contiguous phosphorothioated nucleotides.

Some embodiments include an oligonucleotide comprising: a sense strand having a 5′ end, a 3′ end and a region of complementarity with an antisense strand; an antisense strand having a 5′end, a 3′end and a region of complementarity with the sense strand and a region of complementarity to an mRNA target; and an overhang region at the 3′ end of the sense strand having at least 3 contiguous phosphorothioated nucleotides.

In some embodiments, the oligonucleotide includes two to eight oligonucleotides attached through a linker. The linker may be hydrophobic. In some embodiments, the oligonucleotides independently have substantial chemical stabilization (e.g., at least 40% of the constituent bases are chemically-modified). In some embodiments, the oligonucleotides have full chemical stabilization (i.e., all of the constituent bases are chemically-modified). In some embodiments, the oligonucleotide includes one or more single-stranded phosphorothioated tails, each independently having two to twenty nucleotides. In some embodiments, each single-stranded tail has eight to ten nucleotides.

In certain embodiments, a compound (e.g. moiety attached to the oligonucleotide) includes three properties: (1) a branched structure, (2) full metabolic stabilization, and (3) the presence of a single-stranded tail comprising phosphorothioate linkers. In a particular embodiment, a compound has 2 or 3 branches. The increased overall size of the branched structures promote increased uptake. Also, without being bound by a particular theory of activity, multiple adjacent branches (e.g., 2 or 3) allow each branch to act cooperatively and thus dramatically enhance rates of internalization, trafficking and release. The compound may include an oligonucleotide described herein, as part of the compound.

In certain embodiments, a compound includes the following properties: (1) two or more branched oligonucleotides linked via anon-natural linker (2) substantially chemically stabilized, e.g., wherein more than 40%, optimally 100%, of oligonucleotides are chemically modified (e.g., no RNA and optionally no DNA); and (3) phoshorothioated single oligonucleotides containing at least 3, optimally 5-20 phosphorothioated bonds.

In some embodiments, the oligonucleotide comprises a phosphate at a 5′ end. In some embodiments, the oligonucleotide comprises a phosphate at a 3′ end. In some embodiments, the oligonucleotide comprises a phosphate mimic at a 5′ end. In some embodiments, the oligonucleotide comprises a phosphate mimic at a 3′ end.

The oligonucleotide may include purines. Examples of purines include adenine (A) or guanine (G), or modified versions thereof. The oligonucleotide may include pyrimidines. Examples of pyrimidines include cytosine (C), thymine (T), or uracil (U), or modified versions thereof.

In some embodiments, purines of the oligonucleotide comprise 2′ fluoro modified purines. In some embodiments, purines of the oligonucleotide comprise 2′-O-methyl modified purines. In some embodiments, purines of the oligonucleotide comprise a mixture of 2′ fluoro and 2′-O-methyl modified purines. In some embodiments, all purines of the oligonucleotide comprise 2′ fluoro modified purines. In some embodiments, all purines of the oligonucleotide comprise 2′-O-methyl modified purines. In some embodiments, all purines of the oligonucleotide comprise a mixture of 2′ fluoro and 2′-O-methyl modified purines. 2′-O-methyl may include 2′ O-methyl. Where 2′-O-methyl modifications are described, it is contemplated that a 2′-methyl modification may be included, and vice versa.

In some embodiments, pyrimidines of the oligonucleotide comprise 2′ fluoro modified pyrimidines. In some embodiments, pyrimidines of the oligonucleotide comprise 2′-O-methyl modified pyrimidines. In some embodiments, pyrimidines of the oligonucleotide comprise a mixture of 2′ fluoro and 2′-O-methyl modified pyrimidines. In some embodiments, all pyrimidines of the oligonucleotide comprise 2′ fluoro modified pyrimidines. In some embodiments, all pyrimidines of the oligonucleotide comprise 2′-O-methyl modified pyrimidines. In some embodiments, all pyrimidines of the oligonucleotide comprise a mixture of 2′ fluoro and 2′-O-methyl modified pyrimidines.

In some embodiments, purines of the oligonucleotide comprise 2′ fluoro modified purines, and pyrimidines of the oligonucleotide comprise a mixture of 2′ fluoro and 2′-O-methyl modified pyrimidines. In some embodiments, purines of the oligonucleotide comprise 2′-O-methyl modified purines, and pyrimidines of the oligonucleotide comprise a mixture of 2′ fluoro and 2′-O-methyl modified pyrimidines. In some embodiments, purines of the oligonucleotide comprise 2′ fluoro modified purines, and pyrimidines of the oligonucleotide comprise 2′-O-methyl modified pyrimidines. In some embodiments, purines of the oligonucleotide comprise 2′-O-methyl modified purines, and pyrimidines of the oligonucleotide comprise 2′ fluoro modified pyrimidines. In some embodiments, pyrimidines of the oligonucleotide comprise 2′ fluoro modified pyrimidines, and purines of the oligonucleotide comprise a mixture of 2′ fluoro and 2′-O-methyl modified purines. In some embodiments, pyrimidines of the oligonucleotide comprise 2′-O-methyl modified pyrimidines, and purines of the oligonucleotide comprise a mixture of 2′ fluoro and 2′-O-methyl modified purines. In some embodiments, pyrimidines of the oligonucleotide comprise 2′ fluoro modified pyrimidines, and purines of the oligonucleotide comprise 2′-O-methyl modified purines. In some embodiments, pyrimidines of the oligonucleotide comprise 2′-O-methyl modified pyrimidines, and purines of the oligonucleotide comprise 2′ fluoro modified purines.

In some embodiments, all purines of the oligonucleotide comprise 2′ fluoro modified purines, and all pyrimidines of the oligonucleotide comprise a mixture of 2′ fluoro and 2′-O-methyl modified pyrimidines. In some embodiments, all purines of the oligonucleotide comprise 2′-O-methyl modified purines, and all pyrimidines of the oligonucleotide comprise a mixture of 2′ fluoro and 2′-O-methyl modified pyrimidines. In some embodiments, all purines of the oligonucleotide comprise 2′ fluoro modified purines, and all pyrimidines of the oligonucleotide comprise 2′-O-methyl modified pyrimidines. In some embodiments, all purines of the oligonucleotide comprise 2′-O-methyl modified purines, and all pyrimidines of the oligonucleotide comprise 2′ fluoro modified pyrimidines. In some embodiments, all pyrimidines of the oligonucleotide comprise 2′ fluoro modified pyrimidines, and all purines of the oligonucleotide comprise a mixture of 2′ fluoro and 2′-O-methyl modified purines. In some embodiments all pyrimidines of the oligonucleotide comprise 2′-O-methyl modified pyrimidines, and all purines of the oligonucleotide comprise a mixture of 2′ fluoro and 2′-O-methyl modified purines. In some embodiments all pyrimidines of the oligonucleotide comprise 2′ fluoro modified pyrimidines, and all purines of the oligonucleotide comprise 2′-O-methyl modified purines. In some embodiments, all pyrimidines of the oligonucleotide comprise 2′-O-methyl modified pyrimidines, and all purines of the oligonucleotide comprise 2′ fluoro modified purines.

In some cases, the oligonucleotide comprises a particular modification pattern. In some embodiments, position 9 counting from the 5′ end of the of a strand of the oligonucleotide may have a 2′F modification. In some embodiments, when position 9 of a strand of the oligonucleotide is a pyrimidine, then all purines in a strand of the oligonucleotide have a 2′OMe modification. In some embodiments, when position 9 is the only pyrimidine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are pyrimidines, then both of these pyrimidines are the only two positions with a 2′F modification in a strand of the oligonucleotide. In some embodiments, when position 9 and only two other bases between positions 5 and 11 of a strand of the oligonucleotide are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of a strand of the oligonucleotide, then all combinations of pyrimidines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some embodiments, when position 9 of a strand of the oligonucleotide is a purine, then all purines in a strand of the oligonucleotide have a 2′OMe modification. In some embodiments, when position 9 is the only purine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are purines, then both of these purines are the only two positions with a 2′F modification in a strand of the oligonucleotide. In some embodiments, when position 9 and only two other bases between positions 5 and 11 of a strand of the oligonucleotide are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of a strand of the oligonucleotide, then all combinations of purines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some cases, position 9 of a strand of the oligonucleotide can be a 2′deoxy. In these cases, 2′F and 2′OMe modifications may occur at the other positions of a strand of the oligonucleotide. In some cases, a strand of the oligonucleotide of any of the siRNAs comprises a modification pattern which conforms to these a strand of the oligonucleotide rules.

In some embodiments, position nine of the sense strand comprises a 2′ fluoro-modified pyrimidine. In some embodiments, all purines of the sense strand comprise 2′-O-methyl modified purines.

In some embodiments, 1, 2, 3, 4, or 5 pyrimidines between positions 5 and 11 comprise a 2′flouro-modified pyrimidine, provided there are not three 2′ fluoro-modified pyrimidines in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, the even-numbered positions of the antisense strand comprise 2′flouro-modified nucleotides, 2′-O-methyl modified nucleotides and unmodified deoxyribonucleotide. In some embodiments, position nine of the sense strand comprises a 2′ fluoro-modified pyrimidine; all purines of the sense strand comprises 2′-O-methyl modified purines; 1, 2, 3, 4, or 5 pyrimidines between positions 5 and 11 comprise a 2′flouro-modified pyrimidine, provided there are not three 2′ fluoro-modified pyrimidines in a row; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises a 2′ fluoro-modified purine. In some embodiments, all pyrimidines of the sense strand comprise 2′-O-methyl modified purines. In some embodiments, 1, 2, 3, 4, or 5 purines between positions 5 and 11 comprise a 2′flouro-modified purine, provided there are not three 2′ fluoro-modified purine in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, the even-numbered positions of the antisense strand comprise 2′flouro-modified nucleotides, 2′-O-methyl modified nucleotides and unmodified deoxyribonucleotide. In some embodiments, position nine of the sense strand comprises a 2′ fluoro-modified purine; all pyrimidine of the sense strand comprises 2′-O-methyl modified pyrimidines; 1, 2, 3, 4, or 5 purines between positions 5 and 11 comprise a 2′flouro-modified purines, provided there are not three 2′ fluoro-modified purines in a row; the odd-numbered positions of the antisense strand comprise 2′—O-methyl modified nucleotides; and the even-numbered positions of the antisense strand comprise 2′flouro-modified nucleotides and unmodified deoxyribonucleotides. In some embodiments, there are not three 2′ fluoro-modified purines in a row. In some embodiments, there are not three 2′ fluoro-modified pyrimidines in a row.

In some embodiments, position nine of the sense strand comprises an unmodified deoxyribonucleotide. In some embodiments, positions 5, 7, and 8 of the sense strand comprise 2′fluoro-modifed nucleotides. In some embodiments, all pyrimidines in positions 10 to 21 of the sense strand comprise 2′-O-methyl modified pyrimidines and all purines in positions 10 to 21 of the comprise 2′-O-methyl modified purines or 2′fluoro-modified purines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, the even-numbered positions of the antisense strand comprise 2′flouro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modifed nucleotides; all pyrimidines in positions 10 to 21 of the sense strand comprise 2′-O-methyl modified pyrimidines and all purines in positions 10 to 21 of the comprise 2′-O-methyl modified purines or 2′fluoro-modified purines; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises an unmodified deoxyribonucleotide. In some embodiments, positions 5, 7, and 8 of the sense strand comprise 2′fluoro-modifed nucleotides. In some embodiments, all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, the even-numbered positions of the antisense strand comprise 2′flouro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modifed nucleotides; all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines; the odd-numbered positions of the antisense strand comprise 2′-O-methyl modified nucleotides; and the even-numbered positions of the antisense strand comprise 2′flouro-modified nucleotides and unmodified deoxyribonucleotide.

In some embodiments, the moiety includes a negatively charged group attached at a 5′ end of the oligonucleotide. This may be referred to as a 5′-end group. In some embodiments, the negatively charged group is attached at a 5′ end of an antisense strand of an siRNA disclosed herein. The 5′-end group may be or include a 5′-end phosphorothioate, 5′-end phosphorodithioate, 5′-end vinylphosphonate (5′-VP), 5′-end methylphosphonate, 5′-end cyclopropyl phosphonate, or a 5′-deoxy-5′-C-malonyl. The 5′-end group may comprise 5′-VP. In some embodiments, the 5′-VP comprises a trans-vinylphosphate or cis-vinylphosphate. The 5′-end group may include an extra 5′ phosphate. A combination of 5′-end groups may be used.

In some embodiments, the oligonucleotide includes a negatively charged group. The negatively charged group may aid in cell or tissue penetration. The negatively charged group may be attached at a 5′ or 3′ end (e.g. a 5′ end) of the oligonucleotide. This may be referred to as an end group. The end group may be or include a phosphorothioate, phosphorodithioate, vinylphosphonate, methylphosphonate, cyclopropyl phosphonate, or a deoxy-C-malonyl. The end group may include an extra 5′ phosphate such as an extra 5′ phosphate. A combination of end groups may be used.

In some embodiments, the oligonucleotide includes a phosphate mimic. In some embodiments, the phosphate mimic comprises vinyl phosphonate. In some embodiments, the vinyl phosphonate comprises a trans-vinylphosphate. In some embodiments, the vinyl phosphonate comprises a cis-vinylphosphate. An example of a nucleotide that includes a vinyl phosphonate is shown below.

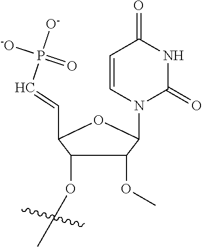

### 5′ Vinylphosphonate 2′ O Methyl Uridine

In some embodiments, the vinyl phosphonate increases the stability of the oligonucleotide. In some embodiments, the vinyl phosphonate increases the accumulation of the oligonucleotide in tissues. In some embodiments, the vinyl phosphonate protects the oligonucleotide from an exonuclease or a phosphatase. In some embodiments, the vinyl phosphonate improves the binding affinity of the oligonucleotide with the siRNA processing machinery.

In some embodiments, the oligonucleotide includes 1 vinyl phosphonate. In some embodiments, the oligonucleotide includes 2 vinyl phosphonates. In some embodiments, the oligonucleotide includes 3 vinyl phosphonates. In some embodiments, the oligonucleotide includes 4 vinyl phosphonates. In some embodiments, the antisense strand of the oligonucleotide comprises a vinyl phosphonate at the 5′ end. In some embodiments, the antisense strand of the oligonucleotide comprises a vinyl phosphonate at the 3′ end. In some embodiments, the sense strand of the oligonucleotide comprises a vinyl phosphonate at the 5′ end. In some embodiments, the sense strand of the oligonucleotide comprises a vinyl phosphonate at the 3′ end.

### 1. Hydrophobic Moieties

In some embodiments, the composition comprises an oligonucleotide that inhibits the expression of MTRES1, wherein the oligonucleotide comprises a moiety attached at a 3′ or 5′ terminus of the oligonucleotide. Examples of moieties include a hydrophobic moiety or a sugar moiety, or a combination thereof. In some embodiments, the oligonucleotide is an siRNA having a sense strand, and the moiety is attached to a 5′ end of the sense strand. In some embodiments, the oligonucleotide is an siRNA having a sense strand, and the moiety is attached to a 3′ end of the sense strand. In some embodiments, the oligonucleotide is an siRNA having an antisense strand, and the moiety is attached to a 5′ end of the antisense strand. In some embodiments, the oligonucleotide is an siRNA having an antisense strand, and the moiety is attached to a 3′ end of the antisense strand. In some embodiments, the oligonucleotide is an ASO, and the moiety is attached to a 5′ end of the ASO. In some embodiments, the oligonucleotide is an ASO, and the moiety is attached to a 3′ end of the ASO.

In some embodiments, the composition comprises an oligonucleotide that inhibits the expression of MTRES1, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of MTRES1, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

In some embodiments, the oligonucleotide comprises a lipophilic moiety attached at a 3′ or 5′ terminus of the oligonucleotide. In some embodiments, the lipophilic moiety comprises cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, a heptadecyl group, palmitic acid, myristic acid, 03-(oleoyl)lithocholic acid, 03-(oleoyl)cholenic acid, ibuprofen, naproxen, dimethoxytrityl, or phenoxazine, or a combination thereof. The lipophilic moiety may include a steroid such as cholesterol. The lipophilic moiety may include retinoic acid. The lipophilic moiety may include cholic acid. The lipophilic moiety may include adamantane acetic acid. The lipophilic moiety may include 1-pyrene butyric acid. The lipophilic moiety may include dihydrotestosterone. The lipophilic moiety may include 1,3-bis-O(hexadecyl)glycerol. The lipophilic moiety may include geranyloxyhexyanol. The lipophilic moiety may include hexadecylglycerol. The lipophilic moiety may include borneol. The lipophilic moiety may include menthol. The lipophilic moiety may include 1,3-propanediol. The lipophilic moiety may include a heptadecyl group. The lipophilic moiety may include palmitic acid. The lipophilic moiety may include myristic acid. The lipophilic moiety may include O3-(oleoyl)lithocholic acid. The lipophilic moiety may include 03-(oleoyl)cholenic acid. The lipophilic moiety may include ibuprofen. The lipophilic moiety may include naproxen. The lipophilic moiety may include dimethoxytrityl. The lipophilic moiety may include phenoxazine.

In some embodiments, the lipophilic moiety comprises a hydrocarbon chain. The hydrocarbon chain may comprise or consist of a C4-C30 hydrocarbon chain. In some embodiments, the lipophilic moiety comprises a lipid.

In some embodiments, the oligonucleotide includes one or more lipophilic monomers, containing one or more lipophilic moieties, conjugated to one or more positions on at least one strand of the oligonucleotide, optionally via a linker or carrier. For instance, some embodiments provide an oligonucleotide comprising: an antisense strand which is complementary to a target gene; a sense strand which is complementary to said antisense strand; and one or more lipophilic monomers, containing one or more lipophilic moieties, conjugated to one or more positions on at least one strand, optionally via a linker or carrier. In some embodiments, the lipophilicity of the lipophilic moiety, measured by octanol-water partition coefficient, log P, exceeds 0.

In some embodiments, the lipophilic moiety is an aliphatic, cyclic such as alicyclic, or polycyclic such as polyalicyclic compound, such as a steroid (e.g., sterol), a linear or branched aliphatic hydrocarbon, or an aromatic. Exemplary lipophilic moieties may include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, 03-(oleoyl)cholenic acid, ibuprofen, naproxen, dimethoxytrityl, or phenoxazine. Suitable lipophilic moieties may also include those containing a saturated or unsaturated C4-C30 hydrocarbon chain (e.g., C4-C30 alkyl or alkenyl), and an optional functional group selected from the group consisting of hydroxyl, amine, carboxylic acid, sulfonate, phosphate, thiol, azide, and alkyne. The functional group may be useful to attach the lipophilic moiety to the oligonucleotide. In some embodiments, the lipophilic moiety contains a saturated or unsaturated C6-C18 hydrocarbon chain (e.g., a linear C6-C18 alkyl or alkenyl). In some embodiments, the lipophilic moiety contains a saturated or unsaturated C16 hydrocarbon chain (e.g., a linear C16 alkyl or alkenyl). In some embodiments, the lipophilic moiety contains two or more carbon-carbon double bonds.

In some embodiments, the composition comprises an oligonucleotide that inhibits the expression of MTRES1, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of MTRES1, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof.

In some embodiments, the composition comprises an oligonucleotide that inhibits the expression of MTRES1, wherein the oligonucleotide comprises a hydrophobic ligand or moiety. In some embodiments, the hydrophobic ligand or moiety comprises cholesterol. In some embodiments, the hydrophobic ligand or moiety comprises a cholesterol derivative. In some embodiments, the hydrophobic ligand or moiety is attached at a 3′ terminus of the oligonucleotide. In some embodiments, the hydrophobic ligand or moiety s attached at a 5′ terminus of the oligonucleotide. In some embodiments, the composition comprises a sense strand, and the hydrophobic ligand or moiety is attached to the sense strand (e.g. attached to a 5′ end of the sense strand, or attached to a 3′ end of the sense strand). In some embodiments, the composition comprises an antisense strand, and the hydrophobic ligand or moiety is attached to the antisense strand (e.g. attached to a 5′ end of the antisense strand, or attached to a 3′ end of the antisense strand). In some embodiments, the composition comprises a hydrophobic ligand or moiety attached at a 3′ or 5′ terminus of the oligonucleotide.

In some embodiments, a hydrophobic moiety is attached to the oligonucleotide (e.g. a sense strand and/or an antisense strand of a siRNA). In some embodiments, a hydrophobic moiety is attached at a 3′ terminus of the oligonucleotide. In some embodiments, a hydrophobic moiety is attached at a 5′ terminus of the oligonucleotide. In some embodiments, the hydrophobic moiety comprises cholesterol. In some embodiments, the hydrophobic moiety includes a cyclohexanyl.

In some embodiments, the composition comprises an oligonucleotide that inhibits the expression of MTRES1, wherein the oligonucleotide comprises a lipid attached at a 3′ or 5′ terminus of the oligonucleotide. In some embodiments, a lipid is attached at a 3′ terminus of the oligonucleotide. In some embodiments, a lipid is attached at a 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof. In some embodiments, the lipid comprises stearyl, lithocholyl, docosanyl, docosahexaenyl, or myristyl. In some embodiments, the lipid comprises cholesterol. In some embodiments, the lipid includes a sterol such as cholesterol. In some embodiments, the lipid comprises stearyl, t-butylphenol, n-butylphenol, octylphenol, dodecylphenol, phenyl n-dodecyl, octadecylbenzamide, hexadecylbenzamide, or octadecylcyclohexyl. In some embodiments, the lipid comprises phenyl para C12.

In some embodiments, the oligonucleotide comprises any aspect of the following structure:

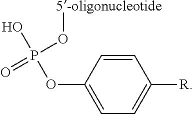

In some embodiments, the oligonucleotide comprises any aspect of the following structure:

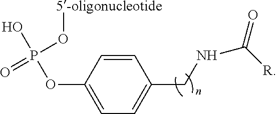

In some embodiments, the oligonucleotide comprises any aspect of the following structure:

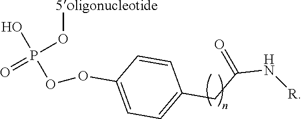

In some embodiments, the oligonucleotide comprises any aspect of the following structure: The aspect included in the oligonucleotide may include the entire structure, or may include the lipid moiety, of any of the structures shown. In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, the alkyl group contains 4-18 carbons. In some embodiments, the lipid moiety comprises an alcohol or ether.

In some embodiments, the lipid includes a fatty acid. In some embodiments, the lipid comprises a lipid depicted in Table 1. The example lipid moieties in Table 1 are shown attached at a 5′ end of an oligonucleotide, in which the 5′ terminal phosphate of the oligonucleotide is shown with the lipid moiety. In some embodiments, a lipid moiety in Table 1 may be attached at a different point of attachment than shown. For example, the point of attachment of any of the lipid moieties in the table may be at a 3′ oligonucleotide end. In some embodiments, the lipid is used for targeting the oligonucleotide to a non-hepatic cell or tissue.

TABLE 1
Hydrophobic moiety examples

| Hydrophobic | Hydrophobic | |
|---|---|---|
| Moiety Description | Moiety Name | Example Conjugation |
| stearyl | ETL3 | 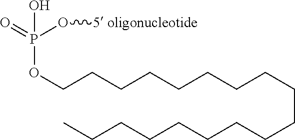 |
| t-butylphenyl | ETL7 | 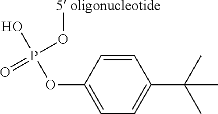 |
| n-butylphenyl | ETL8 | 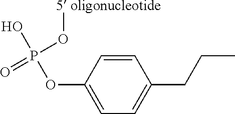 |
| octylphenyl | ETL9 | 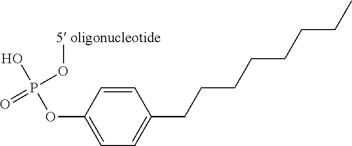 |
| dodecylphenyl | ETL10 | 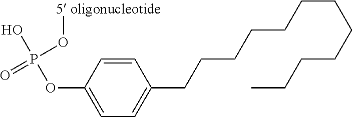 |
| phenyl n-dodecyl | ETL12 | 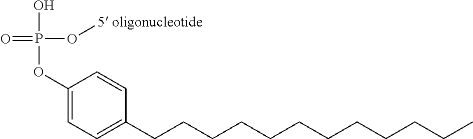 |
| octadecylbenzamide | ETL13 | 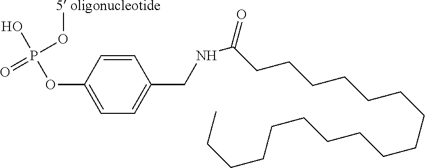 |
| hexadecylbenzamide | ETL15 | 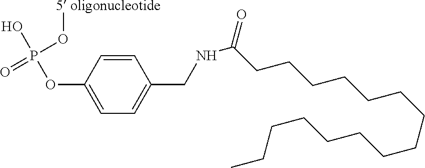 |
| octadecylcyclohexyl | ETL16 | 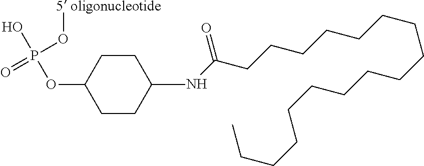 |

In some embodiments, the lipid or lipid moiety includes 16 to 18 carbons. In some embodiments, the lipid includes 16 carbons. In some embodiments, the lipid includes 17 carbons. In some embodiments, the lipid includes 18 carbons. In some embodiments, the lipid moiety includes 16 carbons. In some embodiments, the lipid moiety includes 17 carbons. In some embodiments, the lipid moiety includes 18 carbons.

The hydrophobic moiety may include a linker that comprises a carbocycle. The carbocycle may be six-membered. Some examples of a carbocycle include phenyl or cyclohexyl. The linker may include a phenyl. The linker may include a cyclohexyl. The lipid may be attached to the carbocycle, which may in turn be attached at a phosphate (e.g. 5′ or 3′ phosphate) of the oligonucleotide. In some embodiments, the lipid or hydrocarbon, and the end of the sense are connected to the phenyl or cyclohexyl linker in the 1,4; 1,3; or 1,2 substitution pattern (e.g. the para, meta, or ortho phenyl configuration). In some embodiments, the lipid or hydrocarbon, and the end of the sense are connected to the phenyl or cyclohexyl linker in the 1,4 substitution pattern (e.g. the para phenyl configuration). The lipid may be attached to the carbocycle in the 1,4 substitution pattern relative to the oligonucleotide. The lipid may be attached to the carbocycle in the 1,3 substitution pattern relative to the oligonucleotide. The lipid may be attached to the carbocycle in the 1,2 substitution pattern relative to the oligonucleotide. The lipid may be attached to the carbocycle in the ortho orientation relative to the oligonucleotide. The lipid may be attached to the carbocycle in the para orientation relative to the oligonucleotide. The lipid may be attached to the carbocycle in the meta orientation relative to the oligonucleotide.

The lipid moiety may comprise or consist of the following structure:

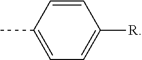

In some embodiments, the lipid moiety comprises or consists of the following structure:

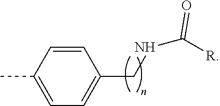

In some embodiments, the lipid moiety comprises the following structure

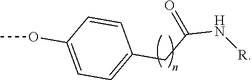

In some embodiments, the lipid moiety comprises or consist of the following structure:

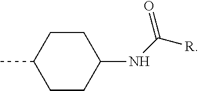

In some embodiments, the dotted line indicates a covalent connection. The covalent connection may between an end of the sense or antisense strand. For example, the connection may be to the 5′ end of the sense strand. In some embodiments, n is 0-3. In some embodiments, n is 1-3. In some embodiments, n is 0. In some embodiments, n is 1. In some embodiments, n is 2. In some embodiments, n is 3. In some embodiments, n is 4. In some embodiments, n is 5. In some embodiments, n is 1, 2, 3, 4, 5, 6, 7, 8, 9, or 10. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons.

The lipid moiety may be attached at a 5′ end of the oligonucleotide. The 5′ end may have one phosphate linking the lipid moiety to a 5′ carbon of a sugar of the oligonucleotide. The 5′ end may have two phosphates linking the lipid moiety to a 5′ carbon of a sugar of the oligonucleotide. The 5′ end may have three phosphates linking the lipid moiety to a 5′ carbon of a sugar of the oligonucleotide. The 5′ end may have one phosphate connected to the 5′ carbon of a sugar of the oligonucleotide, where the one phosphate is connected to the lipid moiety. The 5′ end may have two phosphates connected to the 5′ carbon of a sugar of the oligonucleotide, where the one of the two phosphates is connected to the lipid moiety. The 5′ end may have three phosphates connected to the 5′ carbon of a sugar of the oligonucleotide, where the one of the three phosphates is connected to the lipid moiety. The sugar may include a ribose. The sugar may include a deoxyribose. The sugar may be modified a such as a 2′ modified sugar (e.g. a 2′ O-methyl or 2′ fluoro ribose). A phosphate of the 5′ end may include a modification such as a sulfur in place of an oxygen. Two phosphates of the 5′ end may include a modification such as a sulfur in place of an oxygen. Three phosphates of the 5′ end may include a modification such as a sulfur in place of an oxygen.

In some embodiments, the oligonucleotide includes 1 lipid moiety. In some embodiments, the oligonucleotide includes 2 lipid moieties. In some embodiments, the oligonucleotide includes 3 lipid moieties. In some embodiments, the oligonucleotide includes 4 lipid moieties.

Some embodiments relate to a method of making an oligonucleotide comprising a hydrophobic conjugate. A strategy for making hydrophobic conjugates may include use of a phosphoramidite reagent based upon a 6-membered ring alcohol such as a phenol or cyclohexanol. The phosphoramidite may be reacted to a nucleotide to connect the nucleotide to the hydrophobic moiety, and thereby produce the hydrophobic conjugate. Some examples of phosphoramidite reagents that may be used to produce a hydrophobic conjugate are provided as follows:

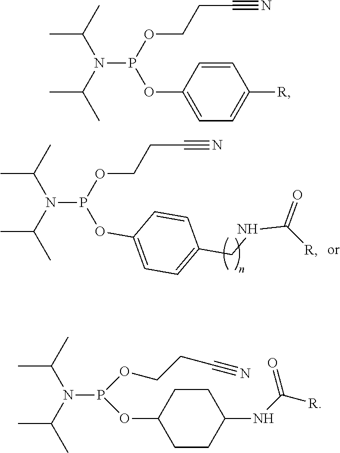

In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons. Any one of the phosphoramidite reagents may be reacted to a 5′ end of an oligonucleotide to produce an oligonucleotide comprising a hydrophobic moiety. In some embodiments, the phosphoramidite reagents is reacted to a 5′ end of a sense strand of an siRNA. The sense strand may then be hybridized to an antisense strand to form a duplex. The hybridization may be performed by incubating the sense and antisense strands in solution at a given temperature. The temperature may be gradually reduced. The temperature may comprise or include a temperature comprising an annealing temperature for the sense and antisense strands. The temperature may be below or include a temperature below the annealing temperature for the sense and antisense strands. The temperature may be below a melting temperature of the sense and antisense strands.

The lipid may be attached to the oligonucleotide by a linker. The linker may include a polyethyleneglycol (e.g. tetraethyleneglycol).

The modifications described herein may be useful for delivery to a cell or tissue, for example, extrahepatic delivery or targeting of an oligonucleotide composition. The modifications described herein may be useful for targeting an oligonucleotide composition to a cell or tissue.

2. Sugar moieties

In some embodiments, the composition comprises an oligonucleotide that inhibits the expression of MTRES1, wherein the oligonucleotide comprises a sugar moiety. The sugar moiety may include an N-acetyl galactose moiety (e.g. an N-acetylgalactosamine (GalNAc) moiety), an N-acetyl glucose moiety (e.g. an N-acetylglucosamine (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages when they target or bind a mannose receptor such as CD206. The sugar moiety may be useful for binding or targeting an asialoglycoprotein receptor such as an asialoglycoprotein receptor of a hepatocyte. The GalNAc moiety may bind to an asialoglycoprotein receptor. The GalNAc moiety may target a hepatocyte.

In some embodiments, the composition comprises an oligonucleotide that inhibits the expression of MTRES1, wherein the oligonucleotide comprises an N-acetylgalactosamine (GalNAc) moiety. GalNAc may be useful for hepatocyte targeting. The GalNAc moiety may include a bivalent or trivalent branched linker. The oligo may be attached to 1, 2 or 3 GalNAcs through a bivalent or trivalent branched linker. The GalNAc moiety may include 1, 2, 3, or more GalNAc molecules. The GalNAc moiety may be attached at a 3′ or 5′ terminus of the oligonucleotide.

In some embodiments, the composition comprises an oligonucleotide that inhibits the expression of MTRES1, wherein the oligonucleotide comprises an N-acetylgalactosamine (GalNAc) ligand for hepatocyte targeting. In some embodiments, the composition comprises GalNAc. In some embodiments, the composition comprises a GalNAc derivative. In some embodiments, the GalNAc ligand is attached at a 3′ terminus of the oligonucleotide. In some embodiments, the GalNAc ligand is attached at a 5′ terminus of the oligonucleotide. In some embodiments, the composition comprises a sense strand, and the GalNAc ligand is attached to the sense strand (e.g. attached to a 5′ end of the sense strand, or attached to a 3′ end of the sense strand). In some embodiments, the composition comprises an antisense strand, and the GalNAc ligand is attached to the antisense strand (e.g. attached to a 5′ end of the antisense strand, or attached to a 3′ end of the antisense strand). In some embodiments, the composition comprises a GalNAc ligand attached at a 3′ or 5′ terminus of the oligonucleotide.

Disclosed herein, in some embodiments, are compositions comprising an oligonucleotide that inhibits the expression of MTRES1, wherein the oligonucleotide comprises a GalNAc moiety. The GalNAc moiety may be included in any formula, structure, or GalNAc moiety shown below. In some embodiments, described herein is a compound (e.g. oligonucleotide) represented by Formula (I) or (II):

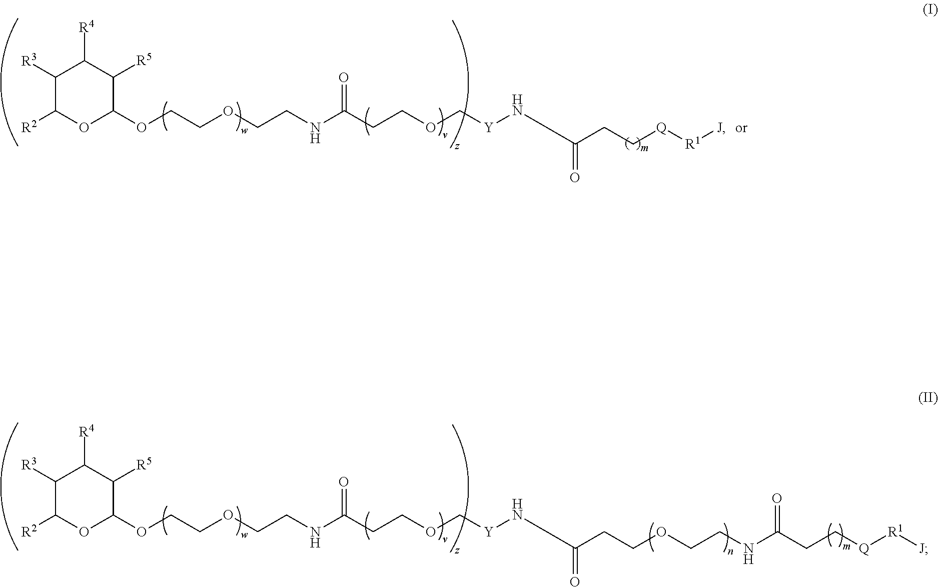

or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
   if z is 3, Y is C
   if z is 2, Y is CR⁶, or
   if z is 1, Y is C(R⁶)₂;
Q is selected from:
   C₃₋₁₀ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R)C(O)OR⁷, —C(O)OR⁷, —OC(O)R⁷, —S(O)R⁷, and C₁₋₆ alkyl, wherein the C₁₋₆ alkyl, is optionally substituted with one or more substituents independently selected from halogen, —CN, —OH, —SH, —NO₂, and —NH₂;
R¹ is a linker selected from:
   —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR)O—, —OP(O)(OR⁷)S—, —OP(O)(O⁷)O—, —SP(O)(O⁻)O—, —OP(S)(O⁻)O—, —OP(O)(S—)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R² is independently selected from: C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; R³ and R⁴ are each independently selected from: —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁵ is independently selected from: —OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
hydrogen;
halogen, —CN, —NO₂, —OR⁷, —SR⁷, —N(R⁷)₂, —C(O)R⁷, —C(O)N(R⁷)₂, —N(R)C(O)R⁷, —N(R⁷)C(O)N(R⁷)₂, —OC(O)N(R⁷)₂, —N(R)C(O)OR⁷, —C(O)OR⁷, —OC(O)R⁷, and —S(O)R⁷; and C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R)C(O)OR⁷, —C(O)OR⁷, —OC(O)R⁷, and —S(O)R⁷; each R⁷ is independently selected from:
hydrogen; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and C₃₋₁₀ carbocycle, and 3- to 10-membered heterocycle, each of which is optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆haloalkyl.

In some embodiments, each w is independently selected from any value from 1 to 10. In some embodiments, each w is independently selected from any value from 1 to 5. In some embodiments, each w is 1. In some embodiments, each v is independently selected from any value from 1 to 10. In some embodiments, each v is independently selected from any value from 1 to 5. In some embodiments, each v is 1. In some embodiments, n is selected from any value from 1 to 10. In some embodiments, n is selected from any value from 1 to 5. In some embodiments, n is 2. In some embodiments, m is selected from any value from 1 to 10. In some embodiments, m is selected from any value from 1 to 5. In some embodiments, m is selected from 1 and 2. In some embodiments, z is 3 and Y is C. In some embodiments, Q is selected from C₅₋₆ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, Q is selected from C₅₋₆ carbocycle optionally substituted with one or more substituents independently selected from halogen, —CN, —OH, —SH, —NO₂, and —NH₂. In some embodiments, Q is selected from phenyl and cyclohexyl, each of which is optionally substituted with one or more substituents independently selected from halogen, —CN, —OH, —SH, —NO₂, and —NH₂. In some embodiments, Q is selected from phenyl. In some embodiments, Q is selected from cyclohexyl. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S—)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂—NR⁷. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(0)O—, —OP(S)(O⁻)O—, —OP(O)(S—)O—, —OP(O)(0-)S—, and —OP(OR⁷)O—. In some embodiments, R¹ is selected from —OP(O)(OR⁷)O—, —OP(S)(OR⁷)O—, —OP(O)(0-)O—, —OP(S)(O)O—, —OP(O)(S—)O—, and —OP(OR⁷)O—. In some embodiments, R¹ is selected from —OP(O)(OR⁷)O— and —OP(OR⁷)O—. In some embodiments, R² is selected from C₁₋₃ alkyl substituted with one or more substituents independently selected from halogen, —OR⁷, —OC(O)R⁷, —SR⁷, —N(R)₂, —C(O)R⁷, and —S(O)R⁷. In some embodiments, R² is selected from C₁₋₃ alkyl substituted with one or more substituents independently selected from —OR⁷, —OC(O)R⁷, —SR⁷, and —N(R⁷)₂. In some embodiments, R² is selected from C₁₋₃ alkyl substituted with one or more substituents independently selected from —OR⁷ and —OC(O)R⁷. In some embodiments, R³ is selected from halogen, —OR⁷, —SR⁷, —N(R⁷)₂, —C(O)R⁷, —OC(O)R⁷, and —S(O)R⁷. In some embodiments, R³ is selected from —OR⁷—SR⁷, —OC(O)R⁷, and —N(R⁷)₂. In some embodiments, R³ is selected from —OR⁷— and —OC(O)R⁷. In some embodiments, R⁴ is selected from halogen, —OR⁷, —SR⁷, —N(R⁷)₂, —C(O)R⁷, —OC(O)R⁷, and —S(O)R⁷. In some embodiments, R⁴ is selected from —OR⁷—SR⁷, —OC(O)R⁷, and —N(R)₂. In some embodiments, R⁴ is selected from —OR⁷— and —OC(O)R⁷. In some embodiments, R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, R⁵ is selected from —OC(O)R⁷ and —N(R⁷)C(O)R⁷. In some embodiments, each R⁷ is independently selected from: hydrogen; and C₁₋₆ alkyl optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3-to 10-membered heterocycle. In some embodiments, each R⁷ is independently selected from C₁₋₆ alkyl optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from C₁₋₆ alkyl optionally substituted with one or more substituents independently selected from halogen, —CN, —OH, and —SH. In some embodiments, w is 1; v is 1; n is 2; m is 1 or 2; z is 3 and Y is C; Q is phenyl or cyclohexyl, each of which is optionally substituted with one or more substituents independently selected from halogen, —CN, —OH, —SH, —NO₂, —NH₂, and C₁₋₃ alkyl; R¹ is selected from —OP(O)(OR O—, —OP(S)(OR O—, —OP(O)(O⁻)O—, —OP(S)(O⁻)O—, —OP(O)(S)O—, and —OP(OR⁷)O—; R² is C₁ alkyl substituted with —OH or —OC(O)CH₃;

R³ is —OH or —OC(O)CH₃; R⁴ is —OH or —OC(O)CH₃; and R⁵ is —NH(O)CH₃. In some embodiments, the compound comprises:

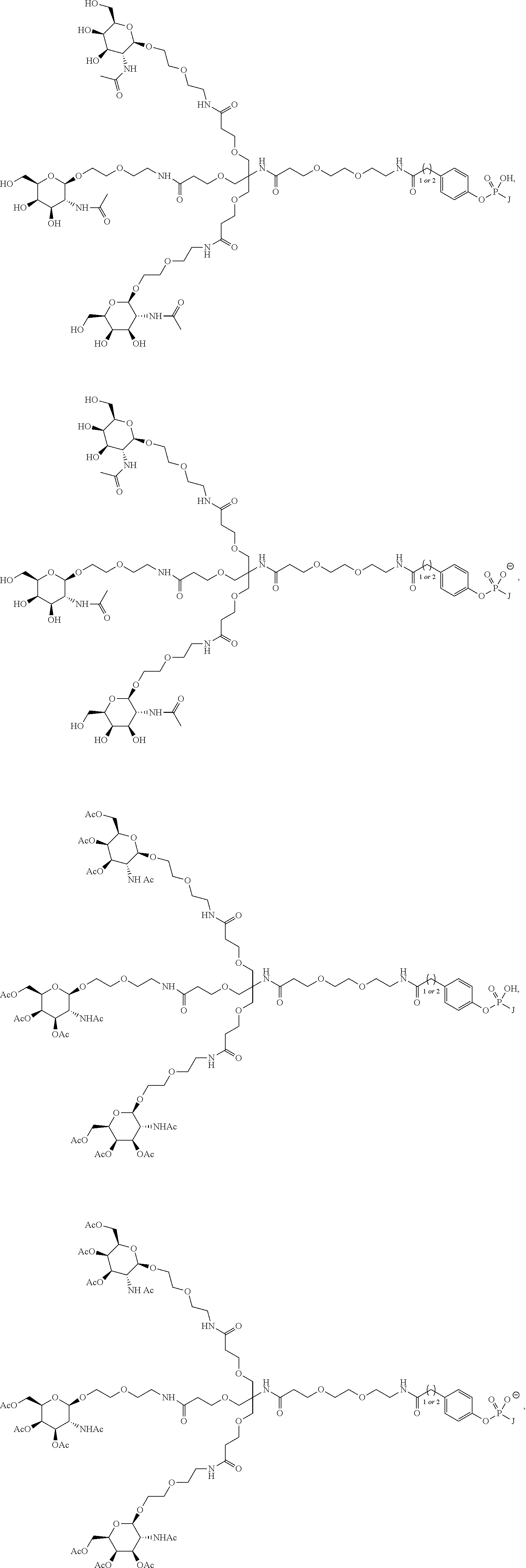

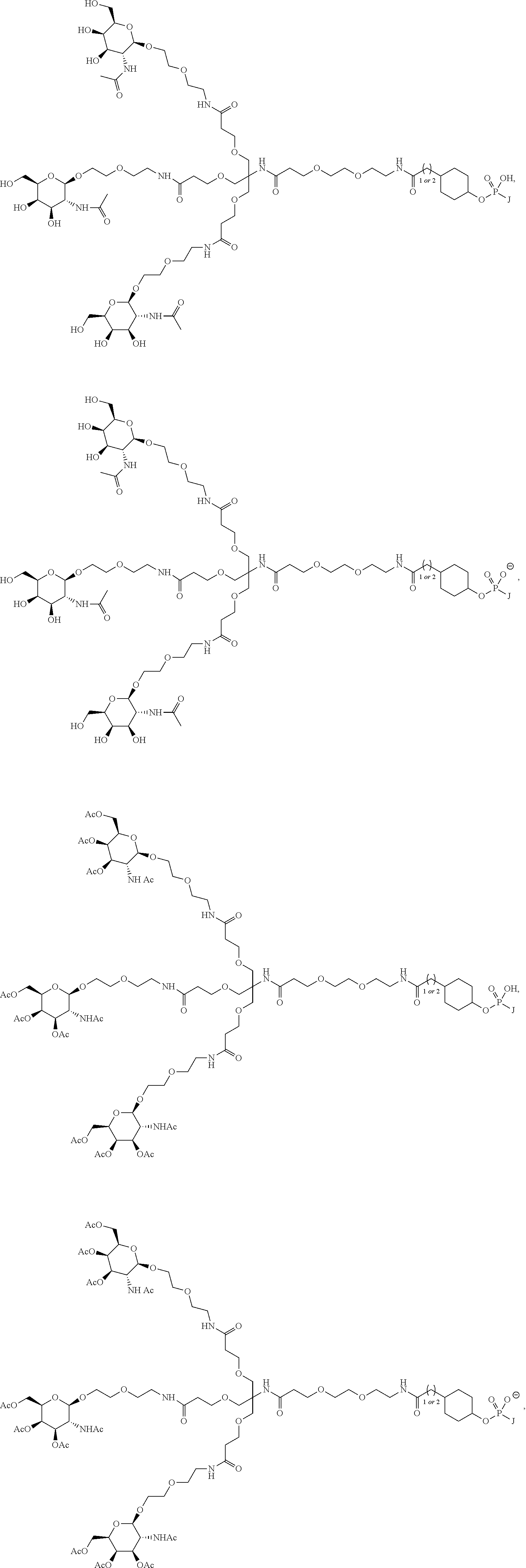

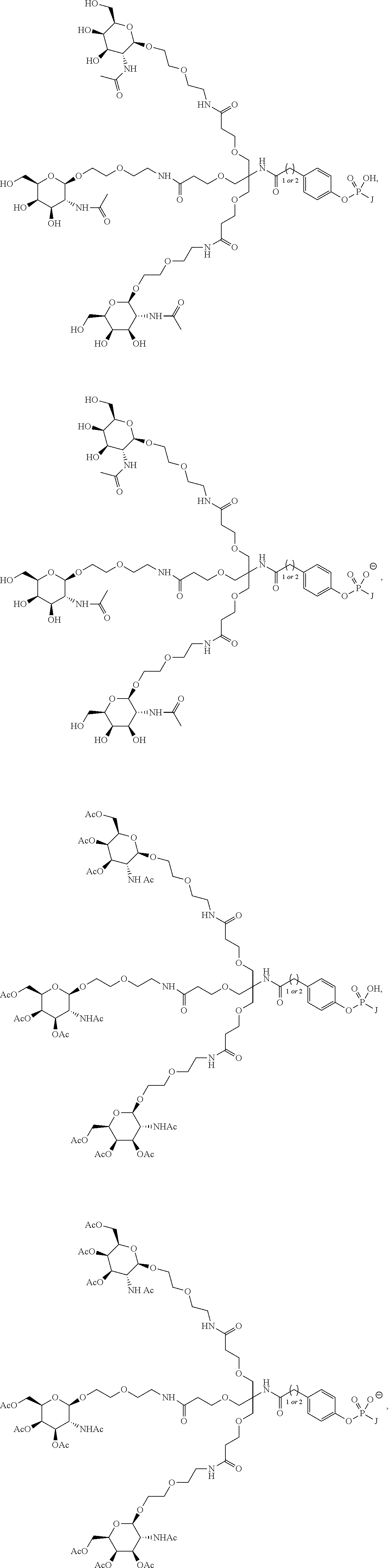

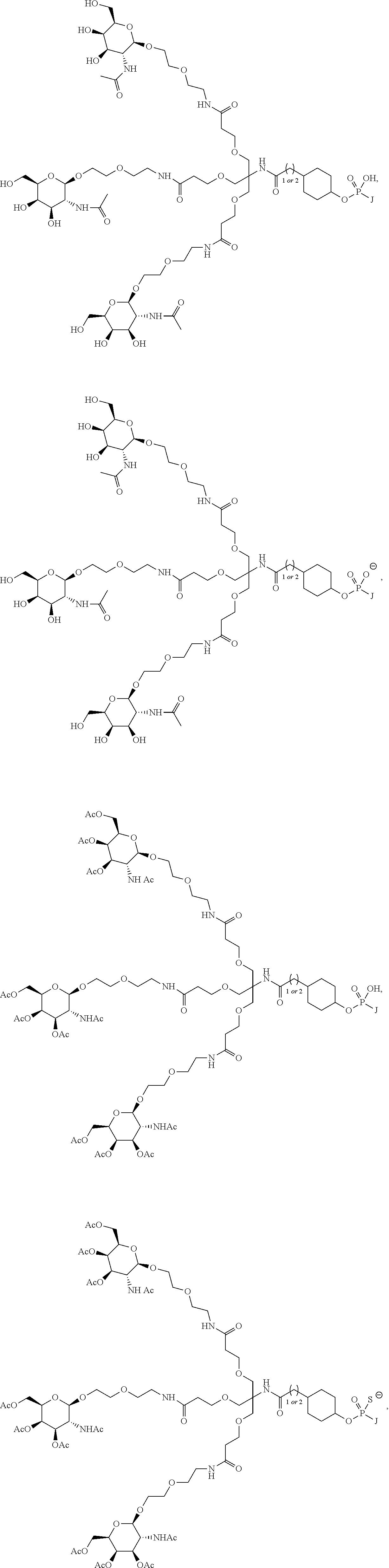

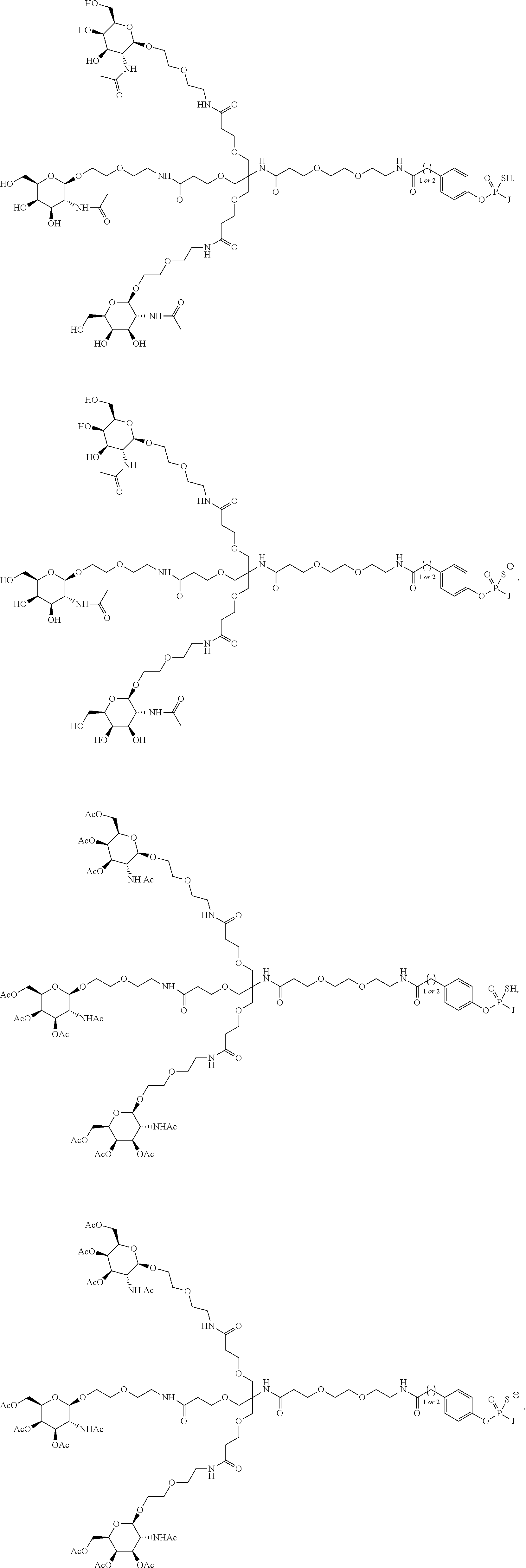

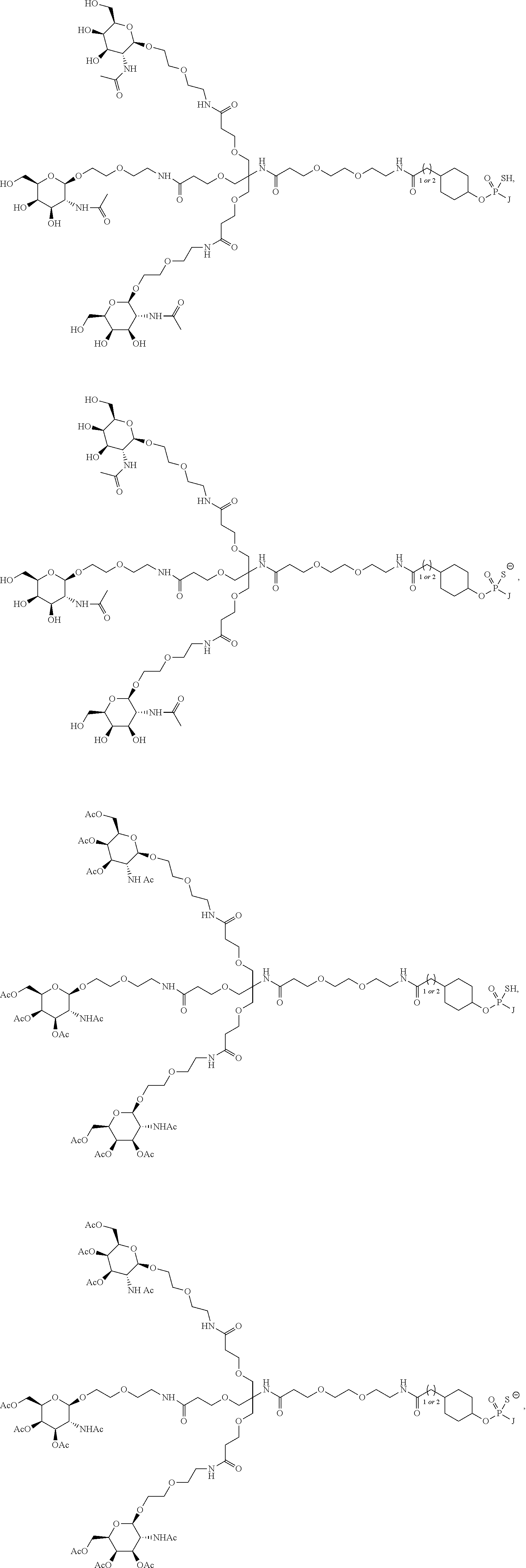

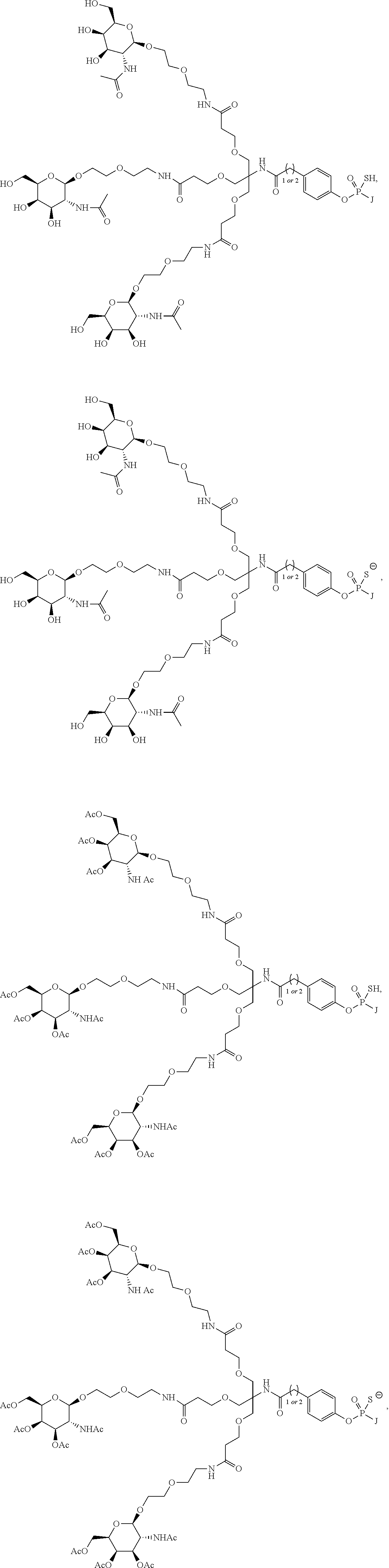

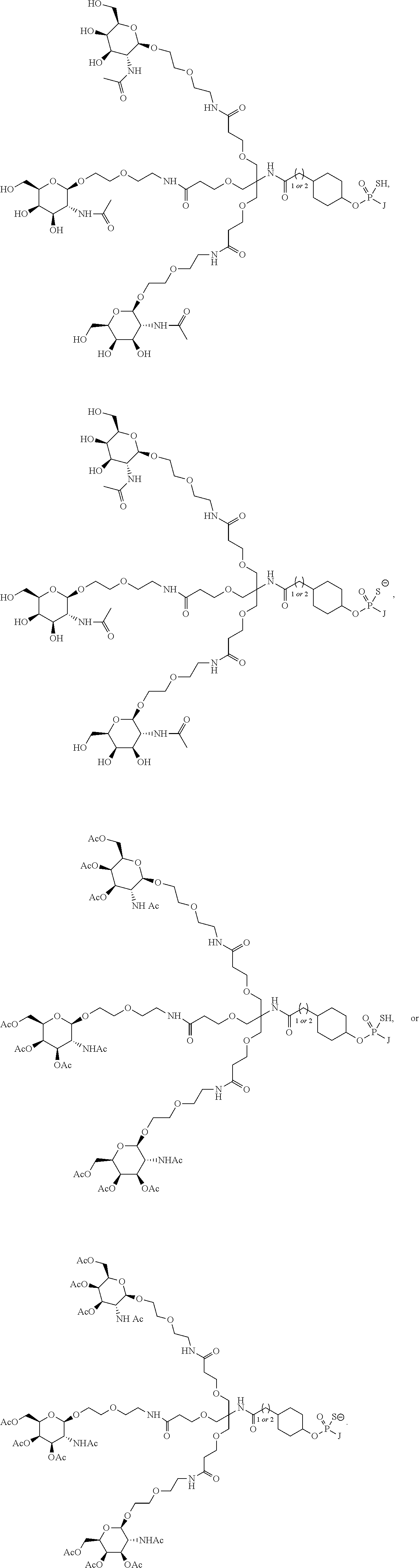

In some embodiments, the oligonucleotide (J) is attached at a 5′ end or a 3′ end of the oligonucleotide. In some embodiments, the oligonucleotide comprises DNA. In some embodiments, the oligonucleotide comprises RNA. In some embodiments, the oligonucleotide comprises one or more modified internucleoside linkages. In some embodiments, the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the compound binds to an asialoglycoprotein receptor. In some embodiments, the compound targets a hepatocyte.

Some embodiments include the following, where J is the oligonucleotide:

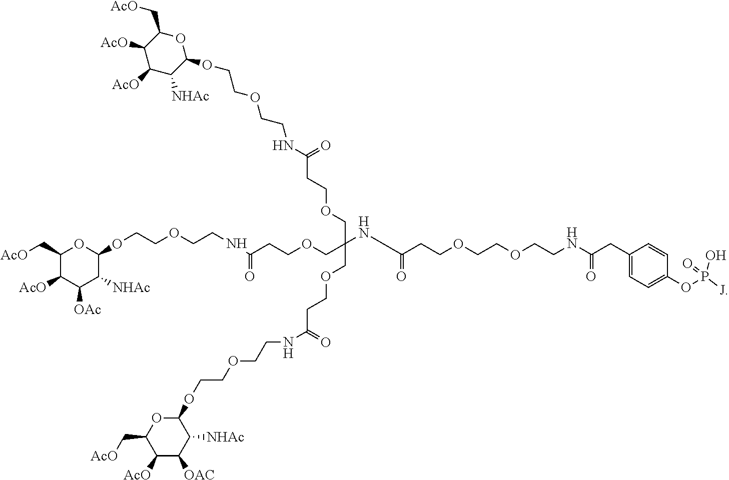

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

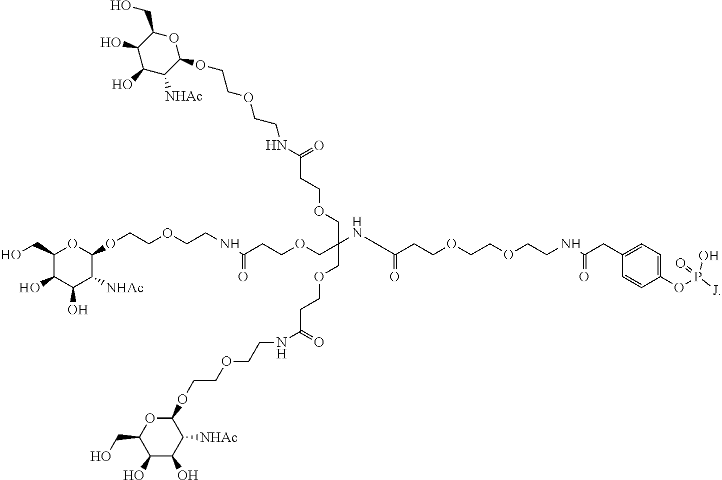

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

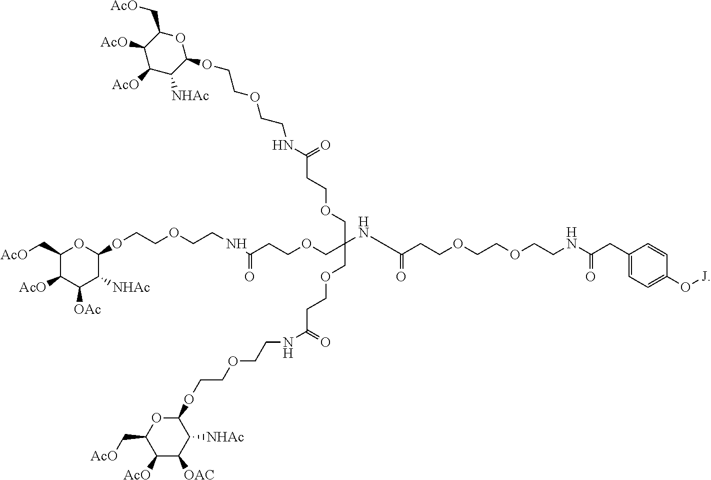

J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

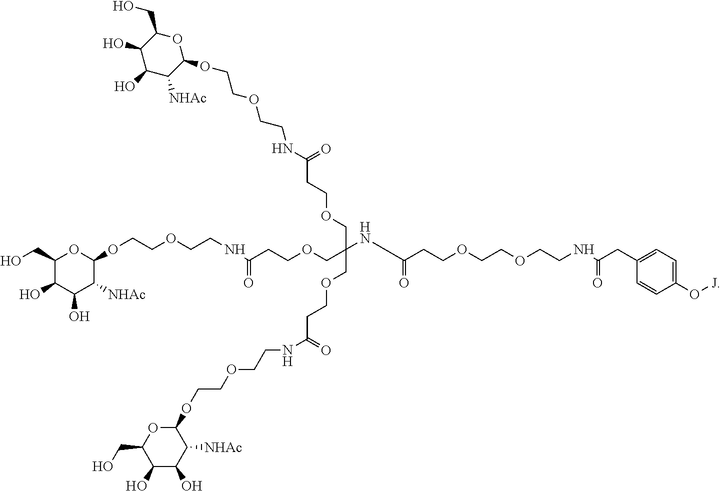

The structure in this compound attached to the oligonucleotide (J) may be referred to as “ETL17,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where the phosphate or “5” indicates a connection to the oligonucleotide:

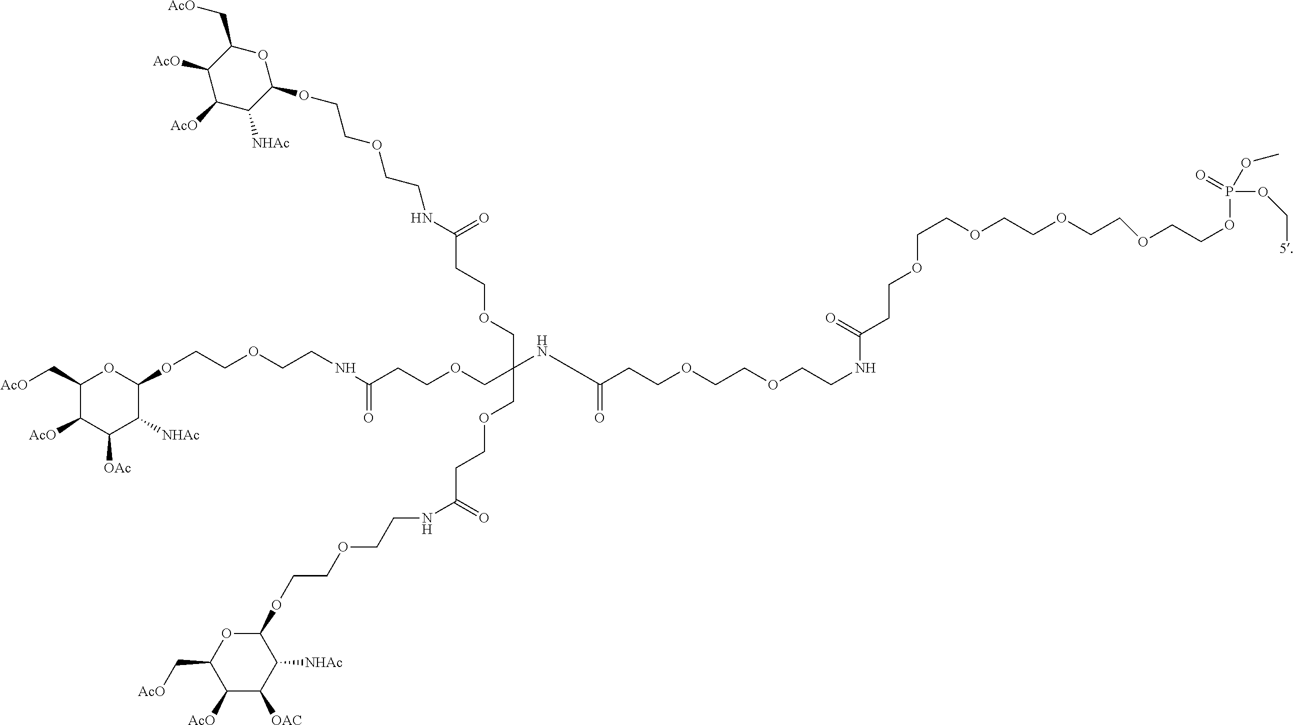

Some embodiments include the following, where the phosphate or “5” indicates a connection to the oligonucleotide:

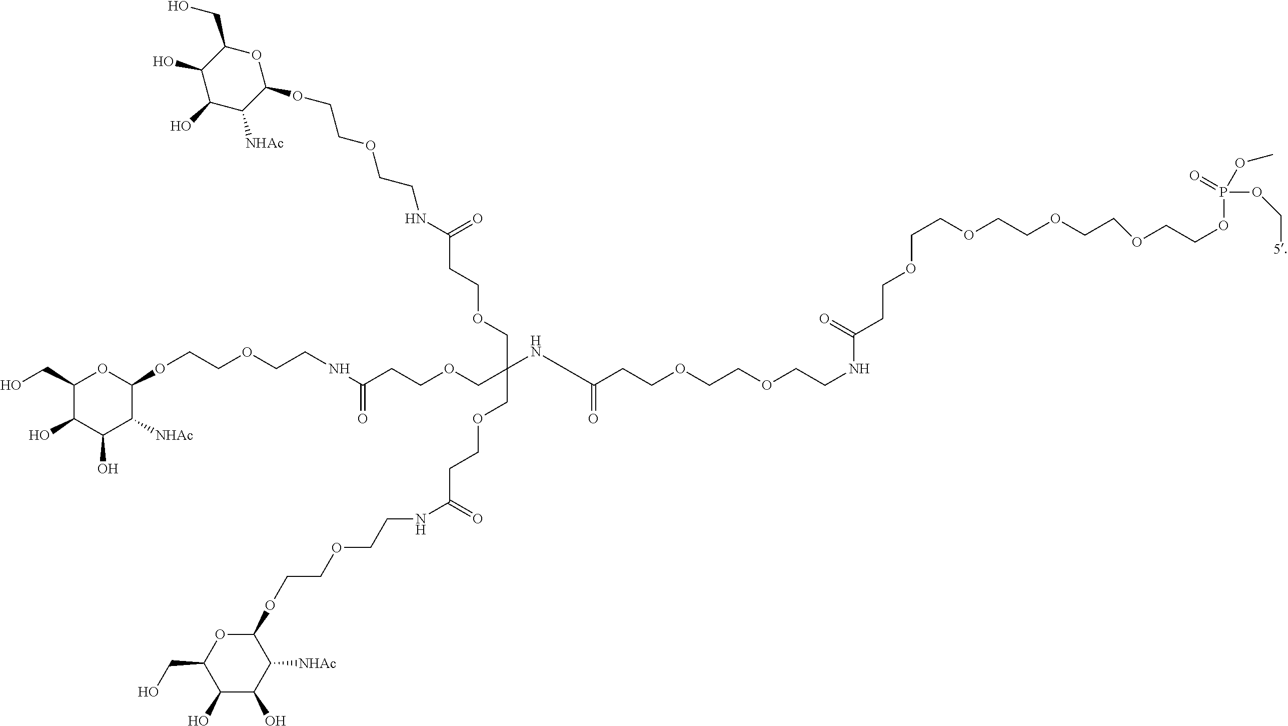

Some embodiments include the following, where J is the oligonucleotide:

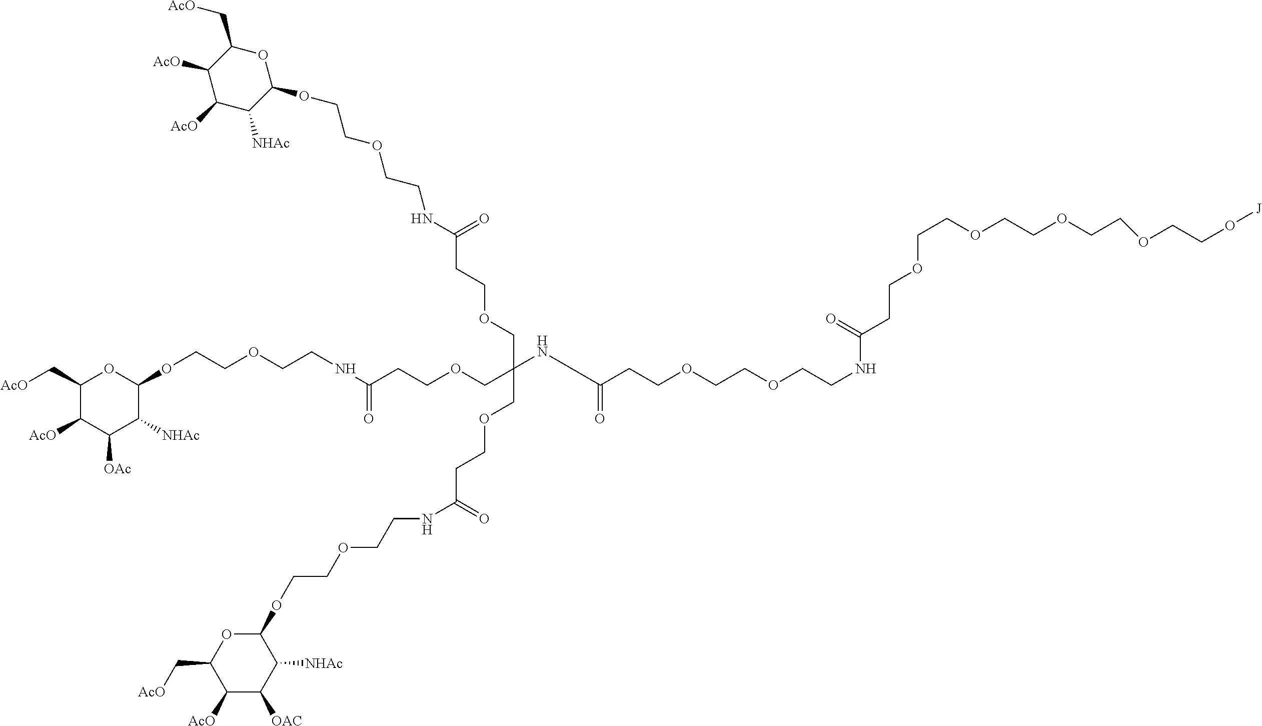

include one or more phosphates or phosphorothioates linking to the oligonucleotide. J may include one or more phosphates linking to the oligonucleotide. J may include a phosphate linking to the oligonucleotide.

J may include one or more phosphorothioates linking to the oligonucleotide. J may include a phosphorothioate linking to the oligonucleotide.

Some embodiments include the following, where J is the oligonucleotide:

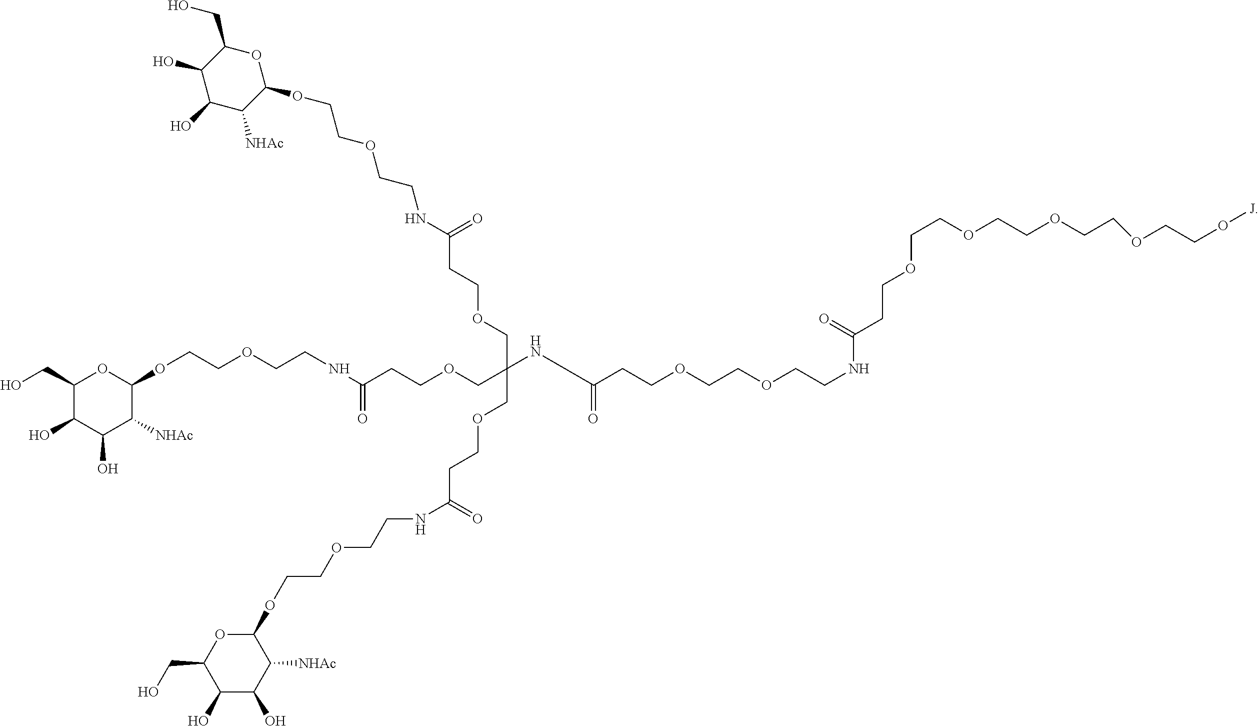

The structure in this compound attached to the oligonucleotide (J) may be referred to as “ETL1,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

3. siRNA modification patterns

In some embodiments, the composition comprises an oligonucleotide that inhibits the expression of MTRES1, wherein the oligonucleotide comprises an siRNA comprising a sense strand and an antisense strand, wherein the sense strand comprises modification pattern 1S: 5′-NfsnsNfnNfnNfNfNfnNfnNfnNfnNfnNfsnsn-3′ (SEQ ID NO: 2444), wherein “Nf” is a 2′ fluoro-modified nucleoside, “n” is a 2′ O-methyl modified nucleoside, and “s” is a phosphorothioate linkage. In some embodiments, the sense strand comprises modification pattern 2S: 5′-nsnsnnNfnNfNfNfnnnnnnnnnnsnsn-3′ (SEQ ID NO: 2445), wherein “Nf” is a 2′ fluoro-modified nucleoside, “n” is a 2′ O-methyl modified nucleoside, and “s” is a phosphorothioate linkage. In some embodiments, the sense strand comprises modification pattern 3S: 5′-nsnsnnNfnNfnNfnnnnnnnnnnsnsn-3′ (SEQ ID NO: 2446), wherein “Nf” is a 2′ fluoro-modified nucleoside, “n” is a 2′ O-methyl modified nucleoside, and “s” is a phosphorothioate linkage. In some embodiments, the sense strand comprises modification pattern 4S: 5′-NfsnsNfnNfnNfNfNfnNfnNfnNfnNfnNfsnsnN-moiety-3′ (SEQ ID NO: 2447), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 5S: 5′-nsnsnnNfnNfNfNfnnnnnnnnnnsnsnN-moiety-3′ (SEQ ID NO: 2448), wherein “Nf” is a 2′ fluoro-modified nucleoside, “n” is a 2′ O-methyl modified nucleoside, “s” is a phosphorothioate linkage, and N comprises one or more nucleosides. In some embodiments, the moiety in modification pattern 4S or 5S is a lipophilic moiety. In some embodiments, the moiety in modification pattern 4S or 5S is a lipid moiety. In some embodiments, the sense strand comprises modification pattern 6S: 5′-NfsnsNfnNfnNfnNfnNfnNfnNfnNfnNfsnsn-3′ (SEQ ID NO: 2449), wherein “Nf” is a2′ fluoro-modified nucleoside, “n” is a2′ O-methyl modified nucleoside, and “s” is a phosphorothioate linkage. In some embodiments, the sense strand comprises modification pattern 7S: 5′-nsnsnnNfNfNfNfNfnnnnnnnnnnsnsn-3′ (SEQ ID NO: 2450), wherein “Nf” is a 2′ fluoro-modified nucleoside, “n” is a 2′ O-methyl modified nucleoside, and “s” is a phosphorothioate linkage. In some embodiments, the sense strand comprises modification pattern 8S: 5′-nsnsnnnNfNfNfNfnnnnnnnnnnsnsn-3′ (SEQ ID NO: 2451), wherein “Nf” is a 2′ fluoro-modified nucleoside, “n” is a 2′ O-methyl modified nucleoside, and “s” is a phosphorothioate linkage. In some embodiments, the sense strand comprises modification pattern 9S: 5′-nsnsnnnnNfNfNfNfnnnnnnnnnsnsn-3′ (SEQ ID NO: 2452), wherein “Nf” is a 2′ fluoro-modified nucleoside, “n” is a 2′ O-methyl modified nucleoside, and “s” is a phosphorothioate linkage. In some embodiments, the sense strand comprises modification pattern 10S: 5′-NfsnsnnNfnNfnNfnNfnNfnNfnNfnnsnsn-3′ (SEQ ID NO: 2525), wherein “Nf” is a 2′ fluoro-modified nucleoside, “n” is a 2′ O-methyl modified nucleoside, and “s” is a phosphorothioate linkage. In some embodiments, the sense strand comprises modification pattern 11S: 5′-nsnsNfnNfnNfnNfnNfnNfnnnNfnNfsnsn-3′ (SEQ ID NO: 2526), wherein “Nf” is a 2′ fluoro-modified nucleoside, “n” is a 2′ O-methyl modified nucleoside, and “s” is a phosphorothioate linkage. In some embodiments, the sense strand comprises modification pattern 12S: 5′-NfsnsNfnNfnNfnNfnNfnnnNfnNfnNfsnsn-3′ (SEQ ID NO: 2527), wherein “Nf” is a 2′ fluoro-modified nucleoside, “n” is a 2′ O-methyl modified nucleoside, and “s” is a phosphorothioate linkage. In some embodiments, the sense strand comprises modification pattern 13S: 5′-nsnsnnnnNfnNfnNfnNfnNfnNfnNfsnsn-3′ (SEQ ID NO: 2528), wherein “Nf” is a 2′ fluoro-modified nucleoside, “n” is a 2′ O-methyl modified nucleoside, and “s” is a phosphorothioate linkage. In some embodiments, the sense strand comprises modification pattern 14S: 5′-snnnnnnNfNfNfNfnnnnnnnnnsnsn-3′ (SEQ ID NO: 2529), wherein “Nf” is a 2′ fluoro-modified nucleoside, “n” is a 2′ O-methyl modified nucleoside, and “s” is a phosphorothioate linkage. In some embodiments, the sense strand comprises modification pattern 15S: 5′-snnnnNfNfNfNfNfnnnnnnnnnnsnsn-3′ (SEQ ID NO: 2530), wherein “Nf” is a 2′ fluoro-modified nucleoside, “n” is a 2′ O-methyl modified nucleoside, and “s” is a phosphorothioate linkage. In some embodiments, the sense strand comprises modification pattern 16S: 5′-snnnnNfnNfNfdNnnnnnnnnnnsnsn-3′ (SEQ ID NO: 2531), wherein “Nf” is a 2′ fluoro-modified nucleoside, “dN” is a 2′ deoxy-modified nucleoside, “n” is a 2′ O-methyl modified nucleoside, and “s” is a phosphorothioate linkage. In some embodiments, the sense strand comprises modification pattern 17S: 5′-snnnnnNfNfnNfnnnnnnnnnnsnsn-3′ (SEQ ID NO: 2532), wherein “Nf” is a 2′ fluoro-modified nucleoside, “n” is a 2′ O-methyl modified nucleoside, and “s” is a phosphorothioate linkage. In some embodiments, the sense strand comprises modification pattern 18S: 5′-snnnnnnNfnNfNfnnnnnnnnnsnsn-3′ (SEQ ID NO: 2533), wherein “Nf” is a 2′ fluoro-modified nucleoside, “n” is a 2′ O-methyl modified nucleoside, and “s” is a phosphorothioate linkage. In some embodiments, the sense strand comprises modification pattern 19S: 5′-snnnnNfnNfnNfnNfnnnnnnnnsnsn-3′ (SEQ ID NO: 2534), wherein “Nf” is a 2′ fluoro-modified nucleoside, “n” is a 2′ O-methyl modified nucleoside, and “s” is a phosphorothioate linkage. In some embodiments, the sense strand comprises modification pattern 20S: 5′-snnnnNfnNfnNfnnnnnnnnnnsnsn-3′ (SEQ ID NO: 2535), wherein “Nf” is a 2′ fluoro-modified nucleoside, “n” is a 2′ O-methyl modified nucleoside, and “s” is a phosphorothioate linkage. In some embodiments, the sense strand comprises modification pattern 21S: 5′-snnnnNfNfnnNfNfnnnnnnnnnsnsn-3′ (SEQ ID NO: 2536), wherein “Nf” is a 2′ fluoro-modified nucleoside, “n” is a 2′ O-methyl modified nucleoside, and “s” is a phosphorothioate linkage. In some embodiments, the sense strand comprises modification pattern 22S: 5′-snnnnNfnNfNfNfNfnnnnnnnnsnsn-3′ (SEQ ID NO: 2537), wherein “Nf” is a 2′ fluoro-modified nucleoside, “n” is a 2′ O-methyl modified nucleoside, and “s” is a phosphorothioate linkage. In some embodiments, the sense strand comprises modification pattern 23S: 5′-snnnnnNfnNfNfnnnnnnnnnnsnsn-3′ (SEQ ID NO: 2538), wherein “Nf” is a 2′ fluoro-modified nucleoside, “n” is a 2′ O-methyl modified nucleoside, and “s” is a phosphorothioate linkage. In some embodiments, the sense strand comprises modification pattern 24S: 5′-snnnnnnnNfNfNfNfnnnnnnnnsnsn-3′ (SEQ ID NO: 2539), wherein “Nf” is a 2′ fluoro-modified nucleoside, “n” is a 2′ O-methyl modified nucleoside, and “s” is a phosphorothioate linkage. In some embodiments, the sense strand comprises modification pattern 25S: 5′-snnnnnNfNfNfNfNfnnnnnnnnnsnsn-3′ (SEQ ID NO: 2540), wherein “Nf” is a 2′ fluoro-modified nucleoside, “n” is a 2′ O-methyl modified nucleoside, and “s” is a phosphorothioate linkage. In some embodiments, the sense strand comprises modification pattern 26S: 5′-snnnnnNfNfNfNfnnnnnnnnnnsnsn-3′ (SEQ ID NO: 2541), wherein “Nf” is a 2′ fluoro-modified nucleoside, “n” is a 2′ O-methyl modified nucleoside, and “s” is a phosphorothioate linkage. In some embodiments, the sense strand comprises modification pattern 27S: 5′-snnnnnnnNfNfnNfnnnnnnnnsnsn-3′ (SEQ ID NO: 2542), wherein “Nf” is a 2′ fluoro-modified nucleoside, “n” is a 2′ O-methyl modified nucleoside, and “s” is a phosphorothioate linkage. In some embodiments, the sense strand comprises modification pattern 28S: 5′-snnnnNfNfnNfNfnNfnnnnnnnnsnsn-3′ (SEQ ID NO: 2543), wherein “Nf” is a 2′ fluoro-modified nucleoside, “n” is a 2′ O-methyl modified nucleoside, and “s” is a phosphorothioate linkage. In some embodiments, the sense strand comprises modification pattern 29S: 5′-snnnnnnnnNfnNfnnnnnnnnsnsn-3′ (SEQ ID NO: 2544), wherein “Nf” is a 2′ fluoro-modified nucleoside, “n” is a 2′ O-methyl modified nucleoside, and “s” is a phosphorothioate linkage. In some embodiments, the sense strand comprises modification pattern 30S: 5′-snnnnNfNfnNfnNfnnnnnnnnsnsn-3′ (SEQ ID NO: 2545), wherein “Nf” is a 2′ fluoro-modified nucleoside, “n” is a 2′ O-methyl modified nucleoside, and “s” is a phosphorothioate linkage. In some embodiments, the sense strand comprises modification pattern 31S: 5′-snnnnNfNfnNfNfnnnnnnnnnnsnsn-3′ (SEQ ID NO: 2546), wherein “Nf” is a 2′ fluoro-modified nucleoside, “n” is a 2′ O-methyl modified nucleoside, and “s” is a phosphorothioate linkage. In some embodiments, the sense strand comprises modification pattern 32S: 5′-snnnnnnNfNfdNNfnnnnnnnnnsnsn-3′ (SEQ ID NO: 2547), wherein “Nf” is a 2′ fluoro-modified nucleoside, “dN” is a 2′ deoxy-modified nucleoside, “n” is a 2′ O-methyl modified nucleoside, and “s” is a phosphorothioate linkage.

In some embodiments, the composition comprises an oligonucleotide that inhibits the expression of MTRES1, wherein the oligonucleotide comprises an siRNA comprising a sense strand and an antisense strand, wherein the antisense strand comprises modification pattern 1AS: 5′-nsNfsnNfnNfnNfnNfnnnNfnNfnNfnsnsn-3′ (SEQ ID NO: 2453), wherein “Nf” is a 2′ fluoro-modified nucleoside, “n” is a 2′ O-methyl modified nucleoside, and “s” is a phosphorothioate linkage. In some embodiments, the antisense strand comprises modification pattern 2AS: 5′-nsNfsnnnNfnNfNfnnnnNfnNfnnnsnsn-3′ (SEQ ID NO: 2454), wherein “Nf” is a 2′ fluoro-modified nucleoside, “n” is a 2′ O-methyl modified nucleoside, and “s” is a phosphorothioate linkage. In some embodiments, the antisense strand comprises modification pattern 3AS: 5′-nsNfsnnnNfnnnnnnnNfnNfnnnsnsn-3′ (SEQ ID NO: 2455), wherein “Nf” is a 2′ fluoro-modified nucleoside, “n” is a 2′ O-methyl modified nucleoside, and “s” is a phosphorothioate linkage. In some embodiments, the antisense strand comprises modification pattern 4AS: 5′-nsNfsnNfnNfnnnnnnnNfnNfnnnsnsn-3′ (SEQ ID NO: 2456), wherein “Nf” is a 2′ fluoro-modified nucleoside, “n” is a 2′ O-methyl modified nucleoside, and “s” is a phosphorothioate linkage. In some embodiments, the antisense strand comprises modification pattern 5AS: 5′-nsNfsnnnnnnnnnnnNfnNfnnnsnsn-3′ (SEQ ID NO: 2457), wherein “Nf” is a 2′ fluoro-modified nucleoside, “n” is a 2′ O-methyl modified nucleoside, and “s” is a phosphorothioate linkage. In some embodiments, the antisense strand comprises modification pattern 6AS: 5′-nsNfsnnnNfnnNfnnnnNfnNfnnnsnsn-3′ (SEQ ID NO: 2458), wherein “Nf” is a 2′ fluoro-modified nucleoside, “n” is a 2′ O-methyl modified nucleoside, and “s” is a phosphorothioate linkage. In some embodiments, the antisense strand comprises modification pattern 7AS: 5′-nsNfsnNfnNfnNfnNfnNfnNfnNfnNfnsnsn-3′ (SEQ ID NO: 2459), wherein “Nf” is a 2′ fluoro-modified nucleoside, “n” is a 2′ O-methyl modified nucleoside, and “s” is a phosphorothioate linkage. In some embodiments, the antisense strand comprises modification pattern 8AS: 5′-nsNfsnnnnnnnnnnnNfnnnnnsnsn-3′ (SEQ ID NO: 2460), wherein “Nf” is a 2′ fluoro-modified nucleoside, “n” is a 2′ O-methyl modified nucleoside, and “s” is a phosphorothioate linkage. In some embodiments, the antisense strand comprises modification pattern 9AS: 5′-nsNfsnnnNfnNfnnnnnNfnNfnnnsnsn-3′ (SEQ ID NO: 2548), wherein “Nf” is a 2′ fluoro-modified nucleoside, “n” is a 2′ O-methyl modified nucleoside, and “s” is a phosphorothioate linkage. In some embodiments, the antisense strand comprises modification pattern 10AS: 5′-nsNfsnNfsnNfnNfnNfnNfnNfnNfnNfnsnsn-3′ (SEQ ID NO: 2549), wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of MTRES1, wherein the oligonucleotide comprises an siRNA comprising a sense strand and an antisense strand, wherein the sense strand comprises pattern 1S and the antisense strand comprises pattern 1AS, 2AS, 3AS, 4AS, 5AS, 6AS, 7AS, 8AS, 9AS or 10AS. In some embodiments, the sense strand comprises pattern 2S and the antisense strand comprises pattern 1AS, 2AS, 3AS, 4AS, 5AS, 6AS, 7AS, 8AS, 9AS or 10AS. In some embodiments, the sense strand comprises pattern 3S and the antisense strand comprises pattern 1AS, 2AS, 3AS, 4AS, 5AS, 6AS, 7AS, 8AS, 9AS or 10AS. In some embodiments, the sense strand comprises pattern 4S and the antisense strand comprises pattern 1AS, 2AS, 3AS, 4AS, 5AS, 6AS, 7AS, 8AS, 9AS or 10AS. In some embodiments, the sense strand comprises pattern 5S and the antisense strand comprises pattern 1AS, 2AS, 3AS, 4AS, 5AS, 6AS, 7AS, 8AS, 9AS or 10AS. In some embodiments, the sense strand comprises pattern 6S and the antisense strand comprises pattern 1AS, 2AS, 3AS, 4AS, 5AS, 6AS, 7AS, 8AS, 9AS or 10AS. In some embodiments, the sense strand comprises pattern 7S and the antisense strand comprises pattern 1AS, 2AS, 3AS, 4AS, 5AS, 6AS, 7AS, 8AS, 9AS or 10AS.

In some embodiments, the sense strand comprises pattern 8S and the antisense strand comprises pattern 1AS, 2AS, 3AS, 4AS, 5AS, 6AS, 7AS, 8AS, 9AS or 10AS. In some embodiments, the sense strand comprises pattern 9S and the antisense strand comprises pattern 1AS, 2AS, 3AS, 4AS, 5AS, 6AS, 7AS, 8AS, 9AS or 10AS. In some embodiments, the sense strand comprises pattern 10S and the antisense strand comprises pattern 1AS, 2AS, 3AS, 4AS, 5AS, 6AS, 7AS, 8AS, 9AS or 10AS. In some embodiments, the sense strand comprises pattern 11S and the antisense strand comprises pattern 1AS, 2AS, 3AS, 4AS, 5AS, 6AS, 7AS, 8AS, 9AS or 10AS. In some embodiments, the sense strand comprises pattern 12S and the antisense strand comprises pattern 1AS, 2AS, 3AS, 4AS, 5AS, 6AS, 7AS, 8AS, 9AS or 10AS. In some embodiments, the sense strand comprises pattern 13S and the antisense strand comprises pattern 1AS, 2AS, 3AS, 4AS, 5AS, 6AS, 7AS, 8AS, 9AS or 10AS. In some embodiments, the sense strand comprises pattern 14S and the antisense strand comprises pattern 1AS, 2AS, 3AS, 4AS, 5AS, 6AS, 7AS, 8AS, 9AS or 10AS. In some embodiments, the sense strand comprises pattern 15S and the antisense strand comprises pattern 1AS, 2AS, 3AS, 4AS, 5AS, 6AS, 7AS, 8AS, 9AS or 10AS. In some embodiments, the sense strand comprises pattern 16S and the antisense strand comprises pattern 1AS, 2AS, 3AS, 4AS, 5AS, 6AS, 7AS, 8AS, 9AS or 10AS. In some embodiments, the sense strand comprises pattern 17S and the antisense strand comprises pattern 1AS, 2AS, 3AS, 4AS, 5AS, 6AS, 7AS, 8AS, 9AS or 10AS. In some embodiments, the sense strand comprises pattern 18S and the antisense strand comprises pattern 1AS, 2AS, 3AS, 4AS, 5AS, 6AS, 7AS, 8AS, 9AS or 10AS. In some embodiments, the sense strand comprises pattern 19S and the antisense strand comprises pattern 1AS, 2AS, 3AS, 4AS, 5AS, 6AS, 7AS, 8AS, 9AS or 10AS. In some embodiments, the sense strand comprises pattern 20S and the antisense strand comprises pattern 1AS, 2AS, 3AS, 4AS, 5AS, 6AS, 7AS, 8AS, 9AS or 10AS. In some embodiments, the sense strand comprises pattern 21S and the antisense strand comprises pattern 1AS, 2AS, 3AS, 4AS, 5AS, 6AS, 7AS, 8AS, 9AS or 10AS. In some embodiments, the sense strand comprises pattern 22S and the antisense strand comprises pattern 1AS, 2AS, 3AS, 4AS, 5AS, 6AS, 7AS, 8AS, 9AS or 10AS. In some embodiments, the sense strand comprises pattern 23S and the antisense strand comprises pattern 1AS, 2AS, 3AS, 4AS, 5AS, 6AS, 7AS, 8AS, 9AS or 10AS. In some embodiments, the sense strand comprises pattern 24S and the antisense strand comprises pattern 1AS, 2AS, 3AS, 4AS, 5AS, 6AS, 7AS, 8AS, 9AS or 10AS. In some embodiments, the sense strand comprises pattern 25S and the antisense strand comprises pattern 1AS, 2AS, 3AS, 4AS, 5AS, 6AS, 7AS, 8AS, 9AS or 10AS. In some embodiments, the sense strand comprises pattern 26S and the antisense strand comprises pattern 1AS, 2AS, 3AS, 4AS, 5AS, 6AS, 7AS, 8AS, 9AS or 10AS. In some embodiments, the sense strand comprises pattern 27S and the antisense strand comprises pattern 1AS, 2AS, 3AS, 4AS, 5AS, 6AS, 7AS, 8AS, 9AS or 10AS. In some embodiments, the sense strand comprises pattern 28S and the antisense strand comprises pattern 1AS, 2AS, 3AS, 4AS, 5AS, 6AS, 7AS, 8AS, 9AS or 10AS. In some embodiments, the sense strand comprises pattern 29S and the antisense strand comprises pattern 1AS, 2AS, 3AS, 4AS, 5AS, 6AS, 7AS, 8AS, 9AS or 10AS. In some embodiments, the sense strand comprises pattern 30S and the antisense strand comprises pattern 1AS, 2AS, 3AS, 4AS, 5AS, 6AS, 7AS, 8AS, 9AS or 10AS. In some embodiments, the sense strand comprises pattern 31S and the antisense strand comprises pattern 1AS, 2AS, 3AS, 4AS, 5AS, 6AS, 7AS, 8AS, 9AS or 10AS. In some embodiments, the sense strand comprises pattern 32S and the antisense strand comprises pattern 1AS, 2AS, 3AS, 4AS, 5AS, 6AS, 7AS, 8AS, 9AS or 10AS.

In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, or 32S and the antisense strand comprises pattern 1AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, or 32S and the antisense strand comprises pattern 2AS. In some embodiments, the sense strand comprises pattern 1S, 2S, 3S, 4S, 5S, 6S, 7S, 8S, 9S, 10S, 1S, 12S, 13S, 14S, 15S, 16S, 17S, 18S, 19S, 20S, 21S, 22S, 23S, 24S, 25S, 26S, 27S, 28S, 29S, 305, 31S, or 32S and the antisense strand comprises pattern 3AS. In some embodiments, the sense strand comprises pattern 1S, 2S, 3S, 4S, 5S, 6S, 7S, 8S, 9S, 10S, 11S, 125, 135, 145, 155, 165, 175, 185, 195, 205, 21S, 22S, 23S, 24S, 25S, 26S, 27S, 285, 295, 305, 31S, or 32S and the antisense strand comprises pattern 4AS. In some embodiments, the sense strand comprises pattern 1S, 2S, 3S, 4S, 5S, 6S, 7S, 8S, 9S, 10S, 11S, 125, 135, 145, 155, 16S, 17S, 18S, 19S, 20S, 21S, 22S, 23S, 24S, 25S, 26S, 27S, 28S, 29S, 305, 31S, or 32S and the antisense strand comprises pattern 5AS. In some embodiments, the sense strand comprises pattern 1S, 2S, 3S, 4S, 5S, 6S, 7S, 8S, 9S, 10S, 11S, 125, 135, 145, 155, 165, 175, 185, 195, 205, 21S, 22S, 23S, 24S, 25S, 26S, 27S, 28S, 29S, 305, 31S, or 32S and the antisense strand comprises pattern 6AS. In some embodiments, the sense strand comprises pattern 1S, 2S, 3S, 4S, 5S, 6S, 7S, 8S, 9S, 10S, 11S, 125, 135, 145, 155, 16S, 17S, 18S, 195, 205, 215, 22S, 23S, 24S, 25S, 26S, 27S, 28S, 29S, 305, 31S, or 32S and the antisense strand comprises pattern 7AS. In some embodiments, the sense strand comprises pattern 1S, 2S, 3S, 4S, 5S, 6S, 7S, 8S, 9S, 10S, 11S, 125, 135, 14S, 15S, 165, 175, 185, 195, 205, 21S, 22S, 23S, 24S, 25S, 26S, 27S, 28S, 29S, 305, 31S, or 32S and the antisense strand comprises pattern 8AS. In some embodiments, the sense strand comprises pattern 1S, 2S, 3S, 4S, 5S, 6S, 7S, 8S, 9S, 10S, S, 125, 135, 145, 155, 165, 175, 185, 19S, 20S, 215, 22S, 23S, 24S, 25S, 26S, 27S, 28S, 29S, 305, 31S, or 32S and the antisense strand comprises pattern 9AS. In some embodiments, the sense strand comprises pattern 1S, 2S, 3S, 4S, 5S, 6S, 7S, 8S, 9S, 10S, 11S, 125, 135, 145, 155, 165, 175, 185, 195, 205, 21S, 22S, 23S, 24S, 25S, 26S, 27S, 28S, 29S, 305, 31S, or 32S and the antisense strand comprises pattern 10AS.

In some embodiments, the sense strand comprises any one of modification patters 1S, 2S, 3S, 4S, 5S, 6S, 7S, 8S, or 9S. In some embodiments, the sense strand comprises any one of modification patters 1S, 2S, 3S, 4S, 5S, 6S, 7S, 8S, 9S, 10S, S, 125, 135, 145, 155, 165, 175, 185, 195, 20S, 21S, 22S, 23S, 24S, 25S, 26S, 27S, 28S, 29S, 305, 315, or 32S. In some embodiments, the sense strand comprises modification pattern 1AS, 2AS, 3AS, 4AS, 5AS, 6AS, 7AS, or 8AS. In some embodiments, the antisense strand comprises modification pattern 1AS, 2AS, 3AS, 4AS, 5AS, 6AS, 7AS, 8AS, 9AS or 10AS. In some embodiments, the antisense strand comprises modification pattern 1AS, 2AS, 3AS, 4AS, 5AS, 6AS, 7AS, or 8AS. In some embodiments, the antisense strand comprises modification pattern 1S, 2S, 3S, 4S, 5S, 6S, 7S, 8S, 9S, 10S, 11S, 12S, 13S, 145, 155, 16S, 17S, 185, 195,205, 215, 22S, 23S, 24S, 25S, 26S, 27S, 28S, 29S, 305, 315, or 32S. In some embodiments, the sense strand or the antisense strand comprises modification pattern ASO1.

In some embodiments, purines of the sense strand comprise 2′ fluoro modified purines. In some embodiments, purines of the sense strand comprise 2′-O-methyl modified purines. In some embodiments, purines of the sense strand comprise a mixture of 2′ fluoro and 2′-O-methyl modified purines. In some embodiments, all purines of the sense strand comprise 2′ fluoro modified purines. In some embodiments, all purines of the sense strand comprise 2′-O-methyl modified purines. In some embodiments, all purines of the sense strand comprise a mixture of 2′ fluoro and 2′-O-methyl modified purines.

In some embodiments, pyrimidines of the sense strand comprise 2′ fluoro modified pyrimidines. In some embodiments, pyrimidines of the sense strand comprise 2′-O-methyl modified pyrimidines. In some embodiments, pyrimidines of the sense strand comprise a mixture of 2′ fluoro and 2′-O-methyl modified pyrimidines. In some embodiments, all pyrimidines of the sense strand comprise 2′ fluoro modified pyrimidines. In some embodiments, all pyrimidines of the sense strand comprise 2′-O-methyl modified pyrimidines. In some embodiments, all pyrimidines of the sense strand comprise a mixture of 2′ fluoro and 2′-O-methyl modified pyrimidines.

In some embodiments, purines of the sense strand comprise 2′ fluoro modified purines, and pyrimidines of the sense strand comprise a mixture of 2′ fluoro and 2′-O-methyl modified pyrimidines. In some embodiments, purines of the sense strand comprise 2′-O-methyl modified purines, and pyrimidines of the sense strand comprise a mixture of 2′ fluoro and 2′-O-methyl modified pyrimidines. In some embodiments, purines of the sense strand comprise 2′ fluoro modified purines, and pyrimidines of the sense strand comprise 2′-O-methyl modified pyrimidines. In some embodiments, purines of the sense strand comprise 2′-O-methyl modified purines, and pyrimidines of the sense strand comprise 2′ fluoro modified pyrimidines. In some embodiments, pyrimidines of the sense strand comprise 2′ fluoro modified pyrimidines, and purines of the sense strand comprise a mixture of 2′ fluoro and 2′-O-methyl modified purines. In some embodiments, pyrimidines of the sense strand comprise 2′-O-methyl modified pyrimidines, and purines of the sense strand comprise a mixture of 2′ fluoro and 2′-O-methyl modified purines. In some embodiments, pyrimidines of the sense strand comprise 2′ fluoro modified pyrimidines, and purines of the sense strand comprise 2′-O-methyl modified purines. In some embodiments, pyrimidines of the sense strand comprise 2′-O-methyl modified pyrimidines, and purines of the sense strand comprise 2′ fluoro modified purines.

In some embodiments, all purines of the sense strand comprise 2′ fluoro modified purines, and all pyrimidines of the sense strand comprise a mixture of 2′ fluoro and 2′-O-methyl modified pyrimidines. In some embodiments, all purines of the sense strand comprise 2′-O-methyl modified purines, and all pyrimidines of the sense strand comprise a mixture of 2′ fluoro and 2′-O-methyl modified pyrimidines. In some embodiments, all purines of the sense strand comprise 2′ fluoro modified purines, and all pyrimidines of the sense strand comprise 2′-O-methyl modified pyrimidines. In some embodiments, all purines of the sense strand comprise 2′-O-methyl modified purines, and all pyrimidines of the sense strand comprise 2′ fluoro modified pyrimidines. In some embodiments, all pyrimidines of the sense strand comprise 2′ fluoro modified pyrimidines, and all purines of the sense strand comprise a mixture of 2′ fluoro and 2′-O-methyl modified purines. In some embodiments, all pyrimidines of the sense strand comprise 2′-O-methyl modified pyrimidines, and all purines of the sense strand comprise a mixture of 2′ fluoro and 2′-O-methyl modified purines. In some embodiments, all pyrimidines of the sense strand comprise 2′ fluoro modified pyrimidines, and all purines of the sense strand comprise 2′-O-methyl modified purines. In some embodiments, all pyrimidines of the sense strand comprise 2′-O-methyl modified pyrimidines, and all purines of the sense strand comprise 2′ fluoro modified purines.

In some embodiments, purines of the antisense strand comprise 2′ fluoro modified purines. In some embodiments, purines of the antisense strand comprise 2′-O-methyl modified purines. In some embodiments, purines of the antisense strand comprise a mixture of 2′ fluoro and 2′-O-methyl modified purines. In some embodiments, all purines of the antisense strand comprise 2′ fluoro modified purines. In some embodiments, all purines of the antisense strand comprise 2′-O-methyl modified purines. In some embodiments, all purines of the antisense strand comprise a mixture of 2′ fluoro and 2′-O-methyl modified purines.

In some embodiments, pyrimidines of the antisense strand comprise 2′ fluoro modified pyrimidines. In some embodiments, pyrimidines of the antisense strand comprise 2′-O-methyl modified pyrimidines. In some embodiments, pyrimidines of the antisense strand comprise a mixture of 2′ fluoro and 2′-O-methyl modified pyrimidines. In some embodiments, all pyrimidines of the antisense strand comprise 2′ fluoro modified pyrimidines. In some embodiments, all pyrimidines of the antisense strand comprise 2′-O-methyl modified pyrimidines. In some embodiments, all pyrimidines of the antisense strand comprise a mixture of 2′ fluoro and 2′-O-methyl modified pyrimidines.

In some embodiments, purines of the antisense strand comprise 2′ fluoro modified purines, and pyrimidines of the antisense strand comprise a mixture of 2′ fluoro and 2′-O-methyl modified pyrimidines. In some embodiments, purines of the antisense strand comprise 2′-O-methyl modified purines, and pyrimidines of the antisense strand comprise a mixture of 2′ fluoro and 2′-O-methyl modified pyrimidines. In some embodiments, purines of the antisense strand comprise 2′ fluoro modified purines, and pyrimidines of the antisense strand comprise 2′-O-methyl modified pyrimidines. In some embodiments, purines of the antisense strand comprise 2′-O-methyl modified purines, and pyrimidines of the antisense strand comprise 2′ fluoro modified pyrimidines. In some embodiments, pyrimidines of the antisense strand comprise 2′ fluoro modified pyrimidines, and purines of the antisense strand comprise a mixture of 2′ fluoro and 2′-O-methyl modified purines. In some embodiments, pyrimidines of the antisense strand comprise 2′-O-methyl modified pyrimidines, and purines of the antisense strand comprise a mixture of 2′ fluoro and 2′-O-methyl modified purines. In some embodiments, pyrimidines of the antisense strand comprise 2′ fluoro modified pyrimidines, and purines of the antisense strand comprise 2′-O-methyl modified purines. In some embodiments, pyrimidines of the antisense strand comprise 2′-O-methyl modified pyrimidines, and purines of the antisense strand comprise 2′ fluoro modified purines.

In some embodiments, all purines of the antisense strand comprise 2′ fluoro modified purines, and all pyrimidines of the antisense strand comprise a mixture of 2′ fluoro and 2′-O-methyl modified pyrimidines. In some embodiments, all purines of the antisense strand comprise 2′-O-methyl modified purines, and all pyrimidines of the antisense strand comprise a mixture of 2′ fluoro and 2′-O-methyl modified pyrimidines. In some embodiments, all purines of the antisense strand comprise 2′ fluoro modified purines, and all pyrimidines of the antisense strand comprise 2′-O-methyl modified pyrimidines. In some embodiments, all purines of the antisense strand comprise 2′-O-methyl modified purines, and all pyrimidines of the antisense strand comprise 2′ fluoro modified pyrimidines. In some embodiments, all pyrimidines of the antisense strand comprise 2′ fluoro modified pyrimidines, and all purines of the antisense strand comprise a mixture of 2′ fluoro and 2′-O-methyl modified purines. In some embodiments, all pyrimidines of the antisense strand comprise 2′-O-methyl modified pyrimidines, and all purines of the antisense strand comprise a mixture of 2′ fluoro and 2′-O-methyl modified purines. In some embodiments, all pyrimidines of the antisense strand comprise 2′ fluoro modified pyrimidines, and all purines of the antisense strand comprise 2′-O-methyl modified purines. In some embodiments, all pyrimidines of the antisense strand comprise 2′-O-methyl modified pyrimidines, and all purines of the antisense strand comprise 2′ fluoro modified purines.

Disclosed herein, in some embodiments, are modified oligonucleotides. The modified oligonucleotide may be an siRNA that includes modifications to the ribose rings, and phosphate linkages. The modifications may be in particular patterns that maximize cell delivery, stability, and efficiency. The siRNA may also include a vinyl phosphonate and a hydrophobic group. These modifications may aid in delivery to a cell or tissue within a subject. The modified oligonucleotide may be used in a method such as a treatment method or a method of reducing gene expression.

In some embodiments, the oligonucleotide comprises a duplex consisting of 21 nucleotide single strands with base pairing between 19 of the base pairs. In some embodiments, the duplex comprises single-stranded 2 nucleotide overhangs are at the 3′ ends of each strand. One strand (antisense strand) is complementary to a MTRES1 mRNA. Each end of the antisense strand has one to two phosphorothioate bonds. The 5′ end has an optional phosphate mimic such as a vinyl phosphonate. In some embodiments, the oligonucleotide is used to knock down a MTRES1 mRNA or a target protein. In some embodiments, the sense strand has the same sequence as the MTRES1 mRNA. In some embodiments, there are 1-2 phosphorothioates at the 3′ end. In some embodiments, there are 1 or no phosphorothioates at the 5′ end. In some embodiments, there is a hydrophobic conjugate of 12 to 25 carbons attached at the 5′ end via a phosphodiester bond.

In some cases, the sense strand of any of the siRNAs comprises siRNA with a particular modification pattern. In some embodiments of the modification pattern, position 9 counting from the 5′ end of the sense strand may have a 2′F modification. In some embodiments, when position 9 of the sense strand is a pyrimidine, then all purines in the sense strand have a 2′OMe modification. In some embodiments, when position 9 is the only pyrimidine between positions 5 and 11 of the sense stand, then position 9 is the only position with a 2′F modification in the sense strand. In some embodiments, when position 9 and only one other base between positions 5 and 11 of the sense strand are pyrimidines, then both of these pyrimidines are the only two positions with a 2′F modification in the sense strand. In some embodiments, when position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of the sense strand, then all combinations of pyrimidines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some embodiments, when position 9 of the sense strand is a purine, then all purines in the sense strand have a 2′OMe modification. In some embodiments, when position 9 is the only purine between positions 5 and 11 of the sense stand, then position 9 is the only position with a 2′F modification in the sense strand. In some embodiments, when position 9 and only one other base between positions 5 and 11 of the sense strand are purines, then both of these purines are the only two positions with a 2′F modification in the sense strand. In some embodiments, when position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of the sense strand, then all combinations of purines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some cases, position 9 of the sense strand can be a 2′deoxy. In these cases, 2′F and 2′OMe modifications may occur at the other positions of the sense strand. In some cases, the sense strand of any of the siRNAs comprises a modification pattern which conforms to these sense strand rules.

In some cases, the sense strand of any of the siRNAs comprises a modification pattern which conforms to these sense strand rules.

Terminal modifications useful for modulating activity include modification of the 5′ end of the antisense strand with phosphate or phosphate analogs. In certain embodiments, the 5′ end of the antisense strand is phosphorylated or includes a phosphoryl analog. Exemplary 5′-phosphate modifications include those which are compatible with RNA-induced silencing complex (RISC) mediated gene silencing. In some embodiments, the 3′ end of the antisense strand is phosphorylated or includes a phosphoryl analog. In some embodiments, the 5′ end of the sense strand is phosphorylated or includes a phosphoryl analog. In some embodiments, the 3′ end of the sense strand is phosphorylated or includes a phosphoryl analog.

In some embodiments, the oligonucleotide comprises a phosphate or phosphate mimic at the 5′ end of the antisense strand. In some embodiment, the phosphate mimic includes a 5′-vinyl phosphonate (VP). In some embodiment, the phosphate mimic is a 5′-VP. In some embodiments, the oligonucleotide comprises a phosphate or phosphate mimic at the 3′ end of the antisense strand. In some embodiments, the oligonucleotide comprises a phosphate or phosphate mimic at the 5′ end of the sense strand. In some embodiments, the oligonucleotide comprises a phosphate or phosphate mimic at the 3′ end of the sense strand.

Disclosed herein, in some embodiments are compositions comprising an oligonucleotide that targets MTRES1 and when administered to a cell decreases expression of MTRES1, wherein the oligonucleotide comprises a small interfering RNA (siRNA) comprising a sense strand and an antisense strand, wherein the sense strand comprises a sense strand sequence described herein in which at least one internucleoside linkage is modified and at least one nucleoside is modified, or an sense strand sequence comprising 1 or 2 nucleoside substitutions, additions, or deletions of the oligonucleotide sequence in which at least one internucleoside linkage is modified and at least one nucleoside is modified, and wherein the antisense strand comprises an antisense strand sequence described herein in which at least one internucleoside linkage is modified and at least one nucleoside is modified, or an oligonucleotide sequence comprising 1 or 2 nucleoside substitutions, additions, or deletions of the antisense strand sequence in which at least one internucleoside linkage is modified and at least one nucleoside is modified. Some embodiments relate to methods that include administering the composition to a subject.

In some embodiments, the siRNA comprises the sense strand and/or the antisense strand sequence of an siRNA in Table 8, or a nucleic acid sequence thereof having 3 or 4 nucleoside substitutions, additions, or deletions. In some embodiments, the siRNA comprises the sense strand and/or the antisense strand sequence of an siRNA in Table 8, or a nucleic acid sequence thereof having 1 or 2 nucleoside substitutions, additions, or deletions. In some embodiments, the siRNA comprises the sense strand and/or the antisense strand sequence of an siRNA in Table 8. The siRNA may include the same internucleoside linkage modifications or nucleoside modifications as those in Table 8. The siRNA may include any different internucleoside linkage modifications or nucleoside modifications different from those in Table 8. The siRNA may include some unmodified internucleoside linkages or nucleosides.

In some embodiments, the siRNA comprises the sense strand and/or the antisense strand sequence of an siRNA in Table 9, or a nucleic acid sequence thereof having 3 or 4 nucleoside substitutions, additions, or deletions. In some embodiments, the siRNA comprises the sense strand and/or the antisense strand sequence of an siRNA in Table 9, or a nucleic acid sequence thereof having 1 or 2 nucleoside substitutions, additions, or deletions. In some embodiments, the siRNA comprises the sense strand and/or the antisense strand sequence of an siRNA in Table 9. The siRNA may include the same internucleoside linkage modifications or nucleoside modifications as those in Table 9. The siRNA may include any different internucleoside linkage modifications or nucleoside modifications different from those in Table 9. The siRNA may include some unmodified internucleoside linkages or nucleosides.

In some embodiments, the siRNA comprises the sense strand and/or the antisense strand sequence of an siRNA in Table 11A, or a nucleic acid sequence thereof having 3 or 4 nucleoside substitutions, additions, or deletions. In some embodiments, the siRNA comprises the sense strand and/or the antisense strand sequence of an siRNA in Table 11A, or a nucleic acid sequence thereof having 1 or 2 nucleoside substitutions, additions, or deletions. In some embodiments, the siRNA comprises the sense strand and/or the antisense strand sequence of an siRNA in Table 11A. The siRNA may include the same internucleoside linkage modifications or nucleoside modifications as those in Table 11A. The siRNA may include any different internucleoside linkage modifications or nucleoside modifications different from those in Table 11A. The siRNA may include some unmodified internucleoside linkages or nucleosides.

In some embodiments, the siRNA comprises the sense strand and/or the antisense strand sequence of an siRNA in Table 13A, or a nucleic acid sequence thereof having 3 or 4 nucleoside substitutions, additions, or deletions. In some embodiments, the siRNA comprises the sense strand and/or the antisense strand sequence of an siRNA in Table 13A, or a nucleic acid sequence thereof having 1 or 2 nucleoside substitutions, additions, or deletions. In some embodiments, the siRNA comprises the sense strand and/or the antisense strand sequence of an siRNA in Table 13A. The siRNA may include the same internucleoside linkage modifications or nucleoside modifications as those in Table 13A. The siRNA may include any different internucleoside linkage modifications or nucleoside modifications different from those in Table 13A. The siRNA may include some unmodified internucleoside linkages or nucleosides.

In some embodiments, the siRNA comprises the sense strand and/or the antisense strand sequence of an siRNA in Table 15A, or a nucleic acid sequence thereof having 3 or 4 nucleoside substitutions, additions, or deletions. In some embodiments, the siRNA comprises the sense strand and/or the antisense strand sequence of an siRNA in Table 15A, or a nucleic acid sequence thereof having 1 or 2 nucleoside substitutions, additions, or deletions. In some embodiments, the siRNA comprises the sense strand and/or the antisense strand sequence of an siRNA in Table 15A. The siRNA may include the same internucleoside linkage modifications or nucleoside modifications as those in Table 15A. The siRNA may include any different internucleoside linkage modifications or nucleoside modifications different from those in Table 15A. The siRNA may include some unmodified internucleoside linkages or nucleosides.

In some embodiments, the siRNA comprises a sense strand having a sequence in accordance with SEQ ID NO: 2472. In some embodiments, the sense strand sequence comprises or consists of sequence at least 75% identical to SEQ ID NO: 2472, at least 80% identical to SEQ ID NO: 2472, at least 85% identical to SEQ ID NO: 2472, at least 90% identical to SEQ ID NO: 2472, or at least 95% identical to SEQ ID NO: 2472. In some embodiments, the sense strand sequence comprises or consists of the sequence of SEQ ID NO 2472, or a sense strand sequence thereof having 1, 2, 3, or 4 nucleoside substitutions, additions, or deletions. In some embodiments, the sense strand sequence comprises or consists of the sequence of SEQ ID NO: 2472, or a sense strand sequence thereof having 1 or 2 nucleoside substitutions, additions, or deletions. In some embodiments, the sense strand sequence comprises or consists of a sequence 100% identical to SEQ ID NO: 2472. The sense strand may comprise a moiety such as a GalNAc moiety or a lipid moiety. In some embodiments, the siRNA comprises an antisense strand having a sequence in accordance with SEQ ID NO: 2489. In some embodiments, the antisense strand sequence comprises or consists of sequence at least 75% identical to SEQ ID NO: 2489, at least 80% identical to SEQ ID NO: 2489, at least 85% identical to SEQ ID NO: 2489, at least 90% identical to SEQ ID NO: 2489, or at least 95% identical to SEQ ID NO: 2489. In some embodiments, the antisense strand sequence comprises or consists of the sequence of SEQ ID NO 2489, or an antisense strand sequence thereof having 1, 2, 3, or 4 nucleoside substitutions, additions, or deletions. In some embodiments, the antisense strand sequence comprises or consists of the sequence of SEQ ID NO: 2489, or an antisense strand sequence thereof having 1 or 2 nucleoside substitutions, additions, or deletions. In some embodiments, the antisense strand sequence comprises or consists of a sequence 100% identical to SEQ ID NO: 2489. The antisense strand may comprise a moiety such as a GalNAc moiety or a lipid moiety.

In some embodiments, the siRNA comprises a sense strand having a sequence in accordance with SEQ ID NO: 2478. In some embodiments, the sense strand sequence comprises or consists of sequence at least 75% identical to SEQ ID NO: 2478, at least 80% identical to SEQ ID NO: 2478, at least 85% identical to SEQ ID NO: 2478, at least 90% identical to SEQ ID NO: 2478, or at least 95% identical to SEQ ID NO: 2478. In some embodiments, the sense strand sequence comprises or consists of the sequence of SEQ ID NO 2478, or a sense strand sequence thereof having 1, 2, 3, or 4 nucleoside substitutions, additions, or deletions. In some embodiments, the sense strand sequence comprises or consists of the sequence of SEQ ID NO: 2478, or a sense strand sequence thereof having 1 or 2 nucleoside substitutions, additions, or deletions. In some embodiments, the sense strand sequence comprises or consists of a sequence 100% identical to SEQ ID NO: 2478. The sense strand may comprise a moiety such as a GalNAc moiety or a lipid moiety. In some embodiments, the siRNA comprises an antisense strand having a sequence in accordance with SEQ ID NO: 2495. In some embodiments, the antisense strand sequence comprises or consists of sequence at least 75% identical to SEQ ID NO: 2495, at least 80% identical to SEQ ID NO: 2495, at least 85% identical to SEQ ID NO: 2495, at least 90% identical to SEQ ID NO: 2495, or at least 95% identical to SEQ ID NO: 2495. In some embodiments, the antisense strand sequence comprises or consists of the sequence of SEQ ID NO 2495, or an antisense strand sequence thereof having 1, 2, 3, or 4 nucleoside substitutions, additions, or deletions. In some embodiments, the antisense strand sequence comprises or consists of the sequence of SEQ ID NO: 2495, or an antisense strand sequence thereof having 1 or 2 nucleoside substitutions, additions, or deletions. In some embodiments, the antisense strand sequence comprises or consists of a sequence 100% identical to SEQ ID NO: 2495. The antisense strand may comprise a moiety such as a GalNAc moiety or a lipid moiety.

In some embodiments, the siRNA comprises a sense strand having a sequence in accordance with SEQ ID NO: 2479. In some embodiments, the sense strand sequence comprises or consists of sequence at least 75% identical to SEQ ID NO: 2479, at least 80% identical to SEQ ID NO: 2479, at least 85% identical to SEQ ID NO: 2479, at least 90% identical to SEQ ID NO: 2479, or at least 95% identical to SEQ ID NO: 2479. In some embodiments, the sense strand sequence comprises or consists of the sequence of SEQ ID NO 2479, or a sense strand sequence thereof having 1, 2, 3, or 4 nucleoside substitutions, additions, or deletions. In some embodiments, the sense strand sequence comprises or consists of the sequence of SEQ ID NO: 2479, or a sense strand sequence thereof having 1 or 2 nucleoside substitutions, additions, or deletions. In some embodiments, the sense strand sequence comprises or consists of a sequence 100% identical to SEQ ID NO: 2479. The sense strand may comprise a moiety such as a GalNAc moiety or a lipid moiety. In some embodiments, the siRNA comprises an antisense strand having a sequence in accordance with SEQ ID NO: 2496. In some embodiments, the antisense strand sequence comprises or consists of sequence at least 75% identical to SEQ ID NO: 2496, at least 80% identical to SEQ ID NO: 2496, at least 85% identical to SEQ ID NO: 2496, at least 90% identical to SEQ ID NO: 2496, or at least 95% identical to SEQ ID NO: 2496. In some embodiments, the antisense strand sequence comprises or consists of the sequence of SEQ ID NO 2496, or an antisense strand sequence thereof having 1, 2, 3, or 4 nucleoside substitutions, additions, or deletions. In some embodiments, the antisense strand sequence comprises or consists of the sequence of SEQ ID NO: 2496, or an antisense strand sequence thereof having 1 or 2 nucleoside substitutions, additions, or deletions. In some embodiments, the antisense strand sequence comprises or consists of a sequence 100% identical to SEQ ID NO: 2496. The antisense strand may comprise a moiety such as a GalNAc moiety or a lipid moiety.

In some embodiments, the siRNA comprises a sense strand having a sequence in accordance with SEQ ID NO: 2480. In some embodiments, the sense strand sequence comprises or consists of sequence at least 75% identical to SEQ ID NO: 2480, at least 80% identical to SEQ ID NO: 2480, at least 85% identical to SEQ ID NO: 2480, at least 90% identical to SEQ ID NO: 2480, or at least 95% identical to SEQ ID NO: 2480. In some embodiments, the sense strand sequence comprises or consists of the sequence of SEQ ID NO 2480, or a sense strand sequence thereof having 1, 2, 3, or 4 nucleoside substitutions, additions, or deletions. In some embodiments, the sense strand sequence comprises or consists of the sequence of SEQ ID NO: 2480, or a sense strand sequence thereof having 1 or 2 nucleoside substitutions, additions, or deletions. In some embodiments, the sense strand sequence comprises or consists of a sequence 100% identical to SEQ ID NO: 2480. The sense strand may comprise a moiety such as a GalNAc moiety or a lipid moiety. In some embodiments, the siRNA comprises an antisense strand having a sequence in accordance with SEQ ID NO: 2497. In some embodiments, the antisense strand sequence comprises or consists of sequence at least 75% identical to SEQ ID NO: 2497, at least 80% identical to SEQ ID NO: 2497, at least 85% identical to SEQ ID NO: 2497, at least 90% identical to SEQ ID NO: 2497, or at least 95% identical to SEQ ID NO: 2497. In some embodiments, the antisense strand sequence comprises or consists of the sequence of SEQ ID NO 2497, or an antisense strand sequence thereof having 1, 2, 3, or 4 nucleoside substitutions, additions, or deletions. In some embodiments, the antisense strand sequence comprises or consists of the sequence of SEQ ID NO: 2497, or an antisense strand sequence thereof having 1 or 2 nucleoside substitutions, additions, or deletions. In some embodiments, the antisense strand sequence comprises or consists of a sequence 100% identical to SEQ ID NO: 2497. The antisense strand may comprise a moiety such as a GalNAc moiety or a lipid moiety.

In some embodiments, the siRNA comprises a sense strand having a sequence in accordance with SEQ ID NO: 2507. In some embodiments, the sense strand sequence comprises or consists of sequence at least 75% identical to SEQ ID NO: 2507, at least 80% identical to SEQ ID NO: 2507, at least 85% identical to SEQ ID NO: 2507, at least 90% identical to SEQ ID NO: 2507, or at least 95% identical to SEQ ID NO: 2507. In some embodiments, the sense strand sequence comprises or consists of the sequence of SEQ ID NO 2507, or a sense strand sequence thereof having 1, 2, 3, or 4 nucleoside substitutions, additions, or deletions. In some embodiments, the sense strand sequence comprises or consists of the sequence of SEQ ID NO: 2507, or a sense strand sequence thereof having 1 or 2 nucleoside substitutions, additions, or deletions. In some embodiments, the sense strand sequence comprises or consists of a sequence 100% identical to SEQ ID NO: 2507. The sense strand may comprise a moiety such as a GalNAc moiety or a lipid moiety. In some embodiments, the siRNA comprises an antisense strand having a sequence in accordance with SEQ ID NO: 2517. In some embodiments, the antisense strand sequence comprises or consists of sequence at least 75% identical to SEQ ID NO: 2517, at least 80% identical to SEQ ID NO: 2517, at least 85% identical to SEQ ID NO: 2517, at least 90% identical to SEQ ID NO: 2517, or at least 95% identical to SEQ ID NO: 2517. In some embodiments, the antisense strand sequence comprises or consists of the sequence of SEQ ID NO 2517, or an antisense strand sequence thereof having 1, 2, 3, or 4 nucleoside substitutions, additions, or deletions. In some embodiments, the antisense strand sequence comprises or consists of the sequence of SEQ ID NO: 2517, or an antisense strand sequence thereof having 1 or 2 nucleoside substitutions, additions, or deletions. In some embodiments, the antisense strand sequence comprises or consists of a sequence 100% identical to SEQ ID NO: 2517. The antisense strand may comprise a moiety such as a GalNAc moiety or a lipid moiety.

4. ASO modification patterns

In some embodiments, the composition comprises an oligonucleotide that inhibits the expression of MTRES1, wherein the oligonucleotide comprises an antisense oligonucleotide (ASO). In some embodiments, the ASO comprises modification pattern ASO1: 5′-nsnsnsnsnsdNsdNsdNsdNsdNsdNsdNsdNsdNsdNsnsnsnsnsn-3′ (SEQ ID NO: 2461), wherein “dN” is any deoxynucleotide, “n” is a 2′O-methyl or 2′O-methoxyethyl-modified nucleoside, and “s” is a phosphorothioate linkage. In some embodiments, the ASO comprises modification pattern 1S1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 1AS, 2AS, 3AS, 4AS, 5AS, 6AS, 7AS, 8AS, 9AS or 10AS.

### D. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

In some embodiments, the composition is formulated to cross the blood brain barrier. In some embodiments, the composition is formulated for central nervous system (CNS) delivery. In some embodiments, the composition includes a lipophilic compound. The lipophilic compound may be useful for crossing the blood brain barrier or for CNS delivery.

### II. METHODS AND USES

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In some embodiments, the administration is systemic. In some embodiments, the administration is intravenous. In some embodiments, the administration is by injection.

### A. Disorders

Some embodiments of the methods described herein include treating a disorder in a subject in need thereof. In some embodiments, the disorder is a neurological disorder. Non-limiting examples of neurological disorders include dementia, Alzheimer's disease, delirium, cognitive decline, vascular dementia, or Parkinson's disease. In some embodiments, the neurological disorder includes cognitive decline. In some embodiments, the neurological disorder includes delirium. In some embodiments, the neurological disorder includes dementia. In some embodiments, the neurological disorder includes vascular dementia. In some embodiments, the neurological disorder includes Alzheimer's disease. In some embodiments, the neurological disorder includes Parkinson's disease. The neurological disorder may include a neurodegenerative disease. The neurological disorder may be characterized by protein aggregation.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the subject is male. In some embodiments, the subject is female.

In some embodiments, the subject is an adult (e.g. at least 18 years old). In some embodiments, the subject is ≥90 years of age. In some embodiments, the subject is ≥85 years of age. In some embodiments, the subject is ≥80 years of age. In some embodiments, the subject is ≥70 years of age. In some embodiments, the subject is ≥60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is ≥0 years of age.

In some embodiments, the subject is ≤100 years of age. In some embodiments, the subject is ≤90 years of age. In some embodiments, the subject is ≤85 years of age. In some embodiments, the subject is ≤80 years of age. In some embodiments, the subject is ≤70 years of age. In some embodiments, the subject is ≤60 years of age. In some embodiments, the subject is ≤50 years of age. In some embodiments, the subject is ≤40 years of age. In some embodiments, the subject is ≤30 years of age. In some embodiments, the subject is ≤20 years of age. In some embodiments, the subject is ≤10 years of age. In some embodiments, the subject is ≤1 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

C. Baseline measurements

Some embodiments of the methods described herein include obtaining a baseline measurement from a subject. For example, in some embodiments, a baseline measurement is obtained from the subject prior to treating the subject. Non-limiting examples of baseline measurements include a baseline cognitive function measurement, a baseline central nervous system (CNS) amyloid plaque measurement, a baseline CNS tau accumulation measurement, a baseline cerebrospinal fluid (CSF) beta-amyloid 42 measurement, a baseline CSF tau measurement, a baseline CSF phospho-tau measurement, a baseline neurofilament light (NfL) measurement. a baseline CSF alpha-synuclein measurement, a baseline Lewy body measurement, a baseline MTRES1 protein measurement, or a baseline MTRES1 mRNA measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device.

In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g. HPLC) assay. In some embodiments, the baseline measurement is obtained by PCR.

In some embodiments, the baseline measurement is a baseline cognitive function measurement. The baseline cognitive function measurement may be obtained directly from the subject. For example, the subject may be administered a test. The test may include a cognitive test such as the Montreal Cognitive Assessment (MoCA), Mini-Mental State Exam (MMSE), or Mini-Cog. The test may include assessment of basic cognitive functions such as memory, language, executive frontal lobe function, apraxia, visuospatial ability, behavior, mood, orientation, or attention. The baseline cognitive function measurement may include a score. The baseline cognitive function measurement may be indicative of mild cognitive impairment, or of severe cognitive impairment. The baseline cognitive function measurement may be indicative of a neurological disorder.

The baseline measurement may include a baseline In some embodiments, the marker of neurodegeneration measurement. Examples of marker of neurodegeneration may include central nervous system (CNS) amyloid plaques, CNS tau accumulation, cerebrospinal fluid (CSF) beta-amyloid 42, CSF tau, CSF phospho-tau, CSF or plasma neurofilament light chain (NfL), Lewy bodies, or CSF alpha-synuclein. Any of these measurements may be reduced in relation to the baseline measurement. Some examples of ways to measure these may include an assay such as a immunoassay, colorimetric assay, or microscopy.

In some embodiments, the baseline measurement is a baseline amyloid plaque measurement. The baseline amyloid plaque measurement may include a central nervous system (CNS) amyloid plaque measurement. In some embodiments, the baseline amyloid plaque measurement includes a baseline concentration or amount. The baseline amyloid plaque measurement may be performed using an imaging device. The imaging device may include a positron emission tomography (PET) device. The baseline amyloid plaque measurement may be performed on a biopsy. The baseline amyloid plaque measurement may be performed using a spinal tap (for example, when the baseline amyloid plaque measurement includes a baseline cerebrospinal fluid (CSF) amyloid plaque measurement). In some embodiments, the baseline amyloid plaque measurement is obtained by an assay such as an immunoassay. The baseline beta amyloid plaque measurement may be indicative of a neurodegenerative disease such as Alzheimer's disease.

In some embodiments, the baseline measurement is a baseline beta-amyloid 42 measurement. The baseline beta-amyloid 42 measurement may include a cerebrospinal fluid (CSF) beta-amyloid 42 measurement. In some embodiments, the baseline beta-amyloid 42 measurement includes a baseline concentration or amount. The baseline beta-amyloid 42 measurement may be performed on a biopsy. The baseline beta-amyloid 42 measurement may be performed using a spinal tap (for example, when the baseline beta-amyloid 42 measurement includes a baseline CSF beta-amyloid 42 measurement). In some embodiments, the baseline beta-amyloid 42 measurement is obtained by an assay such as an immunoassay. The baseline beta-amyloid 42 measurement may be indicative of a neurodegenerative disease such as Alzheimer's disease.

In some embodiments, the baseline measurement is a baseline tau measurement. In some embodiments, the baseline tau measurement includes a baseline concentration or amount. The baseline tau measurement may be performed on a biopsy. In some embodiments, the baseline tau measurement is obtained by an assay such as an immunoassay. The baseline beta tau measurement may be indicative of a neurodegenerative disease such as Alzheimer's disease or Parkinson's disease.

In some embodiments, the baseline tau measurement is a baseline central nervous system (CNS) tau measurement. The baseline tau measurement may include a baseline total tau measurement. The baseline tau measurement may include a baseline unphosphorylated tau measurement. The baseline tau measurement may include a baseline phosphorylated tau (phospho-tau) measurement. In some embodiments, the baseline tau measurement is a baseline tau accumulation measurement. In some embodiments, the baseline tau measurement is a baseline CNS tau accumulation measurement. The baseline CNS tau accumulation measurement may be indicative of a neurodegenerative disease such as Alzheimer's disease or Parkinson's disease.

The baseline tau measurement may include a cerebrospinal fluid (CSF) tau measurement. The baseline CSF tau measurement may be performed after use of a spinal tap. The baseline CSF tau measurement may be indicative of a neurodegenerative disease such as Alzheimer's disease or Parkinson's disease.

The baseline CSF tau measurement may include a baseline CSF phospho-tau measurement. The baseline CSF phospho-tau measurement may include an amount of phospho-tau in relation to total tau or unphosphorylated tau. For example, the baseline CSF phospho-tau measurement may include a phospho-tau/tau ratio. The baseline CSF phospho-tau measurement may be indicative of a neurodegenerative disease such as Alzheimer's disease or Parkinson's disease.

In some embodiments, the baseline neurofilament light chain (NfL) measurement includes a baseline CSF or plasma NfL measurement. The baseline NfL measurement may be a baseline CSF NfL measurement. The baseline NfL measurement may be a baseline plasma NfL measurement. The NfL measurement may include a concentration or an amount. The baseline NfL measurement may be indicative of a neurodegenerative disease such as Alzheimer's disease or Parkinson's disease.

In some embodiments, the baseline measurement is a baseline alpha-synuclein measurement. The baseline alpha-synuclein measurement may include a cerebrospinal fluid (CSF) alpha-synuclein measurement. In some embodiments, the baseline alpha-synuclein measurement includes a baseline concentration or amount. The baseline alpha-synuclein measurement may be performed on a biopsy. The baseline alpha-synuclein measurement may be performed using a spinal tap (for example, when the baseline alpha-synuclein measurement includes a baseline CSF alpha-synuclein measurement). In some embodiments, the baseline alpha-synuclein measurement is obtained by an assay such as an immunoassay.

The baseline alpha-synuclein measurement may be indicative of a neurodegenerative disease such as Parkinson's disease. The baseline alpha-synuclein measurement may be indicative of dementia.

In some embodiments, the baseline measurement is a baseline Lewy body measurement. The baseline Lewy body measurement may include a central nervous system (CNS) Lewy body measurement.

In some embodiments, the baseline Lewy body measurement includes a baseline concentration or amount.

The baseline Lewy body measurement may be performed using an imaging device. The imaging device may include a positron emission tomography (PET) device. The baseline beta Lewy body measurement may be indicative of dementia.

In some embodiments, the baseline measurement is a baseline MTRES1 protein measurement. In some embodiments, the baseline MTRES1 protein measurement comprises a baseline MTRES1 protein level. In some embodiments, the baseline MTRES1 protein level is indicated as a mass or percentage of MTRES1 protein per sample weight. In some embodiments, the baseline MTRES1 protein level is indicated as a mass or percentage of MTRES1 protein per sample volume. In some embodiments, the baseline MTRES1 protein level is indicated as a mass or percentage of MTRES1 protein per total protein within the sample. In some embodiments, the baseline MTRES1 protein measurement is a baseline CNS or CSF MTRES1 protein measurement. In some embodiments, the baseline MTRES1 protein measurement is obtained by an assay such as an immunoassay, a colorimetric assay, or a fluorescence assay.

In some embodiments, the baseline measurement is a baseline MTRES1 mRNA measurement. In some embodiments, the baseline MTRES1 mRNA measurement comprises a baseline MTRES1 mRNA level. In some embodiments, the baseline MTRES1 mRNA level is indicated as an amount or percentage of MTRES1 mRNA per sample weight. In some embodiments, the baseline MTRES1 mRNA level is indicated as an amount or percentage of MTRES1 mRNA per sample volume. In some embodiments, the baseline MTRES1 mRNA level is indicated as an amount or percentage of MTRES1 mRNA per total mRNA within the sample. In some embodiments, the baseline MTRES1 mRNA level is indicated as an amount or percentage of MTRES1 mRNA per total nucleic acids within the sample. In some embodiments, the baseline MTRES1 mRNA level is indicated relative to another mRNA level, such as an mRNA level of a housekeeping gene, within the sample. In some embodiments, the baseline MTRES1 mRNA measurement is a baseline CNS or CSF MTRES1 mRNA measurement. In some embodiments, the baseline MTRES1 mRNA measurement is obtained by an assay such as a polymerase chain reaction (PCR) assay. In some embodiments, the PCR comprises quantitative PCR (qPCR). In some embodiments, the PCR comprises reverse transcription of the MTRES1 mRNA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject.

In some embodiments, the sample comprises a fluid. In some embodiments, the sample is a fluid sample. In some embodiments, the fluid sample is a CSF sample. In some embodiments, the fluid sample includes a central nervous system (CNS) fluid sample. The CNS fluid may include cerebrospinal fluid (CSF). In some embodiments, the fluid sample includes a CSF sample. In some embodiments, the sample is a blood, plasma, or serum sample. In some embodiments, the sample comprises blood. In some embodiments, the sample is a blood sample. In some embodiments, the sample is a whole-blood sample.

In some embodiments, the blood is fractionated or centrifuged. In some embodiments, the sample comprises plasma. In some embodiments, the sample is a plasma sample. A blood sample may be a plasma sample. In some embodiments, the sample comprises serum. In some embodiments, the sample is a serum sample. A blood sample may be a serum sample.

In some embodiments, the sample comprises a tissue. In some embodiments, the sample is a tissue sample. In some embodiments, the tissue comprises central nervous system (CNS) tissue. For example, the baseline MTRES1 mRNA measurement, or the baseline MTRES1 protein measurement, may be obtained in a CNS tissue sample obtained from the patient. The CNS tissue may include brain tissue. The CNS tissue may include nerve tissue. The CNS tissue may include neurons, glia, microglia, astrocytes, or oligodendrocytes, or a combination thereof. The CNS tissue may include neurons. The CNS tissue may include glia. The CNS tissue may include microglia. The CNS tissue may include astrocytes. The CNS tissue may include oligodendrocytes.

In some embodiments, the sample includes cells. In some embodiments, the sample comprises a cell. In some embodiments, the cell comprises a CNS cell. The CNS cell may include a brain cell. The CNS cell may include a nerve cell. The CNS cell may be a neuron, glial cell, microglial cell, astrocyte, or oligodendrocyte. The CNS cell may be a neuron. The CNS cell may be a glial cell. The CNS cell may be a microglial cell. The CNS cell may be an astrocyte. The CNS cell may be an oligodendrocyte.

### D. Effects

In some embodiments, the composition or administration of the composition affects a measurement such as a cognitive function measurement, a central nervous system (CNS) amyloid plaque measurement, a CNS tau accumulation measurement, a cerebrospinal fluid (CSF) beta-amyloid 42 measurement, a CSF tau measurement, a CSF phospho-tau measurement, a NfL measurement, a CSF alpha-synuclein measurement, a Lewy body measurement, a MTRES1 protein measurement, or a MTRES1 mRNA measurement, relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, a chromatography (e.g. HPLC) assay, or a PCR assay. In some embodiments, the measurement is obtained by an assay such as an immunoassay, a colorimetric assay, a fluorescence assay, or a chromatography (e.g. HPLC) assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. For example, an adverse phenotype of a neurological disorder may be reduced upon administration of the composition. The neurological disorder may include dementia, Alzheimer's disease, delirium, cognitive decline, vascular dementia, or Parkinson's disease. In some embodiments, the reduction is measured in a second sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the measurement relative to the baseline measurement. For example, a protective phenotype of a neurological disorder may be increased upon administration of the composition. The neurological disorder may include dementia, Alzheimer's disease, delirium, cognitive decline, vascular dementia, or Parkinson's disease. In some embodiments, the increase is measured in a second sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a cognitive function measurement. The cognitive function measurement may be obtained directly from the subject. For example, the subject may be administered a test. The test may include a cognitive test such as the Montreal Cognitive Assessment (MoCA), Mini-Mental State Exam (MMSE), or Mini-Cog. The test may include assessment of basic cognitive functions such as memory, language, executive frontal lobe function, apraxia, visuospatial ability, behavior, mood, orientation, or attention. The cognitive function measurement may include a score. The cognitive function measurement may be indicative of a lack of cognitive impairment. In some embodiments, the cognitive function measurement is indicative of mild cognitive impairment, and the baseline cognitive function measurement is indicative of severe cognitive impairment. The cognitive function measurement may be indicative of a neurological disorder.

In some embodiments, the composition increases the cognitive function measurement relative to the baseline cognitive function measurement. In some embodiments, the increase is measured directly in the subject after administering the composition to the subject. In some embodiments, the cognitive function measurement is increased by about 2.5% or more, about 5% or more, or about 7.5% or more, relative to the baseline cognitive function measurement. In some embodiments, the cognitive function measurement is increased by about 10% or more, relative to the baseline cognitive function measurement.

In some embodiments, the cognitive function measurement is increased by about 20% or more, about 30% or more, about 40% or more, about 50% or more, about 60% or more, about 70% or more, about 80% or more, about 90% or more, relative to the baseline cognitive function measurement. In some embodiments, the cognitive function measurement is increased by about 100% or more, increased by about 250% or more, increased by about 500% or more, increased by about 750% or more, or increased by about 1000% or more, relative to the baseline cognitive function measurement. In some embodiments, the cognitive function measurement is increased by no more than about 2.5%, no more than about 5%, or no more than about 7.5%, relative to the baseline cognitive function measurement. In some embodiments, the cognitive function measurement is increased by no more than about 10%, relative to the baseline cognitive function measurement. In some embodiments, the cognitive function measurement is increased by no more than about 20%, no more than about 30%, no more than about 40%, no more than about 50%, no more than about 60%, no more than about 70%, no more than about 80%, no more than about 90%, or no more than about 100% relative to the baseline cognitive function measurement. In some embodiments, the cognitive function measurement is increased by no more than about 100%, increased by no more than about 250%, increased by no more than about 500%, increased by no more than about 750%, or increased by no more than about 1000%, relative to the baseline cognitive function measurement. In some embodiments, the cognitive function measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is an amyloid plaque measurement. The amyloid plaque measurement may include a central nervous system (CNS) amyloid plaque measurement. In some embodiments, the amyloid plaque measurement includes a concentration or amount. The amyloid plaque measurement may be performed using an imaging device. The imaging device may include a positron emission tomography (PET) device. The amyloid plaque measurement may be performed on a biopsy.

The amyloid plaque measurement may be performed using a spinal tap (for example, when the amyloid plaque measurement includes a cerebrospinal fluid (CSF) amyloid plaque measurement). In some embodiments, the amyloid plaque measurement is obtained by an assay such as an immunoassay. The beta amyloid plaque measurement may be indicative of a treatment effect of the oligonucleotide on a neurodegenerative disease such as Alzheimer's disease.

In some embodiments, the composition reduces the amyloid plaque measurement relative to the baseline amyloid plaque measurement. In some embodiments, the reduction is measured in a second sample obtained from the subject after administering the composition to the subject. In some embodiments, the reduction is measured directly in the subject after administering the composition to the subject. In some embodiments, the amyloid plaque measurement is decreased by about 2.5% or more, about 5% or more, or about 7.5% or more, relative to the baseline amyloid plaque measurement. In some embodiments, the amyloid plaque measurement is decreased by about 10% or more, relative to the baseline amyloid plaque measurement. In some embodiments, the amyloid plaque measurement is decreased by about 20% or more, about 30% or more, about 40% or more, about 50% or more, about 60⁰% or more, about 70% or more, about 80% or more, about 90% or more, relative to the baseline amyloid plaque measurement. In some embodiments, the amyloid plaque measurement is decreased by no more than about 2.5%, no more than about 5%, or no more than about 7.5%, relative to the baseline amyloid plaque measurement. In some embodiments, the amyloid plaque measurement is decreased by no more than about 10%, relative to the baseline amyloid plaque measurement. In some embodiments, the amyloid plaque measurement is decreased by no more than about 20%, no more than about 30%, no more than about 40%, no more than about 50%, no more than about 60%, no more than about 70%, no more than about 80%, no more than about 90%, or no more than about 100% relative to the baseline amyloid plaque measurement. In some embodiments, the amyloid plaque measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a beta-amyloid 42 measurement. The beta-amyloid 42 measurement may include a cerebrospinal fluid (CSF) beta-amyloid 42 measurement. In some embodiments, the beta-amyloid 42 measurement includes a concentration or amount. The beta-amyloid 42 measurement may be performed on a biopsy. The beta-amyloid 42 measurement may be performed using a spinal tap (for example, when the beta-amyloid 42 measurement includes a CSF beta-amyloid 42 measurement). In some embodiments, the beta-amyloid 42 measurement is obtained by an assay such as an immunoassay. The beta-amyloid 42 measurement may be indicative of a treatment effect of the oligonucleotide on a neurodegenerative disease such as Alzheimer's disease.

In some embodiments, the composition reduces the CSF beta-amyloid 42 measurement relative to the baseline beta-amyloid 42 measurement. In some embodiments, the reduction is measured in a second sample (for example, a CSF sample) obtained from the subject after administering the composition to the subject. In some embodiments, the CSF beta-amyloid 42 measurement is decreased by about 2.5% or more, about 5% or more, or about 7.5% or more, relative to the baseline CSF beta-amyloid 42 measurement. In some embodiments, the CSF beta-amyloid 42 measurement is decreased by about 10% or more, relative to the baseline CSF beta-amyloid 42 measurement. In some embodiments, the CSF beta-amyloid 42 measurement is decreased by about 20% or more, about 30% or more, about 40% or more, about 50% or more, about 60% or more, about 70% or more, about 80% or more, about 90% or more, relative to the baseline CSF beta-amyloid 42 measurement. In some embodiments, the CSF beta-amyloid 42 measurement is decreased by no more than about 2.5%, no more than about 5%, or no more than about 7.5%, relative to the baseline CSF beta-amyloid 42 measurement. In some embodiments, the CSF beta-amyloid 42 measurement is decreased by no more than about 10%, relative to the baseline CSF beta-amyloid 42 measurement. In some embodiments, the CSF beta-amyloid 42 measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF beta-amyloid 42 measurement. In some embodiments, the CSF beta-amyloid 42 measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a tau measurement. In some embodiments, the tau measurement includes a concentration or amount. The tau measurement may be performed on a biopsy. In some embodiments, the tau measurement is obtained by an assay such as an immunoassay. The beta tau measurement may be indicative of a treatment effect of the oligonucleotide on a neurodegenerative disease such as Alzheimer's disease or Parkinson's disease.

In some embodiments, the tau measurement is a central nervous system (CNS) tau measurement. The tau measurement may include a total tau measurement. The tau measurement may include a unphosphorylated tau measurement. The tau measurement may include a phosphorylated tau (phospho-tau) measurement. In some embodiments, the tau measurement is a tau accumulation measurement. In some embodiments, the tau measurement is a CNS tau accumulation measurement. The CNS tau accumulation measurement may be indicative of a treatment effect of the oligonucleotide on a neurodegenerative disease such as Alzheimer's disease or Parkinson's disease.

In some embodiments, the composition reduces the CNS tau accumulation measurement relative to the baseline CNS tau accumulation measurement. In some embodiments, the reduction is measured in a second sample obtained from the subject after administering the composition to the subject. In some embodiments, the CNS tau accumulation measurement is decreased by about 2.5% or more, about 5% or more, or about 7.5% or more, relative to the baseline CNS tau accumulation measurement. In some embodiments, the CNS tau accumulation measurement is decreased by about 10% or more, relative to the baseline CNS tau accumulation measurement. In some embodiments, the CNS tau accumulation measurement is decreased by about 20% or more, about 30% or more, about 40% or more, about 50% or more, about 60% or more, about 70% or more, about 80% or more, about 90% or more, relative to the baseline CNS tau accumulation measurement. In some embodiments, the CNS tau accumulation measurement is decreased by no more than about 2.5%, no more than about 5%, or no more than about 7.5%, relative to the baseline CNS tau accumulation measurement. In some embodiments, the CNS tau accumulation measurement is decreased by no more than about 10%, relative to the baseline CNS tau accumulation measurement. In some embodiments, the CNS tau accumulation measurement is decreased by no more than about 20%, no more than about 30%, no more than about 40%, no more than about 50%, no more than about 60%, no more than about 70%, no more than about 80%, no more than about 90%, or no more than about 100% relative to the baseline CNS tau accumulation measurement. In some embodiments, the CNS tau accumulation measurement is decreased by 2.5%, 5%, 7.5%, 10%, 20%, 30%, 40%, 50%, 60%, 70%, 80%, 90%, or 100%, or by a range defined by any of the two aforementioned percentages.

The tau measurement may include a cerebrospinal fluid (CSF) tau measurement. The CSF tau measurement may be performed after use of a spinal tap. The CSF tau measurement may be indicative of a treatment effect of the oligonucleotide on a neurodegenerative disease such as Alzheimer's disease or Parkinson's disease.

In some embodiments, the composition reduces the CSF tau measurement relative to the baseline CSF tau measurement. In some embodiments, the reduction is measured in a second sample obtained from the subject after administering the composition to the subject. In some embodiments, the reduction is measured in a second CSF sample obtained from the subject after administering the composition to the subject. In some embodiments, the CSF tau measurement is decreased by about 2.5% or more, about 5% or more, or about 7.5% or more, relative to the baseline CSF tau measurement. In some embodiments, the CSF tau measurement is decreased by about 10% or more, relative to the baseline CSF tau measurement. In some embodiments, the CSF tau measurement is decreased by about 20% or more, about 30% or more, about 40% or more, about 50% or more, about 60% or more, about 70% or more, about 80% or more, about 90% or more, relative to the baseline CSF tau measurement. In some embodiments, the CSF tau measurement is decreased by no more than about 2.5%, no more than about 5%, or no more than about 7.5%, relative to the baseline CSF tau measurement. In some embodiments, the CSF tau measurement is decreased by no more than about 10%, relative to the baseline CSF tau measurement. In some embodiments, the CSF tau measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF tau measurement. In some embodiments, the CSF tau measurement is decreased by 2.5%, 5%, 7.5%, 10%, 20%, 30%, 40%, 50%, 60%, 70%, 80%, 90%, or 100%, or by a range defined by any of the two aforementioned percentages.

The CSF tau measurement may include a CSF phospho-tau measurement. The CSF phospho-tau measurement may include an amount of phospho-tau in relation to total tau or unphosphorylated tau. For example, the CSF phospho-tau measurement may include a phospho-tau/tau ratio. The CSF phospho-tau measurement may be indicative of a treatment effect of the oligonucleotide on a neurodegenerative disease such as Alzheimer's disease or Parkinson's disease.

In some embodiments, the composition reduces the CSF phospho-tau measurement relative to the baseline CSF phospho-tau measurement. In some embodiments, the reduction is measured in a second sample obtained from the subject after administering the composition to the subject. In some embodiments, the reduction is measured in a second CSF sample obtained from the subject after administering the composition to the subject. In some embodiments, the CSF phospho-tau measurement is decreased by about 2.5% or more, about 5% or more, or about 7.5% or more, relative to the baseline CSF phospho-tau measurement. In some embodiments, the CSF phospho-tau measurement is decreased by about 10% or more, relative to the baseline CSF phospho-tau measurement. In some embodiments, the CSF phospho-tau measurement is decreased by about 20% or more, about 30% or more, about 40% or more, about 50% or more, about 60% or more, about 70% or more, about 80% or more, about 90% or more, relative to the baseline CSF phospho-tau measurement. In some embodiments, the CSF phospho-tau measurement is decreased by no more than about 2.5%, no more than about 5%, or no more than about 7.5%, relative to the baseline CSF phospho-tau measurement. In some embodiments, the CSF phospho-tau measurement is decreased by no more than about 10%, relative to the baseline CSF phospho-tau measurement. In some embodiments, the CSF phospho-tau measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF phospho-tau measurement. In some embodiments, the CSF phospho-tau measurement is decreased by 2.5%, 5%, 7.5%, 10%, 20%, 30%, 40%, 50⁰%, 60%, 70%, 80%, 90%, or 100%, or by a range defined by any of the two aforementioned percentages.

In some embodiments, the neurofilament light chain (NfL) measurement includes a CSF or plasma NfL measurement. The NfL measurement may be a CSF NfL measurement. The NfL measurement may be a plasma NfL measurement. The NfL measurement may include a concentration or an amount. The NfL measurement may be indicative of a neurodegenerative disease such as Alzheimer's disease or Parkinson's disease.

In some embodiments, the composition reduces the NfL measurement relative to the baseline NfL measurement. In some embodiments, the reduction is measured in a second sample obtained from the subject after administering the composition to the subject. In some embodiments, the NfL measurement is decreased by about 2.5% or more, about 5% or more, or about 7.5% or more, relative to the baseline NfL measurement. In some embodiments, the NfL measurement is decreased by about 10% or more, relative to the baseline NfL measurement. In some embodiments, the NfL measurement is decreased by about 20% or more, about 30% or more, about 40% or more, about 50% or more, about 60% or more, about 70% or more, about 80% or more, about 90% or more, relative to the baseline NfL measurement. In some embodiments, the NfL measurement is decreased by no more than about 2.5%, no more than about 5%, or no more than about 7.5%, relative to the baseline NfL measurement. In some embodiments, the NfL measurement is decreased by no more than about 10%, relative to the baseline NfL measurement. In some embodiments, the NfL measurement is decreased by no more than about 20%, no more than about 30%, no more than about 40%, no more than about 50%, no more than about 60%, no more than about 70%, no more than about 80%, no more than about 90%, or no more than about 100% relative to the baseline NfL measurement. In some embodiments, the NfL measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a alpha-synuclein measurement. The alpha-synuclein measurement may include a cerebrospinal fluid (CSF) alpha-synuclein measurement. In some embodiments, the alpha-synuclein measurement includes a concentration or amount. The alpha-synuclein measurement may be performed on a biopsy. The alpha-synuclein measurement may be performed using a spinal tap (for example, when the alpha-synuclein measurement includes a CSF alpha-synuclein measurement). In some embodiments, the alpha-synuclein measurement is obtained by an assay such as an immunoassay. The alpha-synuclein measurement may be indicative of a treatment effect of the oligonucleotide on a neurodegenerative disease such as Parkinson's disease. The alpha-synuclein measurement may be indicative of a treatment effect of the oligonucleotide on dementia.

In some embodiments, the composition reduces the alpha-synuclein measurement relative to the baseline alpha-synuclein measurement. In some embodiments, the reduction is measured in a second sample obtained from the subject after administering the composition to the subject. In some embodiments, the alpha-synuclein measurement is decreased by about 2.5% or more, about 5% or more, or about 7.5% or more, relative to the baseline alpha-synuclein measurement. In some embodiments, the alpha-synuclein measurement is decreased by about 10% or more, relative to the baseline alpha-synuclein measurement. In some embodiments, the alpha-synuclein measurement is decreased by about 20% or more, about 30% or more, about 40% or more, about 50% or more, about 60% or more, about 70% or more, about 80% or more, about 90% or more, relative to the baseline alpha-synuclein measurement. In some embodiments, the alpha-synuclein measurement is decreased by no more than about 2.5%, no more than about 5%, or no more than about 7.5%, relative to the baseline alpha-synuclein measurement. In some embodiments, the alpha-synuclein measurement is decreased by no more than about 10%, relative to the baseline alpha-synuclein measurement. In some embodiments, the alpha-synuclein measurement is decreased by no more than about 20%, no more than about 30%, no more than about 40%, no more than about 50%, no more than about 60%, no more than about 70%, no more than about 80%, no more than about 90%, or no more than about 100% relative to the baseline alpha-synuclein measurement. In some embodiments, the alpha-synuclein measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a Lewy body measurement. The Lewy body measurement may include a central nervous system (CNS) Lewy body measurement. In some embodiments, the Lewy body measurement includes a concentration or amount. The Lewy body measurement may be performed using an imaging device. The imaging device may include a positron emission tomography (PET) device. The beta Lewy body measurement may be indicative of a treatment effect of the oligonucleotide on dementia.

In some embodiments, the composition reduces the Lewy body measurement relative to the baseline Lewy body measurement. In some embodiments, the reduction is measured in a second sample obtained from the subject after administering the composition to the subject. In some embodiments, the reduction is measured directly in the subject after administering the composition to the subject. In some embodiments, the Lewy body measurement is decreased by about 2.5% or more, about 5% or more, or about 7.5% or more, relative to the baseline Lewy body measurement. In some embodiments, the Lewy body measurement is decreased by about 10% or more, relative to the baseline Lewy body measurement. In some embodiments, the Lewy body measurement is decreased by about 20% or more, about 30% or more, about 40% or more, about 50% or more, about 60% or more, about 70% or more, about 80% or more, about 90% or more, relative to the baseline Lewy body measurement. In some embodiments, the Lewy body measurement is decreased by no more than about 2.5%, no more than about 5%, or no more than about 7.5%, relative to the baseline Lewy body measurement. In some embodiments, the Lewy body measurement is decreased by no more than about 10%, relative to the baseline Lewy body measurement. In some embodiments, the Lewy body measurement is decreased by no more than about 20%, no more than about 30%, no more than about 40%, no more than about 50%, no more than about 60%, no more than about 70%, no more than about 80%, no more than about 90%, or no more than about 100% relative to the baseline Lewy body measurement. In some embodiments, the Lewy body measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an MTRES1 protein measurement. In some embodiments, the MTRES1 protein measurement comprises an MTRES1 protein level. In some embodiments, the MTRES1 protein level is indicated as a mass or percentage of MTRES1 protein per sample weight. In some embodiments, the MTRES1 protein level is indicated as a mass or percentage of MTRES1 protein per sample volume. In some embodiments, the MTRES1 protein level is indicated as a mass or percentage of MTRES1 protein per total protein within the sample. In some embodiments, the MTRES1 protein measurement is a CNS tissue or fluid MTRES1 protein measurement. In some embodiments, the MTRES1 protein measurement is obtained by an assay such as an immunoassay, a colorimetric assay, or a fluorescence assay.

In some embodiments, the composition reduces the MTRES1 protein measurement relative to the baseline MTRES1 protein measurement. In some embodiments, the composition reduces CNS tissue or fluid MTRES1 protein levels relative to the baseline MTRES1 protein measurement. In some embodiments, the reduced MTRES1 protein levels are measured in a second sample obtained from the subject after administering the composition to the subject. In some embodiments, the MTRES1 protein measurement is decreased by about 2.5% or more, about 5% or more, or about 7.5% or more, relative to the baseline MTRES1 protein measurement. In some embodiments, the MTRES1 protein measurement is decreased by about 10% or more, relative to the baseline MTRES1 protein measurement. In some embodiments, the MTRES1 protein measurement is decreased by about 20% or more, about 30% or more, about 40% or more, about 50% or more, about 60% or more, about 70% or more, about 80% or more, about 90% or more, or about 100%, relative to the baseline MTRES1 protein measurement. In some embodiments, the MTRES1 protein measurement is decreased by no more than about 2.5%, no more than about 5%, or no more than about 7.5%, relative to the baseline MTRES1 protein measurement. In some embodiments, the MTRES1 protein measurement is decreased by no more than about 10%, relative to the baseline MTRES1 protein measurement. In some embodiments, the MTRES1 protein measurement is decreased by no more than about 20%, no more than about 30%, no more than about 40%, no more than about 50%, no more than about 60%, no more than about 70%, no more than about 80%, no more than about 90%, or no more than about 100% relative to the baseline MTRES1 protein measurement. In some embodiments, the MTRES1 protein measurement is decreased by 2.5%, 5%, 7.5%, 19%, 20%, 30%, 40%, 50%, 60%, 70%, 80%, 90%, or 100%, or by a range defined by any of the two aforementioned percentages.

In some embodiments, the measurement is an MTRES1 mRNA measurement. In some embodiments, the MTRES1 mRNA measurement comprises an MTRES1 mRNA level. In some embodiments, the MTRES1 mRNA level is indicated as an amount or percentage of MTRES1 mRNA per sample weight. In some embodiments, the MTRES1 mRNA level is indicated as an amount or percentage of MTRES1 mRNA per sample volume. In some embodiments, the MTRES1 mRNA level is indicated as an amount or percentage of MTRES1 mRNA per total mRNA within the sample. In some embodiments, the MTRES1 mRNA level is indicated as an amount or percentage of MTRES1 mRNA per total nucleic acids within the sample. In some embodiments, the MTRES1 mRNA level is indicated relative to another mRNA level, such as an mRNA level of a housekeeping gene, within the sample. In some embodiments, the MTRES1 mRNA measurement is a CNS tissue or fluid MTRES1 mRNA measurement. In some embodiments, the MTRES1 mRNA measurement is obtained by an assay such as a PCR assay. In some embodiments, the PCR comprises qPCR. In some embodiments, the PCR comprises reverse transcription of the MTRES1 mRNA.

In some embodiments, the composition reduces the MTRES1 mRNA measurement relative to the baseline MTRES1 mRNA measurement. In some embodiments, the MTRES1 mRNA measurement is obtained in a second sample obtained from the subject after administering the composition to the subject. In some embodiments, the composition reduces MTRES1 mRNA levels relative to the baseline MTRES1 mRNA levels. In some embodiments, the reduced MTRES1 mRNA levels are measured in a second sample obtained from the subject after administering the composition to the subject. In some embodiments, the second sample is a CNS sample. In some embodiments, the MTRES1 mRNA measurement is reduced by about 2.5% or more, about 5% or more, or about 7.5% or more, relative to the baseline MTRES1 mRNA measurement. In some embodiments, the MTRES1 mRNA measurement is decreased by about 10% or more, relative to the baseline MTRES1 mRNA measurement. In some embodiments, the MTRES1 mRNA measurement is decreased by about 20% or more, about 30% or more, about 40% or more, about 50% or more, about 60% or more, about 70% or more, about 80% or more, about 90% or more, or about 100%, relative to the baseline MTRES1 mRNA measurement. In some embodiments, the MTRES1 mRNA measurement is decreased by no more than about 2.5%, no more than about 5%, or no more than about 7.5%, relative to the baseline MTRES1 mRNA measurement. In some embodiments, the MTRES1 mRNA measurement is decreased by no more than about 10%, relative to the baseline MTRES1 mRNA measurement. In some embodiments, the MTRES1 mRNA measurement is decreased by no more than about 20%, no more than about 30%, no more than about 40%, no more than about 50%, no more than about 60%, no more than about 70%, no more than about 80%, no more than about 90%, or no more than about 100%, relative to the baseline MTRES1 mRNA measurement. In some embodiments, the MTRES1 mRNA measurement is decreased by 2.5%, 5%, 7.5%, 10%, 20%, 30%, 40%, 50%, 60%, 70%, 80%, 90%, or 100% or by a range defined by any of the two aforementioned percentages.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “Cx-y” or “Cx-Cy” when used in conjunction with a chemical moiety, such as alkyl, alkenyl, or alkynyl is meant to include groups that contain from x to y carbons in the chain. For example, the term “C1-6alkyl” refers to substituted or unsubstituted saturated hydrocarbon groups, including straight-chain alkyl and branched-chain alkyl groups that contain from 1 to 6 carbons.

The terms “Cx-yalkenyl” and “Cx-yalkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5-to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2 trifluoroethyl, 1 chloromethyl 2 fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3 benzodioxolyl, benzofuranyl, benzoxazolyl, benzo[d]thiazolyl, benzothiadiazolyl, benzo[b][1,4]dioxepinyl, benzo[b][1,4]oxazinyl, 1,4 benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2 d]pyrimidinyl, benzotriazolyl, benzo[4,6]imidazo[1,2 a]pyridinyl, carbazolyl, cinnolinyl, cyclopenta[d]pyrimidinyl, 6,7 dihydro 5H cyclopenta[4,5]thieno[2,3 d]pyrimidinyl, 5,6 dihydrobenzo[h]quinazolinyl, 5,6 dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2 c]pyridinyl, 5,6,7,8,9,10 hexahydrocycloocta[d]pyrimidinyl, 5,6,7,8,9,10 hexahydrocycloocta[d]pyridazinyl, 5,6,7,8,9,10 hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8 methano 5,6,7,8 tetrahydroquinazolinyl, naphthyridinyl, 1,6 naphthyridinonyl, oxadiazolyl, 2 oxoazepinyl, oxazolyl, oxiranyl, 5,6,6a,7,8,9,10,10a octahydrobenzo[h]quinazolinyl, 1 phenyl 1H pyrrolyl, phenazinyl, phenothiazinyl, phenoxazinyl, phthalazinyl, pteridinyl, purinyl, pyrrolyl, pyrazolyl, pyrazolo[3,4 d]pyrimidinyl, pyridinyl, pyrido[3,2 d]pyrimidinyl, pyrido[3,4 d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8 tetrahydroquinazolinyl, 5,6,7,8 tetrahydrobenzo[4,5]thieno[2,3 d]pyrimidinyl, 6,7,8,9 tetrahydro 5H cyclohepta[4,5]thieno[2,3 d]pyrimidinyl, 5,6,7,8 tetrahydropyrido[4,5 c]pyridazinyl, thiazolyl, thiadiazolyl, triazolyl, tetrazolyl, triazinyl, thieno[2,3 d]pyrimidinyl, thieno[3,2 d]pyrimidinyl, thieno[2,3 c]pyridinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, 2-oxa-6-azaspiro[3.3]heptane, and 1,1 dioxo thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH2 of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO2), imino (═N—H), oximo (═N—OH), hydrazino (═N—NH2), —Rb ORa, —Rb OC(O) Ra, —Rb OC(O) ORa, —Rb OC(O)N(Ra)2, —Rb N(Ra)2, —Rb C(O)Ra, —Rb C(O)ORa, —Rb C(O)N(Ra)2, —Rb O Rc C(O)N(Ra)2, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2), and —Rb S(O)tN(Ra)2 (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO2), imino (═N—H), oximo (═N—OH), hydrazine (═N—NH2), —Rb ORa, —Rb OC(O) Ra, —Rb OC(O) ORa, —Rb OC(O) N(Ra)2, —Rb N(Ra)2, —Rb C(O)Ra, —Rb C(O)ORa, —Rb C(O)N(Ra)2, —Rb O Rc C(O)N(Ra)2, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2) and —Rb S(O)tN(Ra)2 (where t is 1 or 2); wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each Ra, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO2), imino (═N—H), oximo (═N—OH), hydrazine (═N—NH2), —Rb ORa, —Rb OC(O) Ra, —Rb OC(O) ORa, —Rb OC(O) N(Ra)2, —Rb N(Ra)2, —Rb C(O)Ra, —Rb C(O)ORa, —Rb C(O)N(Ra)2, —Rb O Rc C(O)N(Ra)2, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2) and —Rb S(O)tN(Ra)2 (where t is 1 or 2); and wherein each Rb is independently selected from a direct bond or a straight or branched alkylene, alkenylene, or alkynylene chain, and each Rc is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

In some embodiments, a “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

Some embodiments refer to nucleic acid sequence information. It is contemplated that in some embodiments, thymine (T) may be interchanged with uracil (U), or vice versa. For example, some sequences in the sequence listing may recite Ts, but these may be replaced with Us in some embodiments. In some oligonucleotides with nucleic acid sequences that include uracil, the uracil may be replaced with thymine. Similarly, in some oligonucleotides with nucleic acid sequences that include thymine, the thymine may be replaced with uracil. In some embodiments, an oligonucleotide such as an siRNA comprises or consists of RNA. In some embodiments, the oligonucleotide may comprise or consist of DNA. For example, an ASO may include DNA.

Some aspects include sequences with nucleotide modifications or modified internucleoside linkages. Generally, and unless otherwise specified, Nf (e.g. Af, Cf, Gf, Tf, or Uf) refers to a 2′ fluoro-modified nucleoside, dN (e.g. dA, dC, dG, dT, or dU) refers to a 2′ deoxy nucleoside, n (e.g. a, c, g, t, or u) refers to a 2′ O-methyl modified nucleoside, and “s” refers to a phosphorothioate linkage.

The section headings used herein are for organizational purposes only and are not to be construed as limiting the subject matter described.

### VI. EXAMPLES

### Example 1: A Loss of Function Variant in MTRES1 Demonstrates Protective Associations for Dementia and Alzheimer's Disease Related Traits

Variants in MTRES1 were evaluated for associations with dementia, Alzheimer's disease and related traits in approximately 452,000 individuals with genotype data fromthe UK Biobank cohort. rs117058816 is a rare (AAF=0.006) splice donor variant (c.3+1G>A) in MTRES1. This variant is considered to be a loss of function variant that results in a decrease in the abundance or activity of the MTRES1 gene product.

The analyses resulted in identification of dementia and Alzheimer's disease-related associations for the MTRES1 loss of function variant. For example, rs117058816 was associated with decreased risk of Alzheimer's disease, dementia, delirium, and vascular dementia. rs117058816 was also associated with decreased risk of family history of Alzheimer's disease and decreased risk of dementia medication use (Table 1A and 1B).

TABLE 1A
MTRES1 Dementia, Alzheimer's and related trait associations

| | | | | Alzheimer's Disease | | Family History of Alzheimer's | |
|---|---|---|---|---|---|---|---|
| | | | | (n = 2,864) | | Disease (n = 53,344) | |
| Variant | Gene | Function | AAF | P value | OR | P value | OR |
| rs117058816 | MTRES1 | Splice donor; | 0.006 | 2.58E−04 | ↓0.459 | 9.54E−03 | ↓0.893 |
| | | c.3 + 1G > A | | | | | |

TABLE 1B
MTRES1 Dementia, Alzheimer's and related trait associations

| | Dementia | | Anticholinesterase | | Delirium | | Vascular Dementia | |
|---|---|---|---|---|---|---|---|---|
| | (n = 4,009) | | Medication (n = 813) | | (n = 3,901) | | (n = 807) | |
| Variant | P value | OR | P value | OR | P value | OR | P value | OR |
| rs117058816 | 7.92E−07 | ↓0.489 | 8.04E−03 | ↓0.613 | 7.75E−03 | ↓0.667 | 7.44E−04 | ↓0.208 |

These results indicate that loss of function of MTRES1 results in protection from dementia and Alzheimer's disease and related diseases. These results further indicate that therapeutic inhibition of MTRES1 may result in similar disease-protective effects.

Protective variants in MTRES1 result in a reduction of MTRES1 mRNA and MTRES1 protein

Minigene expression constructs encoding for wild type and rs117058816 (c.3+1G>A) MTRES1 proteins were generated. Minigene constructs (<10 kb) are easier to synthesize and have greater transfection efficiency in downstream experiments than constructs that exceed 10 kb in length. The minigene constructs have a portion of internal, intronic sequence removed, but retain all exons and UTRs. Therefore, the pre-mRNA of the exons, reduced introns, and 5′ and 3′ UTRs of the protein coding transcript (ENST00000625458) of MTRES1 was cloned into a pcDNA3.1(+) vector driven by a CMV promoter. Empty vector was used as control. For rs117058816 expression constructs, the A allele replaced the G allele at DNA sequence position chr6:107030108 (human genome build 38). This leads to the loss of a splice donor site (c.3+1G>A).

Transfections of HEK-293 cells were optimized. HEK-293 cells were plated in a 6-well plate in complete growth media and grown for 48 hours followed by a media change. Cells were then transfected with 2 μg of plasmid DNA and 7 μl of TransIT-2020. Cells were incubated for 48 hours, and then harvested.

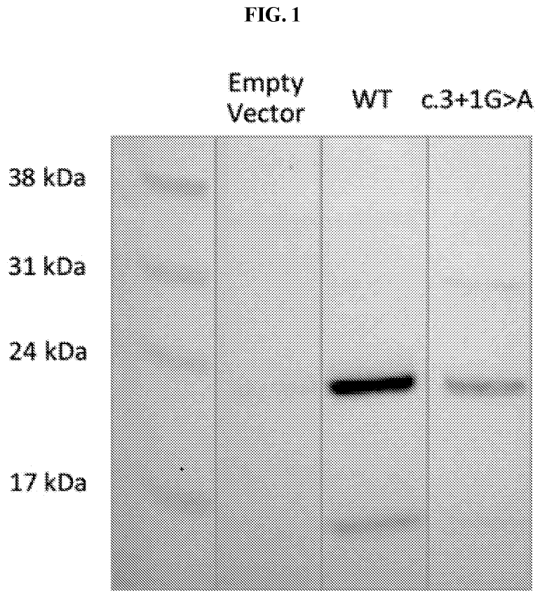

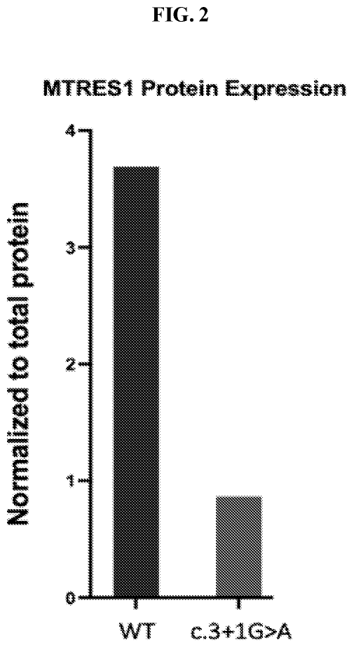

Cell lysates from transfected cells were assayed to evaluate intracellular MTRES1 protein by western blot (FIG. 1). In empty vector transfected HEK-293 cells, a faint band representing endogenous MTRES1 expression was detected by western blot as a band at 24 kDa. In cells transfected with the wild type construct, significant expression of MTRES1 was detected by western blot as a band 24 kDa. In cells transfected with the rs117058816 construct, reduced MTRES1 protein compared with wild type was detected by western blot as a band between 24 kDa. When normalizing to total protein, cells transfected with the rs117058816 construct express approximately 75% less MTRES1 protein compared with cells transfected with the wild type construct (FIG. 2).

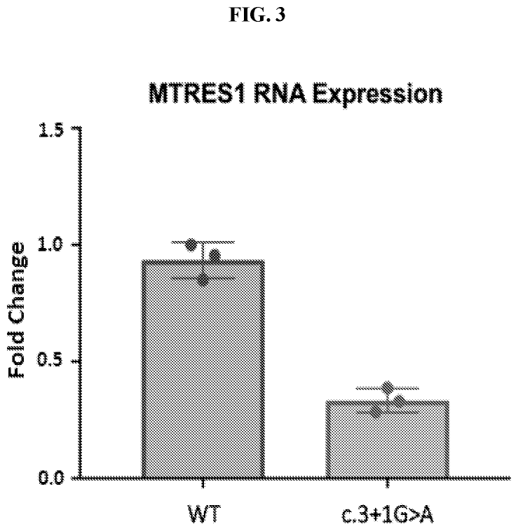

Cell lysates from transfected cells were also assayed to evaluate MTRES1 mRNA by qPCR. Cells transfected with the rs117058816 construct express approximately 60% less MTRES1 mRNA compared with cells transfected with the wild type construct (FIG. 3).

These data provide experimental verification that MTRES1 gene variants associated with protection from dementia and Alzheimer's disease result in loss of MTRES1 protein and MTRES1 mRNA abundance or function. Accordingly, in some cases therapeutic inhibition or modulation of MTRES1 may be an effective genetically-informed method of treatment for these diseases.

### Example 2: Bioinformatic Selection of Sequences in Order to Identify Therapeutic siRNAs to Downmodulate Expression of the MTRES1 mRNA

Screening sets were defined based on bioinformatic analysis. Therapeutic siRNAs were designed to target human MTRES1, and the MTRES1 sequence of at least one toxicology-relevant species, in this case, the non-human primates (NHP) rhesus and cynomolgus monkeys. Drivers for the design of the screening set were predicted specificity of the siRNAs against the transcriptome of the relevant species as well as cross-reactivity between species. Predicted specificity in human, rhesus monkey, cynomolgus monkey, mouse and rat was determined for sense (S) and antisense (AS) strands. These were assigned a “specificity score” which considers the likelihood of unintended downregulation of any other transcript by full or partial complementarity of an siRNA strand (up to 4 mismatches within positions 2-18) as well as the number and positions of mismatches. Thus, off-target(s) for antisense and sense strands of each siRNA were identified. In addition, the number of potential off-targets was used as an additional specificity factor in the specificity score. As identified, siRNAs with high specificity and a low number of predicted off-targets provide a benefit of increased targeting specificity.

In addition to selecting siRNA sequences with high sequence specificity to MTRES1 mRNA, siRNA sequences within the seed region were analyzed for similarity to seed regions of known miRNAs. siRNAs can function in a miRNA like manner via base-pairing with complementary sequences within the 3′-UTR of mRNA molecules. The complementarity typically encompasses the 5′-bases at positions 2-7 of the miRNA (seed region). To circumvent siRNAs to act via functional miRNA binding sites, siRNA strands containing natural miRNA seed regions were avoided. Seed regions identified in miRNAs from human, mouse, rat, rhesus monkey, dog, rabbit and pig are referred to as “conserved”. Combining the “specificity score” with miRNA seed analysis yielded a “specificity category”. This is divided into categories 1-4, with 1 having the highest specificity and 4 having the lowest specificity. Each strand of the siRNA is assigned to a specificity category.

Specificity and species cross-reactivity was assessed for human, cynomolgus monkey, rhesus monkey, mouse and rat MTRES1. The analysis was based on a canonical siRNA design using 19 bases and 17 bases (without considering positions 1 and 19) for cross-reactivity. Full match as well as single mismatch analyses were included.

Analysis of the human Single Nucleotide Polymorphism (SNP) database (NCBI-DB-SNP) to identify siRNAs targeting regions with known SNPs was also carried out to identify siRNAs that may be non-functional in individuals containing the SNP. Information regarding the positions of SNPs withinthe target sequence as well as minor allele frequency (MAF) in case data was obtained in this analysis.

Initial analysis of the relevant MTRES1 mRNA sequence revealed few sequences that fulfil the specificity parameters and at the same time target MTRES1 mRNA in all of the analyzed relevant species. Therefore, it was decided to design independent screening subsets for the therapeutic siRNAs.

The siRNAs in these subsets recognize the human, cynomolgus monkey, rhesus monkey MTRES1 sequences. Therefore, the siRNAs in these subsets can be used to target human MTRES1 in a therapeutic setting.

The number of siRNA sequences that can be derived from human MTRES1 mRNA (ENST00000311381.8, SEQ ID NO: 2443) without consideration of specificity or species cross-reactivity was 1140 (sense and antisense strand sequences included in SEQ ID NOS: 1-2280).

Prioritizing sequences for target specificity, species cross-reactivity, miRNA seed region sequences and SNPs as described above yields subset A. Subset A contains 82 siRNAs whose base sequences are shown in Table 2.

TABLE 2
Sequences in siRNA subset A

| siRNA | SEQ ID | sense strand sequence | SEQ ID | antisense strand sequence |
|---|---|---|---|---|
| Name | NO: | (5′-3′) | NO: | (5′-3′) |
| siRNA 78 | 78 | UAAGCGCCAUGGCUAUGGC | 1218 | GCCAUAGCCAUGGCGCUUA |
| siRNA 81 | 81 | GCGCCAUGGCUAUGGCUAG | 1221 | CUAGCCAUAGCCAUGGCGC |
| siRNA 87 | 87 | UGGCUAUGGCUAGUGUUAA | 1227 | UUAACACUAGCCAUAGCCA |
| siRNA 154 | 154 | GGGUGUUCUCCGAGGGACA | 1294 | UGUCCCUCGGAGAACACCC |
| siRNA 156 | 156 | GUGUUCUCCGAGGGACACC | 1296 | GGUGUCCCUCGGAGAACAC |
| siRNA 158 | 158 | GUUCUCCGAGGGACACCUU | 1298 | AAGGUGUCCCUCGGAGAAC |
| siRNA 178 | 178 | AUCAUACAAACUCUGUACU | 1318 | AGUACAGAGUUUGUAUGAU |
| siRNA 182 | 182 | UACAAACUCUGUACUUCCU | 1322 | AGGAAGUACAGAGUUUGUA |
| siRNA 190 | 190 | CUGUACUUCCUGGAAUCGA | 1330 | UCGAUUCCAGGAAGUACAG |
| siRNA 191 | 191 | UGUACUUCCUGGAAUCGAU | 1331 | AUCGAUUCCAGGAAGUACA |
| siRNA 192 | 192 | GUACUUCCUGGAAUCGAUA | 1332 | UAUCGAUUCCAGGAAGUAC |
| siRNA 193 | 193 | UACUUCCUGGAAUCGAUAC | 1333 | GUAUCGAUUCCAGGAAGUA |
| siRNA 194 | 194 | ACUUCCUGGAAUCGAUACU | 1334 | AGUAUCGAUUCCAGGAAGU |
| siRNA 195 | 195 | CUUCCUGGAAUCGAUACUU | 1335 | AAGUAUCGAUUCCAGGAAG |
| siRNA 197 | 197 | UCCUGGAAUCGAUACUUGU | 1337 | ACAAGUAUCGAUUCCAGGA |
| siRNA 198 | 198 | CCUGGAAUCGAUACUUGUA | 1338 | UACAAGUAUCGAUUCCAGG |
| siRNA 199 | 199 | CUGGAAUCGAUACUUGUAU | 1339 | AUACAAGUAUCGAUUCCAG |
| siRNA 202 | 202 | GAAUCGAUACUUGUAUUUU | 1342 | AAAAUACAAGUAUCGAUUC |
| siRNA 220 | 220 | UUCUAGUACCAAGUUACGU | 1360 | ACGUAACUUGGUACUAGAA |
| siRNA 222 | 222 | CUAGUACCAAGUUACGUGC | 1362 | GCACGUAACUUGGUACUAG |
| siRNA 223 | 223 | UAGUACCAAGUUACGUGCA | 1363 | UGCACGUAACUUGGUACUA |
| siRNA 224 | 224 | AGUACCAAGUUACGUGCAC | 1364 | GUGCACGUAACUUGGUACU |
| siRNA 225 | 225 | GUACCAAGUUACGUGCACC | 1365 | GGUGCACGUAACUUGGUAC |
| siRNA 226 | 226 | UACCAAGUUACGUGCACCA | 1366 | UGGUGCACGUAACUUGGUA |
| siRNA 227 | 227 | ACCAAGUUACGUGCACCAA | 1367 | UUGGUGCACGUAACUUGGU |
| siRNA 228 | 228 | CCAAGUUACGUGCACCAAA | 1368 | UUUGGUGCACGUAACUUGG |
| siRNA 229 | 229 | CAAGUUACGUGCACCAAAU | 1369 | AUUUGGUGCACGUAACUUG |
| siRNA 230 | 230 | AAGUUACGUGCACCAAAUU | 1370 | AAUUUGGUGCACGUAACUU |
| siRNA 231 | 231 | AGUUACGUGCACCAAAUUA | 1371 | UAAUUUGGUGCACGUAACU |
| siRNA 232 | 232 | GUUACGUGCACCAAAUUAU | 1372 | AUAAUUUGGUGCACGUAAC |
| siRNA 233 | 233 | UUACGUGCACCAAAUUAUA | 1373 | UAUAAUUUGGUGCACGUAA |
| SiRNA 235 | 235 | ACGUGCACCAAAUUAUAAA | 1375 | UUUAUAAUUUGGUGCACGU |
| siRNA 331 | 331 | AAGACUCAAAAGUAAUAUA | 1471 | UAUAUUACUUUUGAGUCUU |
| siRNA 358 | 358 | AAAAUCUACUAAAAAGUCU | 1498 | AGACUUUUUAGUAGAUUUU |
| siRNA 360 | 360 | AAUCUACUAAAAAGUCUCU | 1500 | AGAGACUUUUUAGUAGAUU |
| siRNA 361 | 361 | AUCUACUAAAAAGUCUCUG | 1501 | CAGAGACUUUUUAGUAGAU |
| siRNA 362 | 362 | UCUACUAAAAAGUCUCUGC | 1502 | GCAGAGACUUUUUAGUAGA |
| siRNA 528 | 528 | UGAAGACGGGGCUAGAUAU | 1668 | AUAUCUAGCCCCGUCUUCA |
| siRNA 534 | 534 | CGGGGCUAGAUAUUGGGAG | 1674 | CUCCCAAUAUCUAGCCCCG |
| siRNA 539 | 539 | CUAGAUAUUGGGAGAAACA | 1679 | UGUUUCUCCCAAUAUCUAG |
| siRNA 619 | 619 | AAGCAGAACGGUGAAAGUG | 1759 | CACUUUCACCGUUCUGCUU |
| siRNA 620 | 620 | AGCAGAACGGUGAAAGUGG | 1760 | CCACUUUCACCGUUCUGCU |
| siRNA 621 | 621 | GCAGAACGGUGAAAGUGGG | 1761 | CCCACUUUCACCGUUCUGC |
| siRNA 632 | 632 | AAAGUGGGAGAUACAUUGG | 1772 | CCAAUGUAUCUCCCACUUU |
| siRNA 633 | 633 | AAGUGGGAGAUACAUUGGA | 1773 | UCCAAUGUAUCUCCCACUU |
| siRNA 634 | 634 | AGUGGGAGAUACAUUGGAU | 1774 | AUCCAAUGUAUCUCCCACU |
| siRNA 636 | 636 | UGGGAGAUACAUUGGAUCU | 1776 | AGAUCCAAUGUAUCUCCCA |
| siRNA 642 | 642 | AUACAUUGGAUCUUCUCAU | 1782 | AUGAGAAGAUCCAAUGUAU |
| siRNA 645 | 645 | CAUUGGAUCUUCUCAUUGG | 1785 | CCAAUGAGAAGAUCCAAUG |
| siRNA 646 | 646 | AUUGGAUCUUCUCAUUGGA | 1786 | UCCAAUGAGAAGAUCCAAU |
| siRNA 647 | 647 | UUGGAUCUUCUCAUUGGAG | 1787 | CUCCAAUGAGAAGAUCCAA |
| siRNA 648 | 648 | UGGAUCUUCUCAUUGGAGA | 1788 | UCUCCAAUGAGAAGAUCCA |
| siRNA 650 | 650 | GAUCUUCUCAUUGGAGAGG | 1790 | CCUCUCCAAUGAGAAGAUC |
| siRNA 654 | 654 | UUCUCAUUGGAGAGGAUAA | 1794 | UUAUCCUCUCCAAUGAGAA |
| siRNA 656 | 656 | CUCAUUGGAGAGGAUAAAG | 1796 | CUUUAUCCUCUCCAAUGAG |
| siRNA 687 | 687 | AGACAGUUAUGCGGAUUCU | 1827 | AGAAUCCGCAUAACUGUCU |
| siRNA 688 | 688 | GACAGUUAUGCGGAUUCUC | 1828 | GAGAAUCCGCAUAACUGUC |
| siRNA 690 | 690 | CAGUUAUGCGGAUUCUCUU | 1830 | AAGAGAAUCCGCAUAACUG |
| siRNA 693 | 693 | UUAUGCGGAUUCUCUUGAA | 1833 | UUCAAGAGAAUCCGCAUAA |
| siRNA 694 | 694 | UAUGCGGAUUCUCUUGAAA | 1834 | UUUCAAGAGAAUCCGCAUA |
| siRNA 695 | 695 | AUGCGGAUUCUCUUGAAAA | 1835 | UUUUCAAGAGAAUCCGCAU |
| siRNA 745 | 745 | AUACAGAGUGGUGUUACGG | 1885 | CCGUAACACCACUCUGUAU |
| siRNA 746 | 746 | UACAGAGUGGUGUUACGGC | 1886 | GCCGUAACACCACUCUGUA |
| siRNA 748 | 748 | CAGAGUGGUGUUACGGCGG | 1888 | CCGCCGUAACACCACUCUG |
| SiRNA 749 | 749 | AGAGUGGUGUUACGGCGGU | 1889 | ACCGCCGUAACACCACUCU |
| siRNA 751 | 751 | AGUGGUGUUACGGCGGUGG | 1891 | CCACCGCCGUAACACCACU |
| siRNA 752 | 752 | GUGGUGUUACGGCGGUGGA | 1892 | UCCACCGCCGUAACACCAC |
| siRNA 753 | 753 | UGGUGUUACGGCGGUGGAA | 1893 | UUCCACCGCCGUAACACCA |
| siRNA 754 | 754 | GGUGUUACGGCGGUGGAAA | 1894 | UUUCCACCGCCGUAACACC |
| siRNA 755 | 755 | GUGUUACGGCGGUGGAAAA | 1895 | UUUUCCACCGCCGUAACAC |
| siRNA 756 | 756 | UGUUACGGCGGUGGAAAAG | 1896 | CUUUUCCACCGCCGUAACA |
| siRNA 757 | 757 | GUUACGGCGGUGGAAAAGU | 1897 | ACUUUUCCACCGCCGUAAC |
| siRNA 758 | 758 | UUACGGCGGUGGAAAAGUU | 1898 | AACUUUUCCACCGCCGUAA |
| siRNA 759 | 759 | UACGGCGGUGGAAAAGUUU | 1899 | AAACUUUUCCACCGCCGUA |
| siRNA 761 | 761 | CGGCGGUGGAAAAGUUUAA | 1901 | UUAAACUUUUCCACCGCCG |
| siRNA 773 | 773 | AGUUUAAAGUUGCCUAAGA | 1913 | UCUUAGGCAACUUUAAACU |
| siRNA 775 | 775 | UUUAAAGUUGCCUAAGAAG | 1915 | CUUCUUAGGCAACUUUAAA |
| SiRNA 808 | 808 | AAUGGAUUGCUUUUUAGCA | 1948 | UGCUAAAAAGCAAUCCAUU |
| siRNA 810 | 810 | UGGAUUGCUUUUUAGCAAU | 1950 | AUUGCUAAAAAGCAAUCCA |
| siRNA 852 | 852 | GAAGGGGUCACCUGAAAAA | 1992 | UUUUUCAGGUGACCCCUUC |
| siRNA 853 | 853 | AAGGGGUCACCUGAAAAAU | 1993 | AUUUUUCAGGUGACCCCUU |
| siRNA 887 | 887 | AAAUAAAGUUCUCUUAGCG | 2027 | CGCUAAGAGAACUUUAUUU |

The siRNAs in subset A have the following characteristics:
Cross-reactivity: With 19mer in human MTRES1 mRNA, with 17mer/19mer in NHP MTRES1
Specificity category: For human and NHP: AS2 or better, SS3 or better
miRNA seeds: AS+SS strand: seed region not conserved in human, mouse, and rat and not present in >4 species
Off-target frequency: ≤20 human off-targets matched with 2 mismatches in antisense strand
SNPs: siRNA target sites do not harbor SNPs with a MAF≥100 (pos. 2-18)

The siRNA sequences in subset A were selected for more stringent specificity to yield subset B. Subset B includes 73 siRNAs whose base sequences are shown in Table 3.

TABLE 3
Sequences in siRNA subset B

| SEQ ID | sense strand sequence | SEQ ID | antisense strand sequence |
|---|---|---|---|
| NO: | (5′-3′) | NO: | (5′-3′) |
| 78 | UAAGCGCCAUGGCUAUGGC | 1218 | GCCAUAGCCAUGGCGCUUA |
| 81 | GCGCCAUGGCUAUGGCUAG | 1221 | CUAGCCAUAGCCAUGGCGC |
| 87 | UGGCUAUGGCUAGUGUUAA | 1227 | UUAACACUAGCCAUAGCCA |
| 154 | GGGUGUUCUCCGAGGGACA | 1294 | UGUCCCUCGGAGAACACCC |
| 156 | GUGUUCUCCGAGGGACACC | 1296 | GGUGUCCCUCGGAGAACAC |
| 158 | GUUCUCCGAGGGACACCUU | 1298 | AAGGUGUCCCUCGGAGAAC |
| 178 | AUCAUACAAACUCUGUACU | 1318 | AGUACAGAGUUUGUAUGAU |
| 182 | UACAAACUCUGUACUUCCU | 1322 | AGGAAGUACAGAGUUUGUA |
| 190 | CUGUACUUCCUGGAAUCGA | 1330 | UCGAUUCCAGGAAGUACAG |
| 191 | UGUACUUCCUGGAAUCGAU | 1331 | AUCGAUUCCAGGAAGUACA |
| 192 | GUACUUCCUGGAAUCGAUA | 1332 | UAUCGAUUCCAGGAAGUAC |
| 193 | UACUUCCUGGAAUCGAUAC | 1333 | GUAUCGAUUCCAGGAAGUA |
| 195 | CUUCCUGGAAUCGAUACUU | 1335 | AAGUAUCGAUUCCAGGAAG |
| 197 | UCCUGGAAUCGAUACUUGU | 1337 | ACAAGUAUCGAUUCCAGGA |
| 198 | CCUGGAAUCGAUACUUGUA | 1338 | UACAAGUAUCGAUUCCAGG |
| 199 | CUGGAAUCGAUACUUGUAU | 1339 | AUACAAGUAUCGAUUCCAG |
| 202 | GAAUCGAUACUUGUAUUUU | 1342 | AAAAUACAAGUAUCGAUUC |
| 220 | UUCUAGUACCAAGUUACGU | 1360 | ACGUAACUUGGUACUAGAA |
| 222 | CUAGUACCAAGUUACGUGC | 1362 | GCACGUAACUUGGUACUAG |
| 223 | UAGUACCAAGUUACGUGCA | 1363 | UGCACGUAACUUGGUACUA |
| 224 | AGUACCAAGUUACGUGCAC | 1364 | GUGCACGUAACUUGGUACU |
| 225 | GUACCAAGUUACGUGCACC | 1365 | GGUGCACGUAACUUGGUAC |
| 226 | UACCAAGUUACGUGCACCA | 1366 | UGGUGCACGUAACUUGGUA |
| 227 | ACCAAGUUACGUGCACCAA | 1367 | UUGGUGCACGUAACUUGGU |
| 228 | CCAAGUUACGUGCACCAAA | 1368 | UUUGGUGCACGUAACUUGG |
| 229 | CAAGUUACGUGCACCAAAU | 1369 | AUUUGGUGCACGUAACUUG |
| 230 | AAGUUACGUGCACCAAAUU | 1370 | AAUUUGGUGCACGUAACUU |
| 231 | AGUUACGUGCACCAAAUUA | 1371 | UAAUUUGGUGCACGUAACU |
| 232 | GUUACGUGCACCAAAUUAU | 1372 | AUAAUUUGGUGCACGUAAC |
| 233 | UUACGUGCACCAAAUUAUA | 1373 | UAUAAUUUGGUGCACGUAA |
| 235 | ACGUGCACCAAAUUAUAAA | 1375 | UUUAUAAUUUGGUGCACGU |
| 358 | AAAAUCUACUAAAAAGUCU | 1498 | AGACUUUUUAGUAGAUUUU |
| 360 | AAUCUACUAAAAAGUCUCU | 1500 | AGAGACUUUUUAGUAGAUU |
| 362 | UCUACUAAAAAGUCUCUGC | 1502 | GCAGAGACUUUUUAGUAGA |
| 528 | UGAAGACGGGGCUAGAUAU | 1668 | AUAUCUAGCCCCGUCUUCA |
| 534 | CGGGGCUAGAUAUUGGGAG | 1674 | CUCCCAAUAUCUAGCCCCG |
| 539 | CUAGAUAUUGGGAGAAACA | 1679 | UGUUUCUCCCAAUAUCUAG |
| 619 | AAGCAGAACGGUGAAAGUG | 1759 | CACUUUCACCGUUCUGCUU |
| 620 | AGCAGAACGGUGAAAGUGG | 1760 | CCACUUUCACCGUUCUGCU |
| 621 | GCAGAACGGUGAAAGUGGG | 1761 | CCCACUUUCACCGUUCUGC |
| 632 | AAAGUGGGAGAUACAUUGG | 1772 | CCAAUGUAUCUCCCACUUU |
| 633 | AAGUGGGAGAUACAUUGGA | 1773 | UCCAAUGUAUCUCCCACUU |
| 636 | UGGGAGAUACAUUGGAUCU | 1776 | AGAUCCAAUGUAUCUCCCA |
| 642 | AUACAUUGGAUCUUCUCAU | 1782 | AUGAGAAGAUCCAAUGUAU |
| 645 | CAUUGGAUCUUCUCAUUGG | 1785 | CCAAUGAGAAGAUCCAAUG |
| 647 | UUGGAUCUUCUCAUUGGAG | 1787 | CUCCAAUGAGAAGAUCCAA |
| 648 | UGGAUCUUCUCAUUGGAGA | 1788 | UCUCCAAUGAGAAGAUCCA |
| 654 | UUCUCAUUGGAGAGGAUAA | 1794 | UUAUCCUCUCCAAUGAGAA |
| 656 | CUCAUUGGAGAGGAUAAAG | 1796 | CUUUAUCCUCUCCAAUGAG |
| 687 | AGACAGUUAUGCGGAUUCU | 1827 | AGAAUCCGCAUAACUGUCU |
| 688 | GACAGUUAUGCGGAUUCUC | 1828 | GAGAAUCCGCAUAACUGUC |
| 690 | CAGUUAUGCGGAUUCUCUU | 1830 | AAGAGAAUCCGCAUAACUG |
| 693 | UUAUGCGGAUUCUCUUGAA | 1833 | UUCAAGAGAAUCCGCAUAA |
| 694 | UAUGCGGAUUCUCUUGAAA | 1834 | UUUCAAGAGAAUCCGCAUA |
| 695 | AUGCGGAUUCUCUUGAAAA | 1835 | UUUUCAAGAGAAUCCGCAU |
| 745 | AUACAGAGUGGUGUUACGG | 1885 | CCGUAACACCACUCUGUAU |
| 746 | UACAGAGUGGUGUUACGGC | 1886 | GCCGUAACACCACUCUGUA |
| 748 | CAGAGUGGUGUUACGGCGG | 1888 | CCGCCGUAACACCACUCUG |
| 749 | AGAGUGGUGUUACGGCGGU | 1889 | ACCGCCGUAACACCACUCU |
| 751 | AGUGGUGUUACGGCGGUGG | 1891 | CCACCGCCGUAACACCACU |
| 752 | GUGGUGUUACGGCGGUGGA | 1892 | UCCACCGCCGUAACACCAC |
| 753 | UGGUGUUACGGCGGUGGAA | 1893 | UUCCACCGCCGUAACACCA |
| 754 | GGUGUUACGGCGGUGGAAA | 1894 | UUUCCACCGCCGUAACACC |
| 755 | GUGUUACGGCGGUGGAAAA | 1895 | UUUUCCACCGCCGUAACAC |
| 756 | UGUUACGGCGGUGGAAAAG | 1896 | CUUUUCCACCGCCGUAACA |
| 757 | GUUACGGCGGUGGAAAAGU | 1897 | ACUUUUCCACCGCCGUAAC |
| 758 | UUACGGCGGUGGAAAAGUU | 1898 | AACUUUUCCACCGCCGUAA |
| 759 | UACGGCGGUGGAAAAGUUU | 1899 | AAACUUUUCCACCGCCGUA |
| 761 | CGGCGGUGGAAAAGUUUAA | 1901 | UUAAACUUUUCCACCGCCG |
| 773 | AGUUUAAAGUUGCCUAAGA | 1913 | UCUUAGGCAACUUUAAACU |
| 808 | AAUGGAUUGCUUUUUAGCA | 1948 | UGCUAAAAAGCAAUCCAUU |
| 852 | GAAGGGGUCACCUGAAAAA | 1992 | UUUUUCAGGUGACCCCUUC |
| 853 | AAGGGGUCACCUGAAAAAU | 1993 | AUUUUUCAGGUGACCCCUU |

The siRNAs in subset B have the following characteristics:
Cross-reactivity: With 19mer in human MTRES1 mRNA, with 17mer/19mer in NHP MTRES1
Specificity category: For human and NHP: AS2 or better, SS3 or better
miRNA seeds: AS+SS strand: seed region not conserved in human, mouse, and rat and not present in >4 species
Off-target frequency: <15 human off-targets matched with 2 mismatches in antisense strand
SNPs: siRNA target sites do not harbor SNPs with a MAF ≥1% (pos. 2-18)

The siRNA sequences in subset B were further selected for absence of seed regions in the AS strand that are identical to a seed region of known human miRNA to yield subset C. Subset C includes 54 siRNAs whose base sequences are shown in Table 4.

TABLE 4
Sequences in siRNA subset C

| SEQ ID | sense strand sequence | SEQ ID | antisense strand sequence |
|---|---|---|---|
| NO: | (5′-3′) | NO: | (5′-3′) |
| 78 | UAAGCGCCAUGGCUAUGGC | 1218 | GCCAUAGCCAUGGCGCUUA |
| 87 | UGGCUAUGGCUAGUGUUAA | 1227 | UUAACACUAGCCAUAGCCA |
| 154 | GGGUGUUCUCCGAGGGACA | 1294 | UGUCCCUCGGAGAACACCC |
| 158 | GUUCUCCGAGGGACACCUU | 1298 | AAGGUGUCCCUCGGAGAAC |
| 178 | AUCAUACAAACUCUGUACU | 1318 | AGUACAGAGUUUGUAUGAU |
| 182 | UACAAACUCUGUACUUCCU | 1322 | AGGAAGUACAGAGUUUGUA |
| 190 | CUGUACUUCCUGGAAUCGA | 1330 | UCGAUUCCAGGAAGUACAG |
| 191 | UGUACUUCCUGGAAUCGAU | 1331 | AUCGAUUCCAGGAAGUACA |
| 192 | GUACUUCCUGGAAUCGAUA | 1332 | UAUCGAUUCCAGGAAGUAC |
| 193 | UACUUCCUGGAAUCGAUAC | 1333 | GUAUCGAUUCCAGGAAGUA |
| 195 | CUUCCUGGAAUCGAUACUU | 1335 | AAGUAUCGAUUCCAGGAAG |
| 199 | CUGGAAUCGAUACUUGUAU | 1339 | AUACAAGUAUCGAUUCCAG |
| 202 | GAAUCGAUACUUGUAUUUU | 1342 | AAAAUACAAGUAUCGAUUC |
| 220 | UUCUAGUACCAAGUUACGU | 1360 | ACGUAACUUGGUACUAGAA |
| 222 | CUAGUACCAAGUUACGUGC | 1362 | GCACGUAACUUGGUACUAG |
| 223 | UAGUACCAAGUUACGUGCA | 1363 | UGCACGUAACUUGGUACUA |
| 224 | AGUACCAAGUUACGUGCAC | 1364 | GUGCACGUAACUUGGUACU |
| 225 | GUACCAAGUUACGUGCACC | 1365 | GGUGCACGUAACUUGGUAC |
| 226 | UACCAAGUUACGUGCACCA | 1366 | UGGUGCACGUAACUUGGUA |
| 227 | ACCAAGUUACGUGCACCAA | 1367 | UUGGUGCACGUAACUUGGU |
| 228 | CCAAGUUACGUGCACCAAA | 1368 | UUUGGUGCACGUAACUUGG |
| 229 | CAAGUUACGUGCACCAAAU | 1369 | AUUUGGUGCACGUAACUUG |
| 231 | AGUUACGUGCACCAAAUUA | 1371 | UAAUUUGGUGCACGUAACU |
| 233 | UUACGUGCACCAAAUUAUA | 1373 | UAUAAUUUGGUGCACGUAA |
| 235 | ACGUGCACCAAAUUAUAAA | 1375 | UUUAUAAUUUGGUGCACGU |
| 358 | AAAAUCUACUAAAAAGUCU | 1498 | AGACUUUUUAGUAGAUUUU |
| 528 | UGAAGACGGGGCUAGAUAU | 1668 | AUAUCUAGCCCCGUCUUCA |
| 534 | CGGGGCUAGAUAUUGGGAG | 1674 | CUCCCAAUAUCUAGCCCCG |
| 539 | CUAGAUAUUGGGAGAAACA | 1679 | UGUUUCUCCCAAUAUCUAG |
| 619 | AAGCAGAACGGUGAAAGUG | 1759 | CACUUUCACCGUUCUGCUU |
| 620 | AGCAGAACGGUGAAAGUGG | 1760 | CCACUUUCACCGUUCUGCU |
| 621 | GCAGAACGGUGAAAGUGGG | 1761 | CCCACUUUCACCGUUCUGC |
| 632 | AAAGUGGGAGAUACAUUGG | 1772 | CCAAUGUAUCUCCCACUUU |
| 633 | AAGUGGGAGAUACAUUGGA | 1773 | UCCAAUGUAUCUCCCACUU |
| 636 | UGGGAGAUACAUUGGAUCU | 1776 | AGAUCCAAUGUAUCUCCCA |
| 645 | CAUUGGAUCUUCUCAUUGG | 1785 | CCAAUGAGAAGAUCCAAUG |
| 647 | UUGGAUCUUCUCAUUGGAG | 1787 | CUCCAAUGAGAAGAUCCAA |
| 656 | CUCAUUGGAGAGGAUAAAG | 1796 | CUUUAUCCUCUCCAAUGAG |
| 687 | AGACAGUUAUGCGGAUUCU | 1827 | AGAAUCCGCAUAACUGUCU |
| 688 | GACAGUUAUGCGGAUUCUC | 1828 | GAGAAUCCGCAUAACUGUC |
| 745 | AUACAGAGUGGUGUUACGG | 1885 | CCGUAACACCACUCUGUAU |
| 746 | UACAGAGUGGUGUUACGGC | 1886 | GCCGUAACACCACUCUGUA |
| 748 | CAGAGUGGUGUUACGGCGG | 1888 | CCGCCGUAACACCACUCUG |
| 749 | AGAGUGGUGUUACGGCGGU | 1889 | ACCGCCGUAACACCACUCU |
| 751 | AGUGGUGUUACGGCGGUGG | 1891 | CCACCGCCGUAACACCACU |
| 752 | GUGGUGUUACGGCGGUGGA | 1892 | UCCACCGCCGUAACACCAC |
| 753 | UGGUGUUACGGCGGUGGAA | 1893 | UUCCACCGCCGUAACACCA |
| 755 | GUGUUACGGCGGUGGAAAA | 1895 | UUUUCCACCGCCGUAACAC |
| 756 | UGUUACGGCGGUGGAAAAG | 1896 | CUUUUCCACCGCCGUAACA |
| 759 | UACGGCGGUGGAAAAGUUU | 1899 | AAACUUUUCCACCGCCGUA |
| 761 | CGGCGGUGGAAAAGUUUAA | 1901 | UUAAACUUUUCCACCGCCG |
| 773 | AGUUUAAAGUUGCCUAAGA | 1913 | UCUUAGGCAACUUUAAACU |
| 808 | AAUGGAUUGCUUUUUAGCA | 1948 | UGCUAAAAAGCAAUCCAUU |
| 853 | AAGGGGUCACCUGAAAAAU | 1993 | AUUUUUCAGGUGACCCCUU |

The siRNAs in subset C have the following characteristics:
Cross-reactivity: With 19mer in human MTRES1 mRNA, with 17mer/19mer in NHP MTRES1
Specificity category: For human and NHP: AS2 or better, SS3 or better
miRNA seeds: AS+SS strand: seed region not conserved in human, mouse, and rat and not present in >4 species. AS strand: seed region not identical to seed region of known human miRNA
Off-target frequency: ≤15 human off-targets matched with 2 mismatches by antisense strand
SNPs: siRNA target sites do not harbor SNPs with a MAF≥1% (pos. 2-18)

The siRNA sequences in subset C were also selected for absence of seed regions in the AS or S strands that are identical to a seed region of known human miRNA to yield subset D. Subset D includes 35 siRNAs whose base sequences are shown in Table 5.

TABLE 5
Sequences in siRNA subset D

| SEQ ID | sense strand sequence | SEQ ID | antisense strand sequence |
|---|---|---|---|
| NO: | (5′-3′) | NO: | (5′-3′) |
| 87 | UGGCUAUGGCUAGUGUUAA | 1227 | UUAACACUAGCCAUAGCCA |
| 182 | UACAAACUCUGUACUUCCU | 1322 | AGGAAGUACAGAGUUUGUA |
| 190 | CUGUACUUCCUGGAAUCGA | 1330 | UCGAUUCCAGGAAGUACAG |
| 191 | UGUACUUCCUGGAAUCGAU | 1331 | AUCGAUUCCAGGAAGUACA |
| 193 | UACUUCCUGGAAUCGAUAC | 1333 | GUAUCGAUUCCAGGAAGUA |
| 194 | ACUUCCUGGAAUCGAUACU | 1334 | AGUAUCGAUUCCAGGAAGU |
| 202 | GAAUCGAUACUUGUAUUUU | 1342 | AAAAUACAAGUAUCGAUUC |
| 220 | UUCUAGUACCAAGUUACGU | 1360 | ACGUAACUUGGUACUAGAA |
| 222 | CUAGUACCAAGUUACGUGC | 1362 | GCACGUAACUUGGUACUAG |
| 224 | AGUACCAAGUUACGUGCAC | 1364 | GUGCACGUAACUUGGUACU |
| 225 | GUACCAAGUUACGUGCACC | 1365 | GGUGCACGUAACUUGGUAC |
| 226 | UACCAAGUUACGUGCACCA | 1366 | UGGUGCACGUAACUUGGUA |
| 228 | CCAAGUUACGUGCACCAAA | 1368 | UUUGGUGCACGUAACUUGG |
| 229 | CAAGUUACGUGCACCAAAU | 1369 | AUUUGGUGCACGUAACUUG |
| 231 | AGUUACGUGCACCAAAUUA | 1371 | UAAUUUGGUGCACGUAACU |
| 233 | UUACGUGCACCAAAUUAUA | 1373 | UAUAAUUUGGUGCACGUAA |
| 358 | AAAAUCUACUAAAAAGUCU | 1498 | AGACUUUUUAGUAGAUUUU |
| 361 | AUCUACUAAAAAGUCUCUG | 1501 | CAGAGACUUUUUAGUAGAU |
| 528 | UGAAGACGGGGCUAGAUAU | 1668 | AUAUCUAGCCCCGUCUUCA |
| 539 | CUAGAUAUUGGGAGAAACA | 1679 | UGUUUCUCCCAAUAUCUAG |
| 619 | AAGCAGAACGGUGAAAGUG | 1759 | CACUUUCACCGUUCUGCUU |
| 645 | CAUUGGAUCUUCUCAUUGG | 1785 | CCAAUGAGAAGAUCCAAUG |
| 647 | UUGGAUCUUCUCAUUGGAG | 1787 | CUCCAAUGAGAAGAUCCAA |
| 688 | GACAGUUAUGCGGAUUCUC | 1828 | GAGAAUCCGCAUAACUGUC |
| 745 | AUACAGAGUGGUGUUACGG | 1885 | CCGUAACACCACUCUGUAU |
| 751 | AGUGGUGUUACGGCGGUGG | 1891 | CCACCGCCGUAACACCACU |
| 752 | GUGGUGUUACGGCGGUGGA | 1892 | UCCACCGCCGUAACACCAC |
| 755 | GUGUUACGGCGGUGGAAAA | 1895 | UUUUCCACCGCCGUAACAC |
| 756 | UGUUACGGCGGUGGAAAAG | 1896 | CUUUUCCACCGCCGUAACA |
| 759 | UACGGCGGUGGAAAAGUUU | 1899 | AAACUUUUCCACCGCCGUA |
| 761 | CGGCGGUGGAAAAGUUUAA | 1901 | UUAAACUUUUCCACCGCCG |
| 773 | AGUUUAAAGUUGCCUAAGA | 1913 | UCUUAGGCAACUUUAAACU |
| 775 | UUUAAAGUUGCCUAAGAAG | 1915 | CUUCUUAGGCAACUUUAAA |
| 810 | UGGAUUGCUUUUUAGCAAU | 1950 | AUUGCUAAAAAGCAAUCCA |
| 887 | AAAUAAAGUUCUCUUAGCG | 2027 | CGCUAAGAGAACUUUAUUU |

The siRNAs in subset D have the following characteristics:
Cross-reactivity: With 19mer in human MTRES1 mRNA, with 17mer/19mer in NHP MTRES1
Specificity category: For human and NHP: AS2 or better, SS3 or better
miRNA seeds: AS+SS strand: seed region not conserved in human, mouse, and rat and not present in >4 species. AS+SS strand: seed region not identical to seed region of known human miRNA
Off-target frequency: <20 human off-targets matched with 2 mismatches by antisense strand
SNPs: siRNA target sites do not harbor SNPs with a MAF≥1% (pos. 2-18)

The siRNA sequences in subset D were further selected for more stringent specificity to yield subset E. Subset E includes 30 siRNAs whose base sequences are shown in Table 6.

TABLE 6
Sequences in siRNA subset E

| SEQ ID | sense strand sequence | SEQ ID | antisense strand sequence |
|---|---|---|---|
| NO: | (5′-3′) | NO: | (5′-3′) |
| 87 | UGGCUAUGGCUAGUGUUAA | 1227 | UUAACACUAGCCAUAGCCA |
| 182 | UACAAACUCUGUACUUCCU | 1322 | AGGAAGUACAGAGUUUGUA |
| 190 | CUGUACUUCCUGGAAUCGA | 1330 | UCGAUUCCAGGAAGUACAG |
| 191 | UGUACUUCCUGGAAUCGAU | 1331 | AUCGAUUCCAGGAAGUACA |
| 193 | UACUUCCUGGAAUCGAUAC | 1333 | GUAUCGAUUCCAGGAAGUA |
| 202 | GAAUCGAUACUUGUAUUUU | 1342 | AAAAUACAAGUAUCGAUUC |
| 220 | UUCUAGUACCAAGUUACGU | 1360 | ACGUAACUUGGUACUAGAA |
| 222 | CUAGUACCAAGUUACGUGC | 1362 | GCACGUAACUUGGUACUAG |
| 224 | AGUACCAAGUUACGUGCAC | 1364 | GUGCACGUAACUUGGUACU |
| 225 | GUACCAAGUUACGUGCACC | 1365 | GGUGCACGUAACUUGGUAC |
| 226 | UACCAAGUUACGUGCACCA | 1366 | UGGUGCACGUAACUUGGUA |
| 228 | CCAAGUUACGUGCACCAAA | 1368 | UUUGGUGCACGUAACUUGG |
| 229 | CAAGUUACGUGCACCAAAU | 1369 | AUUUGGUGCACGUAACUUG |
| 231 | AGUUACGUGCACCAAAUUA | 1371 | UAAUUUGGUGCACGUAACU |
| 233 | UUACGUGCACCAAAUUAUA | 1373 | UAUAAUUUGGUGCACGUAA |
| 358 | AAAAUCUACUAAAAAGUCU | 1498 | AGACUUUUUAGUAGAUUUU |
| 528 | UGAAGACGGGGCUAGAUAU | 1668 | AUAUCUAGCCCCGUCUUCA |
| 539 | CUAGAUAUUGGGAGAAACA | 1679 | UGUUUCUCCCAAUAUCUAG |
| 619 | AAGCAGAACGGUGAAAGUG | 1759 | CACUUUCACCGUUCUGCUU |
| 645 | CAUUGGAUCUUCUCAUUGG | 1785 | CCAAUGAGAAGAUCCAAUG |
| 647 | UUGGAUCUUCUCAUUGGAG | 1787 | CUCCAAUGAGAAGAUCCAA |
| 688 | GACAGUUAUGCGGAUUCUC | 1828 | GAGAAUCCGCAUAACUGUC |
| 745 | AUACAGAGUGGUGUUACGG | 1885 | CCGUAACACCACUCUGUAU |
| 751 | AGUGGUGUUACGGCGGUGG | 1891 | CCACCGCCGUAACACCACU |
| 752 | GUGGUGUUACGGCGGUGGA | 1892 | UCCACCGCCGUAACACCAC |
| 755 | GUGUUACGGCGGUGGAAAA | 1895 | UUUUCCACCGCCGUAACAC |
| 756 | UGUUACGGCGGUGGAAAAG | 1896 | CUUUUCCACCGCCGUAACA |
| 759 | UACGGCGGUGGAAAAGUUU | 1899 | AAACUUUUCCACCGCCGUA |
| 761 | CGGCGGUGGAAAAGUUUAA | 1901 | UUAAACUUUUCCACCGCCG |
| 773 | AGUUUAAAGUUGCCUAAGA | 1913 | UCUUAGGCAACUUUAAACU |

The siRNAs in subset E have the following characteristics:
Cross-reactivity: With 19mer in human MTRES1 mRNA, with 17mer/19mer in NHP MTRES1
Specificity category: For human and NHP: AS2 or better, SS3 or better
miRNA seeds: AS+SS strand: seed region not conserved in human, mouse, and rat and not present in >4 species. AS+SS strand: seed region not identical to seed region of known human miRNA
Off-target frequency: <15 human off-targets matched with 2 mismatches by antisense strand
SNPs: siRNA target sites do not harbor SNPs with a MAF ≥1% (pos. 2-18)

Subset F includes 54 siRNAs. The siRNAs in subset F include siRNAs from subset A, and are included in Table 7. In some cases, the sense strand of any of the siRNAs of subset F comprises modification pattern 6S (Table 8). In some cases, the antisense strand of any of the siRNAs of subset F comprises modification pattern 7AS (Table 8, “subset G”). In some cases, the sense strand of any of the siRNAs of subset F contains an alternative modification pattern (Table 9, “subset H”). In some cases, the antisense strand of any of the siRNAs of subset F comprises modification pattern 7AS (Table 9). The siRNAs in subset F may comprise any other modification pattern(s). In Table 8 and Table 9, Nf (e.g. Af, Cf, Gf, Tf, or Uf) is a 2′ fluoro-modified nucleoside, n (e.g. a, c, g, t, or u) is a 2′ O-methyl modified nucleoside, and “s” is a phosphorothioate linkage.

TABLE 7
Sequences in siRNA subset F

| SEQ ID | sense strand sequence | SEQ ID | antisense strand sequence |
|---|---|---|---|
| NO: | (5′-3′) | NO: | (5′-3′) |
| 87 | UGGCUAUGGCUAGUGUUAA | 1227 | UUAACACUAGCCAUAGCCA |
| 178 | AUCAUACAAACUCUGUACU | 1318 | AGUACAGAGUUUGUAUGAU |
| 190 | CUGUACUUCCUGGAAUCGA | 1330 | UCGAUUCCAGGAAGUACAG |
| 191 | UGUACUUCCUGGAAUCGAU | 1331 | AUCGAUUCCAGGAAGUACA |
| 192 | GUACUUCCUGGAAUCGAUA | 1332 | UAUCGAUUCCAGGAAGUAC |
| 193 | UACUUCCUGGAAUCGAUAC | 1333 | GUAUCGAUUCCAGGAAGUA |
| 195 | CUUCCUGGAAUCGAUACUU | 1335 | AAGUAUCGAUUCCAGGAAG |
| 199 | CUGGAAUCGAUACUUGUAU | 1339 | AUACAAGUAUCGAUUCCAG |
| 202 | GAAUCGAUACUUGUAUUUU | 1342 | AAAAUACAAGUAUCGAUUC |
| 222 | CUAGUACCAAGUUACGUGC | 1362 | GCACGUAACUUGGUACUAG |
| 223 | UAGUACCAAGUUACGUGCA | 1363 | UGCACGUAACUUGGUACUA |
| 224 | AGUACCAAGUUACGUGCAC | 1364 | GUGCACGUAACUUGGUACU |
| 225 | GUACCAAGUUACGUGCACC | 1365 | GGUGCACGUAACUUGGUAC |
| 226 | UACCAAGUUACGUGCACCA | 1366 | UGGUGCACGUAACUUGGUA |
| 228 | CCAAGUUACGUGCACCAAA | 1368 | UUUGGUGCACGUAACUUGG |
| 229 | CAAGUUACGUGCACCAAAU | 1369 | AUUUGGUGCACGUAACUUG |
| 230 | AAGUUACGUGCACCAAAUU | 1370 | AAUUUGGUGCACGUAACUU |
| 232 | GUUACGUGCACCAAAUUAU | 1372 | AUAAUUUGGUGCACGUAAC |
| 233 | UUACGUGCACCAAAUUAUA | 1373 | UAUAAUUUGGUGCACGUAA |
| 331 | AAGACUCAAAAGUAAUAUA | 1471 | UAUAUUACUUUUGAGUCUU |
| 358 | AAAAUCUACUAAAAAGUCU | 1498 | AGACUUUUUAGUAGAUUUU |
| 362 | UCUACUAAAAAGUCUCUGC | 1502 | GCAGAGACUUUUUAGUAGA |
| 528 | UGAAGACGGGGCUAGAUAU | 1668 | AUAUCUAGCCCCGUCUUCA |
| 539 | CUAGAUAUUGGGAGAAACA | 1679 | UGUUUCUCCCAAUAUCUAG |
| 620 | AGCAGAACGGUGAAAGUGG | 1760 | CCACUUUCACCGUUCUGCU |
| 632 | AAAGUGGGAGAUACAUUGG | 1772 | CCAAUGUAUCUCCCACUUU |
| 633 | AAGUGGGAGAUACAUUGGA | 1773 | UCCAAUGUAUCUCCCACUU |
| 634 | AGUGGGAGAUACAUUGGAU | 1774 | AUCCAAUGUAUCUCCCACU |
| 636 | UGGGAGAUACAUUGGAUCU | 1776 | AGAUCCAAUGUAUCUCCCA |
| 642 | AUACAUUGGAUCUUCUCAU | 1782 | AUGAGAAGAUCCAAUGUAU |
| 645 | CAUUGGAUCUUCUCAUUGG | 1785 | CCAAUGAGAAGAUCCAAUG |
| 646 | AUUGGAUCUUCUCAUUGGA | 1786 | UCCAAUGAGAAGAUCCAAU |
| 647 | UUGGAUCUUCUCAUUGGAG | 1787 | CUCCAAUGAGAAGAUCCAA |
| 648 | UGGAUCUUCUCAUUGGAGA | 1788 | UCUCCAAUGAGAAGAUCCA |
| 650 | GAUCUUCUCAUUGGAGAGG | 1790 | CCUCUCCAAUGAGAAGAUC |
| 654 | UUCUCAUUGGAGAGGAUAA | 1794 | UUAUCCUCUCCAAUGAGAA |
| 656 | CUCAUUGGAGAGGAUAAAG | 1796 | CUUUAUCCUCUCCAAUGAG |
| 687 | AGACAGUUAUGCGGAUUCU | 1827 | AGAAUCCGCAUAACUGUCU |
| 688 | GACAGUUAUGCGGAUUCUC | 1828 | GAGAAUCCGCAUAACUGUC |
| 693 | UUAUGCGGAUUCUCUUGAA | 1833 | UUCAAGAGAAUCCGCAUAA |
| 694 | UAUGCGGAUUCUCUUGAAA | 1834 | UUUCAAGAGAAUCCGCAUA |
| 695 | AUGCGGAUUCUCUUGAAAA | 1835 | UUUUCAAGAGAAUCCGCAU |
| 746 | UACAGAGUGGUGUUACGGC | 1886 | GCCGUAACACCACUCUGUA |
| 755 | GUGUUACGGCGGUGGAAAA | 1895 | UUUUCCACCGCCGUAACAC |
| 756 | UGUUACGGCGGUGGAAAAG | 1896 | CUUUUCCACCGCCGUAACA |
| 757 | GUUACGGCGGUGGAAAAGU | 1897 | ACUUUUCCACCGCCGUAAC |
| 758 | UUACGGCGGUGGAAAAGUU | 1898 | AACUUUUCCACCGCCGUAA |
| 759 | UACGGCGGUGGAAAAGUUU | 1899 | AAACUUUUCCACCGCCGUA |
| 761 | CGGCGGUGGAAAAGUUUAA | 1901 | UUAAACUUUUCCACCGCCG |
| 773 | AGUUUAAAGUUGCCUAAGA | 1913 | UCUUAGGCAACUUUAAACU |
| 775 | UUUAAAGUUGCCUAAGAAG | 1915 | CUUCUUAGGCAACUUUAAA |
| 810 | UGGAUUGCUUUUUAGCAAU | 1950 | AUUGCUAAAAAGCAAUCCA |
| 852 | GAAGGGGUCACCUGAAAAA | 1992 | UUUUUCAGGUGACCCCUUC |
| 887 | AAAUAAAGUUCUCUUAGCG | 2027 | CGCUAAGAGAACUUUAUUU |

TABLE 8
Sequences in siRNA subset G

| SEQ ID | sense strand sequence | SEQ ID | antisense strand sequence |
|---|---|---|---|
| NO: | (5′-3′) | NO: | (5′-3′) |
| 2281 | UfsgsGfcUfaUfgGfcUfaGf | 2335 | usUfsaAfcAfcUfaGfcCfaUf |
| | uGfuUfaAfsusu | | aGfcCfasusu |
| 2282 | AfsusCfaUfaCfaAfaCfuCf | 2336 | usGfsuAfcAfgAfgUfuUfgUf |
| | uGfuAfcAfsusu | | aUfgAfususu |
| 2283 | CfsusGfuAfcUfuCfcUfgGf | 2337 | usCfsgAfuUfcCfaGfgAfaGf |
| | aAfuCfgAfsusu | | uAfcAfgsusu |
| 2284 | UfsgsUfaCfuUfcCfuGfgAf | 2338 | usUfscGfaUfuCfcAfgGfaAf |
| | aUfcGfaAfsusu | | gUfaCfasusu |
| 2285 | GfsusAfcUfuCfcUfgGfaAf | 2339 | usAfsuCfgAfuUfcCfaGfgAf |
| | uCfgAfuAfsusu | | aGfuAfcsusu |
| 2286 | UfsasCfuUfcCfuGfgAfaUf | 2340 | usUfsaUfcGfaUfuCfcAfgGf |
| | CGfaUfaAfsusu | | aAfgUfasusu |
| 2287 | CfsusUfcCfuGfgAfaUfcGf | 2341 | usAfsgUfaUfcGfaUfuCfcAf |
| | aUfaCfuAfsusu | | gGfaAfgsusu |
| 2288 | CfsusGfgAfaUfcGfaUfaCf | 2342 | usUfsaCfaAfgUfaUfcGfaUf |
| | uUfgUfaAfsusu | | uCfcAfgsusu |
| 2289 | GfsasAfuCfgAfuAfcUfuGf | 2343 | usAfsaAfuAfcAfaGfuAfuCf |
| | uAfuUfuAfsusu | | gAfuUfcsusu |
| 2290 | CfsusAfgUfaCfcAfaGfuUf | 2344 | usCfsaCfgUfaAfcUfuGfgUf |
| | aCfgUfgAfsusu | | aCfuAfgsusu |
| 2291 | UfsasGfuAfcCfaAfgUfuAf | 2345 | usGfscAfcGfuAfaCfuUfgGf |
| | cGfuGfcAfsusu | | uAfcUfasusu |
| 2292 | AfsgsUfaCfcAfaGfuUfaCf | 2346 | usUfsgCfaCfgUfaAfcUfuGf |
| | gUfgCfaAfsusu | | gUfaCfususu |
| 2293 | GfsusAfcCfaAfgUfuAfcGf | 2347 | usGfsuGfcAfcGfuAfaCfuUf |
| | uGfcAfcAfsusu | | gGfuAfcsusu |
| 2294 | UfsasCfcAfaGfuUfaCfgUf | 2348 | usGfsgUfgCfaCfgUfaAfcUf |
| | gCfaCfcAfsusu | | uGfgUfasusu |
| 2295 | CfscsAfaGfuUfaCfgUfgCf | 2349 | usUfsuGfgUfgCfaCfgUfaAf |
| | aCfcAfaAfsusu | | cUfuGfgsusu |
| 2296 | CfsasAfgUfuAfcGfuGfcAf | 2350 | usUfsuUfgGfuGfcAfcGfuAf |
| | cCfaAfaAfsusu | | aCfuUfgsusu |
| 2297 | AfsasGfuUfaCfgUfgCfaCf | 2351 | usAfsuUfuGfgUfgCfaCfgUf |
| | cAfaAfuAfsusu | | aAfcUfususu |
| 2298 | GfsusUfaCfgUfgCfaCfcAf | 2352 | usUfsaAfuUfuGfgUfgCfaCf |
| | aAfuUfaAfsusu | | gUfaAfcsusu |
| 2299 | UfsusAfcGfuGfcAfcCfaAf | 2353 | usAfsuAfaUfuUfgGfuGfcAf |
| | aUfuAfuAfsusu | | cGfuAfasusu |
| 2300 | AfsasGfaCfuCfaAfaAfgUf | 2354 | usAfsuAfuUfaCfuUfuUfgAf |
| | aAfuAfuAfsusu | | gUfcUfususu |
| 2301 | AfsasAfaUfcUfaCfuAfaAf | 2355 | usGfsaCfuUfuUfuAfgUfaGf |
| | aAfgUfcAfsusu | | aUfuUfususu |
| 2302 | UfscsUfaCfuAfaAfaAfgUf | 2356 | usCfsaGfaGfaCfuUfuUfuAf |
| | cUfcUfgAfsusu | | gUfaGfasusu |
| 2303 | UfsgsAfaGfaCfgGfgGfcUf | 2357 | usUfsaUfcUfaGfcCfcCfgUf |
| | aGfaUfaAfsusu | | cUfuCfasusu |
| 2304 | CfsusAfgAfuAfuUfgGfgAf | 2358 | usGfsuUfuCfuCfcCfaAfuAf |
| | gAfaAfcAfsusu | | uCfuAfgsusu |
| 2305 | AfsgsCfaGfaAfcGfgUfgAf | 2359 | usCfsaCfuUfuCfaCfcGfuUf |
| | aAfgUfgAfsusu | | cUfgCfususu |
| 2306 | AfsasAfgUfgGfgAfgAfuAf | 2360 | usCfsaAfuGfuAfuCfuCfcCf |
| | cAfuUfgAfsusu | | aCfuUfususu |
| 2307 | AfsasGfuGfgGfaGfaUfaCf | 2361 | usCfscAfaUfgUfaUfcUfcCf |
| | aUfuGfgAfsusu | | cAfcUfususu |
| 2308 | AfsgsUfgGfgAfgAfuAfcAf | 2362 | usUfscCfaAfuGfuAfuCfuCf |
| | uUfgGfaAfsusu | | cCfaCfususu |
| 2309 | UfsgsGfgAfgAfuAfcAfuUf | 2363 | usGfsaUfcCfaAfuGfuAfuCf |
| | gGfaUfcAfsusu | | uCfcCfasusu |
| 2310 | AfsusAfcAfuUfgGfaUfcUf | 2364 | usUfsgAfgAfaGfaUfcCfaAf |
| | uCfuCfaAfsusu | | uGfuAfususu |
| 2311 | CfsasUfuGfgAfuCfuUfcUf | 2365 | usCfsaAfuGfaGfaAfgAfuCf |
| | cAfuUfgAfsusu | | cAfaUfgsusu |
| 2312 | AfsusUfgGfaUfcUfuCfuCf | 2366 | usCfscAfaUfgAfgAfaGfaUf |
| | aUfuGfgAfsusu | | cCfaAfususu |
| 2313 | UfsusGfgAfuCfuUfcUfcAf | 2367 | usUfscCfaAfuGfaGfaAfgAf |
| | uUfgGfaAfsusu | | uCfcAfasusu |
| 2314 | UfsgsGfaUfcUfuCfuCfaUf | 2368 | usCfsuCfcAfaUfgAfgAfaGf |
| | uGfgAfgAfsusu | | aUfcCfasusu |
| 2315 | GfsasUfcUfuCfuCfaUfuGf | 2369 | usCfsuCfuCfcAfaUfgAfgAf |
| | gAfgAfgAfsusu | | aGfaUfcsusu |
| 2316 | UfsusCfuCfaUfuGfgAfgAf | 2370 | usUfsaUfcCfuCfuCfcAfaUf |
| | gGfaUfaAfsusu | | gAfgAfasusu |
| 2317 | CfsusCfaUfuGfgAfgAfgGf | 2371 | usUfsuUfaUfcCfuCfuCfcAf |
| | aUfaAfaAfsusu | | aUfgAfgsusu |
| 2318 | AfsgsAfcAfgUfuAfuGfcGf | 2372 | usGfsaAfuCfcGfcAfuAfaCf |
| | gAfuUfcAfsusu | | uGfuCfususu |
| 2319 | GfsasCfaGfuUfaUfgCfgGf | 2373 | usAfsgAfaUfcCfgCfaUfaAf |
| | aUfuCfuAfsusu | | cUfgUfcsusu |
| 2320 | UfsusAfuGfcGfgAfuUfcUf | 2374 | usUfscAfaGfaGfaAfuCfcGf |
| | cUfuGfaAfsusu | | cAfuAfasusu |
| 2321 | UfsasUfgCfgGfaUfuCfuCf | 2375 | usUfsuCfaAfgAfgAfaUfcCf |
| | uUfgAfaAfsusu | | gCfaUfasusu |
| 2322 | AfsusGfcGfgAfuUfcUfcUf | 2376 | usUfsuUfcAfaGfaGfaAfuCf |
| | uGfaAfaAfsusu | | cGfcAfususu |
| 2323 | UfsasCfaGfaGfuGfgUfgUf | 2377 | usCfscGfuAfaCfaCfcAfcUf |
| | uAfcGfgAfsusu | | cUfgUfasusu |
| 2324 | GfsusGfuUfaCfgGfcGfgUf | 2378 | usUfsuUfcCfaCfcGfcCfgUf |
| | gGfaAfaAfsusu | | aAfcAfcsusu |
| 2325 | UfsgsUfuAfcGfgCfgGfuGf | 2379 | usUfsuUfuCfcAfcCfgCfcGf |
| | gAfaAfaAfsusu | | uAfaCfasusu |
| 2326 | GfsusUfaCfgGfcGfgUfgGf | 2380 | usCfsuUfuUfcCfaCfcGfcCf |
| | aAfaAfgAfsusu | | gUfaAfcsusu |
| 2327 | UfsusAfcGfgCfgGfuGfgAf | 2381 | usAfscUfuUfuCfcAfcCfgCf |
| | aAfaGfuAfsusu | | cGfuAfasusu |
| 2328 | UfsasCfgGfcGfgUfgGfaAf | 2382 | usAfsaCfuUfuUfcCfaCfcGf |
| | aAfgUfuAfsusu | | cCfgUfasusu |
| 2329 | CfsgsGfcGfgUfgGfaAfaAf | 2383 | usUfsaAfaCfuUfuUfcCfaCf |
| | gUfuUfaAfsusu | | cGfcCfgsusu |
| 2330 | AfsgsUfuUfaAfaGfuUfgCf | 2384 | usCfsuUfaGfgCfaAfcUfuUf |
| | cUfaAfgAfsusu | | aAfaCfususu |
| 2331 | UfsusUfaAfaGfuUfgCfcUf | 2385 | usUfsuCfuUfaGfgCfaAfcUf |
| | aAfgAfaAfsusu | | uUfaAfasusu |
| 2332 | UfsgsGfaUfuGfcUfuUfuUf | 2386 | usUfsuGfcUfaAfaAfaGfcAf |
| | aGfcAfaAfsusu | | aUfcCfasusu |
| 2333 | GfsasAfgGfgGfuCfaCfcUf | 2387 | usUfsuUfuCfaGfgUfgAfcCf |
| | gAfaAfaAfsusu | | cCfuUfcsusu |
| 2334 | AfsasAfuAfaAfgUfuCfuCf | 2388 | usGfscUfaAfgAfgAfaCfuUf |
| | uUfaGfcAfsusu | | uAfuUfususu |

TABLE 9
Sequences in siRNA subset H

| siRNA | SEQ ID | sense strand sequence | SEQ ID | antisense strand sequence |
|---|---|---|---|---|
| Name | NO: | (5′-3′) | NO: | (5′-3′) |
| ETD01220 | 2389 | usgsgcuAfuGfGfcuagu | 2335 | usUfsaAfcAfcUfaGfcCfaUfa |
| | | guuaasusu | | GfcCfasusu |
| ETD01221 | 2390 | asuscauAfcAfAfacucu | 2336 | usGfsuAfcAfgAfgUfuUfgUfa |
| | | guacasusu | | UfgAfususu |
| ETD01222 | 2391 | csusguaCfuuCfCfugga | 2337 | usCfsgAfuUfcCfaGfgAfaGfu |
| | | aucgasusu | | AfcAfgsusu |
| ETD01223 | 2392 | usgsuaCfuuCfCfuggaa | 2338 | usUfscGfaUfuCfcAfgGfaAfg |
| | | ucgaasusu | | UfaCfasusu |
| ETD01224 | 2393 | gsusacUfUfccUfggaau | 2339 | usAfsuCfgAfuUfcCfaGfgAfa |
| | | cgauasusu | | GfuAfcsusu |
| ETD01225 | 2394 | usascuuccuGfGfaaucg | 2340 | usUfsaUfcGfaUfuCfcAfgGfa |
| | | auaasusu | | AfgUfasusu |
| ETD01226 | 2395 | csusuccuGfGfAfAfucg | 2341 | usAfsgUfaUfcGfaUfuCfcAfg |
| | | auacuasusu | | GfaAfgsusu |
| ETD01227 | 2396 | csusggAfAfucGfAfuac | 2342 | usUfsaCfaAfgUfaUfcGfaUfu |
| | | uuguaasusu | | CfcAfgsusu |
| ETD01228 | 2397 | gsasaucGfAfuAfcuugu | 2343 | usAfsaAfuAfcAfaGfuAfuCfg |
| | | auuuasusu | | AfuUfcsusu |
| ETD01229 | 2398 | csusaguAfccAfAfguua | 2344 | usCfsaCfgUfaAfcUfuGfgUfa |
| | | cgugasusu | | CfuAfgsusu |
| ETD01230 | 2399 | usasguAfccAfAfguuac | 2345 | usGfscAfcGfuAfaCfuUfgGfu |
| | | gugcasusu | | AfcUfasusu |
| ETD01231 | 2400 | asgsuaccAfaGfuuacgu | 2346 | usUfsgCfaCfgUfaAfcUfuGfg |
| | | gcaasusu | | UfaCfususu |
| ETD01232 | 2401 | gsusacCfaagUfUfacgu | 2347 | usGfsuGfcAfcGfuAfaCfuUfg |
| | | gcacasusu | | GfuAfcsusu |
| ETD01233 | 2402 | usasccaagUfUfaCfgug | 2348 | usGfsgUfgCfaCfgUfaAfcUfu |
| | | caccasusu | | GfgUfasusu |
| ETD01234 | 2403 | cscsaagUfUfaCfgUfgc | 2349 | usUfsuGfgUfgCfaCfgUfaAfc |
| | | accaaasusu | | UfuGfgsusu |
| ETD01235 | 2404 | csasaguuAfcGfuGfcac | 2350 | usUfsuUfgGfuGfcAfcGfuAfa |
| | | caaaasusu | | CfuUfgsusu |
| ETD01236 | 2405 | asasguUfaCfgUfgCfac | 2351 | usAfsuUfuGfgUfgCfaCfgUfa |
| | | caaauasusu | | AfcUfususu |
| ETD01237 | 2406 | gsusuaCfgugCfaCfcaa | 2352 | usUfsaAfuUfuGfgUfgCfaCfg |
| | | auuaasusu | | UfaAfcsusu |
| ETD01238 | 2407 | ususacGfuGfcAfccaaa | 2353 | usAfsuAfaUfuUfgGfuGfcAfc |
| | | uuauasusu | | GfuAfasusu |
| ETD01239 | 2408 | asasgacucAfAfAfAfgu | 2354 | usAfsuAfuUfaCfuUfuUfgAfg |
| | | aauauasusu | | UfcUfususu |
| ETD01240 | 2409 | asasaaUfCfuaCfUfaaa | 2355 | usGfsaCfuUfuUfuAfgUfaGfa |
| | | aagucasusu | | UfuUfususu |
| ETD01241 | 2410 | uscsuacuAfAfAfAfAfg | 2356 | usCfsaGfaGfaCfuUfuUfuAfg |
| | | ucucugasusu | | UfaGfasusu |
| ETD01242 | 2411 | usgsaaGfacGfGfGfGfc | 2357 | usUfsaUfcUfaGfcCfcCfgUfc |
| | | uagauaasusu | | UfuCfasusu |
| ETD01243 | 2412 | csusagaUfaUfUfgggag | 2358 | usGfsuUfuCfuCfcCfaAfuAfu |
| | | aaacasusu | | CfuAfgsusu |
| ETD01244 | 2413 | asgscaGfaacGfGfugaa | 2359 | usCfsaCfuUfuCfaCfcGfuUfc |
| | | agugasusu | | UfgCfususu |
| ETD01245 | 2414 | asasaguGfggAfgAfuac | 2360 | usCfsaAfuGfuAfuCfuCfcCfa |
| | | auugasusu | | CfuUfususu |
| ETD01246 | 2415 | asasguGfgGfaGfauaca | 2361 | usCfscAfaUfgUfaUfcUfcCfc |
| | | uuggasusu | | AfcUfususu |
| ETD01247 | 2416 | asgsugggAfgAfuAfcau | 2362 | usUfscCfaAfuGfuAfuCfuCfc |
| | | uggaasusu | | CfaCfususu |
| ETD01248 | 2417 | usgsggAfgAfuAfcAfuu | 2363 | usGfsaUfcCfaAfuGfuAfuCfu |
| | | ggaucasusu | | CfcCfasusu |
| ETD01249 | 2418 | asusacAfuuGfGfaucuu | 2364 | usUfsgAfgAfaGfaUfcCfaAfu |
| | | cucaasusu | | GfuAfususu |
| ETD01250 | 2419 | csasuuggaUfCfUfUfcu | 2365 | usCfsaAfuGfaGfaAfgAfuCfc |
| | | cauugasusu | | AfaUfgsusu |
| ETD01251 | 2420 | asusuggaUfcUfUfcuca | 2366 | usCfscAfaUfgAfgAfaGfaUfc |
| | | uuggasusu | | CfaAfususu |
| ETD01252 | 2421 | ususggaUfcUfUfcUfca | 2367 | usUfscCfaAfuGfaGfaAfgAfu |
| | | uuggaasusu | | CfcAfasusu |
| ETD01253 | 2422 | usgsgauCfuuCfuCfauu | 2368 | usCfsuCfcAfaUfgAfgAfaGfa |
| | | ggagasusu | | UfcCfasusu |
| ETD01254 | 2423 | gsasucUfuCfuCfauugg | 2369 | usCfsuCfuCfcAfaUfgAfgAfa |
| | | agagasusu | | GfaUfcsusu |
| ETD01255 | 2424 | ususcucAfuuGfGfagag | 2370 | usUfsaUfcCfuCfuCfcAfaUfg |
| | | gauaasusu | | AfgAfasusu |
| ETD01256 | 2425 | csuscauuGfGfAfGfAfg | 2371 | usUfsuUfaUfcCfuCfuCfcAfa |
| | | gauaaaasusu | | UfgAfgsusu |
| ETD01257 | 2426 | asgsacAfGfuuAfuGfcg | 2372 | usGfsaAfuCfcGfcAfuAfaCfu |
| | | gauucasusu | | GfuCfususu |
| ETD01258 | 2427 | gsascagUfUfaUfgcgga | 2373 | usAfsgAfaUfcCfgCfaUfaAfc |
| | | uucuasusu | | UfgUfcsusu |
| ETD01259 | 2428 | ususauGfcGfgAfuucuc | 2374 | usUfscAfaGfaGfaAfuCfcGfc |
| | | uugaasusu | | AfuAfasusu |
| ETD01260 | 2429 | usasugCfggaUfUfcucu | 2375 | usUfsuCfaAfgAfgAfaUfcCfg |
| | | ugaaasusu | | CfaUfasusu |
| ETD01261 | 2430 | asusgcggaUfUfcUfcuu | 2376 | usUfsuUfcAfaGfaGfaAfuCfc |
| | | gaaaasusu | | GfcAfususu |
| ETD01262 | 2431 | usascaGfaGfuGfGfugu | 2377 | usCfscGfuAfaCfaCfcAfcUfc |
| | | uacggasusu | | UfgUfasusu |
| ETD01263 | 2432 | gsusguuacGfGfcGfgug | 2378 | usUfsuUfcCfaCfcGfcCfgUfa |
| | | gaaaasusu | | AfcAfcsusu |
| ETD01264 | 2433 | usgsuuaCfggCfggugga | 2379 | usUfsuUfuCfcAfcCfgCfcGfu |
| | | aaaasusu | | AfaCfasusu |
| ETD01265 | 2434 | gsusuacGfGfcGfGfugg | 2380 | usCfsuUfuUfcCfaCfcGfcCfg |
| | | aaaagasusu | | UfaAfcsusu |
| ETD01266 | 2435 | ususacGfGfcGfGfuGfg | 2381 | usAfscUfuUfuCfcAfcCfgCfc |
| | | aaaaguasusu | | GfuAfasusu |
| ETD01267 | 2436 | usascggCfggUfggaaaa | 2382 | usAfsaCfuUfuUfcCfaCfcGfc |
| | | guuasusu | | CfgUfasusu |
| ETD01268 | 2437 | csgsgcGfGfuGfGfaaaa | 2383 | usUfsaAfaCfuUfuUfcCfaCfc |
| | | guuuaasusu | | GfcCfgsusu |
| ETD01269 | 2438 | asgsuuuAfAfAfGfuugc | 2384 | usCfsuUfaGfgCfaAfcUfuUfa |
| | | cuaagasusu | | AfaCfususu |
| ETD01270 | 2439 | ususuaaagUfUfgCfcua | 2385 | usUfsuCfuUfaGfgCfaAfcUfu |
| | | agaaasusu | | UfaAfasusu |
| ETD01271 | 2440 | usgsgaUfUfgcUfuUfuu | 2386 | usUfsuGfcUfaAfaAfaGfcAfa |
| | | agcaaasusu | | UfcCfasusu |
| ETD01272 | 2441 | gsasaggggUfCfaCfcug | 2387 | usUfsuUfuCfaGfgUfgAfcCfc |
| | | aaaaasusu | | CfuUfcsusu |
| ETD01273 | 2334 | AfsasAfuAfaAfgUfuCf | 2388 | usGfscUfaAfgAfgAfaCfuUfu |
| | | uCfuUfaGfcAfsusu | | AfuUfususu |

Any siRNA among any of subsets A-H may comprise any modification pattern described herein. If a sequence is a different number of nucleotides in length than a modification pattern, the modification pattern may still be used with the appropriate number of additional nucleotides added 5′ or 3′ to match the number of nucleotides in the modification pattern. For example, if a sense or antisense strand of the siRNA among any of subsets A-F comprises 19 nucleotides, and a modification pattern comprises 21 nucleotides, UU may be added onto the 5′ end of the sense or antisense strand.

### Example 3: Screening MTRES1 siRNAs for Activity in Human Cells in Culture

Chemically modified MTRES1 siRNAs in Table 9 were assayed for MTRES1 mRNA knockdown activity in cells in culture. SK-LMS-1 cells (ATCCR HTB-88) were seeded in 96-well tissue culture plates at a cell density of 7,500 cells per well in EMEM (ATCC Catalog No. 30-2003) supplemented with 10% fetal bovine serum and incubated overnight in a water-jacketed, humidified incubator at 37° C. in an atmosphere composed of air plus 500 carbon dioxide. These siRNAs were derived from sequences in siRNA subset F, and were cross reactive for human and non-human primate. The MTRES1 siRNAs were individually transfected into SK-LMS-1 cells in duplicate wells at 10 nM and 1 nM final concentration using 0.3 μL Lipofectamine RNAiMax (Fisher) per well. Silencer Select Negative Control #1 (ThermoFisher, Catalog #4390843) was transfected at 10 nM and 1 nM final concentration as a control. Silencer Select human MTRES1 (ThermoFisher, Catalog #4427037, ID: s27762) was transfected at 10 nM and 1 nM final concentration and used as a positive control. After incubation for 48 hours at 37° C., total RNA was harvested from each well and cDNA prepared using TaqMan® Fast Advanced Cells-to-CT™ Kit (ThermoFisher, Catalog #A35374) according to the manufacturer's instructions. The level of MTRES1 mRNA from each well was measured in triplicate by real-time qPCR on a QuantStudio™ 6 Pro Real-Time PCR System using TaqMan Gene Expression Assay for human MTRES1 (ThermoFisher, assay #Hs00360684_Ml). The level of PPIA mRNA was measured using TaqMan Gene Expression Assay (ThermoFisher, assay #Hs99999904_ml) and used to determine relative MTRES1 mRNA levels in each well using the delta-delta Ct method. All data was normalized to relative MTRES1 mRNA levels in untreated SK-LMS-1 cells. The results are shown in Table 10. The siRNAs ETD01228, ETD01270, ETD01251, ETD01235, ETD01249, ETD01258, ETD01268, ETD01273, ETD01263, ETD01240, ETD01223, ETD01262, ETD01239, ETD01242, ETD01272, ETD01220, ETD01261, ETD01243, ETD01269, ETD01256, ETD01241, ETD01238, ETD01247 and ETD01266 reduced MTRES1 levels by greater than 50% when transfected at 10 nM.

TABLE 10
Knockdown Activity of MTRES1-Specific siRNAs
at 10 nM and 1 nM in Human SK-LMS-1 Cells

| | Sense | Antisense | | |
|---|---|---|---|---|
| | Strand SEQ | Strand SEQ | Relative MTRES1 | |
| siRNA name | ID NO: | ID NO: | mRNA Level | |
| Untreated Cells | — | — | 1.00 | |
| | | | 10 nM | 1 nM |
| | | | siRNA | siRNA |
| Negative | — | — | 0.93 | 1.34 |
| Control siRNA | | | | |
| Positive | — | — | 0.39 | 0.80 |
| Control siRNA | | | | |
| ETD01220 | 2389 | 2335 | 0.34 | 0.87 |
| ETD01221 | 2390 | 2336 | 0.73 | 1.24 |
| ETD01222 | 2391 | 2337 | 1.03 | 1.18 |
| ETD01223 | 2392 | 2338 | 0.39 | 0.57 |
| ETD01224 | 2393 | 2339 | 0.62 | 0.86 |
| ETD01225 | 2394 | 2340 | 1.13 | 1.10 |
| ETD01226 | 2395 | 2341 | 0.50 | 0.69 |
| ETD01227 | 2396 | 2342 | 1.10 | 1.21 |
| ETD01228 | 2397 | 2343 | 0.50 | 0.68 |
| ETD01229 | 2398 | 2344 | 0.52 | 0.96 |
| ETD01230 | 2399 | 2345 | 1.01 | 1.14 |
| ETD01231 | 2400 | 2346 | 0.52 | 1.00 |
| ETD01232 | 2401 | 2347 | 0.78 | 1.01 |
| ETD01233 | 2402 | 2348 | 0.79 | 1.11 |
| ETD01234 | 2403 | 2349 | 0.81 | 0.92 |
| ETD01235 | 2404 | 2350 | 0.44 | 0.75 |
| ETD01236 | 2405 | 2351 | 0.87 | 1.04 |
| ETD01237 | 2406 | 2352 | 0.57 | 0.83 |
| ETD01238 | 2407 | 2353 | 0.28 | 0.49 |
| ETD01239 | 2408 | 2354 | 0.38 | 0.76 |
| ETD01240 | 2409 | 2355 | 0.41 | 0.81 |
| ETD01241 | 2410 | 2356 | 0.29 | 0.59 |
| ETD01242 | 2411 | 2357 | 0.37 | 0.61 |
| ETD01243 | 2412 | 2358 | 0.32 | 0.83 |
| ETD01244 | 2413 | 2359 | 1.00 | 1.15 |
| ETD01245 | 2414 | 2360 | 0.98 | 1.04 |
| ETD01246 | 2415 | 2361 | 0.85 | 1.05 |
| ETD01247 | 2416 | 2362 | 0.26 | 0.52 |
| ETD01248 | 2417 | 2363 | 0.92 | 1.04 |
| ETD01249 | 2418 | 2364 | 0.44 | 0.78 |
| ETD01250 | 2419 | 2365 | 1.04 | 1.10 |
| ETD01251 | 2420 | 2366 | 0.47 | 0.94 |
| ETD01252 | 2421 | 2367 | 0.83 | 1.17 |
| ETD01253 | 2422 | 2368 | 0.87 | 1.04 |
| ETD01254 | 2423 | 2369 | 0.92 | 1.02 |
| ETD01255 | 2424 | 2370 | 0.84 | 1.03 |
| ETD01256 | 2425 | 2371 | 0.29 | 0.57 |
| ETD01257 | 2426 | 2372 | 0.75 | 1.00 |
| ETD01258 | 2427 | 2373 | 0.44 | 0.93 |
| ETD01259 | 2428 | 2374 | 0.55 | 1.00 |
| ETD01260 | 2429 | 2375 | 0.66 | 1.33 |
| ETD01261 | 2430 | 2376 | 0.33 | 0.53 |
| ETD01262 | 2431 | 2377 | 0.39 | 0.92 |
| ETD01263 | 2432 | 2378 | 0.42 | 0.76 |
| ETD01264 | 2433 | 2379 | 1.00 | 1.28 |
| ETD01265 | 2434 | 2380 | 1.00 | 0.94 |
| ETD01266 | 2435 | 2381 | 0.24 | 0.36 |
| ETD01267 | 2436 | 2382 | 0.90 | 1.14 |
| ETD01268 | 2437 | 2383 | 0.44 | 1.06 |
| ETD01269 | 2438 | 2384 | 0.32 | 0.90 |
| ETD01270 | 2439 | 2385 | 0.50 | 0.91 |
| ETD01271 | 2440 | 2386 | 0.52 | 1.15 |
| ETD01272 | 2441 | 2387 | 0.35 | 0.90 |
| ETD01273 | 2442 | 2388 | 0.44 | 1.24 |

### Example 4: Determining the IC50 of MTRES1 siRNAs

The IC50 values for knockdown of MNTRES1 mRNA by select MTRES1 siRNAs will be determined in SK-LMS-1 (ATCC® HTB-88) cells. The siRNAs will be assayed individually at 30 nM, 10 nM, 3 nM, 1 nM and 0.3 nM, or 3 nM, 1 nM, 0.3 nM, 0.1 nM and 0.03 nM, or 30 nM, 10 nM, 3 nM, 1 nM, 0.3 nM, 0.1 nM and 0.03 nM. The SK-LMS-1 cells will be seeded in 96-well tissue culture plates at a cell density of 7,500 cells per well in EMEM (ATCC Catalog No. 30-2003) supplemented with 10% fetal bovine serum and incubated overnight in a water-jacketed, humidified incubator at 37° C. in an atmosphere composed of air plus 5% carbon dioxide. The MTRES1 siRNAs will be individually transfected into SK-LMS-1 cells in triplicate wells using 0.3 μL Lipofectamine RNAiMax (Fisher) per well. After incubation for 48 hours at 37° C., total RNA will be harvested from each well and cDNA prepared using TaqMan® Fast Advanced Cells-to-CT™ Kit (ThermoFisher, Catalog #A35374) according to the manufacturer's instructions. The level of MTRES1 mRNA from each well will be measured in triplicate by real-time qPCR on a QuantStudio™ 6 Pro Real-Time PCR System using TaqMan Gene Expression Assay for human MTRES1 (ThermoFisher, assay #Hs01568158_ml). The level of PPIA mRNA will be measured using TaqMan Gene Expression Assay (ThermoFisher, assay #Hs99999904 ml) and used to determine relative MTRES1 mRNA levels in each well using the delta-delta Ct method. All data will be normalized to relative MTRES1 mRNA levels in untreated SK-LMS-1 cells. Curve fit will be accomplish using the [inhibitor] vs. response (three parameters) function in GraphPad Prism software.

### Example 5: siRNA-Mediated Knockdown of MTRES1 in HCN-2 Cells

siRNAs targeted to MTRES1 mRNA that downregulate levels of MTRES1 mRNA may lead to a decrease in mRNA abundance of mitochondrially expressed NADH-ubiquinone oxidoreductase chain 5 protein (ND5), NADH-ubiquinone oxidoreductase chain 6 protein (ND6), cytochrome b (CYTB), and mitochondrially encoded 12S ribosomal RNA (12S rRNA), when administered to the cultured human neuronal cell line HCN-2 under conditions of ethidium bromide induced mitochondrial stress.

On Day 0, HCN-2 cells are to be seeded at 150,000 cells/mL into a Falcon 24-well tissue culture plate (ThermoFisher Cat. No. 353047) at 0.5 mL per well.

On Day 1, cells are treated with ethidium bromide (100 ng/ml), a well-established mitochondrial DNA replication/transcription inhibitor and stressor. Also on Day 1, MTRES1 siRNA and negative control siRNA master mixes are prepared. The MTRES1 siRNA master mix contains 350 μL of Opti-MEM (ThermoFisher Cat. No. 4427037-s1288 Lot No. AS02B02D) and 3.5 μL of a mixture of two MTRES1 siRNAs (10 μM stock). The negative control siRNA master mix contains 350 μL of Opti-MEM and 3.5 μL of negative control siRNA (ThermoFisher Cat. No. 4390843, 10 μM stock). Next, 3 μL of TransIT-X2 (Mirus Cat. No. MIR-6000) is added to each master mix. The mixes are incubated for 15 minutes to allow transfection complexes to form, then 51 μL of the appropriate master mix+TransIT-X2 is added to duplicate wells of HCN-2 cells with a final siRNA concentration of 10 nM.

On Day 3, 48 hours post transfection, duplicate wells are lysed using the Cells-to-Ct kit according to the manufacturer's protocol (ThermoFisher Cat. No. 4399002) or protein lysis buffer containing protease and phosphatase inhibitors. For the Cells-to-Ct, cells are washed with 50 μL using cold 1×PBS and lysed by adding 49.5 μL of Lysis Solution and 0.5 μL DNase I per well and pipetting up and down 5 times and incubating for 5 minutes at room temperature. Stop Solution (5 μL/well) is added to each well and mixed by pipetting up and down five times and incubating at room temperature for 2 minutes. The reverse transcriptase reaction is performed using 22.5 μL of the lysate according to the manufacturer's protocol. Samples are stored at −80° C. until real-time qPCR is performed in triplicate using TaqMan Gene Expression Assays (Applied Biosystems FAM/MTRES1, FAM/ND5, FAM/ND6, FAM/CYTB and FAM/12srRNA and using a BioRad CFX96 Cat. No. 1855195).

A decrease in MTRES1 mRNA expression in the HCN-2 cells is expected after transfection with the MTRES1 siRNAs compared to MTRES1 mRNA levels in HCN-2 cells transfected with the non-specific control siRNA 48 hours after transfection. There is an expected decrease in abundance of mitochondrial expressed genes ND5, ND6, CYTB and 12s rRNA mRNA. These results will show that the MTRES1 siRNAs elicit knockdown of MTRES1 mRNA in HCN-2 cells, and that the decrease in MTRES1 expression is correlated with a decrease in abundance of mitochondrial expressed genes ND5, ND6, CYTB and 12s rRNA mRNA.

### Example 6: ASO-Mediated Knockdown of MTRES1 in HCN-2 Cells

ASOs targeted to MTRES1 mRNA that downregulate levels of MTRES1 mRNA may lead to a decrease in mRNA abundance of mitochondrial expressed ND5, ND6, CYTB and 12s rRNA, when administered to the cultured human neuronal cell line HCN-2 under conditions of ethidium bromide induced mitochondrial stress.

On Day 0, HCN-2 cells are to be seeded at 150,000 cells/mL into a Falcon 24-well tissue culture plate (ThermoFisher Cat. No. 353047) at 0.5 mL per well.

On Day 1, cells are treated with ethidium bromide (100 ng/ml), a well-established mitochondrial DNA replication/transcription inhibitor and stressor. Also on Day 1, MTRES1 AS O and negative control ASO master mixes are prepared. The MTRES1 ASO master mix contains 350 μL of Opti-MEM (ThermoFisher Cat. No. 4427037-s1288 Lot No. AS02B02D) and 3.5 μL of a mixture of two MTRES1 ASOs (10 μM stock). The negative control ASO master mix contains 350 μL of Opti-MEM and 3.5 μL of negative control ASO (ThermoFisher Cat. No. 4390843, 10 μM stock). Next, 3 μL of TransIT-X2 (Mirus Cat. No. MIR-6000) is added to each master mix. The mixes are incubated for 15 minutes to allow transfection complexes to form, then 51 μL of the appropriate master mix+TransIT-X2 is added to duplicate wells of HCN-2 cells with a final ASO concentration of 10 nM.

On Day 3, 48 hours post transfection, duplicate wells are lysed using the Cells-to-Ct kit according to the manufacturer's protocol (ThermoFisher Cat. No. 4399002) or protein lysis buffer containing protease and phosphatase inhibitors. For the Cells-to-Ct, cells are washed with 50 μL using cold 1×PBS and lysed by adding 49.5 μL of Lysis Solution and 0.5 μL DNase I per well and pipetting up and down 5 times and incubating for 5 minutes at room temperature. Stop Solution (5 μL/well) is added to each well and mixed by pipetting up and down five times and incubating at room temperature for 2 minutes. The reverse transcriptase reaction is performed using 22.5 μL of the lysate according to the manufacturer's protocol. Samples are stored at −80° C. until real-time qPCR is performed in triplicate using TaqMan Gene Expression Assays (Applied Biosystems FAM/MTRES1, FAM/ND5, FAM/ND6, FAM/CYTB and FAM/12srRNA and using a BioRad CFX96 Cat. No. 1855195).

A decrease in MTRES1 mRNA expression in the HCN-2 cells is expected after transfection with the MTRES1 ASOs compared to MTRES1 mRNA levels in HCN-2 cells transfected with the non-specific control ASO 48 hours after transfection. There is an expected decrease in abundance of mitochondrial expressed genes ND5, ND6, CYTB and 12s rRNA mRNA. These results will show that the MTRES1 ASOs elicit knockdown of MTRES1 mRNA in HCN-2 cells, and that the decrease in MTRES1 expression is correlated with a decrease in abundance of mitochondrial expressed genes ND5, ND6, CYTB and 12s rRNA mRNA.

### Example 7: Inhibition of MTRES1 in a Mouse Model for Alzheimer's Disease Using MTRES1 siRNAs or ASOs

In this experiment, a mouse model of Alzheimer's Disease (AD) will be used to evaluate effects of siRNA or ASO inhibition of MTRES1. The model includes Tg2576 mice which express human amyloid beta precursor protein (APP) and presenilin-1 (PSEN1) transgenes with five AD-linked mutations. Cognitive function is measured using a forced swimming test (FST).

Seven-month-old mice are divided into four groups: Group 1—a group treated with non-targeting control siRNA, Group 2—a group treated with non-targeting control ASO, Group 3—a group treated with MTRES1 siRNA1, Group 4—a group treated with MTRES1 ASO1. Each group contains eight rats (4 males, 4 females), Group 5—a group treated with vehicle.

Administration of siRNA, ASO or vehicle is achieved with a 10 μL intracerebroventricular (ICV) injection of siRNA or ASO resuspended in PBS at concentration of 10 μM. On Study Day 0, Group 1 mice will be receive non-targeting control siRNA by ICV, Group 2 mice receive non-targeting control ASO by ICV, Group 3 mice will receive siRNA1 targeting mouse MTRES1 by ICV, Group 4 mice will receive ASO1 targeting mouse MTRES1 by ICV, and Group 5 mice will receive vehicle by ICV. Every other week thereafter animals from each group will be dosed for a total of 4 injections. The behavioral tests are performed 24 hrs after the final injection.

To rule out nonspecific motor effects that could influence the FST results, the potential effect of siRNA or ASO treatment on locomotor activity is assessed. Mice are evaluated using the openfield paradigm (44×44×40 cm) in a sound-attenuated room. The total distance (cm) traveled by each mouse is recorded for 5 min by a video surveillance system (SMART; Panlab SL, Barcelona, Spain) and is used to quantify activity levels. The floor of the open-field apparatus is cleaned with 10% ethanol between tests.

The FST includes a behavioral test useful for screening potential drugs that influence cognition and assessing other manipulations that are expected to affect cognitive related behaviors. On the first day, mice are placed individually in the water and allowed to swim for 15 min. The next day, mice are placed again in the water to observe the duration of immobility for 6 min using a camera. Following a 1-min session of acclimation to the apparatus, all behaviors are recorded for 5 min by a video surveillance system (SMART 2.5.21; Panlab SL). Immobility is defined as motionless floating in the water, only allowing movements necessary for the animal to keep its head above the water. The total immobility time in the FST is recorded as an index of cognitive ability.

Twenty four hours after the behavioral assessment, the mice are sacrificed by cervical dislocation following an intraperitoneal injection of 0.3 ml Nembutal (5 mg/ml) (Sigma Cat. No. 1507002). Brain and spinal cord tissues are removed and placed in RNAlater for mRNA isolation.

mRNA is isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR was performed in triplicate using TaqMan Gene Expression Assays (Applied Biosystems FAM/MTRES1 using a BioRad CFX96 Cat. No. 1855195). A decrease in MTRES1 mRNA expression in the cortical tissue from mice dosed with the MTRES1 siRNA1 or ASO1 is expected compared to MTRES1 mRNA levels in the cortical tissue from mice dosed with the non-specific controls. There is an expected decrease in the total immobility time in the FST in mice that receive the MTRES1 siRNA or ASO compared to the total immobility time in the FST in mice that receive the non-specific control along with no change between treatment groups in the locomotor activity test. These results will show that the MTRES1 siRNA or ASO elicit knockdown of MTRES1 mRNA in cortical tissue, and that the decrease in MTRES1 expression is correlated with a decrease in total immobility time in the FST along with no change in locomotor activity. These results will indicate that administration of an oligonucleotide targeting MTRES1 to a mammalian subject may be used to treat neurological disorder that includes cognitive decline.

### Example 8: Screening siRNAs Targeting Human and Mouse MTRES1 in Mice

Several siRNAs designed to be cross-reactive with human and mouse MTRES1 mRNA were tested for activity in mice. The siRNAs were attached to the GalNAc ligand ETL1. The siRNA sequences are shown in Table 11A, where Nf is a 2′ fluoro-modified nucleoside, n is a 2′ O-methyl modified nucleoside, and “s” is a phosphorothioate linkage.

Six to eight week old female mice (strain ICR, n=3) were given a subcutaneous injection on Day 0 of a single 200 ug dose of a GalNAc-conjugated siRNA or PBS as vehicle control.

Mice were euthanized on Day 14 after injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver MTRES1 mRNA were assessed by RT-qPCR in triplicate on a QuantStudio™ 6 Pro Real-Time PCR System using TaqMan assays for mouseMTRES1 (ThermoFisher, assay #Mm01229834 ml) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean MTRES1 mRNA level in animals receiving PBS. Results are shown in Table 12. Mice injected with ETD01506, ETD01507, ETD01508, and ETD01509 had substantially lower levels in mean liver MTRES1 mRNA on Day 14 relative to mice receiving PBS.

TABLE 11A
Description of Example siRNAs with Sequences

| | Sense | | Antisense | |
|---|---|---|---|---|
| | Strand | Sense Strand Sequence | Strand | |
| siRNA | SEQ ID | (5′-3′) | SEQ ID | Antisense Strand Sequence |
| Name | NO: | with GalNAc moiety | NO: | (5′-3′) |
| ETD01506 | 2463 | [ETL1]UfscsgaUfaCfuUfgUfaUfuU | 2467 | usGfsaAfaAfaUfaCfaAfgUfaUfcG |
| | | fuUfcasusu | | fasusu |
| ETD01507 | 2464 | [ETL1]csusAfcAfaAfgGfuGfaAfcu | 2468 | usCfsugaGfuUfcaccuUfuGfuagsus |
| | | cAfgAfsusu | | u |
| ETD01508 | 2465 | [ETL1]AfsusGfgAfaGfaAfaAfgcaG | 2469 | usGfsuucUfgCfuuuucUfuCfcausus |
| | | faAfcAfsusu | | u |
| ETD01509 | 2466 | [ETL1]csusuucuAfcAfaAfgGfuGfa | 2470 | usGfsuUfcAfcCfuUfuGfuAfgAfa |
| | | AfcAfsusu | | Afgsusu |

TABLE 11B
Example siRNA Base Sequences

| siRNA | SEQ ID | Sense Strand Base Sequence | SEQ ID | Antisense Strand Base Sequence |
|---|---|---|---|---|
| Name | NO: | (5′-3′) | NO: | (5′-3′) |
| ETD01506 | 2550 | UCGAUACUUGUAUUUUUCAUU | 2612 | UGAAAAAUACAAGUAUCGAUU |
| ETD01507 | 2551 | CUACAAAGGUGAACUCAGAUU | 2613 | UCUGAGUUCACCUUUGUAGUU |
| ETD01508 | 2552 | AUGGAAGAAAAGCAGAACAUU | 2614 | UGUUCUGCUUUUCUUCCAUUU |
| ETD01509 | 2553 | CUUUCUACAAAGGUGAACAUU | 2615 | UGUUCACCUUUGUAGAAAGUU |
| | | Sense Strand Base Sequence | | Antisense Strand Base Sequence |
| siRNA | SEQ ID | (5′-3′), | SEQ ID | (5′-3′), |
| Name | NO: | without 3′ overhangs | NO: | without 3′ overhangs |
| ETD01506 | 2554 | UCGAUACUUGUAUUUUUCA | 2616 | UGAAAAAUACAAGUAUCGA |
| ETD01507 | 2555 | CUACAAAGGUGAACUCAGA | 2617 | UCUGAGUUCACCUUUGUAG |
| ETD01508 | 2556 | AUGGAAGAAAAGCAGAACA | 2618 | UGUUCUGCUUUUCUUCCAU |
| ETD01509 | 2557 | CUUUCUACAAAGGUGAACA | 2619 | UGUUCACCUUUGUAGAAAG |

TABLE 12
Relative MTRES1 mRNA Levels in Livers of Mice

| | | | Dose | Mean MTRES1 mRNA |
|---|---|---|---|---|
| Group | n | Treatment | (ug) | (Normalized to Group 1, Day 14) |
| 1 | 3 | PBS | 0 | 1.00 |
| 2 | 3 | ETD01506 | 200 | 0.27 |
| 3 | 3 | ETD01507 | 200 | 0.00 |
| 4 | 3 | ETD01508 | 200 | 0.51 |
| 5 | 3 | ETD01509 | 200 | 0.51 |

### Example 9: Screening of siRNAs Targeting Human MTRES1 mRNA in Mice Transfected with AAV8 -TBG-h-MTRES1

Several siRNAs designed to be cross-reactive with human and cynomolgus monkey MTRES1 mRNA were tested for activity in mice following transfection with an adeno-associated viral vector. The siRNAs were attached to the GalNAc ligand ETL17. The siRNA sequences are shown in Table 13A, where “Nf” is a 2′ fluoro-modified nucleoside, “n” is a 2′ O-methyl modified nucleoside, “d” is a deoxynucleoside, and “s” is a phosphorothioate linkage.

Six to eight week old female mice (C571B1/6) were injected with 10 uL of a recombinant adeno-associated virus 8 (AAV8) vector (8.8×10E12 genome copies/mL) by the retroorbital route on Day −13. The recombinant AAV8 contained the open reading frame and the majority of the 3′UTR of the human MTRES1 sequence (NM 016487.5) under the control of the human thyroxine binding globulin promoter in an AAV2 backbone packaged in AAV8 capsid (AAV8-TBG-h-MTRES1). On Day 0, infected mice (n=4) were given a subcutaneous injection of a single 100 ug dose of a GalNAc-conjugated siRNA or PBS as vehicle control. were euthanized on Day 10 after subcutaneous injection and a liver sample from each was collected and placed in RNAlater (ThermoFisher Catalog #AMV7020) until processing. Total liver RNA was prepared by homogenizing the liver tissue in homogenization buffer (Maxwell RSC simply RNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver MTRES1 mRNA were assessed by RT-qPCR in triplicate on a QuantStudio™ 6 Pro Real-Time PCR System using TaqMan assays for humanMTRES1 (ThermoFisher, assay #Hs01568158_g1) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean MTRES1 mRNA level in animals receiving PBS. Results are shown in Table 14. Mice injected with ETDL1880, 1886, 1887, 1888, 1893 had greatest reductions in mean liver MTRES1 mRNA on Day 10 relative to mice receiving PBS.

TABLE 13A
Example siRNA Sequences

| | Sense | | Antisense | |
|---|---|---|---|---|
| | Strand | Sense Strand Sequence | Strand | |
| siRNA | SEQ ID | (5′-3′) | SEQ ID | Antisense Strand Sequence |
| Name | NO: | with GalNAc moiety | NO: | (5′-3′) |
| ETD01879 | 2471 | [ETL17]suacaaaCfUfCfUfguac | 2488 | usGfsgAfaGfuAfcAfgAfgUfu |
| | | uuccasusu | | UfgUfasusu |
| ETD01880 | 2472 | [ETL17]suguaCfUfUfCfCfugg | 2489 | usUfscGfaUfuCfcAfgGfaAfg |
| | | aaucgaasusu | | UfaCfasusu |
| ETD01881 | 2473 | [ETL17]suacuUfcCfUfdGgaau | 2490 | usUfsaUfcGfaUfuCfcAfgGfa |
| | | cgauaasusu | | AfgUfasusu |
| ETD01882 | 2474 | [ETL17]sgaaucGfAfuAfcuugua | 2491 | usAfsaAfuAfcAfaGfuAfuCfg |
| | | uuuasusu | | AfuUfcsusu |
| ETD01883 | 2475 | [ETL17]suucuagUfaCfCfaaguu | 2492 | usCfsgUfaAfcUfuGfgUfaCfu |
| | | acgasusu | | AfgAfasusu |
| ETD01884 | 2476 | [ETL17]saguuAfcGfuGfcAfcca | 2493 | usAfsaUfuUfgGfuGfcAfcGfu |
| | | aauuasusu | | AfaCfususu |
| ETD01885 | 2477 | [ETL17]suuacGfuGfcAfccaaau | 2494 | usAfsuAfaUfuUfgGfuGfcAfc |
| | | uauasusu | | GfuAfasusu |
| ETD01886 | 2478 | [ETL17]saaaaUfCfuaCfUfaaaa | 2495 | usGfsaCfuUfuUfuAfgUfaGfa |
| | | agucasusu | | UfuUfususu |
| ETD01887 | 2479 | [ETL17]saucuAfcuAfAfAfAfa | 2496 | usAfsgAfgAfcUfuUfuUfaGfu |
| | | gucucuasusu | | AfgAfususu |
| ETD01888 | 2480 | [ETL17]scuagaUfaUfUfgggaga | 2497 | usGfsuUfuCfuCfcCfaAfuAfu |
| | | aacasusu | | CfuAfgsusu |
| ETD01889 | 2481 | [ETL17]scauuggaUfCfUfUfcuc | 2498 | usCfsaAfuGfaGfaAfgAfuCfc |
| | | auugasusu | | AfaUfgsusu |
| ETD01890 | 2482 | [ETL17]suuggaUfCfUfUfCfuc | 2499 | usUfscCfaAfuGfaGfaAfgAfu |
| | | auuggaasusu | | CfcAfasusu |
| ETD01891 | 2483 | [ETL17]sauacAfgAfGfdTggug | 2500 | usCfsgUfaAfcAfcCfaCfuCfu |
| | | uuacgasusu | | GfuAfususu |
| ETD01892 | 2484 | [ETL17]saguuuAfAfAfGfuugc | 2501 | usCfsuUfaGfgCfaAfcUfuUfa |
| | | cuaagasusu | | AfaCfususu |
| ETD01893 | 2485 | [ETL17]suuuaaagUfUfgCfcuaa | 2502 | usUfsuCfuUfaGfgCfaAfcUfu |
| | | gaaasusu | | UfaAfasusu |
| ETD01894 | 2486 | [ETL17]suggaUfUfgCfUfuUfu | 2503 | usUfsuGfcUfaAfaAfaGfcAfa |
| | | uagcaaasusu | | UfcCfasusu |
| ETD01895 | 2487 | [ETL17]saaauAfaAfGfdTucucu | 2504 | usGfscUfaAfgAfgAfaCfuUfu |
| | | uagcasusu | | AfuUfususu |

TABLE 13B
Example siRNA Base Sequences

| siRNA | SEQ ID | Sense Strand Base Sequence | SEQ ID | Antisense Strand Base Sequence |
|---|---|---|---|---|
| Name | NO: | (5′-3′) | NO: | (5′-3′) |
| ETD01879 | 2558 | UACAAACUCUGUACUUCCAUU | 2620 | UGGAAGUACAGAGUUUGUAUU |
| ETD01880 | 2559 | UGUACUUCCUGGAAUCGAAUU | 2621 | UUCGAUUCCAGGAAGUACAUU |
| ETD01881 | 2560 | UACUUCCUGGAAUCGAUAAUU | 2622 | UUAUCGAUUCCAGGAAGUAUU |
| ETD01882 | 2561 | GAAUCGAUACUUGUAUUUAUU | 2623 | UAAAUACAAGUAUCGAUUCUU |
| ETD01883 | 2562 | UUCUAGUACCAAGUUACGAUU | 2624 | UCGUAACUUGGUACUAGAAUU |
| ETD01884 | 2563 | AGUUACGUGCACCAAAUUAUU | 2625 | UAAUUUGGUGCACGUAACUUU |
| ETD01885 | 2564 | UUACGUGCACCAAAUUAUAUU | 2626 | UAUAAUUUGGUGCACGUAAUU |
| ETD01886 | 2565 | AAAAUCUACUAAAAAGUCAUU | 2627 | UGACUUUUUAGUAGAUUUUUU |
| ETD01887 | 2566 | AUCUACUAAAAAGUCUCUAUU | 2628 | UAGAGACUUUUUAGUAGAUUU |
| ETD01888 | 2567 | CUAGAUAUUGGGAGAAACAUU | 2629 | UGUUUCUCCCAAUAUCUAGUU |
| ETD01889 | 2568 | CAUUGGAUCUUCUCAUUGAUU | 2630 | UCAAUGAGAAGAUCCAAUGUU |
| ETD01890 | 2569 | UUGGAUCUUCUCAUUGGAAUU | 2631 | UUCCAAUGAGAAGAUCCAAUU |
| ETD01891 | 2570 | AUACAGAGTGGUGUUACGAUU | 2632 | UCGUAACACCACUCUGUAUUU |
| ETD01892 | 2571 | AGUUUAAAGUUGCCUAAGAUU | 2633 | UCUUAGGCAACUUUAAACUUU |
| ETD01893 | 2572 | UUUAAAGUUGCCUAAGAAAUU | 2634 | UUUCUUAGGCAACUUUAAAUU |
| ETD01894 | 2573 | UGGAUUGCUUUUUAGCAAAUU | 2635 | UUUGCUAAAAAGCAAUCCAUU |
| ETD01895 | 2574 | AAAUAAAGTUCUCUUAGCAUU | 2636 | UGCUAAGAGAACUUUAUUUUU |
| | | Sense Strand Base Sequence | | Antisense Strand Base Sequence |
| siRNA | SEQ ID | (5′-3′), | SEQ ID | (5′-3′), |
| Name | NO: | without 3′ overhangs | NO: | without 3′ overhangs |
| ETD01879 | 2575 | UACAAACUCUGUACUUCCA | 2637 | UGGAAGUACAGAGUUUGUA |
| ETD01880 | 2576 | UGUACUUCCUGGAAUCGAA | 2638 | UUCGAUUCCAGGAAGUACA |
| ETD01881 | 2577 | UACUUCCUGGAAUCGAUAA | 2639 | UUAUCGAUUCCAGGAAGUA |
| ETD01882 | 2578 | GAAUCGAUACUUGUAUUUA | 2640 | UAAAUACAAGUAUCGAUUC |
| ETD01883 | 2579 | UUCUAGUACCAAGUUACGA | 2641 | UCGUAACUUGGUACUAGAA |
| ETD01884 | 2580 | AGUUACGUGCACCAAAUUA | 2642 | UAAUUUGGUGCACGUAACU |
| ETD01885 | 2581 | UUACGUGCACCAAAUUAUA | 2643 | UAUAAUUUGGUGCACGUAA |
| ETD01886 | 2582 | AAAAUCUACUAAAAAGUCA | 2644 | UGACUUUUUAGUAGAUUUU |
| ETD01887 | 2583 | AUCUACUAAAAAGUCUCUA | 2645 | UAGAGACUUUUUAGUAGAU |
| ETD01888 | 2584 | CUAGAUAUUGGGAGAAACA | 2646 | UGUUUCUCCCAAUAUCUAG |
| ETD01889 | 2585 | CAUUGGAUCUUCUCAUUGA | 2647 | UCAAUGAGAAGAUCCAAUG |
| ETD01890 | 2586 | UUGGAUCUUCUCAUUGGAA | 2648 | UUCCAAUGAGAAGAUCCAA |
| ETD01891 | 2587 | AUACAGAGTGGUGUUACGA | 2649 | UCGUAACACCACUCUGUAU |
| ETD01892 | 2588 | AGUUUAAAGUUGCCUAAGA | 2650 | UCUUAGGCAACUUUAAACU |
| ETD01893 | 2589 | UUUAAAGUUGCCUAAGAAA | 2651 | UUUCUUAGGCAACUUUAAA |
| ETD01894 | 2590 | UGGAUUGCUUUUUAGCAAA | 2652 | UUUGCUAAAAAGCAAUCCA |
| ETD01895 | 2591 | AAAUAAAGTUCUCUUAGCA | 2653 | UGCUAAGAGAACUUUAUUU |

TABLE 14
Relative human MTRES1 mRNA Levels in Livers of Mice

| | | | Dose | Mean MTRES1 mRNA |
|---|---|---|---|---|
| Group | n | Treatment | (ug) | (Normalized to Group 1, Day 10) |
| 1 | 4 | PBS | 0 | 1.00 |
| 2 | 4 | ETD01879 | 100 | 0.70 |
| 3 | 4 | ETD01880 | 100 | 0.45 |
| 4 | 4 | ETD01881 | 100 | 0.78 |
| 5 | 4 | ETD01882 | 100 | 2.07 |
| 6 | 4 | ETD01883 | 100 | 1.24 |
| 7 | 4 | ETD01884 | 100 | 1.12 |
| 8 | 4 | ETD01885 | 100 | 0.97 |
| 9 | 4 | ETD01886 | 100 | 0.46 |
| 10 | 4 | ETD01887 | 100 | 0.18 |
| 11 | 4 | ETD01888 | 100 | 0.14 |
| 12 | 4 | ETD01889 | 100 | 0.74 |
| 13 | 4 | ETD01890 | 100 | 1.73 |
| 14 | 4 | ETD01891 | 100 | 3.21 |
| 15 | 4 | ETD01892 | 100 | 2.59 |
| 16 | 4 | ETD01893 | 100 | 0.55 |
| 17 | 4 | ETD01894 | 100 | 1.12 |
| 18 | 4 | ETD01895 | 100 | 0.65 |

### Example 10: Screening siRNAs Targeting Human and Mouse MTRES1 in Mice

Several siRNAs designed to be cross-reactive with human, mouse and cynomolgus monkey MTRES1 mRNA were tested for activity in mice. The siRNAs were attached to the GalNAc ligand ETL1 or ETL17. The siRNA sequences are shown in Table 15A, where Nf is a 2′ fluoro-modified nucleoside, n is a 2′ O-methyl modified nucleoside, “d” is a deoxynucleoside, and “s” is a phosphorothioate linkage.

Six to eight week old female mice (strain ICR, n=3) were given a subcutaneous injection on Day 0 of a single 200 ug dose of a GalNAc-conjugated siRNA or PBS as vehicle control.

Mice were euthanized on Day 10 after injection and a liver sample from each was collected and placed in RNAlater (ThermoFisher Catalog #AM7020) until processing. Total liver RNA was prepared by homogenizing the liver tissue in homogenization buffer (Maxwell RSC simply RNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver MTRES1 mRNA were assessed by RT-qPCR in triplicate on a QuantStudio™ 6 Pro Real-Time PCR System using TaqMan assays for mouse MTRES1 (ThermoFisher, assay #Mm01229834 ml) and the mouse housekeeping gene PPIA (ThermoFisher, assay #Mm02342430_g1) and PerfeCTaR qPCR FastMix®, Low ROX™ (VWR, Catalog #101419-222). Data were normalized to the mean MTRES1 mRNA level in animals receiving PBS. Results are shown in Table 16. Mice injected with ETD01597, ETD01955, ETD01958, and had substantially lower levels in mean liver MTRES1 mRNA on Day 10 relative to mice receiving PBS.

TABLE 15A
Example siRNA Sequences

| | Sense | | Antisense | |
|---|---|---|---|---|
| | Strand | Sense Strand Sequence | Strand | |
| siRNA | SEQ ID | (5′-3′) | SEQ ID | Antisense Strand Sequence |
| Name | NO: | with GalNAc moiety | NO: | (5′-3′) |
| ETD01597 | 2505 | [ETL1]sguaucuccAfgAfauguu | 2515 | usAfsuAfaCfaUfuCfuGfgAfgA |
| | | auasusu | | fuAfcsusu |
| ETD01954 | 2506 | [ETL17]sacuuccuGfGfAfAfuc | 2516 | usGfsuAfsuCfgAfuUfcCfaGfg |
| | | gauacasusu | | AfaGfususu |
| ETD01955 | 2507 | [ETL17]scuuccuGfGfAfAfucg | 2517 | usAfsgUfaUfcGfaUfuCfcAfgG |
| | | auacuasusu | | faAfgsusu |
| ETD01956 | 2508 | [ETL17]scuggAfAfucGfAfuac | 2518 | usUfsaCfaAfgUfaUfcGfaUfuCf |
| | | uuguaasusu | | cAfgsusu |
| ETD01957 | 2509 | [ETL17]sggaaUfCfgaUfaCfuu | 2519 | usAfsaUfaCfaAfgUfaUfcGfaUf |
| | | guauuasusu | | uCfcsusu |
| ETD01958 | 2510 | [ETL17]sgaugCfUfuUfCfuaca | 2520 | usAfscCfuUfuGfuAfgAfaAfgC |
| | | aagguasusu | | faUfcsusu |
| ETD01959 | 2511 | [ETL17]sagaaAfAfgcAfGfaac | 2521 | usUfscAfcCfgUfuCfuGfcUfuU |
| | | ggugaasusu | | fuCfususu |
| ETD01960 | 2512 | [ETL17]saagcagAfAfdCGfgu | 2522 | usAfscUfuUfcAfcCfgUfuCfuG |
| | | gaaaguasusu | | fcUfususu |
| ETD01961 | 2513 | [ETL17]sagugGfGfaGfAfuAf | 2523 | usUfscCfaAfuGfuAfuCfuCfcCf |
| | | cauuggaasusu | | aCfususu |
| ETD01962 | 2514 | [ETL17]sugggAfGfauAfcAfu | 2524 | usGfsaUfcCfaAfuGfuAfuCfuC |
| | | uggaucasusu | | fcCfasusu |

TABLE 15B
Example siRNA Base Sequences

| siRNA | SEQ ID | Sense Strand Base Sequence | SEQ ID | Antisense Strand Base Sequence |
|---|---|---|---|---|
| Name | NO: | (5′ to 3′) | NO: | (5′ to 3′) |
| ETD01597 | 2592 | GUAUCUCCAGAAUGUUAUAUU | 2654 | UAUAACAUUCUGGAGAUACUU |
| ETD01954 | 2593 | ACUUCCUGGAAUCGAUACAUU | 2655 | UGUAUCGAUUCCAGGAAGUUU |
| ETD01955 | 2594 | CUUCCUGGAAUCGAUACUAUU | 2656 | UAGUAUCGAUUCCAGGAAGUU |
| ETD01956 | 2595 | CUGGAAUCGAUACUUGUAAUU | 2657 | UUACAAGUAUCGAUUCCAGUU |
| ETD01957 | 2596 | GGAAUCGAUACUUGUAUUAUU | 2658 | UAAUACAAGUAUCGAUUCCUU |
| ETD01958 | 2597 | GAUGCUUUCUACAAAGGUAUU | 2659 | UACCUUUGUAGAAAGCAUCUU |
| ETD01959 | 2598 | AGAAAAGCAGAACGGUGAAUU | 2660 | UUCACCGUUCUGCUUUUCUUU |
| ETD01960 | 2599 | AAGCAGAACGGUGAAAGUAUU | 2661 | UACUUUCACCGUUCUGCUUUU |
| ETD01961 | 2600 | AGUGGGAGAUACAUUGGAAUU | 2662 | UUCCAAUGUAUCUCCCACUUU |
| ETD01962 | 2601 | UGGGAGAUACAUUGGAUCAUU | 2663 | UGAUCCAAUGUAUCUCCCAUU |
| siRNA | SEQ ID | Sense Strand Base Sequence (5′ to | SEQ ID | Antisense Strand Base Sequence |
| Name | NO: | 3′), | NO: | (5′ to 3′), without 3′ overhangs |
| ETD01597 | 2602 | GUAUCUCCAGAAUGUUAUA | 2664 | UAUAACAUUCUGGAGAUAC |
| ETD01954 | 2603 | ACUUCCUGGAAUCGAUACA | 2665 | UGUAUCGAUUCCAGGAAGU |
| ETD01955 | 2604 | CUUCCUGGAAUCGAUACUA | 2666 | UAGUAUCGAUUCCAGGAAG |
| ETD01956 | 2605 | CUGGAAUCGAUACUUGUAA | 2667 | UUACAAGUAUCGAUUCCAG |
| ETD01957 | 2606 | GGAAUCGAUACUUGUAUUA | 2668 | UAAUACAAGUAUCGAUUCC |
| ETD01958 | 2607 | GAUGCUUUCUACAAAGGUA | 2669 | UACCUUUGUAGAAAGCAUC |
| ETD01959 | 2608 | AGAAAAGCAGAACGGUGAA | 2670 | UUCACCGUUCUGCUUUUCU |
| ETD01960 | 2609 | AAGCAGAACGGUGAAAGUA | 2671 | UACUUUCACCGUUCUGCUU |
| ETD01961 | 2610 | AGUGGGAGAUACAUUGGAA | 2672 | UUCCAAUGUAUCUCCCACU |
| ETD01962 | 2611 | UGGGAGAUACAUUGGAUCA | 2673 | UGAUCCAAUGUAUCUCCCA |

TABLE 16
Relative MTRES1 mRNA Levels in Livers of Mice

| | | | Dose | Mean MTRES1 mRNA |
|---|---|---|---|---|
| Group | n | Treatment | (ug) | (Normalized to Group 1, Day 10) |
| 1 | 3 | PBS | | 1.00 |
| 2 | 3 | ETD01597 | 200 | 0.13 |
| 3 | 3 | ETD01954 | 200 | 1.03 |
| 4 | 3 | ETD01955 | 200 | 0.16 |
| 5 | 3 | ETD01956 | 200 | 0.62 |
| 6 | 3 | ETD01957 | 200 | 0.31 |
| 7 | 3 | ETD01958 | 200 | 0.18 |
| 8 | 3 | ETD01959 | 200 | 0.53 |
| 9 | 3 | ETD01960 | 200 | 0.69 |
| 10 | 3 | ETD01961 | 200 | 0.33 |
| 11 | 3 | ETD01962 | 200 | 0.79 |

### Example 11: Oligonucleotide Synthesis

Oligonucleotides such as siRNAs may be synthesized according to phosphoramidite technology on a solid phase. For example, a K&A oligonucleotide synthesizer may be used. Syntheses may be performed on a solid support made of controlled pore glass (CPG, 500 Å or 600 Å, obtained from AM Chemicals, Oceanside, CA, USA). All 2′-OMe and 2′-F phosphoramidites may be purchased from Hongene Biotech (Union City, CA, USA). All phosphoramidites may be dissolved in anhydrous acetonitrile (100 mM) and molecular sieves (3 Å) may be added. 5-Benzylthio-1H-tetrazole (BTT, 250 mM in acetonitrile) or 5-Ethylthio-1H-tetrazole (ETT, 250 mM in acetonitrile) may be used as activator solution. Coupling times may be 9-18 min (e.g. with a GalNAc such as ETL17), 6 min (e.g. with 2′OMe and 2′F). In order to introduce phosphorothioate linkages, a 100 mM solution of 3-phenyl 1,4-dithiazoline-5-one (POS, obtained from PolyOrg, Inc., Leominster, Mass., USA) in anhydrous acetonitrile may be employed.

After solid phase synthesis, the dried solid support may be treated with a 1:1 volume solution of 40 wt. % methylamine in water and 28% ammonium hydroxide solution (Aldrich) for two hours at 30° C. The solution may be evaporated and the solid residue may be reconstituted in water and purified by anionic exchange HPLC using a TKSgel SuperQ-5PW 13u column. Buffer A may be 20 mM Tris, 5 mM EDTA, pH 9.0 and contained 20% Acetonitrile and buffer B may be the same as buffer A with the addition of 1 M sodium chloride. UV traces at 260 nm may be recorded. Appropriate fractions may be pooled then desalted using Sephadex G-25 medium.

Equimolar amounts of sense and antisense strand may be combined to prepare a duplex. The duplex solution may be prepared in 0.1×PBS (Phosphate-Buffered Saline, 1×, Gibco). The duplex solution may be annealed at 95° C. for 5 min, and cooled to room temperature slowly. Duplex concentration may be determined by measuring the solution absorbance on a UV-Vis spectrometer at 260 nm in 0.1×PBS. For some experiments, a conversion factor may be calculated from an experimentally determined extinction coefficient.

### Example 12: GalNAc Ligand for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. Reagents for GalNAc conjugation to oligonucleotides are shown in Table 17.

TABLE 17
GalNAc Conjugation Reagents

| Type of | |
|---|---|
| conjugation | Structure |
| Solid phase 3′ attachment where squiggly line is rest of oligonucleotide chain and right- most OH is where attachment\grave{ } to solid phase is. | 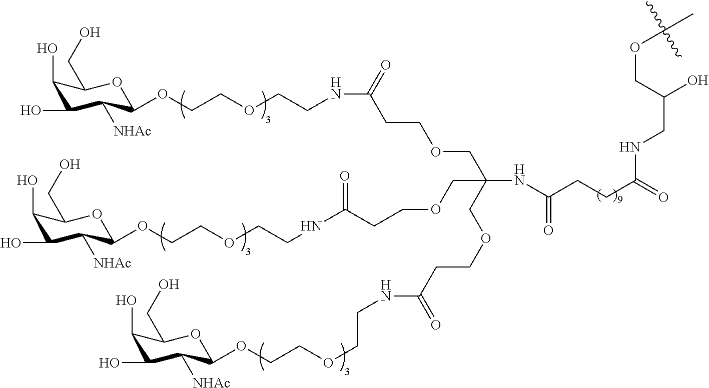 |
| | This GalNAc ligand may be referred to as “GalNAc23” or “GalNAc#23.” |
| Solid phase 5′ attachment phosphoramidite | 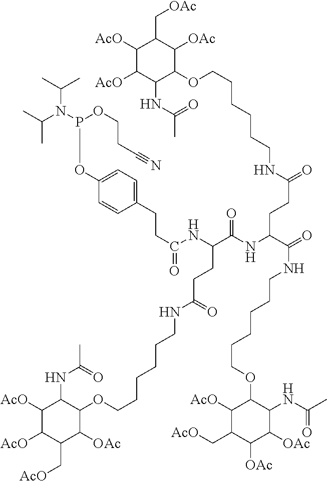 [SMILES: CC(=O)NC1C(OCCCCCCNC(=O)CCC(NC(=O)CCc2ccc(OP(OCCC#N)N(C(C)C)C(C)C)cc2)C(=O)NC(CCC(=O)NCCCCCCOC2C(NC(C)=O)C(OC(C)=O)C(OC(C)=O)C(COC(C)=O)C2OC(C)=O)C(=O)NCCCCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solid phase 5′ attachment Phosphoramidite | 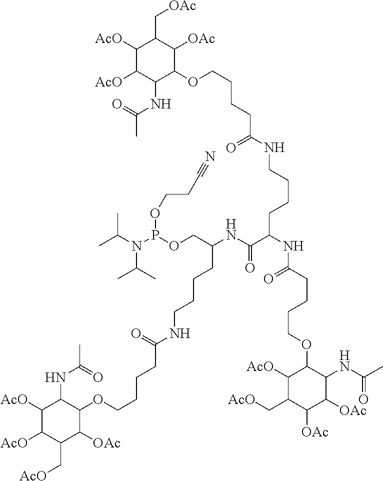 [SMILES: CC(=O)NC1C(OCCCCC(=O)NCCCCC(COP(OCCC#N)N(C(C)C)C(C)C)NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solution phase Carboxylic acid for amide coupling anywhere on oligonucleotide | 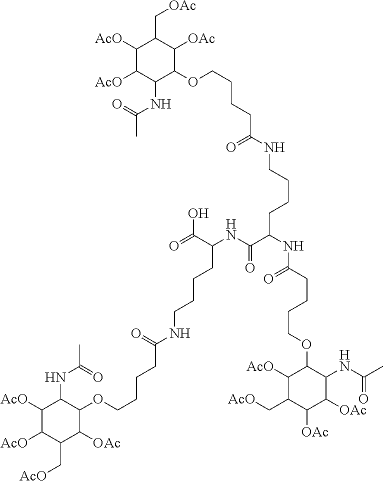 [SMILES: CC(=O)NC1C(OCCCCC(=O)NCCCCC(NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(=O)O)C(OC(C)=O)C(COC(C)=O)C(OC(C)=O)C1OC(C)=O] |
| | Where Ac is an acetyl group or other hydroxyl protecting group that can be removed |
| | under basic, acid or reducing conditions. |

In solution phase conjugation, the oligonucleotide sequence-including a reactive conjugation site—is formed on the resin. The oligonucleotide is then removed from the resin and GalNAc is conjugated to the reactive site.

The carboxy GalNAc derivatives may be coupled to amino-modified oligonucleotides. The peptide coupling conditions are known to the skilled in the art using a carbodiimide coupling agent like DCC (N,N′-Dicyclohexylcarbodiimide), EDC (N-(3-dimethylaminopropyl)-N′-ethylcarbodiimide) or EDC·HCl (N-(3-dimethylaminopropyl)-N′-ethylcarbodiimide hydrochloride and an additive like HOBt (1-hydroxybenztriazole), HOSu (N-hydroxysuccinimide), TBTU (N,N,N′,N′-Tetramethyl-O-(benzotriazol-1-yl)uronium tetrafluoroborate, HBTU (2-(1H-benzotriazol-1-yl)-1,1,3,3-tetramethyluronium hexafluorophosphate) or HOAt (1-Hydroxy-7-azabenzotriazole and common combinations thereof such as TBTU/HOBt or HBTU/HOAt to form activated amine-reactive esters.

Amine groups may be incorporated into oligonucleotides using a number of known, commercially available reagents at the 5′ terminus, 3′ terminus or anywhere in between.

Non-limiting examples of reagents for oligonucleotide synthesis to incorporate an amino group include:
5′ attachment:
6-(4-Monomethoxytritylamino)hexyl-(2-cyanoethyl)-(N,N-diisopropyl)-phosphoramidite CAS Number: 114616-27-2
5′-Amino-Modifier TEG CE-Phosphoramidite
10-(O-trifluoroacetamido-N-ethyl)-triethyleneglycol-1-[(2-cyanoethyl)-(N,N-diisoprol-)]-phosphoramidite
3′ attachment:
3′-Amino-Modifier Serinol CPG
3-Dimethoxytrityloxy-2-(3-(fluorenylmethoxycarbonylamino)propanamido)propyl-1-O-succinyl-long chain alkylamino-CPG (where CPG stands for controlled-pore glass and is the solid support)
3′-Amino-Modifier Serinol Phosphoramidite
3-Dimethoxytrityloxy-2-(3-(fluorenylmethoxycarbonylamino)propanamido)propyl-1-O-(-2-cyanoethyl)-(N,N-diisopropyl)-phosphoramidite

### Internal (Base Modified):

Amino-Modifier C6 dT
5′-Dimethoxytrityl-5-[N-(trifluoroacetylaminohexyl)-3-acrylimido]-2′-deoxyUridine,3′-[((2-cyanoethyl)-(N,N-diisopropyl)]-phosphoramidite. CAS Number: 178925-21-8

Solution phase conjugations may occur after oligonucleotide synthesis via reactions between non-nucleosidic nucleophilic functional groups that are attached to the oligonucleotide and electrophilic GalNAc reagents. Examples of nucleophilic groups include amines and thiols, and examples of electrophilic reagents include activated esters (e.g. N-hydroxysuccinimide, pentafluorophenyl) and maleimides.

### Example 13: GalNAc Ligands for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. A non-limiting example of a phosphoramidite reagent for GalNAc conjugation to a 5′ end oligonucleotide is shown in Table 18.

TABLE 18
GalNAc Conjugation Reagent

| Type of | |
|---|---|
| conjugation | Structure |
| Solid phase 5′ attachment phosphoramidite | 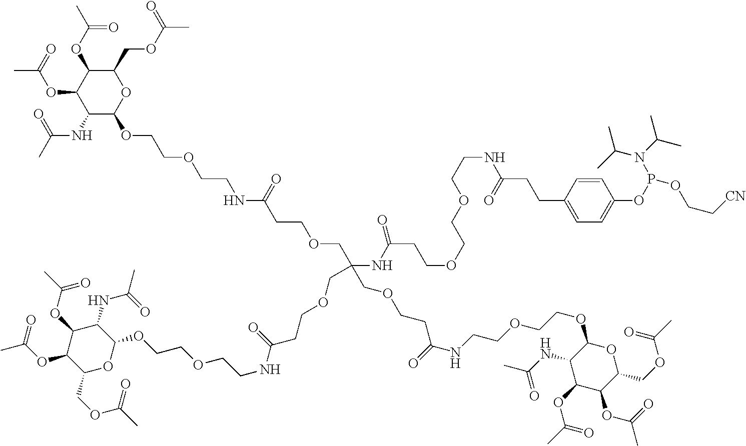 [SMILES: CC(=O)N[C@@H]1[C@H](OCCOCCNC(=O)CCOCC(COCCC(=O)NCCOCCO[C@H]2O[C@H](COC(C)=O)[C@@H](OC(C)=O)[C@@H](OC(C)=O)[C@@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)N(C(C)C)C(C)C)cc2)O[C@H](COC(C)=O)[C@@H](OC(C)=O)[C@H]1OC(C)=O] |

The following includes examples of synthesis reactions used to create a GalNAc moiety:

### Scheme for the Preparation of NAcegal-Linker-TMSOTf

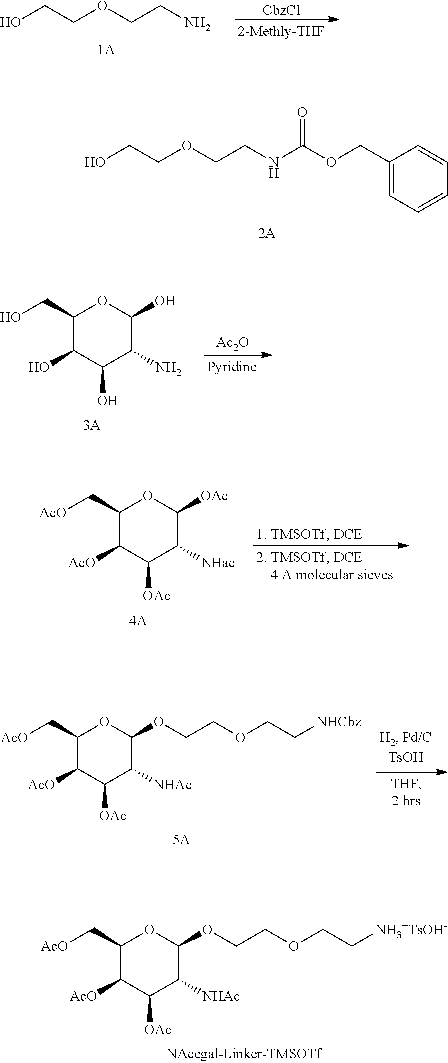

### General Procedure for Preparation of Compound 2A

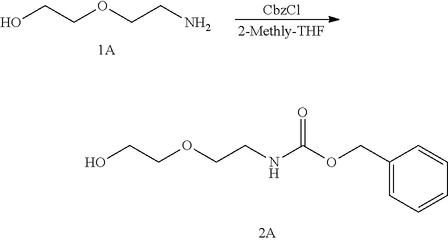

To a solution of Compound 1A (500 g, 4.76 mol, 476 mL) in 2-Methyl-THF (2.00 L) is added CbzCl (406 g, 2.38 mol, 338 mL) in 2-Methyl-THF (750 mL) dropwise at 0° C. The mixture is stirred at 25° C. for 2 hrs under N₂ atmosphere. TLC (DCM:MeOH=20:1, PMA) may indicate CbzCl is consumed completely and one new spot (R_{f}=0.43) formed. The reaction mixture is added HCl/EtOAc (1 N, 180 mL) and stirred for 30 mins, white solid is removed by filtration through celite, the filtrate is concentrated under vacuum to give Compound 2A (540 g, 2.26 mol, 47.5% yield) as a pale yellow oil and used into the next step without further purification. ¹H NMR: δ 7.28-7.41 (m, 5H), 5.55 (br s, 1H), 5.01-5.22 (m, 2H), 3.63-3.80 (m, 2H), 3.46-3.59 (m, 4H), 3.29-3.44 (m, 2H), 2.83-3.02 (m, 1H).

### General Procedure for Preparation of Compound 4A

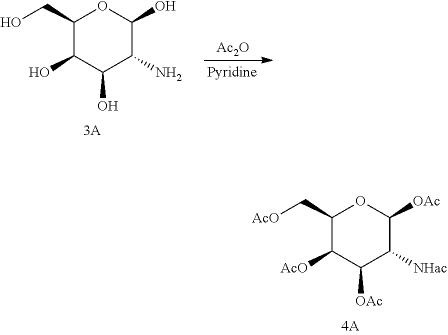

To a solution of Compound 3A (1.00 kg, 4.64 mol, HCl) in pyridine (5.00 L) is added acetyl acetate (4.73 kg, 46.4 mol, 4.34 L) dropwise at 0° C. under N₂ atmosphere. The mixture is stirred at 25° C. for 16 hrs under N₂ atmosphere. TLC (DCM:MeOH=20:1, PMA) indicated Compound 3A is consumed completely and two new spots (R_{f}=0.35) formed. The reaction mixture is added to cold water (30.0 L) and stirred at 0° C. for 0.5 hr, white solid formed, filtered and dried to give Compound 4A (1.55 kg, 3.98 mol, 85.8% yield) as a white solid and used in the next step without further purification. ¹H NMR: δ 7.90 (d, J=9.29 Hz, 1H), 5.64 (d, J=8.78 Hz, 1H), 5.26 (d, J=3.01 Hz, 1H), 5.06 (dd, J=11.29, 3.26 Hz, 1H), 4.22 (t, J=6.15 Hz, 1H), 3.95-4.16 (m, 3H), 2.12 (s, 3H), 2.03 (s, 3H), 1.99 (s, 3H), 1.90 (s, 3H), 1.78 (s, 3H).

### General Procedure for Preparation of Compound 5A

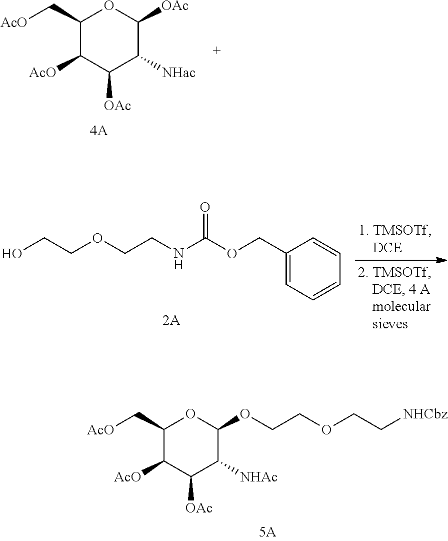

To a solution of Compound 4A (300 g, 771 mmol) in DCE (1.50 L) is added TMSOTf (257 g, 1.16 mol, 209 mL) and stirred for 2 hrs at 60° C., and then stirred for 1 hr at 25° C. Compound 2A (203 g, 848 mmol) is dissolved in DCE (1.50 L) and added 4 Å powder molecular sieves (150 g) stirring for 30 mins under N₂ atmosphere. Then the solution of Compound 4A in DCE is added dropwise to the mixture at 0° C. The mixture is stirred at 25° C. for 16 hrs under N₂ atmosphere. TLC (DCM:MeOH=25:1, PMA) indicated Compound 4A is consumed completely and new spot (R_{f}=0.24) formed. The reaction mixture is filtered and washed with sat. NaHCO₃ (2.00 L), water (2.00 L) and sat. brine (2.00 L). The organic layer is dried over anhydrous Na₂SO4, filtered and concentrated under reduced pressure to give a residue. The residue is triturated with 2-Me-THE/heptane (5/3, v/v, 1.80 L) for 2 hrs, filtered and dried to give Compound 5A (225 g, 389 mmol, 50.3% yield, 98.4% purity) as a white solid. ¹H NMR: δ 7.81 (d, J 9.29 Hz, 1H), 7.20-7.42 (m, 6H), 5.21 (d, J=3.26 Hz, 1H), 4.92-5.05 (m, 3H), 4.55 (d, J=8.28 Hz, 1H), 3.98-4.07 (m, 3H), 3.82-3.93 (m, 1H), 3.71-3.81 (m, 1H), 3.55-3.62 (m, 1H), 3.43-3.53 (m, 2H), 3.37-3.43 (m, 2H), 3.14 (q, J=5.77 Hz, 2H), 2.10 (s, 3H), 1.99 (s, 3H), 1.89 (s, 3H), 1.77 (s, 3H).

### General Procedure for Preparation of NAcegal-Linker-Tosylate Salt

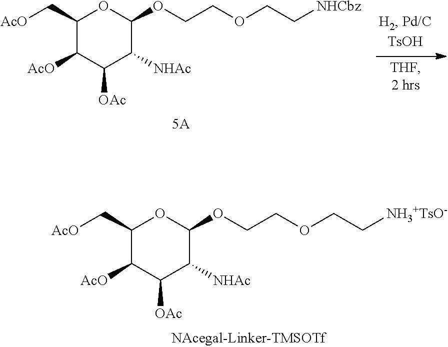

To a solution of Compound 5A (200 g, 352 mmol) in THF (1.0 L) is added dry Pd/C (15.0 g, 10% purity) and TsOH (60.6 g, 352 mmol) under N₂ atmosphere. The suspension is degassed under vacuum and purged with H₂ several times. The mixture is stirred at 25° C. for 3 hrs under H₂ (45 psi) atmosphere. TLC (DCM:MeOH=10:1, PMA) indicated Compound 5A is consumed completely and one new spot (R_{f}=0.04) is formed. The reaction mixture is filtered and concentrated (<40° C.) under reduced pressure to give a residue. Diluted with anhydrous DCM (500 mL, dried overnight with 4 Å molecular sieves (dried at 300° C. for 12 hrs)) and concentrate to give a residue and run Karl Fisher (KF) to check for water content. This is repeated 3 times with anhydrous DCM (500 mL) dilutions and concentration to give NAcegal-Linker-TMSOTf (205 g, 95.8% yield, TsOH salt) as a foamy white solid. ¹H NMR: δ 7.91 (d, J 9.03 Hz, 1H), 7.53-7.86 (m, 2H), 7.49 (d, J=8.03 Hz, 2H), 7.13 (d, J=8.03 Hz, 2H), 5.22 (d, J 3.26 Hz, 1H), 4.98 (dd, J=11.29, 3.26 Hz, 1H), 4.57 (d, J=8.53 Hz, 1H), 3.99-4.05 (m, 3H), 3.8-3.94 (m, 1H), 3.79-3.85 (m, 1H), 3.51-3.62 (m, 5H), 2.96 (br t, J=5.14 Hz, 2H), 2.29 (s, 3H), 2.10 (s, 3H), 2.00 (s, 3H), 1.89 (s, 3H), 1.78 (s, 3H).

### Scheme for the Preparation of TRIS-PEG2-CBZ

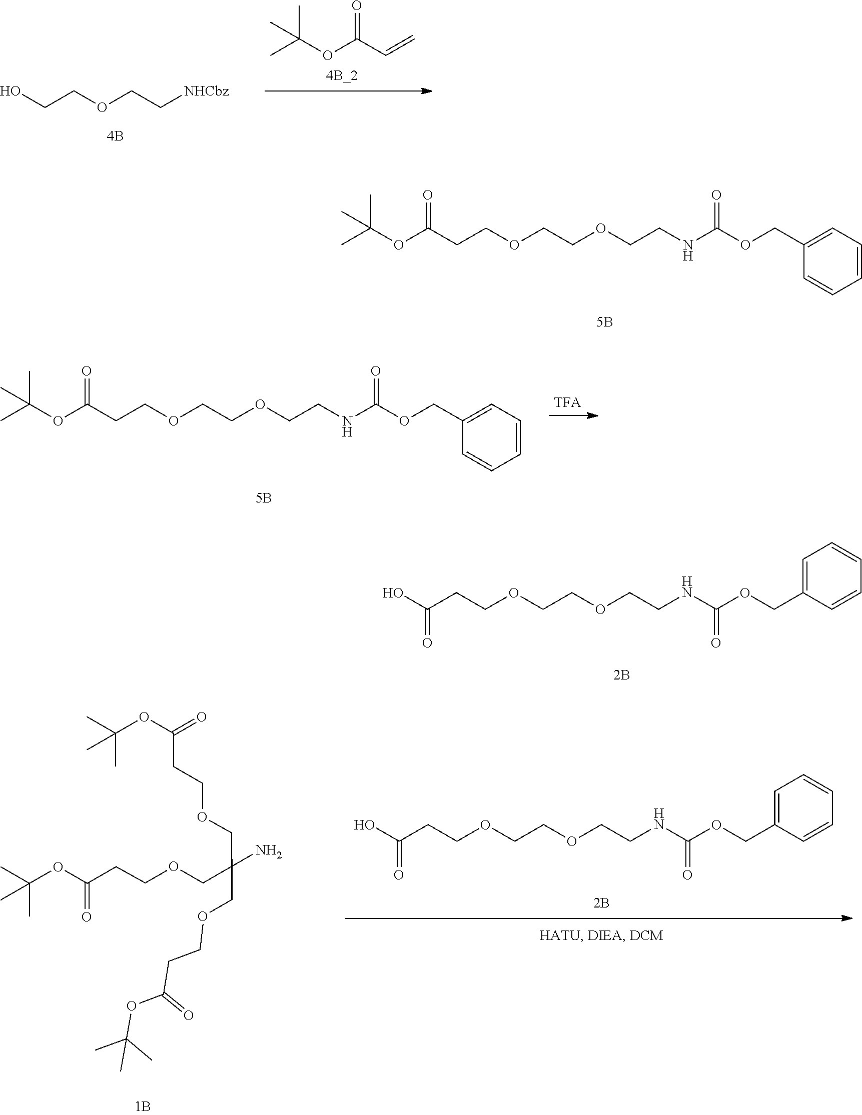

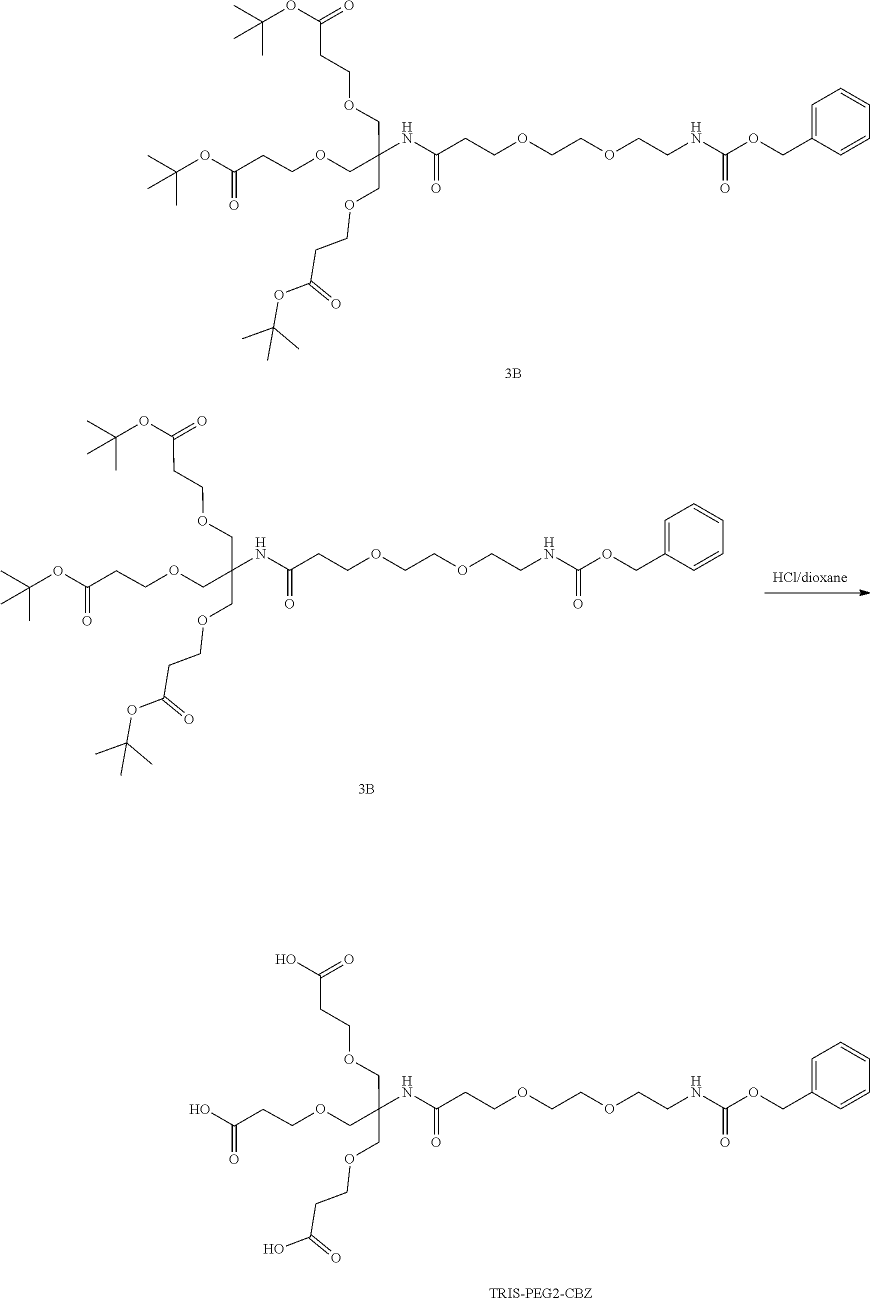 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

### General Procedure for Preparation of Compound 5B

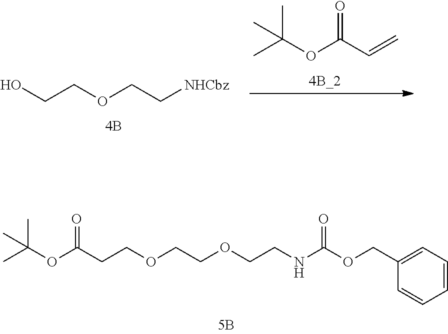

To a solution of Compound 4B (400 g, 1.67 mol, 1.00 eq) and NaOH (10 M, 16.7 mL, 0.10 eq) in THF (2.00 L) is added Compound 4B_2 (1.07 kg, 8.36 mol, 1.20 L, 5.00 eq), the mixture is stirred at 30° C. for 2 hrs. LCMS showed the desired MS is given. Five batches of solution are combined to one batch, then the mixture is diluted with water (6.00 L), extracted with ethyl acetate (3.00 L*3), the combined organic layer is washed with brine (3.00 L), dried over Na₂SO₄, filtered and concentrated under vacuum. The crude is purified by column chromatography (SiO₂, petroleum ether:ethyl acetate=100:1-10:1, R_{f}=0.5) to give Compound 5B (2.36 kg, 6.43 mol, 76.9% yield) as light yellow oil. HNMR: δ 7.31-7.36 (m, 5H), 5.38 (s, 1H), 5.11-5.16 (m, 2H), 3.75 (t, J=6.4 Hz), 3.54-3.62 (m, 6H), 3.39 (d, J=5.2 Hz), 2.61 (t, J=6.0 Hz).

### General Procedure for Preparation of 3-oxo-1-phenyl-2,7,10-trioxa-4-azatridecan-13-oic acid (Compound 2B below)

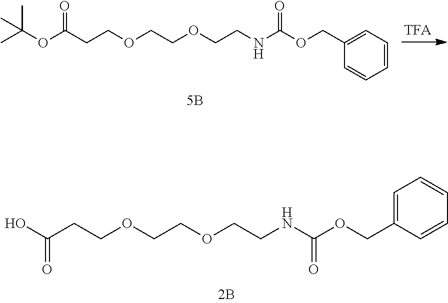 [SMILES: CC(C)(C)OC(=O)CCOCCOCCNC(=O)OCc1ccccc1 ; O=C(O)CCOCCOCCNC(=O)OCc1ccccc1]

To a solution of Compound 5B (741 g, 2.02 mol, 1.00 eq) in DCM (2.80 L) is added TFA (1.43 kg, 12.5 mol, 928 mL, 6.22 eq), the mixture is stirred at 25° C. for 3 hrs. LCMS showed the desired MS is given. The mixture is diluted with DCM (5.00 L), washed with water (3.00 L*3), brine (2.00 L), the combined organic layer is dried over Na₂SO₄, filtered and concentrated under vacuum to give Compound 2B (1800 g, crude) as light yellow oil. HNMR: δ 9.46 (s, 5H), 7.27-7.34 (m, 5H), 6.50-6.65 (m, 1H), 5.71 (s, 1H), 5.10-5.15 (m, 2H), 3.68-3.70 (m, 14H), 3.58-3.61 (m, 6H), 3.39 (s, 2H), 2.55 (s, 6H), 2.44 (s, 2H).

### General Procedure for Preparation of Compound 3B

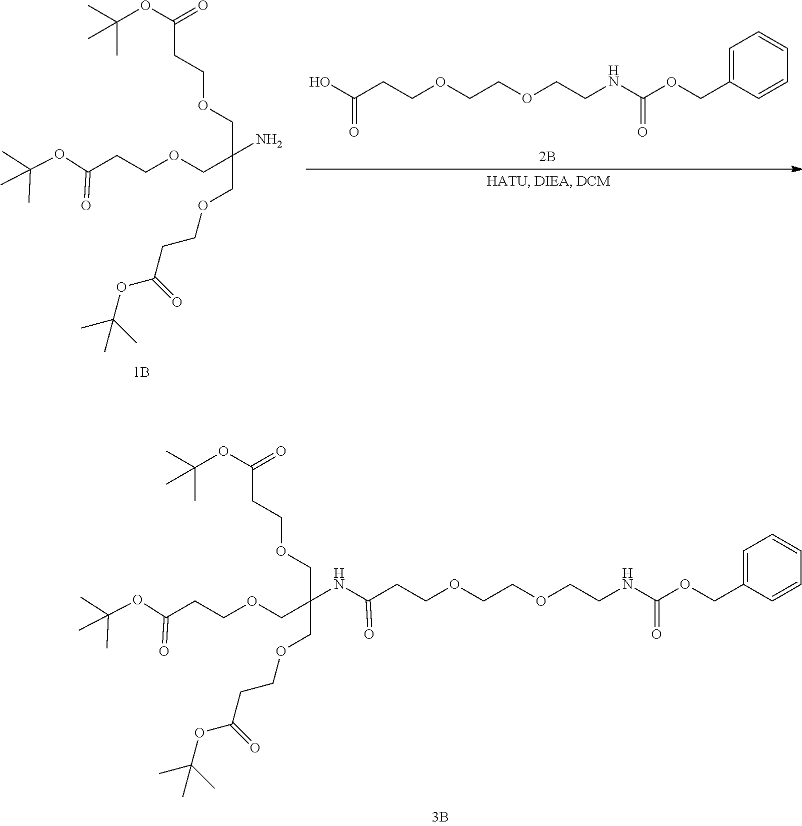 [SMILES: CC(C)(C)OC(=O)CCOCC(N)(COCCC(=O)OC(C)(C)C)COCCC(=O)OC(C)(C)C ; O=C(O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1]

To a solution of Compound 2B (375 g, 999 mmol, 83.0% purity, 1.00 eq) in DCM (1.80 L) is added HATU (570 g, 1.50 mol, 1.50 eq) and DIEA (258 g, 2.00 mol, 348 mL, 2.00 eq) at 0° C., the mixture is stirred at 0° C. for 30 min, then Compound 1B (606 g, 1.20 mol, 1.20 eq) is added, the mixture is stirred at 25° C. for 1 hr. LCMS showed desired MS is given. The mixture is combined to one batch, then the mixture is diluted with DCM (5.00 L), washed with 1 N HCl aqueous solution (2.00 L*2), then the organic layer is washed with saturated Na₂CO₃ aqueous solution (2.00 L*2) and brine (2.00 L), the organic layer is dried over Na₂SO₄, filtered and concentrated under vacuum to give Compound 3B (3.88 kg, crude) as yellow oil.

### General Procedure for Preparation of TRIS-PEG2-CBZ

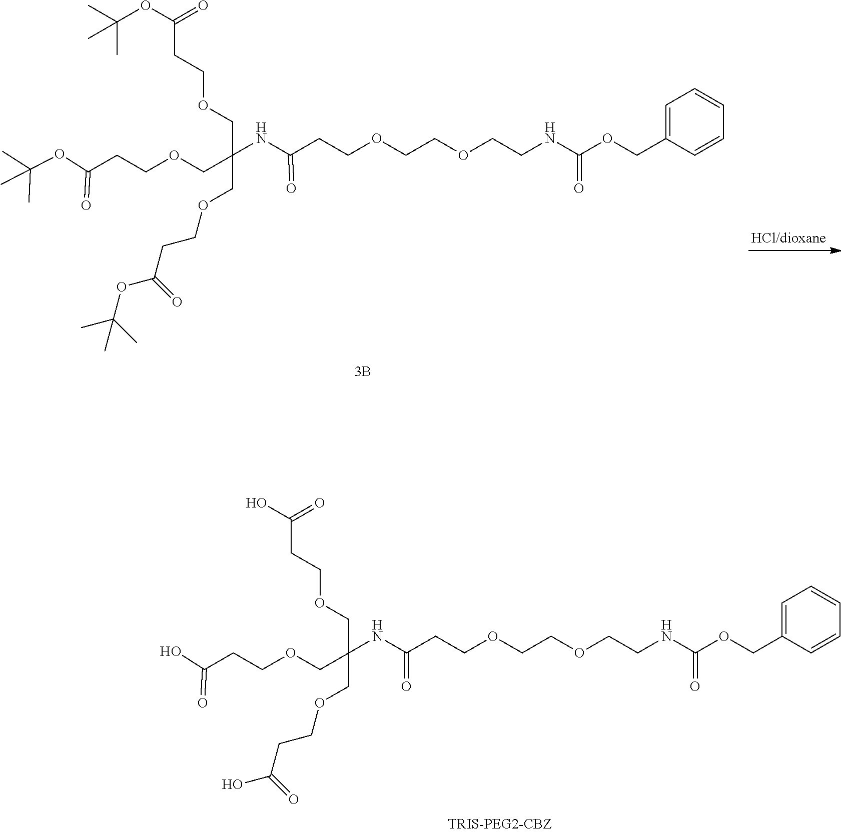 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

A solution of Compound 3B (775 g, 487 mmol, 50.3% purity, 1.00 eq) in HCl/dioxane (4 M, 2.91 L, 23.8 eq) is stirred at 25° C. for 2 hrs. LCMS showed the desired MS is given. The mixture is concentrated under vacuum to give a residue. Then the combined residue is diluted with DCM (5.00 L), adjusted to pH=8 with 2.5 MNaOH aqueous solution, and separated. The aqueous phase is extracted with DCM (3.00 L) again, then the aqueous solution is adjusted to pH=3 with 1 N HCl aqueous solution, then extracted with DCM (5.00 L*2), the combined organic layer is washed with brine (3.00 L), dried over Na₂SO₄, filtered and concentrated under vacuum. The crude is purified by column chromatography (SiO₂, DCM:MeOH=0:1-12:1, 0.1% HOAc, R_{f}=0.4). The residue is diluted with DCM (5.00 L), adjusted to pH=8 with 2.5 M NaOH aqueous solution, separated, the aqueous solution is extracted with DCM (3.00 L) again, then the aqueous solution is adjusted to pH=3 with 6 N HCl aqueous solution, extracted with DCM:MeOH=10:1 (5.00 L*2), the combined organic layer is washed with brine (2.00 L), dried over Na₂SO₄, filtered and concentrated under vacuum to give a residue. Then the residue is diluted with MeCN (5.00 L), concentrated under vacuum, repeat this procedure twice to remove water to give TRIS-PEG2-CBZ (1.25 kg, 1.91 mol, 78.1% yield, 95.8% purity) as light yellow oil. ¹HNMR: 400 MHz, MeOD, δ 7.30-7.35 (5H), 5.07 (s, 2H), 3.65-3.70 (m, 16H), 3.59 (s, 4H), 3.45 (t, J=5.6 Hz), 2.51 (t, J=6.0 Hz), 2.43 (t, 6.4 Hz).

### Scheme for the Preparation of TriNGal-TRIS-Peg2-Phosph 8c

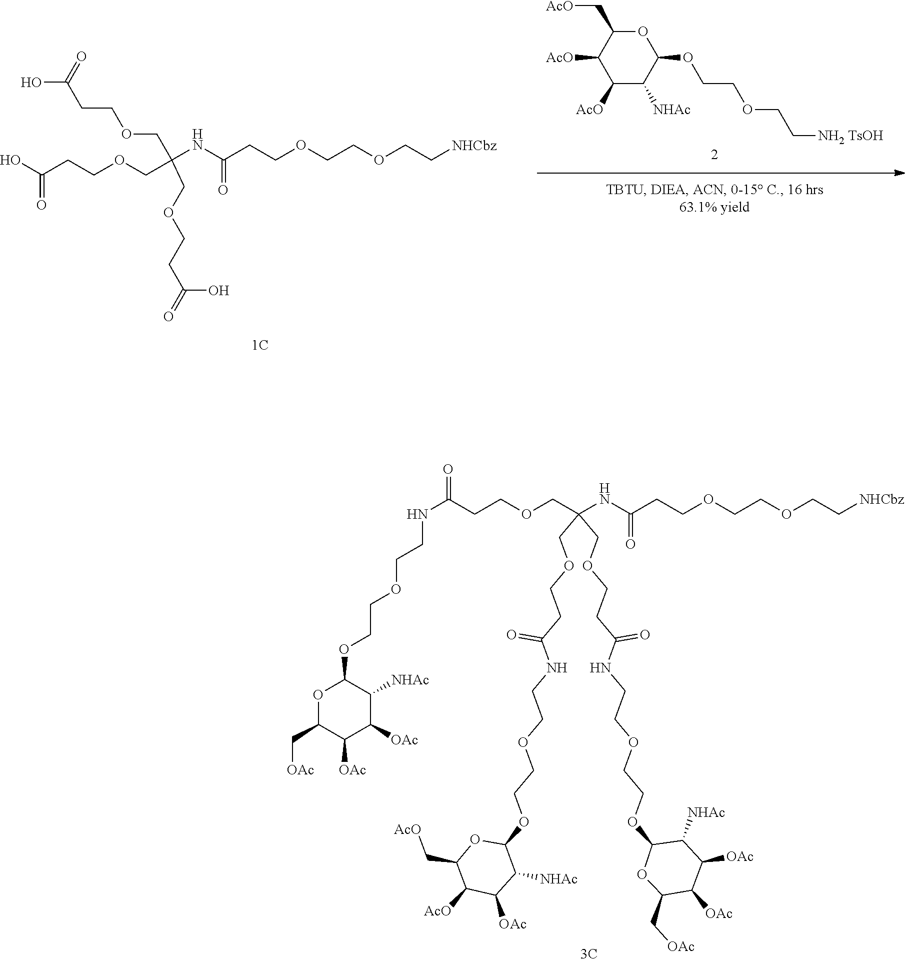

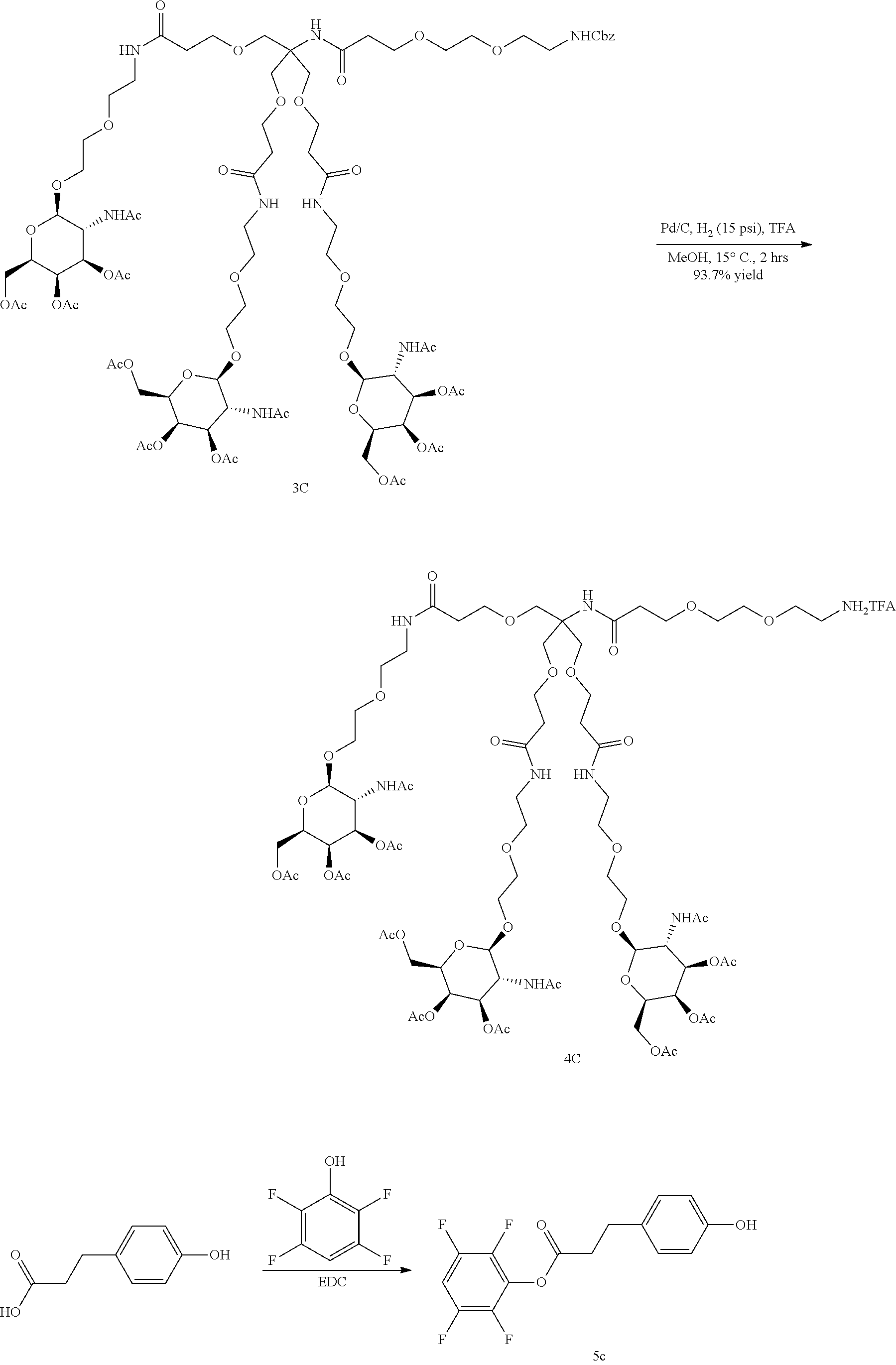

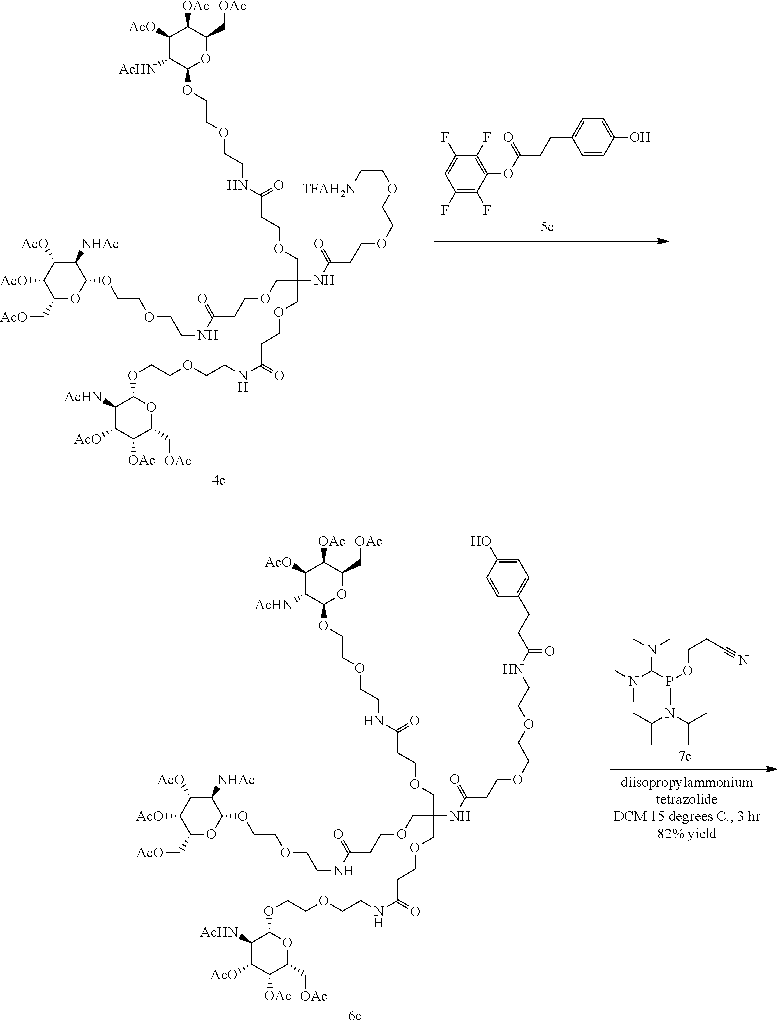

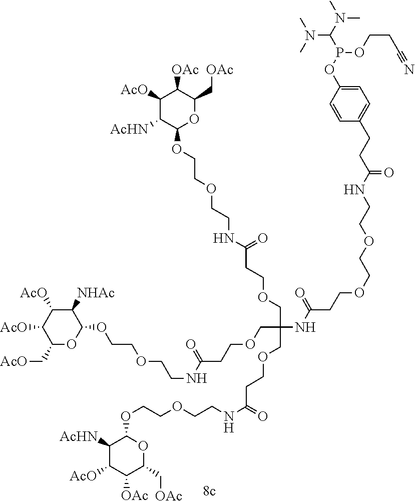 [SMILES: CC(=O)N[C@H]1[C@H](OCCOCCNC(=O)CCOCC(COCCC(=O)NCCOCCO[C@@H]2O[C@H](COC(C)=O)[C@H](OC(C)=O)[C@H](OC(C)=O)[C@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)C(N(C)C)N(C)C)cc2)O[C@H](COC(C)=O)[C@H](OC(C)=O)[C@@H]1OC(C)=O]

### TriGNl-TRIS-Peg2-Phosph 8c

### General Procedure for Preparation of Compound 3C

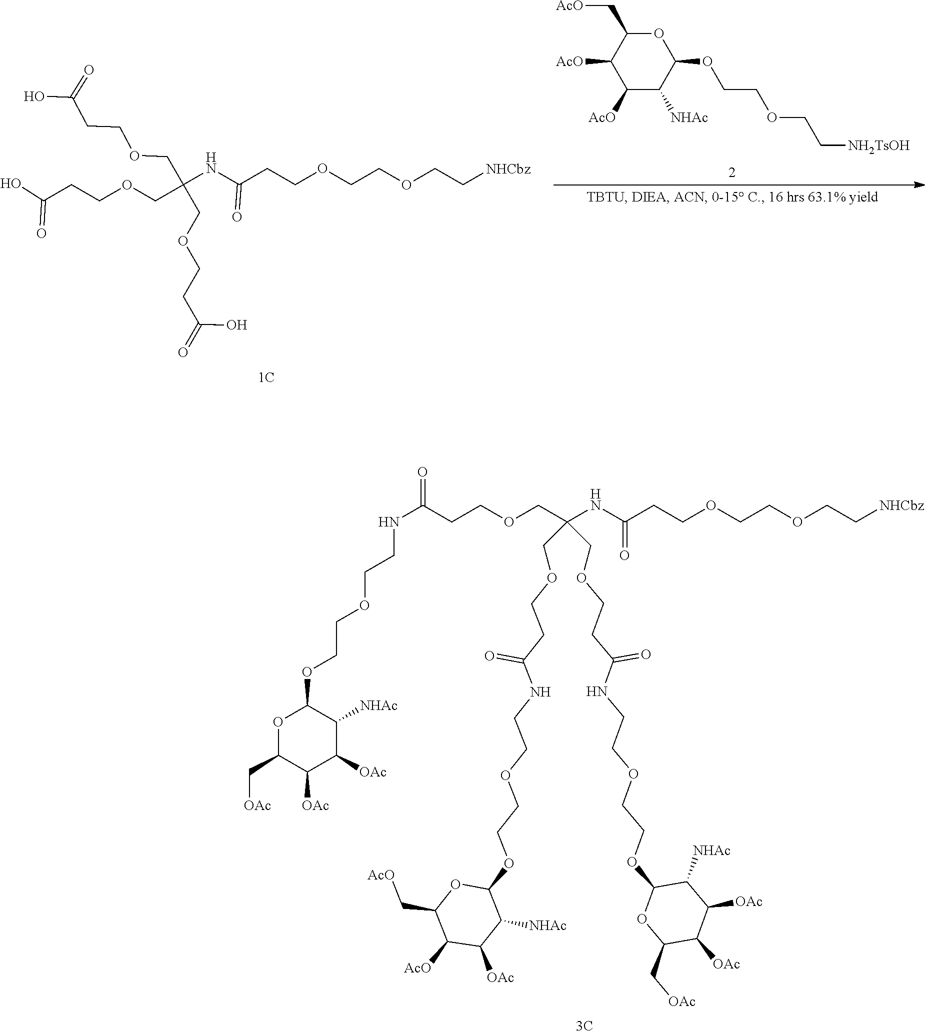

To a solution of Compound 1C (155 g, 245 mmol, 1.00 eq) in ACN (1500 mL) is added TBTU (260 g, 811 mmol, 3.30 eq), DIEA (209 g, 1.62 mol, 282 mL, 6.60 eq) and Compound 2C (492 g, 811 mmol, 3.30 eq, TsOH) at 0° C., the mixture is stirred at 15° C. for 16 hrs. LCMS showed the desired MS is given. The mixture is concentrated under vacuum to give a residue, then the mixture is diluted with DCM (2000 mL), washed with 1 N HCl aqueous solution (700 mL*2), then saturated NaHCO₃ aqueous solution (700 mL*2) and concentrated under vacuum. The crude is purified by column chromatography to give Compound 3C (304 g, 155 mmol, 63.1% yield, 96.0% purity) as a yellow solid.

### General Procedure for Preparation of Compound 4C

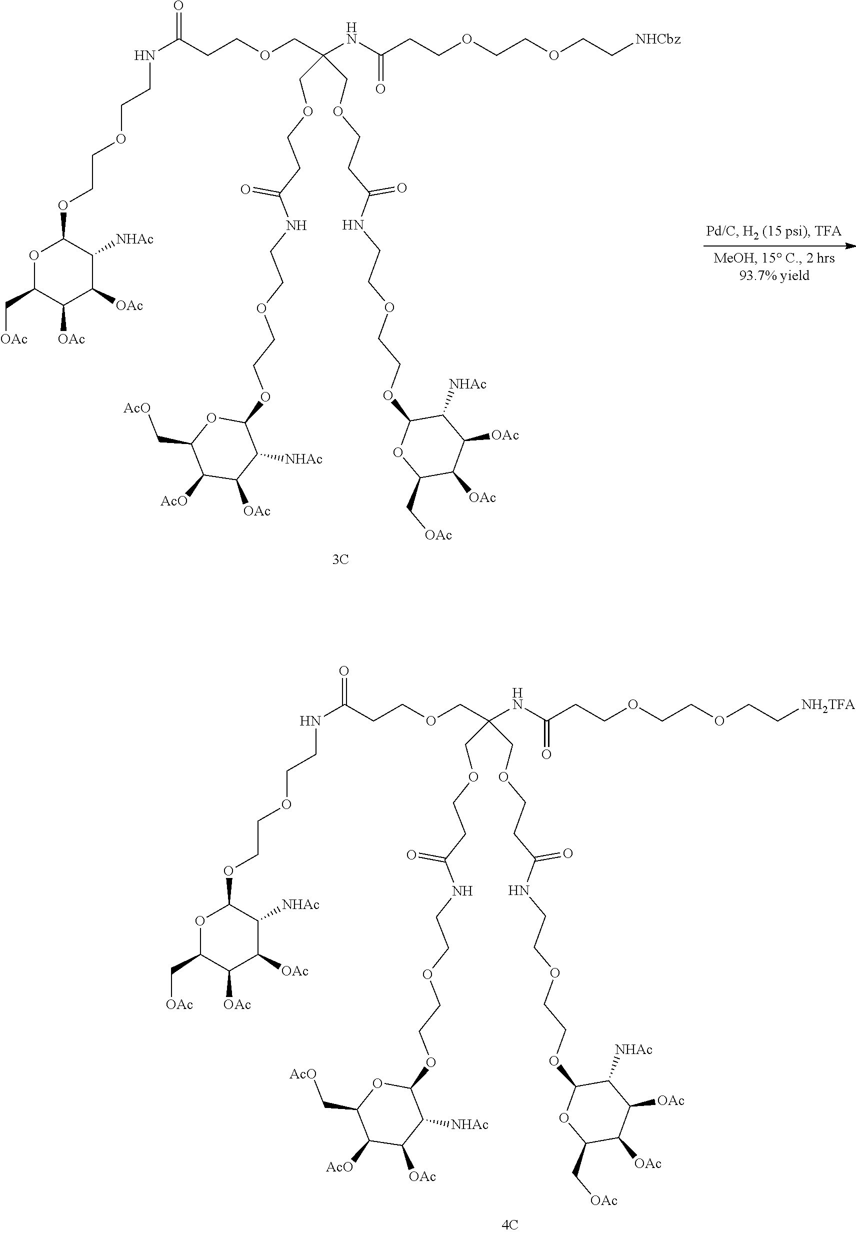

Two batches solution of Compound 3C (55.0 g, 29.2 mmol, 1.00 eq) in MeOH (1600 mL) is added Pd/C (6.60 g, 19.1 mmol, 10.0% purity) and TFA (3.34 g, 29.2 mmol, 2.17 mL, 1.00 eq), the mixture is degassed under vacuum and purged with H₂. The mixture is stirred under H₂ (15 psi) at 15° C. for 2 hours. LCMS showed the desired MS is given. The mixture is filtered and the filtrate is concentrated under vacuum to give Compound 4C (106 g, 54.8 mmol, 93.7% yield, 96.2% purity, TFA) as a white solid.

### General Procedure for Preparation of Compound 5C

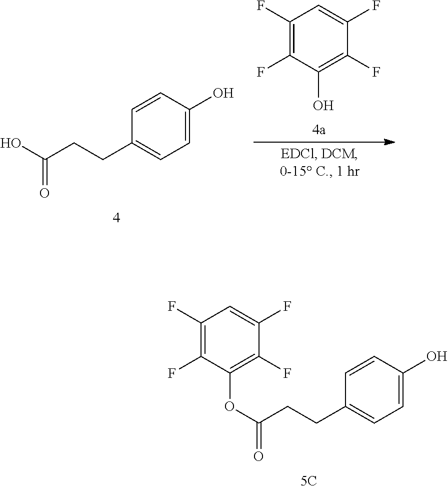 [SMILES: O=C(O)CCc1ccc(O)cc1 ; Oc1c(F)c(F)cc(F)c1F ; O=C(CCc1ccc(O)cc1)Oc1c(F)c(F)cc(F)c1F]

Two batches in parallel. To a solution of EDCI (28.8 g, 150 mmol, 1.00 eq) in DCM (125 mL) is added compound 4a (25.0 g, 150 mmol, 1.00 eq) dropwise at 0° C., then the mixture is added to compound 4 (25.0 g, 150 mmol, 1.00 eq) in DCM (125 mL) at 0° C., then the mixture is stirred at 25° C. for 1 hr. TLC (Petroleum ether:Ethyl acetate=3:1, R_{f}=0.45) showed the reactant is consumed and one new spot is formed. The reaction mixture is diluted with DCM (100 mL) then washed with aq.NaHCO₃ (250 mL*1) and brine (250 mL), dried over Na₂SO₄, filtered and concentrated under reduced pressure to give a residue. The residue is purified by colunn chromatography (SiO₂, Petroleum ether:Ethyl acetate=100 i 1 to 3:1), TLC (SiO₂, Petroleum ether:Ethyl acetate=3:1), R_{f}=0.45, then concentrated under reduced pressure to give a residue. Compound 5C (57.0 g, 176 mmol, 58.4% yield, 96.9% purity) is obtained as colorless oil and confirmed ¹HNMR: EW33072-2-P1A, 400 MHz, DMSO (9.21 (s, 1H), 7.07-7.09 (m, 2H), 6.67-6.70 (m, 2H), 3.02-3.04 (m, 2H), 2.86-2.90 (m, 2H)

### General Procedure for Preparation of Compound 6

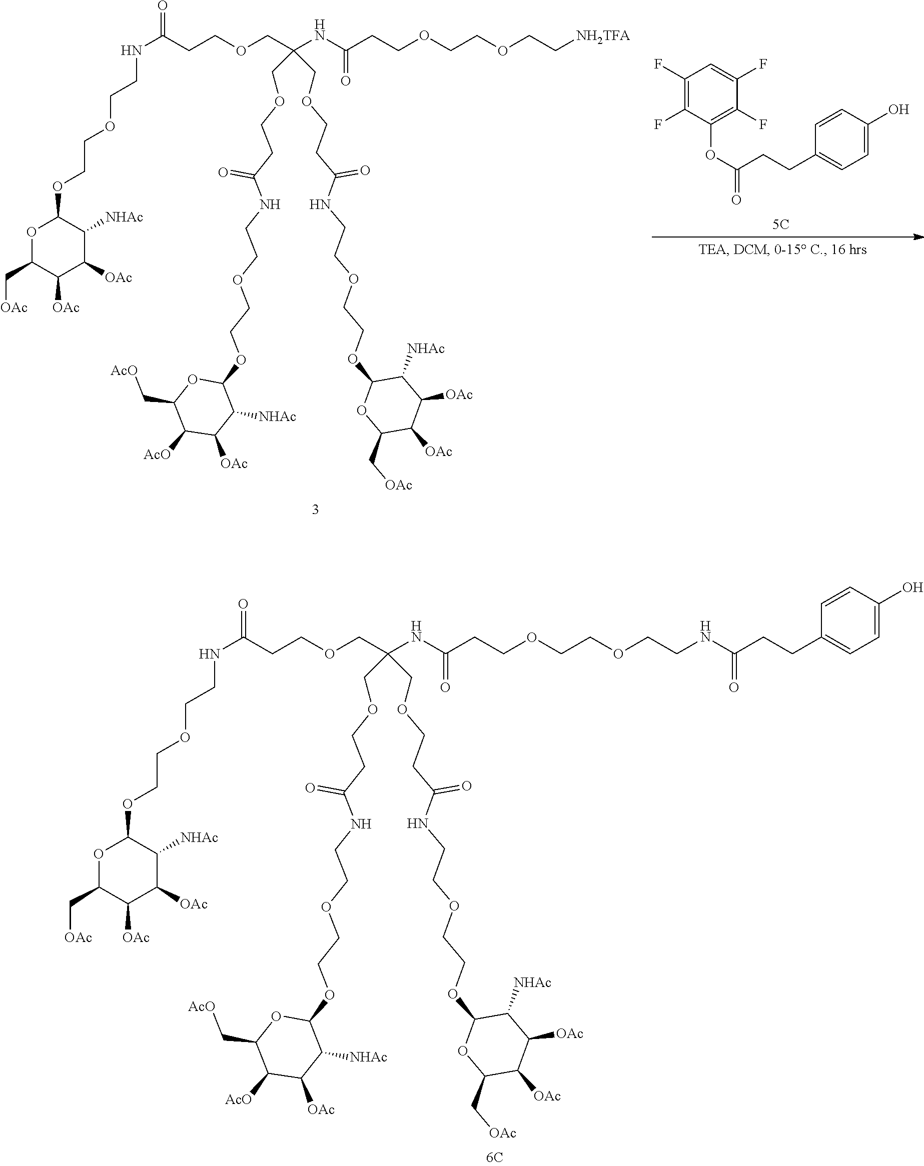

To a mixture of compound 3 (79.0 g, 41.0 mmol, 96.4% purity, 1.00 eq, TFA) and compound 6C (14.2 g, 43.8 mmol, 96.9% purity, 1.07 eq) in DCM (800 mL) is added TEA (16.6 g, 164 mmol, 22.8 mL, 4.00 eq) dropwise at 0° C., the mixture is stirred at 15° C. for 16 hrs. LCMS (EW33072-12-P1B, Rt=0.844 min) showed the desired mass is detected. The reaction mixture is diluted with DCM (400 mL) and washed with aq.NaHCO₃ (400 mL*1) and brine (400 mL*1), then the mixture is diluted with DCM (2.00 L) and washed with 0.7 M Na₂CO₃ (1000 mL*3) and brine (800 mL*3), dried over Na₂SO₄, filtered and concentrated under reduced pressure to give a residue. The residue is used to next step directly without purification. Compound 6 (80.0 g, crude) is obtained as white solid and confirmed via ¹HNMR: EW33072-12-P1A, 400 MHz, MeOD δ 7.02-7.04 (m, 2H), 6.68-6.70 (m, 2H), 5.34-5.35 (s, 3H), 5.07-5.08 (d, J=4.00 Hz, 3H), 4.62-4.64 (d, J=8.00 Hz, 3H), 3.71-4.16 (m, 16H), 3.31-3.70 (m, 44H), 2.80-2.83 (m, 2H), 2.68 (m, 2H), 2.46-2.47 (m, 10H), 2.14 (s, 9H), 2.03 (s, 9H), 1.94-1.95 (d, J=4.00 Hz, 18H).

### General Procedure for Preparation of TriGNal-TRIS-Peg2-Phosph 8c

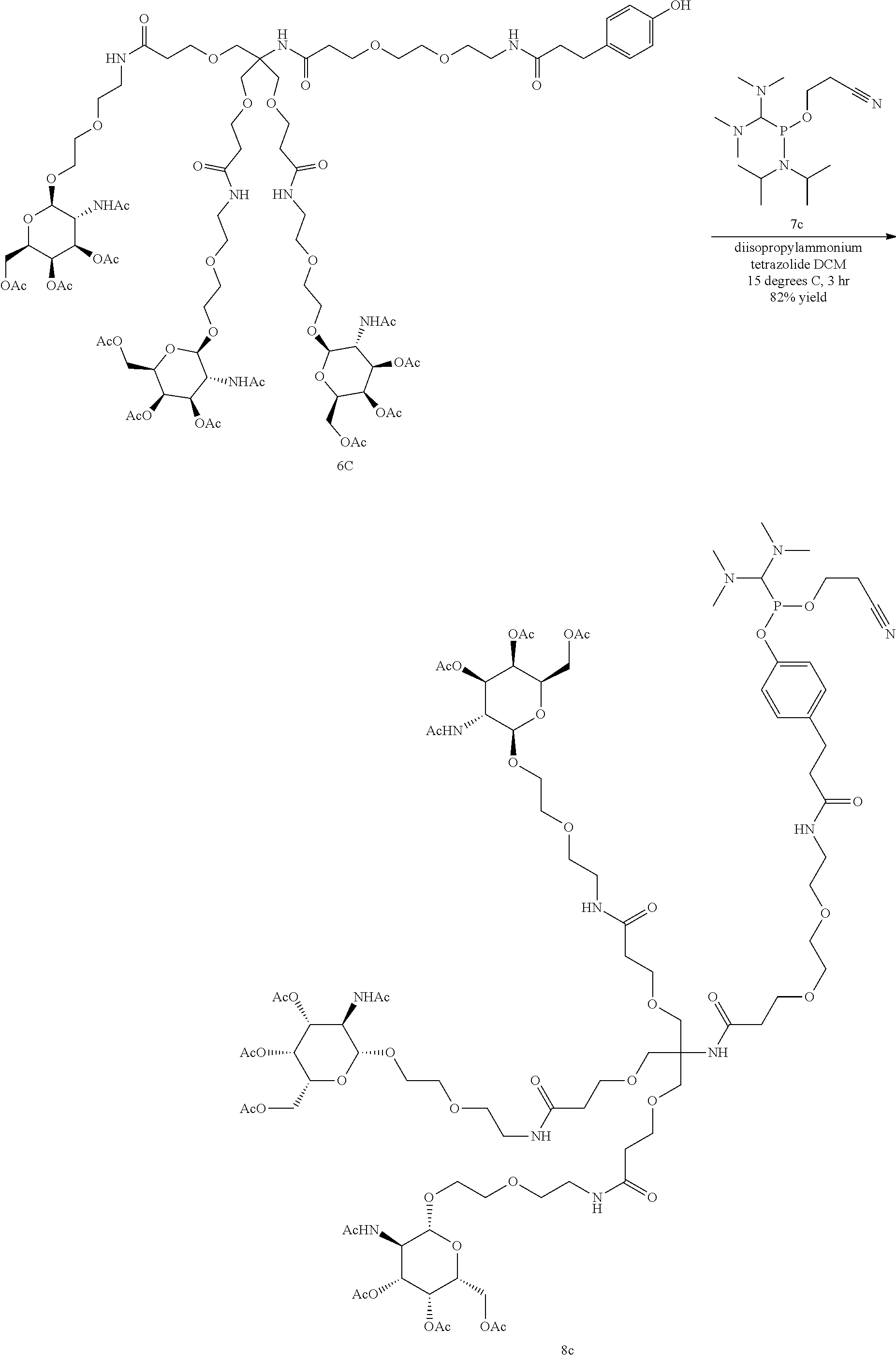

Two batches are synthesized in parallel. To a solution of compound 6C (40.0 g, 21.1 mmol, 1.00 eq in DCM (600 mL) is added diisopropylammonium tetrazolide (3.62 g, 21.1 mmol, 1.00 eq) and compound 7c (6.37 g, 21.1 mmol, 6.71 mL, 1.00 eq) in DCM (8.00 mL) drop-wise, the mixture is stirred at 30° C. for 1 hr, then added compound 7c (3.18 g, 10.6 mmol, 3.35 mL, 0.50 eq) in DCM (8.00 mL) drop-wise, the mixture is stirred at 30° C. for 30 mins, then added compound 7c (3.18 g, 10.6 mmol, 3.35 mL, 0.50 eq) in DCM (8.00 mL) drop-wise, the mixture is stirred at 30° C. for 1.5 hrs. LCMS (EW33072-17-P1C1, Rt=0.921 min) showed the desired MS+1 is detected. LCMS (EW33072-17-P1C2, Rt=0.919 min) showed the desired MS+1 is detected. Two batches are combined for work-up. The mixture is diluted with DCM (1.20 L), washed with saturated NaHCO₃ aqueous solution (1.60 L*2), 3% DMF in H₂O (1.60 L*2), H₂O (1.60 L*3), brine (1.60 L), dried over Na₂SO₄, filtered and concentrated under reduced pressure to give a residue. The residue is purified by column chromatography (SiO₂, DCM:MeOH :TEA=100:3:2) TLC (S102, DCM MeOH=10:1, R_{f}=0.45), then concentrated under reduced pressure to give a residue. Compound 8C (76.0 g, 34.8 mmol, 82.5% yield, 96.0% purity) is obtained as white solid and confirmed via ¹HNMR: EW33072-19-P1C, 400 MHz, MeOD δ 7.13-7.15 (d, J=8.50 Hz, 2H), 6.95-6.97 (dd, J=8.38, 1.13 Hz, 2H), 5.34 (d, J=2.88 Hz, 3H), 0.09 (dd, J=11.26, 3.38 Hz, 3H), 4.64 (d, J=8.50 Hz, 3H), 3.99-4.20 (m, 12H), 3.88-3.98 (m, 5H), 3.66-3.83 (m, 20H), 3.51-3.65 (m, 17H), 3.33-3.50 (m, 9H), 2.87 (t, J=7.63 Hz, 2H), 2.76 (t, J=5.94 Hz, 2H), 2.42-2.50 (m, 10H), 2.14 (s, 9H), 2.03 (s, 9H), 1.94-1.95 (d, J=6.13 Hz, 18H), 1.24-1.26 (d, J=6.75 Hz, 6H), 1.18-1.20 (d, J=6.75 Hz, 6H)

### Example 14: Modification Motif 1

An example MTRES1 siRNA includes a combination of the following modifications:
Position 9 (from 5′ to 3′) of the sense strand is 2′ F.
If position 9 is a pyrimidine then all purines in the Sense Strand are 2′OMe, and 1-5 pyrimidines between positions 5 and 11 are 2′ F provided that there are never three 2′F modifications in a row.
If position 9 is a purine then all pyrimidines in the Sense Strand are 2′OMe, and 1-5 purines between positions 5 and 11 are 2′ F provided that there are never three 2′F modifications in a row.
Antisense strand odd-numbered positions are 2′OMe and even-numbered positions are a mixture of 2′ F, 2′ OMe and 2′ deoxy.

### Example 15: Modification Motif 2

An example MTRES1 siRNA includes a combination of the following modifications:
Position 9 (from 5′ to 3′) of the sense strand is 2′ deoxy.
Sense strand positions 5, 7 and 8 are 2′ F.
All pyrimidines in positions 10-21 are 2′ OMe, and purines are a mixture of 2′ OMe and 2′ F. Alternatively, all purines in positions 10-21 are 2′ OMe and all pyrimidines in positions 10-21 are a mixture of 2′ OMe and 2′ F.
Antisense strand odd-numbered positions are 2′OMe and even-numbered positions are a mixture of 2′ F, 2′OMe and 2′ deoxy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and compositions within the scope of these claims and their equivalents be covered thereby.

### IV. SEQUENCE INFORMATION

Some embodiments include one or more nucleic acid sequences in the following tables:

TABLE 19
Sequence Information

| SEQ ID NO: | Description |
|---|---|
| 1-1140 | MTRES1 siRNA sense strand sequences |
| 1141-2280 | MTRES1 siRNA antisense strand sequences |
| 2281-2334 | Modified MTRES1 siRNA sense strand sequences |
| 2335-2388 | Modified MTRES1 siRNA antisense strand sequences |
| 2389-2442 | Alternatively modified MTRES1 siRNA sense strand |
| | sequences |
| 2443 | Full-length human MTRES1 mRNA sequence (Ensembl |
| | Acc. ENST00000311381.8) (human RNA) |
| 2444-2452 | Modification pattern 1S to 9S |
| 2453-2460 | Modification pattern 1AS to 8AS |
| 2461 | Modification pattern ASO1 |
| 2462 | Full-length human MTRES1 mRNA sequence (Ensembl |
| | Acc. ENST00000625458.1) (human RNA) |
| 2463-2466 | Example modified siRNA sense strand sequences |
| 2467-2470 | Example modified siRNA antisense strand sequences |
| 2471-2487 | Example modified siRNA sense strand sequences |
| 2488-2504 | Example modified siRNA antisense strand sequences |
| 2505-2514 | Example modified siRNA sense strand sequences |
| 2515-2524 | Example modified siRNA antisense strand sequences |
| 2525-2547 | Modification pattern 10S to 32S |
| 2549-2549 | Modification pattern 9AS to 10AS |
| 2550-2611 | Example siRNA sense strand sequences |
| 2612-2673 | Example siRNA antisense strand sequences |

TABLE 20
Sequences

| | SEQ | | SEQ | |
|---|---|---|---|---|
| | ID | sense strand | ID | antisense strand |
| siRNA Name | NO: | sequence (5′-3′) | NO: | sequence (5′-3′) |
| siRNA 1 | 1 | GCGCAGAUAGGGGUAGCCU | 1141 | AGGCUACCCCUAUCUGCGC |
| siRNA 2 | 2 | CGCAGAUAGGGGUAGCCUG | 1142 | CAGGCUACCCCUAUCUGCG |
| siRNA 3 | 3 | GCAGAUAGGGGUAGCCUGG | 1143 | CCAGGCUACCCCUAUCUGC |
| siRNA 4 | 4 | CAGAUAGGGGUAGCCUGGA | 1144 | UCCAGGCUACCCCUAUCUG |
| siRNA 5 | 5 | AGAUAGGGGUAGCCUGGAG | 1145 | CUCCAGGCUACCCCUAUCU |
| siRNA 6 | 6 | GAUAGGGGUAGCCUGGAGG | 1146 | CCUCCAGGCUACCCCUAUC |
| siRNA 7 | 7 | AUAGGGGUAGCCUGGAGGC | 1147 | GCCUCCAGGCUACCCCUAU |
| siRNA 8 | 8 | UAGGGGUAGCCUGGAGGCC | 1148 | GGCCUCCAGGCUACCCCUA |
| siRNA 9 | 9 | AGGGGUAGCCUGGAGGCCU | 1149 | AGGCCUCCAGGCUACCCCU |
| siRNA 10 | 10 | GGGGUAGCCUGGAGGCCUG | 1150 | CAGGCCUCCAGGCUACCCC |
| siRNA 11 | 11 | GGGUAGCCUGGAGGCCUGC | 1151 | GCAGGCCUCCAGGCUACCC |
| siRNA 12 | 12 | GGUAGCCUGGAGGCCUGCA | 1152 | UGCAGGCCUCCAGGCUACC |
| siRNA 13 | 13 | GUAGCCUGGAGGCCUGCAG | 1153 | CUGCAGGCCUCCAGGCUAC |
| siRNA 14 | 14 | UAGCCUGGAGGCCUGCAGU | 1154 | ACUGCAGGCCUCCAGGCUA |
| siRNA 15 | 15 | AGCCUGGAGGCCUGCAGUC | 1155 | GACUGCAGGCCUCCAGGCU |
| siRNA 16 | 16 | GCCUGGAGGCCUGCAGUCC | 1156 | GGACUGCAGGCCUCCAGGC |
| siRNA 17 | 17 | CCUGGAGGCCUGCAGUCCG | 1157 | CGGACUGCAGGCCUCCAGG |
| siRNA 18 | 18 | CUGGAGGCCUGCAGUCCGC | 1158 | GCGGACUGCAGGCCUCCAG |
| siRNA 19 | 19 | UGGAGGCCUGCAGUCCGCG | 1159 | CGCGGACUGCAGGCCUCCA |
| siRNA 20 | 20 | GGAGGCCUGCAGUCCGCGC | 1160 | GCGCGGACUGCAGGCCUCC |
| siRNA 21 | 21 | GAGGCCUGCAGUCCGCGCG | 1161 | CGCGCGGACUGCAGGCCUC |
| siRNA 22 | 22 | AGGCCUGCAGUCCGCGCGG | 1162 | CCGCGCGGACUGCAGGCCU |
| siRNA 23 | 23 | GGCCUGCAGUCCGCGCGGC | 1163 | GCCGCGCGGACUGCAGGCC |
| siRNA 24 | 24 | GCCUGCAGUCCGCGCGGCC | 1164 | GGCCGCGCGGACUGCAGGC |
| siRNA 25 | 25 | CCUGCAGUCCGCGCGGCCG | 1165 | CGGCCGCGCGGACUGCAGG |
| siRNA 26 | 26 | CUGCAGUCCGCGCGGCCGC | 1166 | GCGGCCGCGCGGACUGCAG |
| siRNA 27 | 27 | UGCAGUCCGCGCGGCCGCG | 1167 | CGCGGCCGCGCGGACUGCA |
| siRNA 28 | 28 | GCAGUCCGCGCGGCCGCGG | 1168 | CCGCGGCCGCGCGGACUGC |
| siRNA 29 | 29 | CAGUCCGCGCGGCCGCGGG | 1169 | CCCGCGGCCGCGCGGACUG |
| siRNA 30 | 30 | AGUCCGCGCGGCCGCGGGG | 1170 | CCCCGCGGCCGCGCGGACU |
| siRNA 31 | 31 | GUCCGCGCGGCCGCGGGGA | 1171 | UCCCCGCGGCCGCGCGGAC |
| siRNA 32 | 32 | UCCGCGCGGCCGCGGGGAG | 1172 | CUCCCCGCGGCCGCGCGGA |
| siRNA 33 | 33 | CCGCGCGGCCGCGGGGAGG | 1173 | CCUCCCCGCGGCCGCGCGG |
| siRNA 34 | 34 | CGCGCGGCCGCGGGGAGGG | 1174 | CCCUCCCCGCGGCCGCGCG |
| siRNA 35 | 35 | GCGCGGCCGCGGGGAGGGA | 1175 | UCCCUCCCCGCGGCCGCGC |
| siRNA 36 | 36 | CGCGGCCGCGGGGAGGGAC | 1176 | GUCCCUCCCCGCGGCCGCG |
| siRNA 37 | 37 | GCGGCCGCGGGGAGGGACG | 1177 | CGUCCCUCCCCGCGGCCGC |
| siRNA 38 | 38 | CGGCCGCGGGGAGGGACGA | 1178 | UCGUCCCUCCCCGCGGCCG |
| siRNA 39 | 39 | GGCCGCGGGGAGGGACGAG | 1179 | CUCGUCCCUCCCCGCGGCC |
| siRNA 40 | 40 | GCCGCGGGGAGGGACGAGA | 1180 | UCUCGUCCCUCCCCGCGGC |
| siRNA 41 | 41 | CCGCGGGGAGGGACGAGAG | 1181 | CUCUCGUCCCUCCCCGCGG |
| siRNA 42 | 42 | CGCGGGGAGGGACGAGAGG | 1182 | CCUCUCGUCCCUCCCCGCG |
| siRNA 43 | 43 | GCGGGGAGGGACGAGAGGG | 1183 | CCCUCUCGUCCCUCCCCGC |
| siRNA 44 | 44 | CGGGGAGGGACGAGAGGGC | 1184 | GCCCUCUCGUCCCUCCCCG |
| siRNA 45 | 45 | GGGGAGGGACGAGAGGGCC | 1185 | GGCCCUCUCGUCCCUCCCC |
| siRNA 46 | 46 | GGGAGGGACGAGAGGGCCU | 1186 | AGGCCCUCUCGUCCCUCCC |
| siRNA 47 | 47 | GGAGGGACGAGAGGGCCUG | 1187 | CAGGCCCUCUCGUCCCUCC |
| siRNA 48 | 48 | GAGGGACGAGAGGGCCUGA | 1188 | UCAGGCCCUCUCGUCCCUC |
| siRNA 49 | 49 | AGGGACGAGAGGGCCUGAC | 1189 | GUCAGGCCCUCUCGUCCCU |
| siRNA 50 | 50 | GGGACGAGAGGGCCUGACG | 1190 | CGUCAGGCCCUCUCGUCCC |
| siRNA 51 | 51 | GGACGAGAGGGCCUGACGU | 1191 | ACGUCAGGCCCUCUCGUCC |
| siRNA 52 | 52 | GACGAGAGGGCCUGACGUA | 1192 | UACGUCAGGCCCUCUCGUC |
| siRNA 53 | 53 | ACGAGAGGGCCUGACGUAC | 1193 | GUACGUCAGGCCCUCUCGU |
| siRNA 54 | 54 | CGAGAGGGCCUGACGUACA | 1194 | UGUACGUCAGGCCCUCUCG |
| siRNA 55 | 55 | GAGAGGGCCUGACGUACAG | 1195 | CUGUACGUCAGGCCCUCUC |
| siRNA 56 | 56 | AGAGGGCCUGACGUACAGA | 1196 | UCUGUACGUCAGGCCCUCU |
| siRNA 57 | 57 | GAGGGCCUGACGUACAGAU | 1197 | AUCUGUACGUCAGGCCCUC |
| siRNA 58 | 58 | AGGGCCUGACGUACAGAUU | 1198 | AAUCUGUACGUCAGGCCCU |
| siRNA 59 | 59 | GGGCCUGACGUACAGAUUA | 1199 | UAAUCUGUACGUCAGGCCC |
| siRNA 60 | 60 | GGCCUGACGUACAGAUUAU | 1200 | AUAAUCUGUACGUCAGGCC |
| siRNA 61 | 61 | GCCUGACGUACAGAUUAUA | 1201 | UAUAAUCUGUACGUCAGGC |
| siRNA 62 | 62 | CCUGACGUACAGAUUAUAA | 1202 | UUAUAAUCUGUACGUCAGG |
| siRNA 63 | 63 | CUGACGUACAGAUUAUAAG | 1203 | CUUAUAAUCUGUACGUCAG |
| siRNA 64 | 64 | UGACGUACAGAUUAUAAGC | 1204 | GCUUAUAAUCUGUACGUCA |
| siRNA 65 | 65 | GACGUACAGAUUAUAAGCG | 1205 | CGCUUAUAAUCUGUACGUC |
| siRNA 66 | 66 | ACGUACAGAUUAUAAGCGC | 1206 | GCGCUUAUAAUCUGUACGU |
| siRNA 67 | 67 | CGUACAGAUUAUAAGCGCC | 1207 | GGCGCUUAUAAUCUGUACG |
| siRNA 68 | 68 | GUACAGAUUAUAAGCGCCA | 1208 | UGGCGCUUAUAAUCUGUAC |
| siRNA 69 | 69 | UACAGAUUAUAAGCGCCAU | 1209 | AUGGCGCUUAUAAUCUGUA |
| siRNA 70 | 70 | ACAGAUUAUAAGCGCCAUG | 1210 | CAUGGCGCUUAUAAUCUGU |
| siRNA 71 | 71 | CAGAUUAUAAGCGCCAUGG | 1211 | CCAUGGCGCUUAUAAUCUG |
| siRNA 72 | 72 | AGAUUAUAAGCGCCAUGGC | 1212 | GCCAUGGCGCUUAUAAUCU |
| siRNA 73 | 73 | GAUUAUAAGCGCCAUGGCU | 1213 | AGCCAUGGCGCUUAUAAUC |
| siRNA 74 | 74 | AUUAUAAGCGCCAUGGCUA | 1214 | UAGCCAUGGCGCUUAUAAU |
| siRNA 75 | 75 | UUAUAAGCGCCAUGGCUAU | 1215 | AUAGCCAUGGCGCUUAUAA |
| siRNA 76 | 76 | UAUAAGCGCCAUGGCUAUG | 1216 | CAUAGCCAUGGCGCUUAUA |
| siRNA 77 | 77 | AUAAGCGCCAUGGCUAUGG | 1217 | CCAUAGCCAUGGCGCUUAU |
| siRNA 78 | 78 | UAAGCGCCAUGGCUAUGGC | 1218 | GCCAUAGCCAUGGCGCUUA |
| siRNA 79 | 79 | AAGCGCCAUGGCUAUGGCU | 1219 | AGCCAUAGCCAUGGCGCUU |
| siRNA 80 | 80 | AGCGCCAUGGCUAUGGCUA | 1220 | UAGCCAUAGCCAUGGCGCU |
| siRNA 81 | 81 | GCGCCAUGGCUAUGGCUAG | 1221 | CUAGCCAUAGCCAUGGCGC |
| siRNA 82 | 82 | CGCCAUGGCUAUGGCUAGU | 1222 | ACUAGCCAUAGCCAUGGCG |
| siRNA 83 | 83 | GCCAUGGCUAUGGCUAGUG | 1223 | CACUAGCCAUAGCCAUGGC |
| siRNA 84 | 84 | CCAUGGCUAUGGCUAGUGU | 1224 | ACACUAGCCAUAGCCAUGG |
| siRNA 85 | 85 | CAUGGCUAUGGCUAGUGUU | 1225 | AACACUAGCCAUAGCCAUG |
| siRNA 86 | 86 | AUGGCUAUGGCUAGUGUUA | 1226 | UAACACUAGCCAUAGCCAU |
| siRNA 87 | 87 | UGGCUAUGGCUAGUGUUAA | 1227 | UUAACACUAGCCAUAGCCA |
| siRNA 88 | 88 | GGCUAUGGCUAGUGUUAAA | 1228 | UUUAACACUAGCCAUAGCC |
| siRNA 89 | 89 | GCUAUGGCUAGUGUUAAAU | 1229 | AUUUAACACUAGCCAUAGC |
| siRNA 90 | 90 | CUAUGGCUAGUGUUAAAUU | 1230 | AAUUUAACACUAGCCAUAG |
| siRNA 91 | 91 | UAUGGCUAGUGUUAAAUUG | 1231 | CAAUUUAACACUAGCCAUA |
| siRNA 92 | 92 | AUGGCUAGUGUUAAAUUGC | 1232 | GCAAUUUAACACUAGCCAU |
| siRNA 93 | 93 | UGGCUAGUGUUAAAUUGCU | 1233 | AGCAAUUUAACACUAGCCA |
| siRNA 94 | 94 | GGCUAGUGUUAAAUUGCUU | 1234 | AAGCAAUUUAACACUAGCC |
| siRNA 95 | 95 | GCUAGUGUUAAAUUGCUUG | 1235 | CAAGCAAUUUAACACUAGC |
| siRNA 96 | 96 | CUAGUGUUAAAUUGCUUGC | 1236 | GCAAGCAAUUUAACACUAG |
| siRNA 97 | 97 | UAGUGUUAAAUUGCUUGCC | 1237 | GGCAAGCAAUUUAACACUA |
| siRNA 98 | 98 | AGUGUUAAAUUGCUUGCCG | 1238 | CGGCAAGCAAUUUAACACU |
| siRNA 99 | 99 | GUGUUAAAUUGCUUGCCGG | 1239 | CCGGCAAGCAAUUUAACAC |
| siRNA 100 | 100 | UGUUAAAUUGCUUGCCGGU | 1240 | ACCGGCAAGCAAUUUAACA |
| siRNA 101 | 101 | GUUAAAUUGCUUGCCGGUG | 1241 | CACCGGCAAGCAAUUUAAC |
| siRNA 102 | 102 | UUAAAUUGCUUGCCGGUGU | 1242 | ACACCGGCAAGCAAUUUAA |
| siRNA 103 | 103 | UAAAUUGCUUGCCGGUGUU | 1243 | AACACCGGCAAGCAAUUUA |
| siRNA 104 | 104 | AAAUUGCUUGCCGGUGUUU | 1244 | AAACACCGGCAAGCAAUUU |
| siRNA 105 | 105 | AAUUGCUUGCCGGUGUUUU | 1245 | AAAACACCGGCAAGCAAUU |
| siRNA 106 | 106 | AUUGCUUGCCGGUGUUUUA | 1246 | UAAAACACCGGCAAGCAAU |
| siRNA 107 | 107 | UUGCUUGCCGGUGUUUUAA | 1247 | UUAAAACACCGGCAAGCAA |
| siRNA 108 | 108 | UGCUUGCCGGUGUUUUAAG | 1248 | CUUAAAACACCGGCAAGCA |
| siRNA 109 | 109 | GCUUGCCGGUGUUUUAAGA | 1249 | UCUUAAAACACCGGCAAGC |
| siRNA 110 | 110 | CUUGCCGGUGUUUUAAGAA | 1250 | UUCUUAAAACACCGGCAAG |
| siRNA 111 | 111 | UUGCCGGUGUUUUAAGAAA | 1251 | UUUCUUAAAACACCGGCAA |
| siRNA 112 | 112 | UGCCGGUGUUUUAAGAAAG | 1252 | CUUUCUUAAAACACCGGCA |
| siRNA 113 | 113 | GCCGGUGUUUUAAGAAAGC | 1253 | GCUUUCUUAAAACACCGGC |
| siRNA 114 | 114 | CCGGUGUUUUAAGAAAGCC | 1254 | GGCUUUCUUAAAACACCGG |
| siRNA 115 | 115 | CGGUGUUUUAAGAAAGCCA | 1255 | UGGCUUUCUUAAAACACCG |
| siRNA 116 | 116 | GGUGUUUUAAGAAAGCCAG | 1256 | CUGGCUUUCUUAAAACACC |
| siRNA 117 | 117 | GUGUUUUAAGAAAGCCAGA | 1257 | UCUGGCUUUCUUAAAACAC |
| siRNA 118 | 118 | UGUUUUAAGAAAGCCAGAU | 1258 | AUCUGGCUUUCUUAAAACA |
| siRNA 119 | 119 | GUUUUAAGAAAGCCAGAUG | 1259 | CAUCUGGCUUUCUUAAAAC |
| siRNA 120 | 120 | UUUUAAGAAAGCCAGAUGC | 1260 | GCAUCUGGCUUUCUUAAAA |
| siRNA 121 | 121 | UUUAAGAAAGCCAGAUGCC | 1261 | GGCAUCUGGCUUUCUUAAA |
| SIRNA 122 | 122 | UUAAGAAAGCCAGAUGCCU | 1262 | AGGCAUCUGGCUUUCUUAA |
| siRNA 123 | 123 | UAAGAAAGCCAGAUGCCUG | 1263 | CAGGCAUCUGGCUUUCUUA |
| siRNA 124 | 124 | AAGAAAGCCAGAUGCCUGG | 1264 | CCAGGCAUCUGGCUUUCUU |
| siRNA 125 | 125 | AGAAAGCCAGAUGCCUGGA | 1265 | UCCAGGCAUCUGGCUUUCU |
| siRNA 126 | 126 | GAAAGCCAGAUGCCUGGAU | 1266 | AUCCAGGCAUCUGGCUUUC |
| siRNA 127 | 127 | AAAGCCAGAUGCCUGGAUU | 1267 | AAUCCAGGCAUCUGGCUUU |
| siRNA 128 | 128 | AAGCCAGAUGCCUGGAUUG | 1268 | CAAUCCAGGCAUCUGGCUU |
| siRNA 129 | 129 | AGCCAGAUGCCUGGAUUGG | 1269 | CCAAUCCAGGCAUCUGGCU |
| siRNA 130 | 130 | GCCAGAUGCCUGGAUUGGA | 1270 | UCCAAUCCAGGCAUCUGGC |
| siRNA 131 | 131 | CCAGAUGCCUGGAUUGGAC | 1271 | GUCCAAUCCAGGCAUCUGG |
| siRNA 132 | 132 | CAGAUGCCUGGAUUGGACU | 1272 | AGUCCAAUCCAGGCAUCUG |
| siRNA 133 | 133 | AGAUGCCUGGAUUGGACUC | 1273 | GAGUCCAAUCCAGGCAUCU |
| siRNA 134 | 134 | GAUGCCUGGAUUGGACUCU | 1274 | AGAGUCCAAUCCAGGCAUC |
| siRNA 135 | 135 | AUGCCUGGAUUGGACUCUG | 1275 | CAGAGUCCAAUCCAGGCAU |
| siRNA 136 | 136 | UGCCUGGAUUGGACUCUGG | 1276 | CCAGAGUCCAAUCCAGGCA |
| siRNA 137 | 137 | GCCUGGAUUGGACUCUGGG | 1277 | CCCAGAGUCCAAUCCAGGC |
| siRNA 138 | 138 | CCUGGAUUGGACUCUGGGG | 1278 | CCCCAGAGUCCAAUCCAGG |
| siRNA 139 | 139 | CUGGAUUGGACUCUGGGGU | 1279 | ACCCCAGAGUCCAAUCCAG |
| siRNA 140 | 140 | UGGAUUGGACUCUGGGGUG | 1280 | CACCCCAGAGUCCAAUCCA |
| siRNA 141 | 141 | GGAUUGGACUCUGGGGUGU | 1281 | ACACCCCAGAGUCCAAUCC |
| siRNA 142 | 142 | GAUUGGACUCUGGGGUGUU | 1282 | AACACCCCAGAGUCCAAUC |
| siRNA 143 | 143 | AUUGGACUCUGGGGUGUUC | 1283 | GAACACCCCAGAGUCCAAU |
| siRNA 144 | 144 | UUGGACUCUGGGGUGUUCU | 1284 | AGAACACCCCAGAGUCCAA |
| siRNA 145 | 145 | UGGACUCUGGGGUGUUCUC | 1285 | GAGAACACCCCAGAGUCCA |
| siRNA 146 | 146 | GGACUCUGGGGUGUUCUCC | 1286 | GGAGAACACCCCAGAGUCC |
| siRNA 147 | 147 | GACUCUGGGGUGUUCUCCG | 1287 | CGGAGAACACCCCAGAGUC |
| siRNA 148 | 148 | ACUCUGGGGUGUUCUCCGA | 1288 | UCGGAGAACACCCCAGAGU |
| siRNA 149 | 149 | CUCUGGGGUGUUCUCCGAG | 1289 | CUCGGAGAACACCCCAGAG |
| siRNA 150 | 150 | UCUGGGGUGUUCUCCGAGG | 1290 | CCUCGGAGAACACCCCAGA |
| siRNA 151 | 151 | CUGGGGUGUUCUCCGAGGG | 1291 | CCCUCGGAGAACACCCCAG |
| siRNA 152 | 152 | UGGGGUGUUCUCCGAGGGA | 1292 | UCCCUCGGAGAACACCCCA |
| siRNA 153 | 153 | GGGGUGUUCUCCGAGGGAC | 1293 | GUCCCUCGGAGAACACCCC |
| siRNA 154 | 154 | GGGUGUUCUCCGAGGGACA | 1294 | UGUCCCUCGGAGAACACCC |
| siRNA 155 | 155 | GGUGUUCUCCGAGGGACAC | 1295 | GUGUCCCUCGGAGAACACC |
| siRNA 156 | 156 | GUGUUCUCCGAGGGACACC | 1296 | GGUGUCCCUCGGAGAACAC |
| siRNA 157 | 157 | UGUUCUCCGAGGGACACCU | 1297 | AGGUGUCCCUCGGAGAACA |
| siRNA 158 | 158 | GUUCUCCGAGGGACACCUU | 1298 | AAGGUGUCCCUCGGAGAAC |
| siRNA 159 | 159 | UUCUCCGAGGGACACCUUC | 1299 | GAAGGUGUCCCUCGGAGAA |
| siRNA 160 | 160 | UCUCCGAGGGACACCUUCA | 1300 | UGAAGGUGUCCCUCGGAGA |
| siRNA 161 | 161 | CUCCGAGGGACACCUUCAU | 1301 | AUGAAGGUGUCCCUCGGAG |
| siRNA 162 | 162 | UCCGAGGGACACCUUCAUC | 1302 | GAUGAAGGUGUCCCUCGGA |
| siRNA 163 | 163 | CCGAGGGACACCUUCAUCA | 1303 | UGAUGAAGGUGUCCCUCGG |
| siRNA 164 | 164 | CGAGGGACACCUUCAUCAU | 1304 | AUGAUGAAGGUGUCCCUCG |
| siRNA 165 | 165 | GAGGGACACCUUCAUCAUA | 1305 | UAUGAUGAAGGUGUCCCUC |
| siRNA 166 | 166 | AGGGACACCUUCAUCAUAC | 1306 | GUAUGAUGAAGGUGUCCCU |
| siRNA 167 | 167 | GGGACACCUUCAUCAUACA | 1307 | UGUAUGAUGAAGGUGUCCC |
| siRNA 168 | 168 | GGACACCUUCAUCAUACAA | 1308 | UUGUAUGAUGAAGGUGUCC |
| siRNA 169 | 169 | GACACCUUCAUCAUACAAA | 1309 | UUUGUAUGAUGAAGGUGUC |
| siRNA 170 | 170 | ACACCUUCAUCAUACAAAC | 1310 | GUUUGUAUGAUGAAGGUGU |
| siRNA 171 | 171 | CACCUUCAUCAUACAAACU | 1311 | AGUUUGUAUGAUGAAGGUG |
| siRNA 172 | 172 | ACCUUCAUCAUACAAACUC | 1312 | GAGUUUGUAUGAUGAAGGU |
| siRNA 173 | 173 | CCUUCAUCAUACAAACUCU | 1313 | AGAGUUUGUAUGAUGAAGG |
| siRNA 174 | 174 | CUUCAUCAUACAAACUCUG | 1314 | CAGAGUUUGUAUGAUGAAG |
| siRNA 175 | 175 | UUCAUCAUACAAACUCUGU | 1315 | ACAGAGUUUGUAUGAUGAA |
| siRNA 176 | 176 | UCAUCAUACAAACUCUGUA | 1316 | UACAGAGUUUGUAUGAUGA |
| siRNA 177 | 177 | CAUCAUACAAACUCUGUAC | 1317 | GUACAGAGUUUGUAUGAUG |
| siRNA 178 | 178 | AUCAUACAAACUCUGUACU | 1318 | AGUACAGAGUUUGUAUGAU |
| siRNA 179 | 179 | UCAUACAAACUCUGUACUU | 1319 | AAGUACAGAGUUUGUAUGA |
| siRNA 180 | 180 | CAUACAAACUCUGUACUUC | 1320 | GAAGUACAGAGUUUGUAUG |
| siRNA 181 | 181 | AUACAAACUCUGUACUUCC | 1321 | GGAAGUACAGAGUUUGUAU |
| siRNA 182 | 182 | UACAAACUCUGUACUUCCU | 1322 | AGGAAGUACAGAGUUUGUA |
| siRNA 183 | 183 | ACAAACUCUGUACUUCCUG | 1323 | CAGGAAGUACAGAGUUUGU |
| siRNA 184 | 184 | CAAACUCUGUACUUCCUGG | 1324 | CCAGGAAGUACAGAGUUUG |
| siRNA 185 | 185 | AAACUCUGUACUUCCUGGA | 1325 | UCCAGGAAGUACAGAGUUU |
| siRNA 186 | 186 | AACUCUGUACUUCCUGGAA | 1326 | UUCCAGGAAGUACAGAGUU |
| siRNA 187 | 187 | ACUCUGUACUUCCUGGAAU | 1327 | AUUCCAGGAAGUACAGAGU |
| siRNA 188 | 188 | CUCUGUACUUCCUGGAAUC | 1328 | GAUUCCAGGAAGUACAGAG |
| siRNA 189 | 189 | UCUGUACUUCCUGGAAUCG | 1329 | CGAUUCCAGGAAGUACAGA |
| siRNA 190 | 190 | CUGUACUUCCUGGAAUCGA | 1330 | UCGAUUCCAGGAAGUACAG |
| siRNA 191 | 191 | UGUACUUCCUGGAAUCGAU | 1331 | AUCGAUUCCAGGAAGUACA |
| siRNA 192 | 192 | GUACUUCCUGGAAUCGAUA | 1332 | UAUCGAUUCCAGGAAGUAC |
| siRNA 193 | 193 | UACUUCCUGGAAUCGAUAC | 1333 | GUAUCGAUUCCAGGAAGUA |
| siRNA 194 | 194 | ACUUCCUGGAAUCGAUACU | 1334 | AGUAUCGAUUCCAGGAAGU |
| siRNA 195 | 195 | CUUCCUGGAAUCGAUACUU | 1335 | AAGUAUCGAUUCCAGGAAG |
| siRNA 196 | 196 | UUCCUGGAAUCGAUACUUG | 1336 | CAAGUAUCGAUUCCAGGAA |
| siRNA 197 | 197 | UCCUGGAAUCGAUACUUGU | 1337 | ACAAGUAUCGAUUCCAGGA |
| siRNA 198 | 198 | CCUGGAAUCGAUACUUGUA | 1338 | UACAAGUAUCGAUUCCAGG |
| siRNA 199 | 199 | CUGGAAUCGAUACUUGUAU | 1339 | AUACAAGUAUCGAUUCCAG |
| siRNA 200 | 200 | UGGAAUCGAUACUUGUAUU | 1340 | AAUACAAGUAUCGAUUCCA |
| siRNA 201 | 201 | GGAAUCGAUACUUGUAUUU | 1341 | AAAUACAAGUAUCGAUUCC |
| siRNA 202 | 202 | GAAUCGAUACUUGUAUUUU | 1342 | AAAAUACAAGUAUCGAUUC |
| siRNA 203 | 203 | AAUCGAUACUUGUAUUUUU | 1343 | AAAAAUACAAGUAUCGAUU |
| siRNA 204 | 204 | AUCGAUACUUGUAUUUUUC | 1344 | GAAAAAUACAAGUAUCGAU |
| siRNA 205 | 205 | UCGAUACUUGUAUUUUUCU | 1345 | AGAAAAAUACAAGUAUCGA |
| siRNA 206 | 206 | CGAUACUUGUAUUUUUCUA | 1346 | UAGAAAAAUACAAGUAUCG |
| siRNA 207 | 207 | GAUACUUGUAUUUUUCUAG | 1347 | CUAGAAAAAUACAAGUAUC |
| siRNA 208 | 208 | AUACUUGUAUUUUUCUAGU | 1348 | ACUAGAAAAAUACAAGUAU |
| siRNA 209 | 209 | UACUUGUAUUUUUCUAGUA | 1349 | UACUAGAAAAAUACAAGUA |
| siRNA 210 | 210 | ACUUGUAUUUUUCUAGUAC | 1350 | GUACUAGAAAAAUACAAGU |
| siRNA 211 | 211 | CUUGUAUUUUUCUAGUACC | 1351 | GGUACUAGAAAAAUACAAG |
| siRNA 212 | 212 | UUGUAUUUUUCUAGUACCA | 1352 | UGGUACUAGAAAAAUACAA |
| siRNA 213 | 213 | UGUAUUUUUCUAGUACCAA | 1353 | UUGGUACUAGAAAAAUACA |
| siRNA 214 | 214 | GUAUUUUUCUAGUACCAAG | 1354 | CUUGGUACUAGAAAAAUAC |
| siRNA 215 | 215 | UAUUUUUCUAGUACCAAGU | 1355 | ACUUGGUACUAGAAAAAUA |
| siRNA 216 | 216 | AUUUUUCUAGUACCAAGUU | 1356 | AACUUGGUACUAGAAAAAU |
| siRNA 217 | 217 | UUUUUCUAGUACCAAGUUA | 1357 | UAACUUGGUACUAGAAAAA |
| siRNA 218 | 218 | UUUUCUAGUACCAAGUUAC | 1358 | GUAACUUGGUACUAGAAAA |
| siRNA 219 | 219 | UUUCUAGUACCAAGUUACG | 1359 | CGUAACUUGGUACUAGAAA |
| siRNA 220 | 220 | UUCUAGUACCAAGUUACGU | 1360 | ACGUAACUUGGUACUAGAA |
| siRNA 221 | 221 | UCUAGUACCAAGUUACGUG | 1361 | CACGUAACUUGGUACUAGA |
| siRNA 222 | 222 | CUAGUACCAAGUUACGUGC | 1362 | GCACGUAACUUGGUACUAG |
| siRNA 223 | 223 | UAGUACCAAGUUACGUGCA | 1363 | UGCACGUAACUUGGUACUA |
| siRNA 224 | 224 | AGUACCAAGUUACGUGCAC | 1364 | GUGCACGUAACUUGGUACU |
| siRNA 225 | 225 | GUACCAAGUUACGUGCACC | 1365 | GGUGCACGUAACUUGGUAC |
| siRNA 226 | 226 | UACCAAGUUACGUGCACCA | 1366 | UGGUGCACGUAACUUGGUA |
| siRNA 227 | 227 | ACCAAGUUACGUGCACCAA | 1367 | UUGGUGCACGUAACUUGGU |
| siRNA 228 | 228 | CCAAGUUACGUGCACCAAA | 1368 | UUUGGUGCACGUAACUUGG |
| siRNA 229 | 229 | CAAGUUACGUGCACCAAAU | 1369 | AUUUGGUGCACGUAACUUG |
| siRNA 230 | 230 | AAGUUACGUGCACCAAAUU | 1370 | AAUUUGGUGCACGUAACUU |
| siRNA 231 | 231 | AGUUACGUGCACCAAAUUA | 1371 | UAAUUUGGUGCACGUAACU |
| siRNA 232 | 232 | GUUACGUGCACCAAAUUAU | 1372 | AUAAUUUGGUGCACGUAAC |
| siRNA 233 | 233 | UUACGUGCACCAAAUUAUA | 1373 | UAUAAUUUGGUGCACGUAA |
| siRNA 234 | 234 | UACGUGCACCAAAUUAUAA | 1374 | UUAUAAUUUGGUGCACGUA |
| siRNA 235 | 235 | ACGUGCACCAAAUUAUAAA | 1375 | UUUAUAAUUUGGUGCACGU |
| siRNA 236 | 236 | CGUGCACCAAAUUAUAAAA | 1376 | UUUUAUAAUUUGGUGCACG |
| siRNA 237 | 237 | GUGCACCAAAUUAUAAAAC | 1377 | GUUUUAUAAUUUGGUGCAC |
| siRNA 238 | 238 | UGCACCAAAUUAUAAAACA | 1378 | UGUUUUAUAAUUUGGUGCA |
| siRNA 239 | 239 | GCACCAAAUUAUAAAACAC | 1379 | GUGUUUUAUAAUUUGGUGC |
| siRNA 240 | 240 | CACCAAAUUAUAAAACACU | 1380 | AGUGUUUUAUAAUUUGGUG |
| siRNA 241 | 241 | ACCAAAUUAUAAAACACUU | 1381 | AAGUGUUUUAUAAUUUGGU |
| siRNA 242 | 242 | CCAAAUUAUAAAACACUUU | 1382 | AAAGUGUUUUAUAAUUUGG |
| siRNA 243 | 243 | CAAAUUAUAAAACACUUUU | 1383 | AAAAGUGUUUUAUAAUUUG |
| siRNA 244 | 244 | AAAUUAUAAAACACUUUUU | 1384 | AAAAAGUGUUUUAUAAUUU |
| siRNA 245 | 245 | AAUUAUAAAACACUUUUUU | 1385 | AAAAAAGUGUUUUAUAAUU |
| siRNA 246 | 246 | AUUAUAAAACACUUUUUUA | 1386 | UAAAAAAGUGUUUUAUAAU |
| siRNA 247 | 247 | UUAUAAAACACUUUUUUAU | 1387 | AUAAAAAAGUGUUUUAUAA |
| siRNA 248 | 248 | UAUAAAACACUUUUUUAUA | 1388 | UAUAAAAAAGUGUUUUAUA |
| siRNA 249 | 249 | AUAAAACACUUUUUUAUAA | 1389 | UUAUAAAAAAGUGUUUUAU |
| siRNA 250 | 250 | UAAAACACUUUUUUAUAAU | 1390 | AUUAUAAAAAAGUGUUUUA |
| siRNA 251 | 251 | AAAACACUUUUUUAUAAUA | 1391 | UAUUAUAAAAAAGUGUUUU |
| siRNA 252 | 252 | AAACACUUUUUUAUAAUAU | 1392 | AUAUUAUAAAAAAGUGUUU |
| siRNA 253 | 253 | AACACUUUUUUAUAAUAUU | 1393 | AAUAUUAUAAAAAAGUGUU |
| siRNA 254 | 254 | ACACUUUUUUAUAAUAUUU | 1394 | AAAUAUUAUAAAAAAGUGU |
| siRNA 255 | 255 | CACUUUUUUAUAAUAUUUU | 1395 | AAAAUAUUAUAAAAAAGUG |
| siRNA 256 | 256 | ACUUUUUUAUAAUAUUUUC | 1396 | GAAAAUAUUAUAAAAAAGU |
| siRNA 257 | 257 | CUUUUUUAUAAUAUUUUCU | 1397 | AGAAAAUAUUAUAAAAAAG |
| siRNA 258 | 258 | UUUUUUAUAAUAUUUUCUC | 1398 | GAGAAAAUAUUAUAAAAAA |
| siRNA 259 | 259 | UUUUUAUAAUAUUUUCUCA | 1399 | UGAGAAAAUAUUAUAAAAA |
| siRNA 260 | 260 | UUUUAUAAUAUUUUCUCAC | 1400 | GUGAGAAAAUAUUAUAAAA |
| siRNA 261 | 261 | UUUAUAAUAUUUUCUCACU | 1401 | AGUGAGAAAAUAUUAUAAA |
| siRNA 262 | 262 | UUAUAAUAUUUUCUCACUG | 1402 | CAGUGAGAAAAUAUUAUAA |
| siRNA 263 | 263 | UAUAAUAUUUUCUCACUGA | 1403 | UCAGUGAGAAAAUAUUAUA |
| siRNA 264 | 264 | AUAAUAUUUUCUCACUGAG | 1404 | CUCAGUGAGAAAAUAUUAU |
| siRNA 265 | 265 | UAAUAUUUUCUCACUGAGA | 1405 | UCUCAGUGAGAAAAUAUUA |
| siRNA 266 | 266 | AAUAUUUUCUCACUGAGAC | 1406 | GUCUCAGUGAGAAAAUAUU |
| siRNA 267 | 267 | AUAUUUUCUCACUGAGACU | 1407 | AGUCUCAGUGAGAAAAUAU |
| siRNA 268 | 268 | UAUUUUCUCACUGAGACUC | 1408 | GAGUCUCAGUGAGAAAAUA |
| siRNA 269 | 269 | AUUUUCUCACUGAGACUCC | 1409 | GGAGUCUCAGUGAGAAAAU |
| siRNA 270 | 270 | UUUUCUCACUGAGACUCCC | 1410 | GGGAGUCUCAGUGAGAAAA |
| siRNA 271 | 271 | UUUCUCACUGAGACUCCCA | 1411 | UGGGAGUCUCAGUGAGAAA |
| siRNA 272 | 272 | UUCUCACUGAGACUCCCAG | 1412 | CUGGGAGUCUCAGUGAGAA |
| siRNA 273 | 273 | UCUCACUGAGACUCCCAGG | 1413 | CCUGGGAGUCUCAGUGAGA |
| siRNA 274 | 274 | CUCACUGAGACUCCCAGGG | 1414 | CCCUGGGAGUCUCAGUGAG |
| siRNA 275 | 275 | UCACUGAGACUCCCAGGGC | 1415 | GCCCUGGGAGUCUCAGUGA |
| siRNA 276 | 276 | CACUGAGACUCCCAGGGCU | 1416 | AGCCCUGGGAGUCUCAGUG |
| siRNA 277 | 277 | ACUGAGACUCCCAGGGCUU | 1417 | AAGCCCUGGGAGUCUCAGU |
| siRNA 278 | 278 | CUGAGACUCCCAGGGCUUU | 1418 | AAAGCCCUGGGAGUCUCAG |
| siRNA 279 | 279 | UGAGACUCCCAGGGCUUUU | 1419 | AAAAGCCCUGGGAGUCUCA |
| siRNA 280 | 280 | GAGACUCCCAGGGCUUUUA | 1420 | UAAAAGCCCUGGGAGUCUC |
| siRNA 281 | 281 | AGACUCCCAGGGCUUUUAC | 1421 | GUAAAAGCCCUGGGAGUCU |
| siRNA 282 | 282 | GACUCCCAGGGCUUUUACU | 1422 | AGUAAAAGCCCUGGGAGUC |
| siRNA 283 | 283 | ACUCCCAGGGCUUUUACUA | 1423 | UAGUAAAAGCCCUGGGAGU |
| siRNA 284 | 284 | CUCCCAGGGCUUUUACUAU | 1424 | AUAGUAAAAGCCCUGGGAG |
| siRNA 285 | 285 | UCCCAGGGCUUUUACUAUC | 1425 | GAUAGUAAAAGCCCUGGGA |
| siRNA 286 | 286 | CCCAGGGCUUUUACUAUCU | 1426 | AGAUAGUAAAAGCCCUGGG |
| siRNA 287 | 287 | CCAGGGCUUUUACUAUCUC | 1427 | GAGAUAGUAAAAGCCCUGG |
| siRNA 288 | 288 | CAGGGCUUUUACUAUCUCC | 1428 | GGAGAUAGUAAAAGCCCUG |
| siRNA 289 | 289 | AGGGCUUUUACUAUCUCCA | 1429 | UGGAGAUAGUAAAAGCCCU |
| siRNA 290 | 290 | GGGCUUUUACUAUCUCCAG | 1430 | CUGGAGAUAGUAAAAGCCC |
| siRNA 291 | 291 | GGCUUUUACUAUCUCCAGA | 1431 | UCUGGAGAUAGUAAAAGCC |
| siRNA 292 | 292 | GCUUUUACUAUCUCCAGAA | 1432 | UUCUGGAGAUAGUAAAAGC |
| siRNA 293 | 293 | CUUUUACUAUCUCCAGAAU | 1433 | AUUCUGGAGAUAGUAAAAG |
| siRNA 294 | 294 | UUUUACUAUCUCCAGAAUG | 1434 | CAUUCUGGAGAUAGUAAAA |
| siRNA 295 | 295 | UUUACUAUCUCCAGAAUGU | 1435 | ACAUUCUGGAGAUAGUAAA |
| siRNA 296 | 296 | UUACUAUCUCCAGAAUGUA | 1436 | UACAUUCUGGAGAUAGUAA |
| siRNA 297 | 297 | UACUAUCUCCAGAAUGUAU | 1437 | AUACAUUCUGGAGAUAGUA |
| siRNA 298 | 298 | ACUAUCUCCAGAAUGUAUU | 1438 | AAUACAUUCUGGAGAUAGU |
| siRNA 299 | 299 | CUAUCUCCAGAAUGUAUUU | 1439 | AAAUACAUUCUGGAGAUAG |
| siRNA 300 | 300 | UAUCUCCAGAAUGUAUUUU | 1440 | AAAAUACAUUCUGGAGAUA |
| siRNA 301 | 301 | AUCUCCAGAAUGUAUUUUU | 1441 | AAAAAUACAUUCUGGAGAU |
| siRNA 302 | 302 | UCUCCAGAAUGUAUUUUUC | 1442 | GAAAAAUACAUUCUGGAGA |
| siRNA 303 | 303 | CUCCAGAAUGUAUUUUUCC | 1443 | GGAAAAAUACAUUCUGGAG |
| siRNA 304 | 304 | UCCAGAAUGUAUUUUUCCU | 1444 | AGGAAAAAUACAUUCUGGA |
| siRNA 305 | 305 | CCAGAAUGUAUUUUUCCUU | 1445 | AAGGAAAAAUACAUUCUGG |
| siRNA 306 | 306 | CAGAAUGUAUUUUUCCUUU | 1446 | AAAGGAAAAAUACAUUCUG |
| siRNA 307 | 307 | AGAAUGUAUUUUUCCUUUU | 1447 | AAAAGGAAAAAUACAUUCU |
| siRNA 308 | 308 | GAAUGUAUUUUUCCUUUUU | 1448 | AAAAAGGAAAAAUACAUUC |
| siRNA 309 | 309 | AAUGUAUUUUUCCUUUUUC | 1449 | GAAAAAGGAAAAAUACAUU |
| siRNA 310 | 310 | AUGUAUUUUUCCUUUUUCC | 1450 | GGAAAAAGGAAAAAUACAU |
| siRNA 311 | 311 | UGUAUUUUUCCUUUUUCCG | 1451 | CGGAAAAAGGAAAAAUACA |
| siRNA 312 | 312 | GUAUUUUUCCUUUUUCCGU | 1452 | ACGGAAAAAGGAAAAAUAC |
| siRNA 313 | 313 | UAUUUUUCCUUUUUCCGUA | 1453 | UACGGAAAAAGGAAAAAUA |
| siRNA 314 | 314 | AUUUUUCCUUUUUCCGUAA | 1454 | UUACGGAAAAAGGAAAAAU |
| siRNA 315 | 315 | UUUUUCCUUUUUCCGUAAG | 1455 | CUUACGGAAAAAGGAAAAA |
| siRNA 316 | 316 | UUUUCCUUUUUCCGUAAGA | 1456 | UCUUACGGAAAAAGGAAAA |
| siRNA 317 | 317 | UUUCCUUUUUCCGUAAGAC | 1457 | GUCUUACGGAAAAAGGAAA |
| siRNA 318 | 318 | UUCCUUUUUCCGUAAGACU | 1458 | AGUCUUACGGAAAAAGGAA |
| siRNA 319 | 319 | UCCUUUUUCCGUAAGACUC | 1459 | GAGUCUUACGGAAAAAGGA |
| siRNA 320 | 320 | CCUUUUUCCGUAAGACUCA | 1460 | UGAGUCUUACGGAAAAAGG |
| siRNA 321 | 321 | CUUUUUCCGUAAGACUCAA | 1461 | UUGAGUCUUACGGAAAAAG |
| siRNA 322 | 322 | UUUUUCCGUAAGACUCAAA | 1462 | UUUGAGUCUUACGGAAAAA |
| siRNA 323 | 323 | UUUUCCGUAAGACUCAAAA | 1463 | UUUUGAGUCUUACGGAAAA |
| siRNA 324 | 324 | UUUCCGUAAGACUCAAAAG | 1464 | CUUUUGAGUCUUACGGAAA |
| siRNA 325 | 325 | UUCCGUAAGACUCAAAAGU | 1465 | ACUUUUGAGUCUUACGGAA |
| siRNA 326 | 326 | UCCGUAAGACUCAAAAGUA | 1466 | UACUUUUGAGUCUUACGGA |
| siRNA 327 | 327 | CCGUAAGACUCAAAAGUAA | 1467 | UUACUUUUGAGUCUUACGG |
| siRNA 328 | 328 | CGUAAGACUCAAAAGUAAU | 1468 | AUUACUUUUGAGUCUUACG |
| siRNA 329 | 329 | GUAAGACUCAAAAGUAAUA | 1469 | UAUUACUUUUGAGUCUUAC |
| siRNA 330 | 330 | UAAGACUCAAAAGUAAUAU | 1470 | AUAUUACUUUUGAGUCUUA |
| siRNA 331 | 331 | AAGACUCAAAAGUAAUAUA | 1471 | UAUAUUACUUUUGAGUCUU |
| siRNA 332 | 332 | AGACUCAAAAGUAAUAUAA | 1472 | UUAUAUUACUUUUGAGUCU |
| siRNA 333 | 333 | GACUCAAAAGUAAUAUAAG | 1473 | CUUAUAUUACUUUUGAGUC |
| siRNA 334 | 334 | ACUCAAAAGUAAUAUAAGG | 1474 | CCUUAUAUUACUUUUGAGU |
| siRNA 335 | 335 | CUCAAAAGUAAUAUAAGGU | 1475 | ACCUUAUAUUACUUUUGAG |
| siRNA 336 | 336 | UCAAAAGUAAUAUAAGGUC | 1476 | GACCUUAUAUUACUUUUGA |
| siRNA 337 | 337 | CAAAAGUAAUAUAAGGUCU | 1477 | AGACCUUAUAUUACUUUUG |
| siRNA 338 | 338 | AAAAGUAAUAUAAGGUCUA | 1478 | UAGACCUUAUAUUACUUUU |
| siRNA 339 | 339 | AAAGUAAUAUAAGGUCUAC | 1479 | GUAGACCUUAUAUUACUUU |
| siRNA 340 | 340 | AAGUAAUAUAAGGUCUACA | 1480 | UGUAGACCUUAUAUUACUU |
| siRNA 341 | 341 | AGUAAUAUAAGGUCUACAA | 1481 | UUGUAGACCUUAUAUUACU |
| siRNA 342 | 342 | GUAAUAUAAGGUCUACAAA | 1482 | UUUGUAGACCUUAUAUUAC |
| siRNA 343 | 343 | UAAUAUAAGGUCUACAAAA | 1483 | UUUUGUAGACCUUAUAUUA |
| siRNA 344 | 344 | AAUAUAAGGUCUACAAAAU | 1484 | AUUUUGUAGACCUUAUAUU |
| siRNA 345 | 345 | AUAUAAGGUCUACAAAAUC | 1485 | GAUUUUGUAGACCUUAUAU |
| siRNA 346 | 346 | UAUAAGGUCUACAAAAUCU | 1486 | AGAUUUUGUAGACCUUAUA |
| siRNA 347 | 347 | AUAAGGUCUACAAAAUCUA | 1487 | UAGAUUUUGUAGACCUUAU |
| siRNA 348 | 348 | UAAGGUCUACAAAAUCUAC | 1488 | GUAGAUUUUGUAGACCUUA |
| siRNA 349 | 349 | AAGGUCUACAAAAUCUACU | 1489 | AGUAGAUUUUGUAGACCUU |
| siRNA 350 | 350 | AGGUCUACAAAAUCUACUA | 1490 | UAGUAGAUUUUGUAGACCU |
| siRNA 351 | 351 | GGUCUACAAAAUCUACUAA | 1491 | UUAGUAGAUUUUGUAGACC |
| siRNA 352 | 352 | GUCUACAAAAUCUACUAAA | 1492 | UUUAGUAGAUUUUGUAGAC |
| siRNA 353 | 353 | UCUACAAAAUCUACUAAAA | 1493 | UUUUAGUAGAUUUUGUAGA |
| siRNA 354 | 354 | CUACAAAAUCUACUAAAAA | 1494 | UUUUUAGUAGAUUUUGUAG |
| siRNA 355 | 355 | UACAAAAUCUACUAAAAAG | 1495 | CUUUUUAGUAGAUUUUGUA |
| siRNA 356 | 356 | ACAAAAUCUACUAAAAAGU | 1496 | ACUUUUUAGUAGAUUUUGU |
| siRNA 357 | 357 | CAAAAUCUACUAAAAAGUC | 1497 | GACUUUUUAGUAGAUUUUG |
| siRNA 358 | 358 | AAAAUCUACUAAAAAGUCU | 1498 | AGACUUUUUAGUAGAUUUU |
| siRNA 359 | 359 | AAAUCUACUAAAAAGUCUC | 1499 | GAGACUUUUUAGUAGAUUU |
| siRNA 360 | 360 | AAUCUACUAAAAAGUCUCU | 1500 | AGAGACUUUUUAGUAGAUU |
| siRNA 361 | 361 | AUCUACUAAAAAGUCUCUG | 1501 | CAGAGACUUUUUAGUAGAU |
| siRNA 362 | 362 | UCUACUAAAAAGUCUCUGC | 1502 | GCAGAGACUUUUUAGUAGA |
| siRNA 363 | 363 | CUACUAAAAAGUCUCUGCA | 1503 | UGCAGAGACUUUUUAGUAG |
| siRNA 364 | 364 | UACUAAAAAGUCUCUGCAA | 1504 | UUGCAGAGACUUUUUAGUA |
| siRNA 365 | 365 | ACUAAAAAGUCUCUGCAAA | 1505 | UUUGCAGAGACUUUUUAGU |
| siRNA 366 | 366 | CUAAAAAGUCUCUGCAAAA | 1506 | UUUUGCAGAGACUUUUUAG |
| siRNA 367 | 367 | UAAAAAGUCUCUGCAAAAA | 1507 | UUUUUGCAGAGACUUUUUA |
| siRNA 368 | 368 | AAAAAGUCUCUGCAAAAAG | 1508 | CUUUUUGCAGAGACUUUUU |
| siRNA 369 | 369 | AAAAGUCUCUGCAAAAAGU | 1509 | ACUUUUUGCAGAGACUUUU |
| siRNA 370 | 370 | AAAGUCUCUGCAAAAAGUA | 1510 | UACUUUUUGCAGAGACUUU |
| siRNA 371 | 371 | AAGUCUCUGCAAAAAGUAG | 1511 | CUACUUUUUGCAGAGACUU |
| siRNA 372 | 372 | AGUCUCUGCAAAAAGUAGA | 1512 | UCUACUUUUUGCAGAGACU |
| siRNA 373 | 373 | GUCUCUGCAAAAAGUAGAU | 1513 | AUCUACUUUUUGCAGAGAC |
| siRNA 374 | 374 | UCUCUGCAAAAAGUAGAUG | 1514 | CAUCUACUUUUUGCAGAGA |
| siRNA 375 | 375 | CUCUGCAAAAAGUAGAUGA | 1515 | UCAUCUACUUUUUGCAGAG |
| siRNA 376 | 376 | UCUGCAAAAAGUAGAUGAA | 1516 | UUCAUCUACUUUUUGCAGA |
| siRNA 377 | 377 | CUGCAAAAAGUAGAUGAAG | 1517 | CUUCAUCUACUUUUUGCAG |
| siRNA 378 | 378 | UGCAAAAAGUAGAUGAAGA | 1518 | UCUUCAUCUACUUUUUGCA |
| siRNA 379 | 379 | GCAAAAAGUAGAUGAAGAG | 1519 | CUCUUCAUCUACUUUUUGC |
| siRNA 380 | 380 | CAAAAAGUAGAUGAAGAGG | 1520 | CCUCUUCAUCUACUUUUUG |
| siRNA 381 | 381 | AAAAAGUAGAUGAAGAGGA | 1521 | UCCUCUUCAUCUACUUUUU |
| siRNA 382 | 382 | AAAAGUAGAUGAAGAGGAC | 1522 | GUCCUCUUCAUCUACUUUU |
| siRNA 383 | 383 | AAAGUAGAUGAAGAGGACU | 1523 | AGUCCUCUUCAUCUACUUU |
| siRNA 384 | 384 | AAGUAGAUGAAGAGGACUC | 1524 | GAGUCCUCUUCAUCUACUU |
| siRNA 385 | 385 | AGUAGAUGAAGAGGACUCU | 1525 | AGAGUCCUCUUCAUCUACU |
| SiRNA 386 | 386 | GUAGAUGAAGAGGACUCUG | 1526 | CAGAGUCCUCUUCAUCUAC |
| siRNA 387 | 387 | UAGAUGAAGAGGACUCUGA | 1527 | UCAGAGUCCUCUUCAUCUA |
| siRNA 388 | 388 | AGAUGAAGAGGACUCUGAU | 1528 | AUCAGAGUCCUCUUCAUCU |
| siRNA 389 | 389 | GAUGAAGAGGACUCUGAUG | 1529 | CAUCAGAGUCCUCUUCAUC |
| siRNA 390 | 390 | AUGAAGAGGACUCUGAUGA | 1530 | UCAUCAGAGUCCUCUUCAU |
| siRNA 391 | 391 | UGAAGAGGACUCUGAUGAA | 1531 | UUCAUCAGAGUCCUCUUCA |
| siRNA 392 | 392 | GAAGAGGACUCUGAUGAAG | 1532 | CUUCAUCAGAGUCCUCUUC |
| siRNA 393 | 393 | AAGAGGACUCUGAUGAAGA | 1533 | UCUUCAUCAGAGUCCUCUU |
| siRNA 394 | 394 | AGAGGACUCUGAUGAAGAA | 1534 | UUCUUCAUCAGAGUCCUCU |
| siRNA 395 | 395 | GAGGACUCUGAUGAAGAAA | 1535 | UUUCUUCAUCAGAGUCCUC |
| siRNA 396 | 396 | AGGACUCUGAUGAAGAAAG | 1536 | CUUUCUUCAUCAGAGUCCU |
| siRNA 397 | 397 | GGACUCUGAUGAAGAAAGC | 1537 | GCUUUCUUCAUCAGAGUCC |
| siRNA 398 | 398 | GACUCUGAUGAAGAAAGCC | 1538 | GGCUUUCUUCAUCAGAGUC |
| siRNA 399 | 399 | ACUCUGAUGAAGAAAGCCA | 1539 | UGGCUUUCUUCAUCAGAGU |
| siRNA 400 | 400 | CUCUGAUGAAGAAAGCCAU | 1540 | AUGGCUUUCUUCAUCAGAG |
| siRNA 401 | 401 | UCUGAUGAAGAAAGCCAUC | 1541 | GAUGGCUUUCUUCAUCAGA |
| siRNA 402 | 402 | CUGAUGAAGAAAGCCAUCA | 1542 | UGAUGGCUUUCUUCAUCAG |
| siRNA 403 | 403 | UGAUGAAGAAAGCCAUCAU | 1543 | AUGAUGGCUUUCUUCAUCA |
| siRNA 404 | 404 | GAUGAAGAAAGCCAUCAUG | 1544 | CAUGAUGGCUUUCUUCAUC |
| siRNA 405 | 405 | AUGAAGAAAGCCAUCAUGA | 1545 | UCAUGAUGGCUUUCUUCAU |
| siRNA 406 | 406 | UGAAGAAAGCCAUCAUGAU | 1546 | AUCAUGAUGGCUUUCUUCA |
| siRNA 407 | 407 | GAAGAAAGCCAUCAUGAUG | 1547 | CAUCAUGAUGGCUUUCUUC |
| siRNA 408 | 408 | AAGAAAGCCAUCAUGAUGA | 1548 | UCAUCAUGAUGGCUUUCUU |
| siRNA 409 | 409 | AGAAAGCCAUCAUGAUGAG | 1549 | CUCAUCAUGAUGGCUUUCU |
| siRNA 410 | 410 | GAAAGCCAUCAUGAUGAGA | 1550 | UCUCAUCAUGAUGGCUUUC |
| siRNA 411 | 411 | AAAGCCAUCAUGAUGAGAU | 1551 | AUCUCAUCAUGAUGGCUUU |
| siRNA 412 | 412 | AAGCCAUCAUGAUGAGAUG | 1552 | CAUCUCAUCAUGAUGGCUU |
| siRNA 413 | 413 | AGCCAUCAUGAUGAGAUGA | 1553 | UCAUCUCAUCAUGAUGGCU |
| siRNA 414 | 414 | GCCAUCAUGAUGAGAUGAG | 1554 | CUCAUCUCAUCAUGAUGGC |
| siRNA 415 | 415 | CCAUCAUGAUGAGAUGAGU | 1555 | ACUCAUCUCAUCAUGAUGG |
| siRNA 416 | 416 | CAUCAUGAUGAGAUGAGUG | 1556 | CACUCAUCUCAUCAUGAUG |
| siRNA 417 | 417 | AUCAUGAUGAGAUGAGUGA | 1557 | UCACUCAUCUCAUCAUGAU |
| siRNA 418 | 418 | UCAUGAUGAGAUGAGUGAG | 1558 | CUCACUCAUCUCAUCAUGA |
| SiRNA 419 | 419 | CAUGAUGAGAUGAGUGAGC | 1559 | GCUCACUCAUCUCAUCAUG |
| siRNA 420 | 420 | AUGAUGAGAUGAGUGAGCA | 1560 | UGCUCACUCAUCUCAUCAU |
| siRNA 421 | 421 | UGAUGAGAUGAGUGAGCAG | 1561 | CUGCUCACUCAUCUCAUCA |
| siRNA 422 | 422 | GAUGAGAUGAGUGAGCAGG | 1562 | CCUGCUCACUCAUCUCAUC |
| siRNA 423 | 423 | AUGAGAUGAGUGAGCAGGA | 1563 | UCCUGCUCACUCAUCUCAU |
| siRNA 424 | 424 | UGAGAUGAGUGAGCAGGAA | 1564 | UUCCUGCUCACUCAUCUCA |
| siRNA 425 | 425 | GAGAUGAGUGAGCAGGAAG | 1565 | CUUCCUGCUCACUCAUCUC |
| siRNA 426 | 426 | AGAUGAGUGAGCAGGAAGA | 1566 | UCUUCCUGCUCACUCAUCU |
| siRNA 427 | 427 | GAUGAGUGAGCAGGAAGAG | 1567 | CUCUUCCUGCUCACUCAUC |
| siRNA 428 | 428 | AUGAGUGAGCAGGAAGAGG | 1568 | CCUCUUCCUGCUCACUCAU |
| siRNA 429 | 429 | UGAGUGAGCAGGAAGAGGA | 1569 | UCCUCUUCCUGCUCACUCA |
| siRNA 430 | 430 | GAGUGAGCAGGAAGAGGAG | 1570 | CUCCUCUUCCUGCUCACUC |
| siRNA 431 | 431 | AGUGAGCAGGAAGAGGAGC | 1571 | GCUCCUCUUCCUGCUCACU |
| siRNA 432 | 432 | GUGAGCAGGAAGAGGAGCU | 1572 | AGCUCCUCUUCCUGCUCAC |
| siRNA 433 | 433 | UGAGCAGGAAGAGGAGCUU | 1573 | AAGCUCCUCUUCCUGCUCA |
| siRNA 434 | 434 | GAGCAGGAAGAGGAGCUUG | 1574 | CAAGCUCCUCUUCCUGCUC |
| siRNA 435 | 435 | AGCAGGAAGAGGAGCUUGA | 1575 | UCAAGCUCCUCUUCCUGCU |
| siRNA 436 | 436 | GCAGGAAGAGGAGCUUGAG | 1576 | CUCAAGCUCCUCUUCCUGC |
| siRNA 437 | 437 | CAGGAAGAGGAGCUUGAGG | 1577 | CCUCAAGCUCCUCUUCCUG |
| siRNA 438 | 438 | AGGAAGAGGAGCUUGAGGA | 1578 | UCCUCAAGCUCCUCUUCCU |
| siRNA 439 | 439 | GGAAGAGGAGCUUGAGGAU | 1579 | AUCCUCAAGCUCCUCUUCC |
| siRNA 440 | 440 | GAAGAGGAGCUUGAGGAUG | 1580 | CAUCCUCAAGCUCCUCUUC |
| siRNA 441 | 441 | AAGAGGAGCUUGAGGAUGA | 1581 | UCAUCCUCAAGCUCCUCUU |
| siRNA 442 | 442 | AGAGGAGCUUGAGGAUGAU | 1582 | AUCAUCCUCAAGCUCCUCU |
| siRNA 443 | 443 | GAGGAGCUUGAGGAUGAUC | 1583 | GAUCAUCCUCAAGCUCCUC |
| siRNA 444 | 444 | AGGAGCUUGAGGAUGAUCC | 1584 | GGAUCAUCCUCAAGCUCCU |
| siRNA 445 | 445 | GGAGCUUGAGGAUGAUCCU | 1585 | AGGAUCAUCCUCAAGCUCC |
| siRNA 446 | 446 | GAGCUUGAGGAUGAUCCUA | 1586 | UAGGAUCAUCCUCAAGCUC |
| siRNA 447 | 447 | AGCUUGAGGAUGAUCCUAC | 1587 | GUAGGAUCAUCCUCAAGCU |
| siRNA 448 | 448 | GCUUGAGGAUGAUCCUACU | 1588 | AGUAGGAUCAUCCUCAAGC |
| siRNA 449 | 449 | CUUGAGGAUGAUCCUACUG | 1589 | CAGUAGGAUCAUCCUCAAG |
| siRNA 450 | 450 | UUGAGGAUGAUCCUACUGU | 1590 | ACAGUAGGAUCAUCCUCAA |
| siRNA 451 | 451 | UGAGGAUGAUCCUACUGUA | 1591 | UACAGUAGGAUCAUCCUCA |
| siRNA 452 | 452 | GAGGAUGAUCCUACUGUAG | 1592 | CUACAGUAGGAUCAUCCUC |
| siRNA 453 | 453 | AGGAUGAUCCUACUGUAGU | 1593 | ACUACAGUAGGAUCAUCCU |
| siRNA 454 | 454 | GGAUGAUCCUACUGUAGUC | 1594 | GACUACAGUAGGAUCAUCC |
| siRNA 455 | 455 | GAUGAUCCUACUGUAGUCA | 1595 | UGACUACAGUAGGAUCAUC |
| siRNA 456 | 456 | AUGAUCCUACUGUAGUCAA | 1596 | UUGACUACAGUAGGAUCAU |
| siRNA 457 | 457 | UGAUCCUACUGUAGUCAAA | 1597 | UUUGACUACAGUAGGAUCA |
| siRNA 458 | 458 | GAUCCUACUGUAGUCAAAA | 1598 | UUUUGACUACAGUAGGAUC |
| siRNA 459 | 459 | AUCCUACUGUAGUCAAAAA | 1599 | UUUUUGACUACAGUAGGAU |
| siRNA 460 | 460 | UCCUACUGUAGUCAAAAAC | 1600 | GUUUUUGACUACAGUAGGA |
| siRNA 461 | 461 | CCUACUGUAGUCAAAAACU | 1601 | AGUUUUUGACUACAGUAGG |
| siRNA 462 | 462 | CUACUGUAGUCAAAAACUA | 1602 | UAGUUUUUGACUACAGUAG |
| siRNA 463 | 463 | UACUGUAGUCAAAAACUAU | 1603 | AUAGUUUUUGACUACAGUA |
| siRNA 464 | 464 | ACUGUAGUCAAAAACUAUA | 1604 | UAUAGUUUUUGACUACAGU |
| siRNA 465 | 465 | CUGUAGUCAAAAACUAUAA | 1605 | UUAUAGUUUUUGACUACAG |
| siRNA 466 | 466 | UGUAGUCAAAAACUAUAAA | 1606 | UUUAUAGUUUUUGACUACA |
| siRNA 467 | 467 | GUAGUCAAAAACUAUAAAG | 1607 | CUUUAUAGUUUUUGACUAC |
| siRNA 468 | 468 | UAGUCAAAAACUAUAAAGA | 1608 | UCUUUAUAGUUUUUGACUA |
| siRNA 469 | 469 | AGUCAAAAACUAUAAAGAC | 1609 | GUCUUUAUAGUUUUUGACU |
| siRNA 470 | 470 | GUCAAAAACUAUAAAGACC | 1610 | GGUCUUUAUAGUUUUUGAC |
| siRNA 471 | 471 | UCAAAAACUAUAAAGACCU | 1611 | AGGUCUUUAUAGUUUUUGA |
| siRNA 472 | 472 | CAAAAACUAUAAAGACCUG | 1612 | CAGGUCUUUAUAGUUUUUG |
| siRNA 473 | 473 | AAAAACUAUAAAGACCUGG | 1613 | CCAGGUCUUUAUAGUUUUU |
| siRNA 474 | 474 | AAAACUAUAAAGACCUGGA | 1614 | UCCAGGUCUUUAUAGUUUU |
| siRNA 475 | 475 | AAACUAUAAAGACCUGGAA | 1615 | UUCCAGGUCUUUAUAGUUU |
| siRNA 476 | 476 | AACUAUAAAGACCUGGAAA | 1616 | UUUCCAGGUCUUUAUAGUU |
| siRNA 477 | 477 | ACUAUAAAGACCUGGAAAA | 1617 | UUUUCCAGGUCUUUAUAGU |
| siRNA 478 | 478 | CUAUAAAGACCUGGAAAAA | 1618 | UUUUUCCAGGUCUUUAUAG |
| siRNA 479 | 479 | UAUAAAGACCUGGAAAAAG | 1619 | CUUUUUCCAGGUCUUUAUA |
| siRNA 480 | 480 | AUAAAGACCUGGAAAAAGC | 1620 | GCUUUUUCCAGGUCUUUAU |
| siRNA 481 | 481 | UAAAGACCUGGAAAAAGCA | 1621 | UGCUUUUUCCAGGUCUUUA |
| siRNA 482 | 482 | AAAGACCUGGAAAAAGCAG | 1622 | CUGCUUUUUCCAGGUCUUU |
| siRNA 483 | 483 | AAGACCUGGAAAAAGCAGU | 1623 | ACUGCUUUUUCCAGGUCUU |
| siRNA 484 | 484 | AGACCUGGAAAAAGCAGUU | 1624 | AACUGCUUUUUCCAGGUCU |
| siRNA 485 | 485 | GACCUGGAAAAAGCAGUUC | 1625 | GAACUGCUUUUUCCAGGUC |
| siRNA 486 | 486 | ACCUGGAAAAAGCAGUUCA | 1626 | UGAACUGCUUUUUCCAGGU |
| siRNA 487 | 487 | CCUGGAAAAAGCAGUUCAG | 1627 | CUGAACUGCUUUUUCCAGG |
| siRNA 488 | 488 | CUGGAAAAAGCAGUUCAGU | 1628 | ACUGAACUGCUUUUUCCAG |
| siRNA 489 | 489 | UGGAAAAAGCAGUUCAGUC | 1629 | GACUGAACUGCUUUUUCCA |
| siRNA 490 | 490 | GGAAAAAGCAGUUCAGUCU | 1630 | AGACUGAACUGCUUUUUCC |
| siRNA 491 | 491 | GAAAAAGCAGUUCAGUCUU | 1631 | AAGACUGAACUGCUUUUUC |
| siRNA 492 | 492 | AAAAAGCAGUUCAGUCUUU | 1632 | AAAGACUGAACUGCUUUUU |
| siRNA 493 | 493 | AAAAGCAGUUCAGUCUUUU | 1633 | AAAAGACUGAACUGCUUUU |
| siRNA 494 | 494 | AAAGCAGUUCAGUCUUUUC | 1634 | GAAAAGACUGAACUGCUUU |
| siRNA 495 | 495 | AAGCAGUUCAGUCUUUUCG | 1635 | CGAAAAGACUGAACUGCUU |
| siRNA 496 | 496 | AGCAGUUCAGUCUUUUCGG | 1636 | CCGAAAAGACUGAACUGCU |
| siRNA 497 | 497 | GCAGUUCAGUCUUUUCGGU | 1637 | ACCGAAAAGACUGAACUGC |
| siRNA 498 | 498 | CAGUUCAGUCUUUUCGGUA | 1638 | UACCGAAAAGACUGAACUG |
| siRNA 499 | 499 | AGUUCAGUCUUUUCGGUAU | 1639 | AUACCGAAAAGACUGAACU |
| SIRNA 500 | 500 | GUUCAGUCUUUUCGGUAUG | 1640 | CAUACCGAAAAGACUGAAC |
| siRNA 501 | 501 | UUCAGUCUUUUCGGUAUGA | 1641 | UCAUACCGAAAAGACUGAA |
| siRNA 502 | 502 | UCAGUCUUUUCGGUAUGAU | 1642 | AUCAUACCGAAAAGACUGA |
| siRNA 503 | 503 | CAGUCUUUUCGGUAUGAUG | 1643 | CAUCAUACCGAAAAGACUG |
| siRNA 504 | 504 | AGUCUUUUCGGUAUGAUGU | 1644 | ACAUCAUACCGAAAAGACU |
| siRNA 505 | 505 | GUCUUUUCGGUAUGAUGUU | 1645 | AACAUCAUACCGAAAAGAC |
| siRNA 506 | 506 | UCUUUUCGGUAUGAUGUUG | 1646 | CAACAUCAUACCGAAAAGA |
| siRNA 507 | 507 | CUUUUCGGUAUGAUGUUGU | 1647 | ACAACAUCAUACCGAAAAG |
| siRNA 508 | 508 | UUUUCGGUAUGAUGUUGUC | 1648 | GACAACAUCAUACCGAAAA |
| siRNA 509 | 509 | UUUCGGUAUGAUGUUGUCC | 1649 | GGACAACAUCAUACCGAAA |
| siRNA 510 | 510 | UUCGGUAUGAUGUUGUCCU | 1650 | AGGACAACAUCAUACCGAA |
| siRNA 511 | 511 | UCGGUAUGAUGUUGUCCUG | 1651 | CAGGACAACAUCAUACCGA |
| siRNA 512 | 512 | CGGUAUGAUGUUGUCCUGA | 1652 | UCAGGACAACAUCAUACCG |
| siRNA 513 | 513 | GGUAUGAUGUUGUCCUGAA | 1653 | UUCAGGACAACAUCAUACC |
| siRNA 514 | 514 | GUAUGAUGUUGUCCUGAAG | 1654 | CUUCAGGACAACAUCAUAC |
| siRNA 515 | 515 | UAUGAUGUUGUCCUGAAGA | 1655 | UCUUCAGGACAACAUCAUA |
| siRNA 516 | 516 | AUGAUGUUGUCCUGAAGAC | 1656 | GUCUUCAGGACAACAUCAU |
| siRNA 517 | 517 | UGAUGUUGUCCUGAAGACG | 1657 | CGUCUUCAGGACAACAUCA |
| siRNA 518 | 518 | GAUGUUGUCCUGAAGACGG | 1658 | CCGUCUUCAGGACAACAUC |
| siRNA 519 | 519 | AUGUUGUCCUGAAGACGGG | 1659 | CCCGUCUUCAGGACAACAU |
| siRNA 520 | 520 | UGUUGUCCUGAAGACGGGG | 1660 | CCCCGUCUUCAGGACAACA |
| siRNA 521 | 521 | GUUGUCCUGAAGACGGGGC | 1661 | GCCCCGUCUUCAGGACAAC |
| siRNA 522 | 522 | UUGUCCUGAAGACGGGGCU | 1662 | AGCCCCGUCUUCAGGACAA |
| siRNA 523 | 523 | UGUCCUGAAGACGGGGCUA | 1663 | UAGCCCCGUCUUCAGGACA |
| siRNA 524 | 524 | GUCCUGAAGACGGGGCUAG | 1664 | CUAGCCCCGUCUUCAGGAC |
| siRNA 525 | 525 | UCCUGAAGACGGGGCUAGA | 1665 | UCUAGCCCCGUCUUCAGGA |
| siRNA 526 | 526 | CCUGAAGACGGGGCUAGAU | 1666 | AUCUAGCCCCGUCUUCAGG |
| siRNA 527 | 527 | CUGAAGACGGGGCUAGAUA | 1667 | UAUCUAGCCCCGUCUUCAG |
| siRNA 528 | 528 | UGAAGACGGGGCUAGAUAU | 1668 | AUAUCUAGCCCCGUCUUCA |
| siRNA 529 | 529 | GAAGACGGGGCUAGAUAUU | 1669 | AAUAUCUAGCCCCGUCUUC |
| siRNA 530 | 530 | AAGACGGGGCUAGAUAUUG | 1670 | CAAUAUCUAGCCCCGUCUU |
| siRNA 531 | 531 | AGACGGGGCUAGAUAUUGG | 1671 | CCAAUAUCUAGCCCCGUCU |
| siRNA 532 | 532 | GACGGGGCUAGAUAUUGGG | 1672 | CCCAAUAUCUAGCCCCGUC |
| siRNA 533 | 533 | ACGGGGCUAGAUAUUGGGA | 1673 | UCCCAAUAUCUAGCCCCGU |
| siRNA 534 | 534 | CGGGGCUAGAUAUUGGGAG | 1674 | CUCCCAAUAUCUAGCCCCG |
| siRNA 535 | 535 | GGGGCUAGAUAUUGGGAGA | 1675 | UCUCCCAAUAUCUAGCCCC |
| siRNA 536 | 536 | GGGCUAGAUAUUGGGAGAA | 1676 | UUCUCCCAAUAUCUAGCCC |
| siRNA 537 | 537 | GGCUAGAUAUUGGGAGAAA | 1677 | UUUCUCCCAAUAUCUAGCC |
| siRNA 538 | 538 | GCUAGAUAUUGGGAGAAAC | 1678 | GUUUCUCCCAAUAUCUAGC |
| siRNA 539 | 539 | CUAGAUAUUGGGAGAAACA | 1679 | UGUUUCUCCCAAUAUCUAG |
| siRNA 540 | 540 | UAGAUAUUGGGAGAAACAA | 1680 | UUGUUUCUCCCAAUAUCUA |
| siRNA 541 | 541 | AGAUAUUGGGAGAAACAAA | 1681 | UUUGUUUCUCCCAAUAUCU |
| siRNA 542 | 542 | GAUAUUGGGAGAAACAAAG | 1682 | CUUUGUUUCUCCCAAUAUC |
| siRNA 543 | 543 | AUAUUGGGAGAAACAAAGU | 1683 | ACUUUGUUUCUCCCAAUAU |
| siRNA 544 | 544 | UAUUGGGAGAAACAAAGUG | 1684 | CACUUUGUUUCUCCCAAUA |
| siRNA 545 | 545 | AUUGGGAGAAACAAAGUGG | 1685 | CCACUUUGUUUCUCCCAAU |
| siRNA 546 | 546 | UUGGGAGAAACAAAGUGGA | 1686 | UCCACUUUGUUUCUCCCAA |
| siRNA 547 | 547 | UGGGAGAAACAAAGUGGAA | 1687 | UUCCACUUUGUUUCUCCCA |
| siRNA 548 | 548 | GGGAGAAACAAAGUGGAAG | 1688 | CUUCCACUUUGUUUCUCCC |
| siRNA 549 | 549 | GGAGAAACAAAGUGGAAGA | 1689 | UCUUCCACUUUGUUUCUCC |
| siRNA 550 | 550 | GAGAAACAAAGUGGAAGAU | 1690 | AUCUUCCACUUUGUUUCUC |
| siRNA 551 | 551 | AGAAACAAAGUGGAAGAUG | 1691 | CAUCUUCCACUUUGUUUCU |
| siRNA 552 | 552 | GAAACAAAGUGGAAGAUGC | 1692 | GCAUCUUCCACUUUGUUUC |
| siRNA 553 | 553 | AAACAAAGUGGAAGAUGCU | 1693 | AGCAUCUUCCACUUUGUUU |
| siRNA 554 | 554 | AACAAAGUGGAAGAUGCUU | 1694 | AAGCAUCUUCCACUUUGUU |
| siRNA 555 | 555 | ACAAAGUGGAAGAUGCUUU | 1695 | AAAGCAUCUUCCACUUUGU |
| siRNA 556 | 556 | CAAAGUGGAAGAUGCUUUC | 1696 | GAAAGCAUCUUCCACUUUG |
| siRNA 557 | 557 | AAAGUGGAAGAUGCUUUCU | 1697 | AGAAAGCAUCUUCCACUUU |
| siRNA 558 | 558 | AAGUGGAAGAUGCUUUCUA | 1698 | UAGAAAGCAUCUUCCACUU |
| siRNA 559 | 559 | AGUGGAAGAUGCUUUCUAC | 1699 | GUAGAAAGCAUCUUCCACU |
| siRNA 560 | 560 | GUGGAAGAUGCUUUCUACA | 1700 | UGUAGAAAGCAUCUUCCAC |
| siRNA 561 | 561 | UGGAAGAUGCUUUCUACAA | 1701 | UUGUAGAAAGCAUCUUCCA |
| siRNA 562 | 562 | GGAAGAUGCUUUCUACAAA | 1702 | UUUGUAGAAAGCAUCUUCC |
| siRNA 563 | 563 | GAAGAUGCUUUCUACAAAG | 1703 | CUUUGUAGAAAGCAUCUUC |
| siRNA 564 | 564 | AAGAUGCUUUCUACAAAGG | 1704 | CCUUUGUAGAAAGCAUCUU |
| siRNA 565 | 565 | AGAUGCUUUCUACAAAGGU | 1705 | ACCUUUGUAGAAAGCAUCU |
| siRNA 566 | 566 | GAUGCUUUCUACAAAGGUG | 1706 | CACCUUUGUAGAAAGCAUC |
| siRNA 567 | 567 | AUGCUUUCUACAAAGGUGA | 1707 | UCACCUUUGUAGAAAGCAU |
| siRNA 568 | 568 | UGCUUUCUACAAAGGUGAA | 1708 | UUCACCUUUGUAGAAAGCA |
| siRNA 569 | 569 | GCUUUCUACAAAGGUGAAC | 1709 | GUUCACCUUUGUAGAAAGC |
| siRNA 570 | 570 | CUUUCUACAAAGGUGAACU | 1710 | AGUUCACCUUUGUAGAAAG |
| siRNA 571 | 571 | UUUCUACAAAGGUGAACUC | 1711 | GAGUUCACCUUUGUAGAAA |
| siRNA 572 | 572 | UUCUACAAAGGUGAACUCA | 1712 | UGAGUUCACCUUUGUAGAA |
| siRNA 573 | 573 | UCUACAAAGGUGAACUCAG | 1713 | CUGAGUUCACCUUUGUAGA |
| siRNA 574 | 574 | CUACAAAGGUGAACUCAGG | 1714 | CCUGAGUUCACCUUUGUAG |
| siRNA 575 | 575 | UACAAAGGUGAACUCAGGC | 1715 | GCCUGAGUUCACCUUUGUA |
| siRNA 576 | 576 | ACAAAGGUGAACUCAGGCU | 1716 | AGCCUGAGUUCACCUUUGU |
| siRNA 577 | 577 | CAAAGGUGAACUCAGGCUG | 1717 | CAGCCUGAGUUCACCUUUG |
| siRNA 578 | 578 | AAAGGUGAACUCAGGCUGA | 1718 | UCAGCCUGAGUUCACCUUU |
| siRNA 579 | 579 | AAGGUGAACUCAGGCUGAA | 1719 | UUCAGCCUGAGUUCACCUU |
| siRNA 580 | 580 | AGGUGAACUCAGGCUGAAU | 1720 | AUUCAGCCUGAGUUCACCU |
| siRNA 581 | 581 | GGUGAACUCAGGCUGAAUG | 1721 | CAUUCAGCCUGAGUUCACC |
| siRNA 582 | 582 | GUGAACUCAGGCUGAAUGA | 1722 | UCAUUCAGCCUGAGUUCAC |
| siRNA 583 | 583 | UGAACUCAGGCUGAAUGAG | 1723 | CUCAUUCAGCCUGAGUUCA |
| siRNA 584 | 584 | GAACUCAGGCUGAAUGAGG | 1724 | CCUCAUUCAGCCUGAGUUC |
| siRNA 585 | 585 | AACUCAGGCUGAAUGAGGA | 1725 | UCCUCAUUCAGCCUGAGUU |
| siRNA 586 | 586 | ACUCAGGCUGAAUGAGGAA | 1726 | UUCCUCAUUCAGCCUGAGU |
| siRNA 587 | 587 | CUCAGGCUGAAUGAGGAAA | 1727 | UUUCCUCAUUCAGCCUGAG |
| siRNA 588 | 588 | UCAGGCUGAAUGAGGAAAA | 1728 | UUUUCCUCAUUCAGCCUGA |
| siRNA 589 | 589 | CAGGCUGAAUGAGGAAAAA | 1729 | UUUUUCCUCAUUCAGCCUG |
| siRNA 590 | 590 | AGGCUGAAUGAGGAAAAAU | 1730 | AUUUUUCCUCAUUCAGCCU |
| siRNA 591 | 591 | GGCUGAAUGAGGAAAAAUU | 1731 | AAUUUUUCCUCAUUCAGCC |
| siRNA 592 | 592 | GCUGAAUGAGGAAAAAUUA | 1732 | UAAUUUUUCCUCAUUCAGC |
| siRNA 593 | 593 | CUGAAUGAGGAAAAAUUAU | 1733 | AUAAUUUUUCCUCAUUCAG |
| siRNA 594 | 594 | UGAAUGAGGAAAAAUUAUG | 1734 | CAUAAUUUUUCCUCAUUCA |
| siRNA 595 | 595 | GAAUGAGGAAAAAUUAUGG | 1735 | CCAUAAUUUUUCCUCAUUC |
| siRNA 596 | 596 | AAUGAGGAAAAAUUAUGGA | 1736 | UCCAUAAUUUUUCCUCAUU |
| siRNA 597 | 597 | AUGAGGAAAAAUUAUGGAA | 1737 | UUCCAUAAUUUUUCCUCAU |
| siRNA 598 | 598 | UGAGGAAAAAUUAUGGAAG | 1738 | CUUCCAUAAUUUUUCCUCA |
| siRNA 599 | 599 | GAGGAAAAAUUAUGGAAGA | 1739 | UCUUCCAUAAUUUUUCCUC |
| siRNA 600 | 600 | AGGAAAAAUUAUGGAAGAA | 1740 | UUCUUCCAUAAUUUUUCCU |
| siRNA 601 | 601 | GGAAAAAUUAUGGAAGAAA | 1741 | UUUCUUCCAUAAUUUUUCC |
| siRNA 602 | 602 | GAAAAAUUAUGGAAGAAAA | 1742 | UUUUCUUCCAUAAUUUUUC |
| siRNA 603 | 603 | AAAAAUUAUGGAAGAAAAG | 1743 | CUUUUCUUCCAUAAUUUUU |
| siRNA 604 | 604 | AAAAUUAUGGAAGAAAAGC | 1744 | GCUUUUCUUCCAUAAUUUU |
| siRNA 605 | 605 | AAAUUAUGGAAGAAAAGCA | 1745 | UGCUUUUCUUCCAUAAUUU |
| siRNA 606 | 606 | AAUUAUGGAAGAAAAGCAG | 1746 | CUGCUUUUCUUCCAUAAUU |
| siRNA 607 | 607 | AUUAUGGAAGAAAAGCAGA | 1747 | UCUGCUUUUCUUCCAUAAU |
| siRNA 608 | 608 | UUAUGGAAGAAAAGCAGAA | 1748 | UUCUGCUUUUCUUCCAUAA |
| siRNA 609 | 609 | UAUGGAAGAAAAGCAGAAC | 1749 | GUUCUGCUUUUCUUCCAUA |
| SIRNA 610 | 610 | AUGGAAGAAAAGCAGAACG | 1750 | CGUUCUGCUUUUCUUCCAU |
| siRNA 611 | 611 | UGGAAGAAAAGCAGAACGG | 1751 | CCGUUCUGCUUUUCUUCCA |
| siRNA 612 | 612 | GGAAGAAAAGCAGAACGGU | 1752 | ACCGUUCUGCUUUUCUUCC |
| siRNA 613 | 613 | GAAGAAAAGCAGAACGGUG | 1753 | CACCGUUCUGCUUUUCUUC |
| siRNA 614 | 614 | AAGAAAAGCAGAACGGUGA | 1754 | UCACCGUUCUGCUUUUCUU |
| siRNA 615 | 615 | AGAAAAGCAGAACGGUGAA | 1755 | UUCACCGUUCUGCUUUUCU |
| siRNA 616 | 616 | GAAAAGCAGAACGGUGAAA | 1756 | UUUCACCGUUCUGCUUUUC |
| siRNA 617 | 617 | AAAAGCAGAACGGUGAAAG | 1757 | CUUUCACCGUUCUGCUUUU |
| siRNA 618 | 618 | AAAGCAGAACGGUGAAAGU | 1758 | ACUUUCACCGUUCUGCUUU |
| siRNA 619 | 619 | AAGCAGAACGGUGAAAGUG | 1759 | CACUUUCACCGUUCUGCUU |
| siRNA 620 | 620 | AGCAGAACGGUGAAAGUGG | 1760 | CCACUUUCACCGUUCUGCU |
| siRNA 621 | 621 | GCAGAACGGUGAAAGUGGG | 1761 | CCCACUUUCACCGUUCUGC |
| siRNA 622 | 622 | CAGAACGGUGAAAGUGGGA | 1762 | UCCCACUUUCACCGUUCUG |
| siRNA 623 | 623 | AGAACGGUGAAAGUGGGAG | 1763 | CUCCCACUUUCACCGUUCU |
| siRNA 624 | 624 | GAACGGUGAAAGUGGGAGA | 1764 | UCUCCCACUUUCACCGUUC |
| siRNA 625 | 625 | AACGGUGAAAGUGGGAGAU | 1765 | AUCUCCCACUUUCACCGUU |
| siRNA 626 | 626 | ACGGUGAAAGUGGGAGAUA | 1766 | UAUCUCCCACUUUCACCGU |
| siRNA 627 | 627 | CGGUGAAAGUGGGAGAUAC | 1767 | GUAUCUCCCACUUUCACCG |
| siRNA 628 | 628 | GGUGAAAGUGGGAGAUACA | 1768 | UGUAUCUCCCACUUUCACC |
| siRNA 629 | 629 | GUGAAAGUGGGAGAUACAU | 1769 | AUGUAUCUCCCACUUUCAC |
| siRNA 630 | 630 | UGAAAGUGGGAGAUACAUU | 1770 | AAUGUAUCUCCCACUUUCA |
| siRNA 631 | 631 | GAAAGUGGGAGAUACAUUG | 1771 | CAAUGUAUCUCCCACUUUC |
| siRNA 632 | 632 | AAAGUGGGAGAUACAUUGG | 1772 | CCAAUGUAUCUCCCACUUU |
| siRNA 633 | 633 | AAGUGGGAGAUACAUUGGA | 1773 | UCCAAUGUAUCUCCCACUU |
| siRNA 634 | 634 | AGUGGGAGAUACAUUGGAU | 1774 | AUCCAAUGUAUCUCCCACU |
| siRNA 635 | 635 | GUGGGAGAUACAUUGGAUC | 1775 | GAUCCAAUGUAUCUCCCAC |
| siRNA 636 | 636 | UGGGAGAUACAUUGGAUCU | 1776 | AGAUCCAAUGUAUCUCCCA |
| siRNA 637 | 637 | GGGAGAUACAUUGGAUCUU | 1777 | AAGAUCCAAUGUAUCUCCC |
| siRNA 638 | 638 | GGAGAUACAUUGGAUCUUC | 1778 | GAAGAUCCAAUGUAUCUCC |
| siRNA 639 | 639 | GAGAUACAUUGGAUCUUCU | 1779 | AGAAGAUCCAAUGUAUCUC |
| siRNA 640 | 640 | AGAUACAUUGGAUCUUCUC | 1780 | GAGAAGAUCCAAUGUAUCU |
| siRNA 641 | 641 | GAUACAUUGGAUCUUCUCA | 1781 | UGAGAAGAUCCAAUGUAUC |
| siRNA 642 | 642 | AUACAUUGGAUCUUCUCAU | 1782 | AUGAGAAGAUCCAAUGUAU |
| siRNA 643 | 643 | UACAUUGGAUCUUCUCAUU | 1783 | AAUGAGAAGAUCCAAUGUA |
| siRNA 644 | 644 | ACAUUGGAUCUUCUCAUUG | 1784 | CAAUGAGAAGAUCCAAUGU |
| siRNA 645 | 645 | CAUUGGAUCUUCUCAUUGG | 1785 | CCAAUGAGAAGAUCCAAUG |
| siRNA 646 | 646 | AUUGGAUCUUCUCAUUGGA | 1786 | UCCAAUGAGAAGAUCCAAU |
| siRNA 647 | 647 | UUGGAUCUUCUCAUUGGAG | 1787 | CUCCAAUGAGAAGAUCCAA |
| siRNA 648 | 648 | UGGAUCUUCUCAUUGGAGA | 1788 | UCUCCAAUGAGAAGAUCCA |
| siRNA 649 | 649 | GGAUCUUCUCAUUGGAGAG | 1789 | CUCUCCAAUGAGAAGAUCC |
| siRNA 650 | 650 | GAUCUUCUCAUUGGAGAGG | 1790 | CCUCUCCAAUGAGAAGAUC |
| siRNA 651 | 651 | AUCUUCUCAUUGGAGAGGA | 1791 | UCCUCUCCAAUGAGAAGAU |
| siRNA 652 | 652 | UCUUCUCAUUGGAGAGGAU | 1792 | AUCCUCUCCAAUGAGAAGA |
| siRNA 653 | 653 | CUUCUCAUUGGAGAGGAUA | 1793 | UAUCCUCUCCAAUGAGAAG |
| siRNA 654 | 654 | UUCUCAUUGGAGAGGAUAA | 1794 | UUAUCCUCUCCAAUGAGAA |
| siRNA 655 | 655 | UCUCAUUGGAGAGGAUAAA | 1795 | UUUAUCCUCUCCAAUGAGA |
| siRNA 656 | 656 | CUCAUUGGAGAGGAUAAAG | 1796 | CUUUAUCCUCUCCAAUGAG |
| siRNA 657 | 657 | UCAUUGGAGAGGAUAAAGA | 1797 | UCUUUAUCCUCUCCAAUGA |
| siRNA 658 | 658 | CAUUGGAGAGGAUAAAGAA | 1798 | UUCUUUAUCCUCUCCAAUG |
| siRNA 659 | 659 | AUUGGAGAGGAUAAAGAAG | 1799 | CUUCUUUAUCCUCUCCAAU |
| siRNA 660 | 660 | UUGGAGAGGAUAAAGAAGC | 1800 | GCUUCUUUAUCCUCUCCAA |
| siRNA 661 | 661 | UGGAGAGGAUAAAGAAGCA | 1801 | UGCUUCUUUAUCCUCUCCA |
| siRNA 662 | 662 | GGAGAGGAUAAAGAAGCAG | 1802 | CUGCUUCUUUAUCCUCUCC |
| siRNA 663 | 663 | GAGAGGAUAAAGAAGCAGG | 1803 | CCUGCUUCUUUAUCCUCUC |
| siRNA 664 | 664 | AGAGGAUAAAGAAGCAGGA | 1804 | UCCUGCUUCUUUAUCCUCU |
| siRNA 665 | 665 | GAGGAUAAAGAAGCAGGAA | 1805 | UUCCUGCUUCUUUAUCCUC |
| siRNA 666 | 666 | AGGAUAAAGAAGCAGGAAC | 1806 | GUUCCUGCUUCUUUAUCCU |
| siRNA 667 | 667 | GGAUAAAGAAGCAGGAACA | 1807 | UGUUCCUGCUUCUUUAUCC |
| siRNA 668 | 668 | GAUAAAGAAGCAGGAACAG | 1808 | CUGUUCCUGCUUCUUUAUC |
| siRNA 669 | 669 | AUAAAGAAGCAGGAACAGA | 1809 | UCUGUUCCUGCUUCUUUAU |
| siRNA 670 | 670 | UAAAGAAGCAGGAACAGAG | 1810 | CUCUGUUCCUGCUUCUUUA |
| siRNA 671 | 671 | AAAGAAGCAGGAACAGAGA | 1811 | UCUCUGUUCCUGCUUCUUU |
| siRNA 672 | 672 | AAGAAGCAGGAACAGAGAC | 1812 | GUCUCUGUUCCUGCUUCUU |
| siRNA 673 | 673 | AGAAGCAGGAACAGAGACA | 1813 | UGUCUCUGUUCCUGCUUCU |
| siRNA 674 | 674 | GAAGCAGGAACAGAGACAG | 1814 | CUGUCUCUGUUCCUGCUUC |
| siRNA 675 | 675 | AAGCAGGAACAGAGACAGU | 1815 | ACUGUCUCUGUUCCUGCUU |
| siRNA 676 | 676 | AGCAGGAACAGAGACAGUU | 1816 | AACUGUCUCUGUUCCUGCU |
| siRNA 677 | 677 | GCAGGAACAGAGACAGUUA | 1817 | UAACUGUCUCUGUUCCUGC |
| siRNA 678 | 678 | CAGGAACAGAGACAGUUAU | 1818 | AUAACUGUCUCUGUUCCUG |
| siRNA 679 | 679 | AGGAACAGAGACAGUUAUG | 1819 | CAUAACUGUCUCUGUUCCU |
| siRNA 680 | 680 | GGAACAGAGACAGUUAUGC | 1820 | GCAUAACUGUCUCUGUUCC |
| siRNA 681 | 681 | GAACAGAGACAGUUAUGCG | 1821 | CGCAUAACUGUCUCUGUUC |
| siRNA 682 | 682 | AACAGAGACAGUUAUGCGG | 1822 | CCGCAUAACUGUCUCUGUU |
| siRNA 683 | 683 | ACAGAGACAGUUAUGCGGA | 1823 | UCCGCAUAACUGUCUCUGU |
| siRNA 684 | 684 | CAGAGACAGUUAUGCGGAU | 1824 | AUCCGCAUAACUGUCUCUG |
| siRNA 685 | 685 | AGAGACAGUUAUGCGGAUU | 1825 | AAUCCGCAUAACUGUCUCU |
| siRNA 686 | 686 | GAGACAGUUAUGCGGAUUC | 1826 | GAAUCCGCAUAACUGUCUC |
| siRNA 687 | 687 | AGACAGUUAUGCGGAUUCU | 1827 | AGAAUCCGCAUAACUGUCU |
| siRNA 688 | 688 | GACAGUUAUGCGGAUUCUC | 1828 | GAGAAUCCGCAUAACUGUC |
| siRNA 689 | 689 | ACAGUUAUGCGGAUUCUCU | 1829 | AGAGAAUCCGCAUAACUGU |
| siRNA 690 | 690 | CAGUUAUGCGGAUUCUCUU | 1830 | AAGAGAAUCCGCAUAACUG |
| siRNA 691 | 691 | AGUUAUGCGGAUUCUCUUG | 1831 | CAAGAGAAUCCGCAUAACU |
| siRNA 692 | 692 | GUUAUGCGGAUUCUCUUGA | 1832 | UCAAGAGAAUCCGCAUAAC |
| siRNA 693 | 693 | UUAUGCGGAUUCUCUUGAA | 1833 | UUCAAGAGAAUCCGCAUAA |
| siRNA 694 | 694 | UAUGCGGAUUCUCUUGAAA | 1834 | UUUCAAGAGAAUCCGCAUA |
| siRNA 695 | 695 | AUGCGGAUUCUCUUGAAAA | 1835 | UUUUCAAGAGAAUCCGCAU |
| siRNA 696 | 696 | UGCGGAUUCUCUUGAAAAA | 1836 | UUUUUCAAGAGAAUCCGCA |
| siRNA 697 | 697 | GCGGAUUCUCUUGAAAAAA | 1837 | UUUUUUCAAGAGAAUCCGC |
| siRNA 698 | 698 | CGGAUUCUCUUGAAAAAAG | 1838 | CUUUUUUCAAGAGAAUCCG |
| siRNA 699 | 699 | GGAUUCUCUUGAAAAAAGU | 1839 | ACUUUUUUCAAGAGAAUCC |
| siRNA 700 | 700 | GAUUCUCUUGAAAAAAGUG | 1840 | CACUUUUUUCAAGAGAAUC |
| siRNA 701 | 701 | AUUCUCUUGAAAAAAGUGU | 1841 | ACACUUUUUUCAAGAGAAU |
| siRNA 702 | 702 | UUCUCUUGAAAAAAGUGUU | 1842 | AACACUUUUUUCAAGAGAA |
| siRNA 703 | 703 | UCUCUUGAAAAAAGUGUUU | 1843 | AAACACUUUUUUCAAGAGA |
| siRNA 704 | 704 | CUCUUGAAAAAAGUGUUUG | 1844 | CAAACACUUUUUUCAAGAG |
| siRNA 705 | 705 | UCUUGAAAAAAGUGUUUGA | 1845 | UCAAACACUUUUUUCAAGA |
| siRNA 706 | 706 | CUUGAAAAAAGUGUUUGAA | 1846 | UUCAAACACUUUUUUCAAG |
| siRNA 707 | 707 | UUGAAAAAAGUGUUUGAAG | 1847 | CUUCAAACACUUUUUUCAA |
| siRNA 708 | 708 | UGAAAAAAGUGUUUGAAGA | 1848 | UCUUCAAACACUUUUUUCA |
| siRNA 709 | 709 | GAAAAAAGUGUUUGAAGAG | 1849 | CUCUUCAAACACUUUUUUC |
| siRNA 710 | 710 | AAAAAAGUGUUUGAAGAGA | 1850 | UCUCUUCAAACACUUUUUU |
| siRNA 711 | 711 | AAAAAGUGUUUGAAGAGAA | 1851 | UUCUCUUCAAACACUUUUU |
| siRNA 712 | 712 | AAAAGUGUUUGAAGAGAAG | 1852 | CUUCUCUUCAAACACUUUU |
| siRNA 713 | 713 | AAAGUGUUUGAAGAGAAGA | 1853 | UCUUCUCUUCAAACACUUU |
| siRNA 714 | 714 | AAGUGUUUGAAGAGAAGAC | 1854 | GUCUUCUCUUCAAACACUU |
| siRNA 715 | 715 | AGUGUUUGAAGAGAAGACU | 1855 | AGUCUUCUCUUCAAACACU |
| siRNA 716 | 716 | GUGUUUGAAGAGAAGACUG | 1856 | CAGUCUUCUCUUCAAACAC |
| siRNA 717 | 717 | UGUUUGAAGAGAAGACUGA | 1857 | UCAGUCUUCUCUUCAAACA |
| siRNA 718 | 718 | GUUUGAAGAGAAGACUGAA | 1858 | UUCAGUCUUCUCUUCAAAC |
| siRNA 719 | 719 | UUUGAAGAGAAGACUGAAA | 1859 | UUUCAGUCUUCUCUUCAAA |
| siRNA 720 | 720 | UUGAAGAGAAGACUGAAAG | 1860 | CUUUCAGUCUUCUCUUCAA |
| siRNA 721 | 721 | UGAAGAGAAGACUGAAAGU | 1861 | ACUUUCAGUCUUCUCUUCA |
| siRNA 722 | 722 | GAAGAGAAGACUGAAAGUG | 1862 | CACUUUCAGUCUUCUCUUC |
| siRNA 723 | 723 | AAGAGAAGACUGAAAGUGA | 1863 | UCACUUUCAGUCUUCUCUU |
| siRNA 724 | 724 | AGAGAAGACUGAAAGUGAA | 1864 | UUCACUUUCAGUCUUCUCU |
| siRNA 725 | 725 | GAGAAGACUGAAAGUGAAA | 1865 | UUUCACUUUCAGUCUUCUC |
| siRNA 726 | 726 | AGAAGACUGAAAGUGAAAA | 1866 | UUUUCACUUUCAGUCUUCU |
| siRNA 727 | 727 | GAAGACUGAAAGUGAAAAA | 1867 | UUUUUCACUUUCAGUCUUC |
| siRNA 728 | 728 | AAGACUGAAAGUGAAAAAU | 1868 | AUUUUUCACUUUCAGUCUU |
| siRNA 729 | 729 | AGACUGAAAGUGAAAAAUA | 1869 | UAUUUUUCACUUUCAGUCU |
| siRNA 730 | 730 | GACUGAAAGUGAAAAAUAC | 1870 | GUAUUUUUCACUUUCAGUC |
| siRNA 731 | 731 | ACUGAAAGUGAAAAAUACA | 1871 | UGUAUUUUUCACUUUCAGU |
| siRNA 732 | 732 | CUGAAAGUGAAAAAUACAG | 1872 | CUGUAUUUUUCACUUUCAG |
| siRNA 733 | 733 | UGAAAGUGAAAAAUACAGA | 1873 | UCUGUAUUUUUCACUUUCA |
| siRNA 734 | 734 | GAAAGUGAAAAAUACAGAG | 1874 | CUCUGUAUUUUUCACUUUC |
| siRNA 735 | 735 | AAAGUGAAAAAUACAGAGU | 1875 | ACUCUGUAUUUUUCACUUU |
| siRNA 736 | 736 | AAGUGAAAAAUACAGAGUG | 1876 | CACUCUGUAUUUUUCACUU |
| siRNA 737 | 737 | AGUGAAAAAUACAGAGUGG | 1877 | CCACUCUGUAUUUUUCACU |
| siRNA 738 | 738 | GUGAAAAAUACAGAGUGGU | 1878 | ACCACUCUGUAUUUUUCAC |
| siRNA 739 | 739 | UGAAAAAUACAGAGUGGUG | 1879 | CACCACUCUGUAUUUUUCA |
| siRNA 740 | 740 | GAAAAAUACAGAGUGGUGU | 1880 | ACACCACUCUGUAUUUUUC |
| siRNA 741 | 741 | AAAAAUACAGAGUGGUGUU | 1881 | AACACCACUCUGUAUUUUU |
| siRNA 742 | 742 | AAAAUACAGAGUGGUGUUA | 1882 | UAACACCACUCUGUAUUUU |
| siRNA 743 | 743 | AAAUACAGAGUGGUGUUAC | 1883 | GUAACACCACUCUGUAUUU |
| siRNA 744 | 744 | AAUACAGAGUGGUGUUACG | 1884 | CGUAACACCACUCUGUAUU |
| siRNA 745 | 745 | AUACAGAGUGGUGUUACGG | 1885 | CCGUAACACCACUCUGUAU |
| siRNA 746 | 746 | UACAGAGUGGUGUUACGGC | 1886 | GCCGUAACACCACUCUGUA |
| siRNA 747 | 747 | ACAGAGUGGUGUUACGGCG | 1887 | CGCCGUAACACCACUCUGU |
| siRNA 748 | 748 | CAGAGUGGUGUUACGGCGG | 1888 | CCGCCGUAACACCACUCUG |
| siRNA 749 | 749 | AGAGUGGUGUUACGGCGGU | 1889 | ACCGCCGUAACACCACUCU |
| siRNA 750 | 750 | GAGUGGUGUUACGGCGGUG | 1890 | CACCGCCGUAACACCACUC |
| siRNA 751 | 751 | AGUGGUGUUACGGCGGUGG | 1891 | CCACCGCCGUAACACCACU |
| siRNA 752 | 752 | GUGGUGUUACGGCGGUGGA | 1892 | UCCACCGCCGUAACACCAC |
| siRNA 753 | 753 | UGGUGUUACGGCGGUGGAA | 1893 | UUCCACCGCCGUAACACCA |
| siRNA 754 | 754 | GGUGUUACGGCGGUGGAAA | 1894 | UUUCCACCGCCGUAACACC |
| siRNA 755 | 755 | GUGUUACGGCGGUGGAAAA | 1895 | UUUUCCACCGCCGUAACAC |
| siRNA 756 | 756 | UGUUACGGCGGUGGAAAAG | 1896 | CUUUUCCACCGCCGUAACA |
| siRNA 757 | 757 | GUUACGGCGGUGGAAAAGU | 1897 | ACUUUUCCACCGCCGUAAC |
| SIRNA 758 | 758 | UUACGGCGGUGGAAAAGUU | 1898 | AACUUUUCCACCGCCGUAA |
| siRNA 759 | 759 | UACGGCGGUGGAAAAGUUU | 1899 | AAACUUUUCCACCGCCGUA |
| siRNA 760 | 760 | ACGGCGGUGGAAAAGUUUA | 1900 | UAAACUUUUCCACCGCCGU |
| siRNA 761 | 761 | CGGCGGUGGAAAAGUUUAA | 1901 | UUAAACUUUUCCACCGCCG |
| siRNA 762 | 762 | GGCGGUGGAAAAGUUUAAA | 1902 | UUUAAACUUUUCCACCGCC |
| siRNA 763 | 763 | GCGGUGGAAAAGUUUAAAG | 1903 | CUUUAAACUUUUCCACCGC |
| siRNA 764 | 764 | CGGUGGAAAAGUUUAAAGU | 1904 | ACUUUAAACUUUUCCACCG |
| siRNA 765 | 765 | GGUGGAAAAGUUUAAAGUU | 1905 | AACUUUAAACUUUUCCACC |
| siRNA 766 | 766 | GUGGAAAAGUUUAAAGUUG | 1906 | CAACUUUAAACUUUUCCAC |
| siRNA 767 | 767 | UGGAAAAGUUUAAAGUUGC | 1907 | GCAACUUUAAACUUUUCCA |
| siRNA 768 | 768 | GGAAAAGUUUAAAGUUGCC | 1908 | GGCAACUUUAAACUUUUCC |
| siRNA 769 | 769 | GAAAAGUUUAAAGUUGCCU | 1909 | AGGCAACUUUAAACUUUUC |
| siRNA 770 | 770 | AAAAGUUUAAAGUUGCCUA | 1910 | UAGGCAACUUUAAACUUUU |
| siRNA 771 | 771 | AAAGUUUAAAGUUGCCUAA | 1911 | UUAGGCAACUUUAAACUUU |
| siRNA 772 | 772 | AAGUUUAAAGUUGCCUAAG | 1912 | CUUAGGCAACUUUAAACUU |
| siRNA 773 | 773 | AGUUUAAAGUUGCCUAAGA | 1913 | UCUUAGGCAACUUUAAACU |
| siRNA 774 | 774 | GUUUAAAGUUGCCUAAGAA | 1914 | UUCUUAGGCAACUUUAAAC |
| siRNA 775 | 775 | UUUAAAGUUGCCUAAGAAG | 1915 | CUUCUUAGGCAACUUUAAA |
| siRNA 776 | 776 | UUAAAGUUGCCUAAGAAGA | 1916 | UCUUCUUAGGCAACUUUAA |
| siRNA 777 | 777 | UAAAGUUGCCUAAGAAGAG | 1917 | CUCUUCUUAGGCAACUUUA |
| siRNA 778 | 778 | AAAGUUGCCUAAGAAGAGA | 1918 | UCUCUUCUUAGGCAACUUU |
| siRNA 779 | 779 | AAGUUGCCUAAGAAGAGAA | 1919 | UUCUCUUCUUAGGCAACUU |
| siRNA 780 | 780 | AGUUGCCUAAGAAGAGAAU | 1920 | AUUCUCUUCUUAGGCAACU |
| siRNA 781 | 781 | GUUGCCUAAGAAGAGAAUG | 1921 | CAUUCUCUUCUUAGGCAAC |
| siRNA 782 | 782 | UUGCCUAAGAAGAGAAUGU | 1922 | ACAUUCUCUUCUUAGGCAA |
| siRNA 783 | 783 | UGCCUAAGAAGAGAAUGUC | 1923 | GACAUUCUCUUCUUAGGCA |
| siRNA 784 | 784 | GCCUAAGAAGAGAAUGUCU | 1924 | AGACAUUCUCUUCUUAGGC |
| siRNA 785 | 785 | CCUAAGAAGAGAAUGUCUA | 1925 | UAGACAUUCUCUUCUUAGG |
| siRNA 786 | 786 | CUAAGAAGAGAAUGUCUAA | 1926 | UUAGACAUUCUCUUCUUAG |
| siRNA 787 | 787 | UAAGAAGAGAAUGUCUAAA | 1927 | UUUAGACAUUCUCUUCUUA |
| siRNA 788 | 788 | AAGAAGAGAAUGUCUAAAU | 1928 | AUUUAGACAUUCUCUUCUU |
| siRNA 789 | 789 | AGAAGAGAAUGUCUAAAUA | 1929 | UAUUUAGACAUUCUCUUCU |
| siRNA 790 | 790 | GAAGAGAAUGUCUAAAUAA | 1930 | UUAUUUAGACAUUCUCUUC |
| siRNA 791 | 791 | AAGAGAAUGUCUAAAUAAA | 1931 | UUUAUUUAGACAUUCUCUU |
| siRNA 792 | 792 | AGAGAAUGUCUAAAUAAAU | 1932 | AUUUAUUUAGACAUUCUCU |
| siRNA 793 | 793 | GAGAAUGUCUAAAUAAAUG | 1933 | CAUUUAUUUAGACAUUCUC |
| siRNA 794 | 794 | AGAAUGUCUAAAUAAAUGG | 1934 | CCAUUUAUUUAGACAUUCU |
| siRNA 795 | 795 | GAAUGUCUAAAUAAAUGGA | 1935 | UCCAUUUAUUUAGACAUUC |
| siRNA 796 | 796 | AAUGUCUAAAUAAAUGGAU | 1936 | AUCCAUUUAUUUAGACAUU |
| siRNA 797 | 797 | AUGUCUAAAUAAAUGGAUU | 1937 | AAUCCAUUUAUUUAGACAU |
| siRNA 798 | 798 | UGUCUAAAUAAAUGGAUUG | 1938 | CAAUCCAUUUAUUUAGACA |
| siRNA 799 | 799 | GUCUAAAUAAAUGGAUUGC | 1939 | GCAAUCCAUUUAUUUAGAC |
| siRNA 800 | 800 | UCUAAAUAAAUGGAUUGCU | 1940 | AGCAAUCCAUUUAUUUAGA |
| siRNA 801 | 801 | CUAAAUAAAUGGAUUGCUU | 1941 | AAGCAAUCCAUUUAUUUAG |
| siRNA 802 | 802 | UAAAUAAAUGGAUUGCUUU | 1942 | AAAGCAAUCCAUUUAUUUA |
| siRNA 803 | 803 | AAAUAAAUGGAUUGCUUUU | 1943 | AAAAGCAAUCCAUUUAUUU |
| siRNA 804 | 804 | AAUAAAUGGAUUGCUUUUU | 1944 | AAAAAGCAAUCCAUUUAUU |
| siRNA 805 | 805 | AUAAAUGGAUUGCUUUUUA | 1945 | UAAAAAGCAAUCCAUUUAU |
| siRNA 806 | 806 | UAAAUGGAUUGCUUUUUAG | 1946 | CUAAAAAGCAAUCCAUUUA |
| siRNA 807 | 807 | AAAUGGAUUGCUUUUUAGC | 1947 | GCUAAAAAGCAAUCCAUUU |
| siRNA 808 | 808 | AAUGGAUUGCUUUUUAGCA | 1948 | UGCUAAAAAGCAAUCCAUU |
| siRNA 809 | 809 | AUGGAUUGCUUUUUAGCAA | 1949 | UUGCUAAAAAGCAAUCCAU |
| siRNA 810 | 810 | UGGAUUGCUUUUUAGCAAU | 1950 | AUUGCUAAAAAGCAAUCCA |
| siRNA 811 | 811 | GGAUUGCUUUUUAGCAAUA | 1951 | UAUUGCUAAAAAGCAAUCC |
| siRNA 812 | 812 | GAUUGCUUUUUAGCAAUAG | 1952 | CUAUUGCUAAAAAGCAAUC |
| siRNA 813 | 813 | AUUGCUUUUUAGCAAUAGA | 1953 | UCUAUUGCUAAAAAGCAAU |
| siRNA 814 | 814 | UUGCUUUUUAGCAAUAGAG | 1954 | CUCUAUUGCUAAAAAGCAA |
| siRNA 815 | 815 | UGCUUUUUAGCAAUAGAGC | 1955 | GCUCUAUUGCUAAAAAGCA |
| siRNA 816 | 816 | GCUUUUUAGCAAUAGAGCU | 1956 | AGCUCUAUUGCUAAAAAGC |
| siRNA 817 | 817 | CUUUUUAGCAAUAGAGCUG | 1957 | CAGCUCUAUUGCUAAAAAG |
| siRNA 818 | 818 | UUUUUAGCAAUAGAGCUGC | 1958 | GCAGCUCUAUUGCUAAAAA |
| siRNA 819 | 819 | UUUUAGCAAUAGAGCUGCU | 1959 | AGCAGCUCUAUUGCUAAAA |
| siRNA 820 | 820 | UUUAGCAAUAGAGCUGCUU | 1960 | AAGCAGCUCUAUUGCUAAA |
| siRNA 821 | 821 | UUAGCAAUAGAGCUGCUUU | 1961 | AAAGCAGCUCUAUUGCUAA |
| siRNA 822 | 822 | UAGCAAUAGAGCUGCUUUC | 1962 | GAAAGCAGCUCUAUUGCUA |
| SIRNA 823 | 823 | AGCAAUAGAGCUGCUUUCU | 1963 | AGAAAGCAGCUCUAUUGCU |
| siRNA 824 | 824 | GCAAUAGAGCUGCUUUCUA | 1964 | UAGAAAGCAGCUCUAUUGC |
| siRNA 825 | 825 | CAAUAGAGCUGCUUUCUAG | 1965 | CUAGAAAGCAGCUCUAUUG |
| siRNA 826 | 826 | AAUAGAGCUGCUUUCUAGU | 1966 | ACUAGAAAGCAGCUCUAUU |
| siRNA 827 | 827 | AUAGAGCUGCUUUCUAGUG | 1967 | CACUAGAAAGCAGCUCUAU |
| siRNA 828 | 828 | UAGAGCUGCUUUCUAGUGG | 1968 | CCACUAGAAAGCAGCUCUA |
| siRNA 829 | 829 | AGAGCUGCUUUCUAGUGGU | 1969 | ACCACUAGAAAGCAGCUCU |
| siRNA 830 | 830 | GAGCUGCUUUCUAGUGGUA | 1970 | UACCACUAGAAAGCAGCUC |
| siRNA 831 | 831 | AGCUGCUUUCUAGUGGUAA | 1971 | UUACCACUAGAAAGCAGCU |
| siRNA 832 | 832 | GCUGCUUUCUAGUGGUAAA | 1972 | UUUACCACUAGAAAGCAGC |
| siRNA 833 | 833 | CUGCUUUCUAGUGGUAAAG | 1973 | CUUUACCACUAGAAAGCAG |
| siRNA 834 | 834 | UGCUUUCUAGUGGUAAAGG | 1974 | CCUUUACCACUAGAAAGCA |
| siRNA 835 | 835 | GCUUUCUAGUGGUAAAGGA | 1975 | UCCUUUACCACUAGAAAGC |
| siRNA 836 | 836 | CUUUCUAGUGGUAAAGGAA | 1976 | UUCCUUUACCACUAGAAAG |
| siRNA 837 | 837 | UUUCUAGUGGUAAAGGAAG | 1977 | CUUCCUUUACCACUAGAAA |
| siRNA 838 | 838 | UUCUAGUGGUAAAGGAAGG | 1978 | CCUUCCUUUACCACUAGAA |
| siRNA 839 | 839 | UCUAGUGGUAAAGGAAGGG | 1979 | CCCUUCCUUUACCACUAGA |
| siRNA 840 | 840 | CUAGUGGUAAAGGAAGGGG | 1980 | CCCCUUCCUUUACCACUAG |
| siRNA 841 | 841 | UAGUGGUAAAGGAAGGGGU | 1981 | ACCCCUUCCUUUACCACUA |
| siRNA 842 | 842 | AGUGGUAAAGGAAGGGGUC | 1982 | GACCCCUUCCUUUACCACU |
| siRNA 843 | 843 | GUGGUAAAGGAAGGGGUCA | 1983 | UGACCCCUUCCUUUACCAC |
| siRNA 844 | 844 | UGGUAAAGGAAGGGGUCAC | 1984 | GUGACCCCUUCCUUUACCA |
| siRNA 845 | 845 | GGUAAAGGAAGGGGUCACC | 1985 | GGUGACCCCUUCCUUUACC |
| siRNA 846 | 846 | GUAAAGGAAGGGGUCACCU | 1986 | AGGUGACCCCUUCCUUUAC |
| siRNA 847 | 847 | UAAAGGAAGGGGUCACCUG | 1987 | CAGGUGACCCCUUCCUUUA |
| siRNA 848 | 848 | AAAGGAAGGGGUCACCUGA | 1988 | UCAGGUGACCCCUUCCUUU |
| siRNA 849 | 849 | AAGGAAGGGGUCACCUGAA | 1989 | UUCAGGUGACCCCUUCCUU |
| siRNA 850 | 850 | AGGAAGGGGUCACCUGAAA | 1990 | UUUCAGGUGACCCCUUCCU |
| siRNA 851 | 851 | GGAAGGGGUCACCUGAAAA | 1991 | UUUUCAGGUGACCCCUUCC |
| siRNA 852 | 852 | GAAGGGGUCACCUGAAAAA | 1992 | UUUUUCAGGUGACCCCUUC |
| siRNA 853 | 853 | AAGGGGUCACCUGAAAAAU | 1993 | AUUUUUCAGGUGACCCCUU |
| siRNA 854 | 854 | AGGGGUCACCUGAAAAAUA | 1994 | UAUUUUUCAGGUGACCCCU |
| siRNA 855 | 855 | GGGGUCACCUGAAAAAUAG | 1995 | CUAUUUUUCAGGUGACCCC |
| siRNA 856 | 856 | GGGUCACCUGAAAAAUAGG | 1996 | CCUAUUUUUCAGGUGACCC |
| siRNA 857 | 857 | GGUCACCUGAAAAAUAGGA | 1997 | UCCUAUUUUUCAGGUGACC |
| siRNA 858 | 858 | GUCACCUGAAAAAUAGGAC | 1998 | GUCCUAUUUUUCAGGUGAC |
| siRNA 859 | 859 | UCACCUGAAAAAUAGGACA | 1999 | UGUCCUAUUUUUCAGGUGA |
| siRNA 860 | 860 | CACCUGAAAAAUAGGACAU | 2000 | AUGUCCUAUUUUUCAGGUG |
| siRNA 861 | 861 | ACCUGAAAAAUAGGACAUU | 2001 | AAUGUCCUAUUUUUCAGGU |
| siRNA 862 | 862 | CCUGAAAAAUAGGACAUUU | 2002 | AAAUGUCCUAUUUUUCAGG |
| siRNA 863 | 863 | CUGAAAAAUAGGACAUUUU | 2003 | AAAAUGUCCUAUUUUUCAG |
| siRNA 864 | 864 | UGAAAAAUAGGACAUUUUU | 2004 | AAAAAUGUCCUAUUUUUCA |
| siRNA 865 | 865 | GAAAAAUAGGACAUUUUUA | 2005 | UAAAAAUGUCCUAUUUUUC |
| siRNA 866 | 866 | AAAAAUAGGACAUUUUUAU | 2006 | AUAAAAAUGUCCUAUUUUU |
| siRNA 867 | 867 | AAAAUAGGACAUUUUUAUU | 2007 | AAUAAAAAUGUCCUAUUUU |
| siRNA 868 | 868 | AAAUAGGACAUUUUUAUUA | 2008 | UAAUAAAAAUGUCCUAUUU |
| siRNA 869 | 869 | AAUAGGACAUUUUUAUUAA | 2009 | UUAAUAAAAAUGUCCUAUU |
| siRNA 870 | 870 | AUAGGACAUUUUUAUUAAA | 2010 | UUUAAUAAAAAUGUCCUAU |
| siRNA 871 | 871 | UAGGACAUUUUUAUUAAAA | 2011 | UUUUAAUAAAAAUGUCCUA |
| siRNA 872 | 872 | AGGACAUUUUUAUUAAAAU | 2012 | AUUUUAAUAAAAAUGUCCU |
| siRNA 873 | 873 | GGACAUUUUUAUUAAAAUA | 2013 | UAUUUUAAUAAAAAUGUCC |
| siRNA 874 | 874 | GACAUUUUUAUUAAAAUAA | 2014 | UUAUUUUAAUAAAAAUGUC |
| siRNA 875 | 875 | ACAUUUUUAUUAAAAUAAA | 2015 | UUUAUUUUAAUAAAAAUGU |
| siRNA 876 | 876 | CAUUUUUAUUAAAAUAAAG | 2016 | CUUUAUUUUAAUAAAAAUG |
| siRNA 877 | 877 | AUUUUUAUUAAAAUAAAGU | 2017 | ACUUUAUUUUAAUAAAAAU |
| siRNA 878 | 878 | UUUUUAUUAAAAUAAAGUU | 2018 | AACUUUAUUUUAAUAAAAA |
| siRNA 879 | 879 | UUUUAUUAAAAUAAAGUUC | 2019 | GAACUUUAUUUUAAUAAAA |
| siRNA 880 | 880 | UUUAUUAAAAUAAAGUUCU | 2020 | AGAACUUUAUUUUAAUAAA |
| siRNA 881 | 881 | UUAUUAAAAUAAAGUUCUC | 2021 | GAGAACUUUAUUUUAAUAA |
| siRNA 882 | 882 | UAUUAAAAUAAAGUUCUCU | 2022 | AGAGAACUUUAUUUUAAUA |
| siRNA 883 | 883 | AUUAAAAUAAAGUUCUCUU | 2023 | AAGAGAACUUUAUUUUAAU |
| siRNA 884 | 884 | UUAAAAUAAAGUUCUCUUA | 2024 | UAAGAGAACUUUAUUUUAA |
| siRNA 885 | 885 | UAAAAUAAAGUUCUCUUAG | 2025 | CUAAGAGAACUUUAUUUUA |
| siRNA 886 | 886 | AAAAUAAAGUUCUCUUAGC | 2026 | GCUAAGAGAACUUUAUUUU |
| siRNA 887 | 887 | AAAUAAAGUUCUCUUAGCG | 2027 | CGCUAAGAGAACUUUAUUU |
| siRNA 888 | 888 | AAUAAAGUUCUCUUAGCGU | 2028 | ACGCUAAGAGAACUUUAUU |
| siRNA 889 | 889 | AUAAAGUUCUCUUAGCGUU | 2029 | AACGCUAAGAGAACUUUAU |
| siRNA 890 | 890 | UAAAGUUCUCUUAGCGUUU | 2030 | AAACGCUAAGAGAACUUUA |
| siRNA 891 | 891 | AAAGUUCUCUUAGCGUUUG | 2031 | CAAACGCUAAGAGAACUUU |
| siRNA 892 | 892 | AAGUUCUCUUAGCGUUUGU | 2032 | ACAAACGCUAAGAGAACUU |
| siRNA 893 | 893 | AGUUCUCUUAGCGUUUGUG | 2033 | CACAAACGCUAAGAGAACU |
| siRNA 894 | 894 | GUUCUCUUAGCGUUUGUGG | 2034 | CCACAAACGCUAAGAGAAC |
| siRNA 895 | 895 | UUCUCUUAGCGUUUGUGGA | 2035 | UCCACAAACGCUAAGAGAA |
| siRNA 896 | 896 | UCUCUUAGCGUUUGUGGAA | 2036 | UUCCACAAACGCUAAGAGA |
| siRNA 897 | 897 | CUCUUAGCGUUUGUGGAAU | 2037 | AUUCCACAAACGCUAAGAG |
| siRNA 898 | 898 | UCUUAGCGUUUGUGGAAUC | 2038 | GAUUCCACAAACGCUAAGA |
| siRNA 899 | 899 | CUUAGCGUUUGUGGAAUCU | 2039 | AGAUUCCACAAACGCUAAG |
| siRNA 900 | 900 | UUAGCGUUUGUGGAAUCUG | 2040 | CAGAUUCCACAAACGCUAA |
| siRNA 901 | 901 | UAGCGUUUGUGGAAUCUGC | 2041 | GCAGAUUCCACAAACGCUA |
| siRNA 902 | 902 | AGCGUUUGUGGAAUCUGCC | 2042 | GGCAGAUUCCACAAACGCU |
| siRNA 903 | 903 | GCGUUUGUGGAAUCUGCCG | 2043 | CGGCAGAUUCCACAAACGC |
| siRNA 904 | 904 | CGUUUGUGGAAUCUGCCGA | 2044 | UCGGCAGAUUCCACAAACG |
| siRNA 905 | 905 | GUUUGUGGAAUCUGCCGAG | 2045 | CUCGGCAGAUUCCACAAAC |
| siRNA 906 | 906 | UUUGUGGAAUCUGCCGAGC | 2046 | GCUCGGCAGAUUCCACAAA |
| siRNA 907 | 907 | UUGUGGAAUCUGCCGAGCC | 2047 | GGCUCGGCAGAUUCCACAA |
| siRNA 908 | 908 | UGUGGAAUCUGCCGAGCCA | 2048 | UGGCUCGGCAGAUUCCACA |
| siRNA 909 | 909 | GUGGAAUCUGCCGAGCCAU | 2049 | AUGGCUCGGCAGAUUCCAC |
| siRNA 910 | 910 | UGGAAUCUGCCGAGCCAUU | 2050 | AAUGGCUCGGCAGAUUCCA |
| siRNA 911 | 911 | GGAAUCUGCCGAGCCAUUU | 2051 | AAAUGGCUCGGCAGAUUCC |
| siRNA 912 | 912 | GAAUCUGCCGAGCCAUUUU | 2052 | AAAAUGGCUCGGCAGAUUC |
| siRNA 913 | 913 | AAUCUGCCGAGCCAUUUUG | 2053 | CAAAAUGGCUCGGCAGAUU |
| siRNA 914 | 914 | AUCUGCCGAGCCAUUUUGU | 2054 | ACAAAAUGGCUCGGCAGAU |
| siRNA 915 | 915 | UCUGCCGAGCCAUUUUGUG | 2055 | CACAAAAUGGCUCGGCAGA |
| siRNA 916 | 916 | CUGCCGAGCCAUUUUGUGG | 2056 | CCACAAAAUGGCUCGGCAG |
| siRNA 917 | 917 | UGCCGAGCCAUUUUGUGGA | 2057 | UCCACAAAAUGGCUCGGCA |
| siRNA 918 | 918 | GCCGAGCCAUUUUGUGGAA | 2058 | UUCCACAAAAUGGCUCGGC |
| siRNA 919 | 919 | CCGAGCCAUUUUGUGGAAA | 2059 | UUUCCACAAAAUGGCUCGG |
| siRNA 920 | 920 | CGAGCCAUUUUGUGGAAAU | 2060 | AUUUCCACAAAAUGGCUCG |
| siRNA 921 | 921 | GAGCCAUUUUGUGGAAAUU | 2061 | AAUUUCCACAAAAUGGCUC |
| siRNA 922 | 922 | AGCCAUUUUGUGGAAAUUG | 2062 | CAAUUUCCACAAAAUGGCU |
| siRNA 923 | 923 | GCCAUUUUGUGGAAAUUGG | 2063 | CCAAUUUCCACAAAAUGGC |
| siRNA 924 | 924 | CCAUUUUGUGGAAAUUGGG | 2064 | CCCAAUUUCCACAAAAUGG |
| siRNA 925 | 925 | CAUUUUGUGGAAAUUGGGA | 2065 | UCCCAAUUUCCACAAAAUG |
| siRNA 926 | 926 | AUUUUGUGGAAAUUGGGAU | 2066 | AUCCCAAUUUCCACAAAAU |
| SIRNA 927 | 927 | UUUUGUGGAAAUUGGGAUC | 2067 | GAUCCCAAUUUCCACAAAA |
| siRNA 928 | 928 | UUUGUGGAAAUUGGGAUCC | 2068 | GGAUCCCAAUUUCCACAAA |
| siRNA 929 | 929 | UUGUGGAAAUUGGGAUCCA | 2069 | UGGAUCCCAAUUUCCACAA |
| siRNA 930 | 930 | UGUGGAAAUUGGGAUCCAU | 2070 | AUGGAUCCCAAUUUCCACA |
| siRNA 931 | 931 | GUGGAAAUUGGGAUCCAUA | 2071 | UAUGGAUCCCAAUUUCCAC |
| siRNA 932 | 932 | UGGAAAUUGGGAUCCAUAU | 2072 | AUAUGGAUCCCAAUUUCCA |
| siRNA 933 | 933 | GGAAAUUGGGAUCCAUAUC | 2073 | GAUAUGGAUCCCAAUUUCC |
| siRNA 934 | 934 | GAAAUUGGGAUCCAUAUCU | 2074 | AGAUAUGGAUCCCAAUUUC |
| siRNA 935 | 935 | AAAUUGGGAUCCAUAUCUG | 2075 | CAGAUAUGGAUCCCAAUUU |
| siRNA 936 | 936 | AAUUGGGAUCCAUAUCUGG | 2076 | CCAGAUAUGGAUCCCAAUU |
| siRNA 937 | 937 | AUUGGGAUCCAUAUCUGGA | 2077 | UCCAGAUAUGGAUCCCAAU |
| siRNA 938 | 938 | UUGGGAUCCAUAUCUGGAG | 2078 | CUCCAGAUAUGGAUCCCAA |
| siRNA 939 | 939 | UGGGAUCCAUAUCUGGAGA | 2079 | UCUCCAGAUAUGGAUCCCA |
| siRNA 940 | 940 | GGGAUCCAUAUCUGGAGAC | 2080 | GUCUCCAGAUAUGGAUCCC |
| siRNA 941 | 941 | GGAUCCAUAUCUGGAGACA | 2081 | UGUCUCCAGAUAUGGAUCC |
| siRNA 942 | 942 | GAUCCAUAUCUGGAGACAC | 2082 | GUGUCUCCAGAUAUGGAUC |
| siRNA 943 | 943 | AUCCAUAUCUGGAGACACU | 2083 | AGUGUCUCCAGAUAUGGAU |
| siRNA 944 | 944 | UCCAUAUCUGGAGACACUU | 2084 | AAGUGUCUCCAGAUAUGGA |
| siRNA 945 | 945 | CCAUAUCUGGAGACACUUC | 2085 | GAAGUGUCUCCAGAUAUGG |
| siRNA 946 | 946 | CAUAUCUGGAGACACUUCC | 2086 | GGAAGUGUCUCCAGAUAUG |
| siRNA 947 | 947 | AUAUCUGGAGACACUUCCC | 2087 | GGGAAGUGUCUCCAGAUAU |
| siRNA 948 | 948 | UAUCUGGAGACACUUCCCA | 2088 | UGGGAAGUGUCUCCAGAUA |
| siRNA 949 | 949 | AUCUGGAGACACUUCCCAA | 2089 | UUGGGAAGUGUCUCCAGAU |
| siRNA 950 | 950 | UCUGGAGACACUUCCCAAG | 2090 | CUUGGGAAGUGUCUCCAGA |
| siRNA 951 | 951 | CUGGAGACACUUCCCAAGG | 2091 | CCUUGGGAAGUGUCUCCAG |
| siRNA 952 | 952 | UGGAGACACUUCCCAAGGC | 2092 | GCCUUGGGAAGUGUCUCCA |
| siRNA 953 | 953 | GGAGACACUUCCCAAGGCC | 2093 | GGCCUUGGGAAGUGUCUCC |
| siRNA 954 | 954 | GAGACACUUCCCAAGGCCU | 2094 | AGGCCUUGGGAAGUGUCUC |
| siRNA 955 | 955 | AGACACUUCCCAAGGCCUG | 2095 | CAGGCCUUGGGAAGUGUCU |
| siRNA 956 | 956 | GACACUUCCCAAGGCCUGC | 2096 | GCAGGCCUUGGGAAGUGUC |
| siRNA 957 | 957 | ACACUUCCCAAGGCCUGCC | 2097 | GGCAGGCCUUGGGAAGUGU |
| siRNA 958 | 958 | CACUUCCCAAGGCCUGCCU | 2098 | AGGCAGGCCUUGGGAAGUG |
| siRNA 959 | 959 | ACUUCCCAAGGCCUGCCUC | 2099 | GAGGCAGGCCUUGGGAAGU |
| siRNA 960 | 960 | CUUCCCAAGGCCUGCCUCA | 2100 | UGAGGCAGGCCUUGGGAAG |
| siRNA 961 | 961 | UUCCCAAGGCCUGCCUCAC | 2101 | GUGAGGCAGGCCUUGGGAA |
| siRNA 962 | 962 | UCCCAAGGCCUGCCUCACC | 2102 | GGUGAGGCAGGCCUUGGGA |
| siRNA 963 | 963 | CCCAAGGCCUGCCUCACCU | 2103 | AGGUGAGGCAGGCCUUGGG |
| siRNA 964 | 964 | CCAAGGCCUGCCUCACCUC | 2104 | GAGGUGAGGCAGGCCUUGG |
| siRNA 965 | 965 | CAAGGCCUGCCUCACCUCC | 2105 | GGAGGUGAGGCAGGCCUUG |
| siRNA 966 | 966 | AAGGCCUGCCUCACCUCCA | 2106 | UGGAGGUGAGGCAGGCCUU |
| siRNA 967 | 967 | AGGCCUGCCUCACCUCCAC | 2107 | GUGGAGGUGAGGCAGGCCU |
| siRNA 968 | 968 | GGCCUGCCUCACCUCCACC | 2108 | GGUGGAGGUGAGGCAGGCC |
| siRNA 969 | 969 | GCCUGCCUCACCUCCACCC | 2109 | GGGUGGAGGUGAGGCAGGC |
| siRNA 970 | 970 | CCUGCCUCACCUCCACCCC | 2110 | GGGGUGGAGGUGAGGCAGG |
| siRNA 971 | 971 | CUGCCUCACCUCCACCCCC | 2111 | GGGGGUGGAGGUGAGGCAG |
| siRNA 972 | 972 | UGCCUCACCUCCACCCCCU | 2112 | AGGGGGUGGAGGUGAGGCA |
| siRNA 973 | 973 | GCCUCACCUCCACCCCCUG | 2113 | CAGGGGGUGGAGGUGAGGC |
| siRNA 974 | 974 | CCUCACCUCCACCCCCUGC | 2114 | GCAGGGGGUGGAGGUGAGG |
| siRNA 975 | 975 | CUCACCUCCACCCCCUGCC | 2115 | GGCAGGGGGUGGAGGUGAG |
| siRNA 976 | 976 | UCACCUCCACCCCCUGCCC | 2116 | GGGCAGGGGGUGGAGGUGA |
| siRNA 977 | 977 | CACCUCCACCCCCUGCCCA | 2117 | UGGGCAGGGGGUGGAGGUG |
| siRNA 978 | 978 | ACCUCCACCCCCUGCCCAC | 2118 | GUGGGCAGGGGGUGGAGGU |
| siRNA 979 | 979 | CCUCCACCCCCUGCCCACC | 2119 | GGUGGGCAGGGGGUGGAGG |
| siRNA 980 | 980 | CUCCACCCCCUGCCCACCU | 2120 | AGGUGGGCAGGGGGUGGAG |
| siRNA 981 | 981 | UCCACCCCCUGCCCACCUU | 2121 | AAGGUGGGCAGGGGGUGGA |
| siRNA 982 | 982 | CCACCCCCUGCCCACCUUG | 2122 | CAAGGUGGGCAGGGGGUGG |
| siRNA 983 | 983 | CACCCCCUGCCCACCUUGA | 2123 | UCAAGGUGGGCAGGGGGUG |
| siRNA 984 | 984 | ACCCCCUGCCCACCUUGAU | 2124 | AUCAAGGUGGGCAGGGGGU |
| siRNA 985 | 985 | CCCCCUGCCCACCUUGAUC | 2125 | GAUCAAGGUGGGCAGGGGG |
| siRNA 986 | 986 | CCCCUGCCCACCUUGAUCC | 2126 | GGAUCAAGGUGGGCAGGGG |
| siRNA 987 | 987 | CCCUGCCCACCUUGAUCCA | 2127 | UGGAUCAAGGUGGGCAGGG |
| siRNA 988 | 988 | CCUGCCCACCUUGAUCCAU | 2128 | AUGGAUCAAGGUGGGCAGG |
| siRNA 989 | 989 | CUGCCCACCUUGAUCCAUG | 2129 | CAUGGAUCAAGGUGGGCAG |
| siRNA 990 | 990 | UGCCCACCUUGAUCCAUGC | 2130 | GCAUGGAUCAAGGUGGGCA |
| siRNA 991 | 991 | GCCCACCUUGAUCCAUGCU | 2131 | AGCAUGGAUCAAGGUGGGC |
| siRNA 992 | 992 | CCCACCUUGAUCCAUGCUC | 2132 | GAGCAUGGAUCAAGGUGGG |
| siRNA 993 | 993 | CCACCUUGAUCCAUGCUCC | 2133 | GGAGCAUGGAUCAAGGUGG |
| siRNA 994 | 994 | CACCUUGAUCCAUGCUCCU | 2134 | AGGAGCAUGGAUCAAGGUG |
| siRNA 995 | 995 | ACCUUGAUCCAUGCUCCUU | 2135 | AAGGAGCAUGGAUCAAGGU |
| siRNA 996 | 996 | CCUUGAUCCAUGCUCCUUU | 2136 | AAAGGAGCAUGGAUCAAGG |
| siRNA 997 | 997 | CUUGAUCCAUGCUCCUUUG | 2137 | CAAAGGAGCAUGGAUCAAG |
| siRNA 998 | 998 | UUGAUCCAUGCUCCUUUGA | 2138 | UCAAAGGAGCAUGGAUCAA |
| siRNA 999 | 999 | UGAUCCAUGCUCCUUUGAC | 2139 | GUCAAAGGAGCAUGGAUCA |
| siRNA 1000 | 1000 | GAUCCAUGCUCCUUUGACC | 2140 | GGUCAAAGGAGCAUGGAUC |
| siRNA 1001 | 1001 | AUCCAUGCUCCUUUGACCU | 2141 | AGGUCAAAGGAGCAUGGAU |
| siRNA 1002 | 1002 | UCCAUGCUCCUUUGACCUC | 2142 | GAGGUCAAAGGAGCAUGGA |
| siRNA 1003 | 1003 | CCAUGCUCCUUUGACCUCC | 2143 | GGAGGUCAAAGGAGCAUGG |
| siRNA 1004 | 1004 | CAUGCUCCUUUGACCUCCU | 2144 | AGGAGGUCAAAGGAGCAUG |
| siRNA 1005 | 1005 | AUGCUCCUUUGACCUCCUC | 2145 | GAGGAGGUCAAAGGAGCAU |
| siRNA 1006 | 1006 | UGCUCCUUUGACCUCCUCG | 2146 | CGAGGAGGUCAAAGGAGCA |
| siRNA 1007 | 1007 | GCUCCUUUGACCUCCUCGU | 2147 | ACGAGGAGGUCAAAGGAGC |
| siRNA 1008 | 1008 | CUCCUUUGACCUCCUCGUG | 2148 | CACGAGGAGGUCAAAGGAG |
| siRNA 1009 | 1009 | UCCUUUGACCUCCUCGUGU | 2149 | ACACGAGGAGGUCAAAGGA |
| siRNA 1010 | 1010 | CCUUUGACCUCCUCGUGUG | 2150 | CACACGAGGAGGUCAAAGG |
| siRNA 1011 | 1011 | CUUUGACCUCCUCGUGUGA | 2151 | UCACACGAGGAGGUCAAAG |
| siRNA 1012 | 1012 | UUUGACCUCCUCGUGUGAG | 2152 | CUCACACGAGGAGGUCAAA |
| siRNA 1013 | 1013 | UUGACCUCCUCGUGUGAGA | 2153 | UCUCACACGAGGAGGUCAA |
| siRNA 1014 | 1014 | UGACCUCCUCGUGUGAGAA | 2154 | UUCUCACACGAGGAGGUCA |
| siRNA 1015 | 1015 | GACCUCCUCGUGUGAGAAC | 2155 | GUUCUCACACGAGGAGGUC |
| siRNA 1016 | 1016 | ACCUCCUCGUGUGAGAACC | 2156 | GGUUCUCACACGAGGAGGU |
| SIRNA 1017 | 1017 | CCUCCUCGUGUGAGAACCC | 2157 | GGGUUCUCACACGAGGAGG |
| SiRNA 1018 | 1018 | CUCCUCGUGUGAGAACCCC | 2158 | GGGGUUCUCACACGAGGAG |
| siRNA 1019 | 1019 | UCCUCGUGUGAGAACCCCU | 2159 | AGGGGUUCUCACACGAGGA |
| siRNA 1020 | 1020 | CCUCGUGUGAGAACCCCUU | 2160 | AAGGGGUUCUCACACGAGG |
| siRNA 1021 | 1021 | CUCGUGUGAGAACCCCUUU | 2161 | AAAGGGGUUCUCACACGAG |
| siRNA 1022 | 1022 | UCGUGUGAGAACCCCUUUG | 2162 | CAAAGGGGUUCUCACACGA |
| siRNA 1023 | 1023 | CGUGUGAGAACCCCUUUGC | 2163 | GCAAAGGGGUUCUCACACG |
| siRNA 1024 | 1024 | GUGUGAGAACCCCUUUGCC | 2164 | GGCAAAGGGGUUCUCACAC |
| siRNA 1025 | 1025 | UGUGAGAACCCCUUUGCCA | 2165 | UGGCAAAGGGGUUCUCACA |
| siRNA 1026 | 1026 | GUGAGAACCCCUUUGCCAG | 2166 | CUGGCAAAGGGGUUCUCAC |
| siRNA 1027 | 1027 | UGAGAACCCCUUUGCCAGA | 2167 | UCUGGCAAAGGGGUUCUCA |
| SiRNA 1028 | 1028 | GAGAACCCCUUUGCCAGAG | 2168 | CUCUGGCAAAGGGGUUCUC |
| siRNA 1029 | 1029 | AGAACCCCUUUGCCAGAGU | 2169 | ACUCUGGCAAAGGGGUUCU |
| siRNA 1030 | 1030 | GAACCCCUUUGCCAGAGUG | 2170 | CACUCUGGCAAAGGGGUUC |
| siRNA 1031 | 1031 | AACCCCUUUGCCAGAGUGA | 2171 | UCACUCUGGCAAAGGGGUU |
| siRNA 1032 | 1032 | ACCCCUUUGCCAGAGUGAG | 2172 | CUCACUCUGGCAAAGGGGU |
| siRNA 1033 | 1033 | CCCCUUUGCCAGAGUGAGA | 2173 | UCUCACUCUGGCAAAGGGG |
| siRNA 1034 | 1034 | CCCUUUGCCAGAGUGAGAC | 2174 | GUCUCACUCUGGCAAAGGG |
| siRNA 1035 | 1035 | CCUUUGCCAGAGUGAGACG | 2175 | CGUCUCACUCUGGCAAAGG |
| siRNA 1036 | 1036 | CUUUGCCAGAGUGAGACGU | 2176 | ACGUCUCACUCUGGCAAAG |
| siRNA 1037 | 1037 | UUUGCCAGAGUGAGACGUG | 2177 | CACGUCUCACUCUGGCAAA |
| siRNA 1038 | 1038 | UUGCCAGAGUGAGACGUGU | 2178 | ACACGUCUCACUCUGGCAA |
| siRNA 1039 | 1039 | UGCCAGAGUGAGACGUGUG | 2179 | CACACGUCUCACUCUGGCA |
| siRNA 1040 | 1040 | GCCAGAGUGAGACGUGUGC | 2180 | GCACACGUCUCACUCUGGC |
| siRNA 1041 | 1041 | CCAGAGUGAGACGUGUGCA | 2181 | UGCACACGUCUCACUCUGG |
| siRNA 1042 | 1042 | CAGAGUGAGACGUGUGCAG | 2182 | CUGCACACGUCUCACUCUG |
| siRNA 1043 | 1043 | AGAGUGAGACGUGUGCAGA | 2183 | UCUGCACACGUCUCACUCU |
| siRNA 1044 | 1044 | GAGUGAGACGUGUGCAGAA | 2184 | UUCUGCACACGUCUCACUC |
| siRNA 1045 | 1045 | AGUGAGACGUGUGCAGAAU | 2185 | AUUCUGCACACGUCUCACU |
| siRNA 1046 | 1046 | GUGAGACGUGUGCAGAAUG | 2186 | CAUUCUGCACACGUCUCAC |
| siRNA 1047 | 1047 | UGAGACGUGUGCAGAAUGA | 2187 | UCAUUCUGCACACGUCUCA |
| siRNA 1048 | 1048 | GAGACGUGUGCAGAAUGAA | 2188 | UUCAUUCUGCACACGUCUC |
| siRNA 1049 | 1049 | AGACGUGUGCAGAAUGAAC | 2189 | GUUCAUUCUGCACACGUCU |
| siRNA 1050 | 1050 | GACGUGUGCAGAAUGAACU | 2190 | AGUUCAUUCUGCACACGUC |
| siRNA 1051 | 1051 | ACGUGUGCAGAAUGAACUA | 2191 | UAGUUCAUUCUGCACACGU |
| siRNA 1052 | 1052 | CGUGUGCAGAAUGAACUAA | 2192 | UUAGUUCAUUCUGCACACG |
| siRNA 1053 | 1053 | GUGUGCAGAAUGAACUAAG | 2193 | CUUAGUUCAUUCUGCACAC |
| siRNA 1054 | 1054 | UGUGCAGAAUGAACUAAGC | 2194 | GCUUAGUUCAUUCUGCACA |
| siRNA 1055 | 1055 | GUGCAGAAUGAACUAAGCC | 2195 | GGCUUAGUUCAUUCUGCAC |
| siRNA 1056 | 1056 | UGCAGAAUGAACUAAGCCC | 2196 | GGGCUUAGUUCAUUCUGCA |
| siRNA 1057 | 1057 | GCAGAAUGAACUAAGCCCC | 2197 | GGGGCUUAGUUCAUUCUGC |
| siRNA 1058 | 1058 | CAGAAUGAACUAAGCCCCA | 2198 | UGGGGCUUAGUUCAUUCUG |
| siRNA 1059 | 1059 | AGAAUGAACUAAGCCCCAG | 2199 | CUGGGGCUUAGUUCAUUCU |
| siRNA 1060 | 1060 | GAAUGAACUAAGCCCCAGA | 2200 | UCUGGGGCUUAGUUCAUUC |
| siRNA 1061 | 1061 | AAUGAACUAAGCCCCAGAG | 2201 | CUCUGGGGCUUAGUUCAUU |
| siRNA 1062 | 1062 | AUGAACUAAGCCCCAGAGG | 2202 | CCUCUGGGGCUUAGUUCAU |
| siRNA 1063 | 1063 | UGAACUAAGCCCCAGAGGG | 2203 | CCCUCUGGGGCUUAGUUCA |
| siRNA 1064 | 1064 | GAACUAAGCCCCAGAGGGU | 2204 | ACCCUCUGGGGCUUAGUUC |
| siRNA 1065 | 1065 | AACUAAGCCCCAGAGGGUU | 2205 | AACCCUCUGGGGCUUAGUU |
| siRNA 1066 | 1066 | ACUAAGCCCCAGAGGGUUU | 2206 | AAACCCUCUGGGGCUUAGU |
| siRNA 1067 | 1067 | CUAAGCCCCAGAGGGUUUU | 2207 | AAAACCCUCUGGGGCUUAG |
| siRNA 1068 | 1068 | UAAGCCCCAGAGGGUUUUA | 2208 | UAAAACCCUCUGGGGCUUA |
| siRNA 1069 | 1069 | AAGCCCCAGAGGGUUUUAA | 2209 | UUAAAACCCUCUGGGGCUU |
| siRNA 1070 | 1070 | AGCCCCAGAGGGUUUUAAU | 2210 | AUUAAAACCCUCUGGGGCU |
| siRNA 1071 | 1071 | GCCCCAGAGGGUUUUAAUG | 2211 | CAUUAAAACCCUCUGGGGC |
| siRNA 1072 | 1072 | CCCCAGAGGGUUUUAAUGG | 2212 | CCAUUAAAACCCUCUGGGG |
| siRNA 1073 | 1073 | CCCAGAGGGUUUUAAUGGC | 2213 | GCCAUUAAAACCCUCUGGG |
| siRNA 1074 | 1074 | CCAGAGGGUUUUAAUGGCU | 2214 | AGCCAUUAAAACCCUCUGG |
| siRNA 1075 | 1075 | CAGAGGGUUUUAAUGGCUU | 2215 | AAGCCAUUAAAACCCUCUG |
| siRNA 1076 | 1076 | AGAGGGUUUUAAUGGCUUG | 2216 | CAAGCCAUUAAAACCCUCU |
| siRNA 1077 | 1077 | GAGGGUUUUAAUGGCUUGC | 2217 | GCAAGCCAUUAAAACCCUC |
| siRNA 1078 | 1078 | AGGGUUUUAAUGGCUUGCC | 2218 | GGCAAGCCAUUAAAACCCU |
| siRNA 1079 | 1079 | GGGUUUUAAUGGCUUGCCU | 2219 | AGGCAAGCCAUUAAAACCC |
| siRNA 1080 | 1080 | GGUUUUAAUGGCUUGCCUG | 2220 | CAGGCAAGCCAUUAAAACC |
| siRNA 1081 | 1081 | GUUUUAAUGGCUUGCCUGC | 2221 | GCAGGCAAGCCAUUAAAAC |
| siRNA 1082 | 1082 | UUUUAAUGGCUUGCCUGCU | 2222 | AGCAGGCAAGCCAUUAAAA |
| siRNA 1083 | 1083 | UUUAAUGGCUUGCCUGCUG | 2223 | CAGCAGGCAAGCCAUUAAA |
| siRNA 1084 | 1084 | UUAAUGGCUUGCCUGCUGU | 2224 | ACAGCAGGCAAGCCAUUAA |
| siRNA 1085 | 1085 | UAAUGGCUUGCCUGCUGUU | 2225 | AACAGCAGGCAAGCCAUUA |
| siRNA 1086 | 1086 | AAUGGCUUGCCUGCUGUUU | 2226 | AAACAGCAGGCAAGCCAUU |
| siRNA 1087 | 1087 | AUGGCUUGCCUGCUGUUUC | 2227 | GAAACAGCAGGCAAGCCAU |
| siRNA 1088 | 1088 | UGGCUUGCCUGCUGUUUCC | 2228 | GGAAACAGCAGGCAAGCCA |
| siRNA 1089 | 1089 | GGCUUGCCUGCUGUUUCCC | 2229 | GGGAAACAGCAGGCAAGCC |
| siRNA 1090 | 1090 | GCUUGCCUGCUGUUUCCCA | 2230 | UGGGAAACAGCAGGCAAGC |
| siRNA 1091 | 1091 | CUUGCCUGCUGUUUCCCAC | 2231 | GUGGGAAACAGCAGGCAAG |
| siRNA 1092 | 1092 | UUGCCUGCUGUUUCCCACA | 2232 | UGUGGGAAACAGCAGGCAA |
| siRNA 1093 | 1093 | UGCCUGCUGUUUCCCACAU | 2233 | AUGUGGGAAACAGCAGGCA |
| siRNA 1094 | 1094 | GCCUGCUGUUUCCCACAUA | 2234 | UAUGUGGGAAACAGCAGGC |
| siRNA 1095 | 1095 | CCUGCUGUUUCCCACAUAA | 2235 | UUAUGUGGGAAACAGCAGG |
| siRNA 1096 | 1096 | CUGCUGUUUCCCACAUAAA | 2236 | UUUAUGUGGGAAACAGCAG |
| siRNA 1097 | 1097 | UGCUGUUUCCCACAUAAAC | 2237 | GUUUAUGUGGGAAACAGCA |
| siRNA 1098 | 1098 | GCUGUUUCCCACAUAAACU | 2238 | AGUUUAUGUGGGAAACAGC |
| siRNA 1099 | 1099 | CUGUUUCCCACAUAAACUA | 2239 | UAGUUUAUGUGGGAAACAG |
| siRNA 1100 | 1100 | UGUUUCCCACAUAAACUAC | 2240 | GUAGUUUAUGUGGGAAACA |
| siRNA 1101 | 1101 | GUUUCCCACAUAAACUACC | 2241 | GGUAGUUUAUGUGGGAAAC |
| siRNA 1102 | 1102 | UUUCCCACAUAAACUACCU | 2242 | AGGUAGUUUAUGUGGGAAA |
| siRNA 1103 | 1103 | UUCCCACAUAAACUACCUC | 2243 | GAGGUAGUUUAUGUGGGAA |
| siRNA 1104 | 1104 | UCCCACAUAAACUACCUCA | 2244 | UGAGGUAGUUUAUGUGGGA |
| siRNA 1105 | 1105 | CCCACAUAAACUACCUCAG | 2245 | CUGAGGUAGUUUAUGUGGG |
| siRNA 1106 | 1106 | CCACAUAAACUACCUCAGG | 2246 | CCUGAGGUAGUUUAUGUGG |
| siRNA 1107 | 1107 | CACAUAAACUACCUCAGGA | 2247 | UCCUGAGGUAGUUUAUGUG |
| siRNA 1108 | 1108 | ACAUAAACUACCUCAGGAG | 2248 | CUCCUGAGGUAGUUUAUGU |
| siRNA 1109 | 1109 | CAUAAACUACCUCAGGAGU | 2249 | ACUCCUGAGGUAGUUUAUG |
| siRNA 1110 | 1110 | AUAAACUACCUCAGGAGUC | 2250 | GACUCCUGAGGUAGUUUAU |
| siRNA 1111 | 1111 | UAAACUACCUCAGGAGUCA | 2251 | UGACUCCUGAGGUAGUUUA |
| siRNA 1112 | 1112 | AAACUACCUCAGGAGUCAC | 2252 | GUGACUCCUGAGGUAGUUU |
| siRNA 1113 | 1113 | AACUACCUCAGGAGUCACU | 2253 | AGUGACUCCUGAGGUAGUU |
| siRNA 1114 | 1114 | ACUACCUCAGGAGUCACUG | 2254 | CAGUGACUCCUGAGGUAGU |
| siRNA 1115 | 1115 | CUACCUCAGGAGUCACUGU | 2255 | ACAGUGACUCCUGAGGUAG |
| siRNA 1116 | 1116 | UACCUCAGGAGUCACUGUA | 2256 | UACAGUGACUCCUGAGGUA |
| siRNA 1117 | 1117 | ACCUCAGGAGUCACUGUAA | 2257 | UUACAGUGACUCCUGAGGU |
| siRNA 1118 | 1118 | CCUCAGGAGUCACUGUAAA | 2258 | UUUACAGUGACUCCUGAGG |
| siRNA 1119 | 1119 | CUCAGGAGUCACUGUAAAA | 2259 | UUUUACAGUGACUCCUGAG |
| siRNA 1120 | 1120 | UCAGGAGUCACUGUAAAAU | 2260 | AUUUUACAGUGACUCCUGA |
| siRNA 1121 | 1121 | CAGGAGUCACUGUAAAAUA | 2261 | UAUUUUACAGUGACUCCUG |
| siRNA 1122 | 1122 | AGGAGUCACUGUAAAAUAA | 2262 | UUAUUUUACAGUGACUCCU |
| siRNA 1123 | 1123 | GGAGUCACUGUAAAAUAAA | 2263 | UUUAUUUUACAGUGACUCC |
| siRNA 1124 | 1124 | GAGUCACUGUAAAAUAAAC | 2264 | GUUUAUUUUACAGUGACUC |
| siRNA 1125 | 1125 | AGUCACUGUAAAAUAAACU | 2265 | AGUUUAUUUUACAGUGACU |
| siRNA 1126 | 1126 | GUCACUGUAAAAUAAACUG | 2266 | CAGUUUAUUUUACAGUGAC |
| siRNA 1127 | 1127 | UCACUGUAAAAUAAACUGG | 2267 | CCAGUUUAUUUUACAGUGA |
| siRNA 1128 | 1128 | CACUGUAAAAUAAACUGGC | 2268 | GCCAGUUUAUUUUACAGUG |
| siRNA 1129 | 1129 | ACUGUAAAAUAAACUGGCC | 2269 | GGCCAGUUUAUUUUACAGU |
| siRNA 1130 | 1130 | CUGUAAAAUAAACUGGCCU | 2270 | AGGCCAGUUUAUUUUACAG |
| siRNA 1131 | 1131 | UGUAAAAUAAACUGGCCUU | 2271 | AAGGCCAGUUUAUUUUACA |
| siRNA 1132 | 1132 | GUAAAAUAAACUGGCCUUG | 2272 | CAAGGCCAGUUUAUUUUAC |
| siRNA 1133 | 1133 | UAAAAUAAACUGGCCUUGU | 2273 | ACAAGGCCAGUUUAUUUUA |
| siRNA 1134 | 1134 | AAAAUAAACUGGCCUUGUU | 2274 | AACAAGGCCAGUUUAUUUU |
| siRNA 1135 | 1135 | AAAUAAACUGGCCUUGUUG | 2275 | CAACAAGGCCAGUUUAUUU |
| siRNA 1136 | 1136 | AAUAAACUGGCCUUGUUGU | 2276 | ACAACAAGGCCAGUUUAUU |
| siRNA 1137 | 1137 | AUAAACUGGCCUUGUUGUC | 2277 | GACAACAAGGCCAGUUUAU |
| siRNA 1138 | 1138 | UAAACUGGCCUUGUUGUCU | 2278 | AGACAACAAGGCCAGUUUA |
| siRNA 1139 | 1139 | AAACUGGCCUUGUUGUCUU | 2279 | AAGACAACAAGGCCAGUUU |
| siRNA 1140 | 1140 | AACUGGCCUUGUUGUCUUA | 2280 | UAAGACAACAAGGCCAGUU |

TABLE 21
Additional Sequences

| SEQ ID | |
|---|---|
| NO: | 5′ to 3′ Sequence |
| 2443 | GGGGTGGGGAGGGAGCGAGAGGAATCCGACCCTGTCTCAGCCCACAGCCTCCGAGGTCTCCAAGTAAAGGGAAGGA |
| | TCTTTAGCTGCATTAGACTTCAAAGCGTTTAGACCAGTTTCTCCATCTTACGGAGCGGTGAACGGGCTCAGGAATGTG |
| | GAGCGTTTCCTGGCGTCAAGCAGGTCAAAGTCAGCGCTGCTTTTTTTACAGACACTGCTTTTCTTACAGTCTTCGACTA |
| | TAAACTCTACAAGAATAGGAATCTTCGTATTTTTTTCCTCTGCTGAATTCCTAGTGCCCAGATTAGTGCTTGGCACATG |
| | ATTATAAGCGCCATGGCTATGGCTAGTGTTAAATTGCTTGCCGGTGTTTTAAGAAAGCCAGATGCCTGGATTGGACTC |
| | TGGGGTGTTCTCCGAGGGACACCTTCATCATACAAACTCTGTACTTCCTGGAATCGATACTTGTATTTTTCTAGTACCA |
| | AGTTACGTGCACCAAATTATAAAACACTTTTTTATAATATTTTCTCACTGAGACTCCCAGGGCTTTTACTATCTCCAGA |
| | ATGTATTTTTCCTTTTTCCGTAAGACTCAAAAGTAATATAAGGTCTACAAAATCTACTAAAAAGTCTCTGCAAAAAGT |
| | AGATGAAGAGGACTCTGATGAAGAAAGCCATCATGATGAGATGAGTGAGCAGGAAGAGGAGCTTGAGGATGATCCT |
| | ACTGTAGTCAAAAACTATAAAGACCTGGAAAAAGCAGTTCAGTCTTTTCGGTATGATGTTGTCCTGAAGACGGGGCT |
| | AGATATTGGGAGAAACAAAGTGGAAGATGCTTTCTACAAAGGTGAACTCAGGCTGAATGAGGAAAAATTATGGAAG |
| | AAAAGCAGAACGGTGAAAGTGGGAGATACATTGGATCTTCTCATTGGAGAGGATAAAGAAGCAGGAACAGAGACAG |
| | TTATGCGGATTCTCTTGAAAAAAGTGTTTGAAGAGAAGACTGAAAGTGAAAAATACAGAGTGGTGTTACGGCGGTGG |
| | AAAAGTTTAAAGTTGCCTAAGAAGAGAATGTCTAAATAAATGGATTGCTTTTTAGCAATAGAGCTGCTTTCTAGTGGT |
| | AAAGGAAGGGGTCACCTGAAAAATAGGACATTTTTATTAAAATAAAGTTCTCTTAGCGTT |
| 2462 | GGGGTGGGGAGGGAGCGAGAGGAATCCGACCCTGTCTCAGCCCACAGCCTCCGAGGTCTCCAAGTAAAGGGAAGGA |
| | TCTTTAGCTGCATTAGACTTCAAAGCGTTTAGACCAGTTTCTCCATCTTACGGAGCGGTGAACGGGCTCAGGAATGTG |
| | GAGCGTTTCCTGGCGTCAAGCAGGTCAAAGTCAGCGCTGCTTTTTTTACAGACACTGCTTTTCTTACAGTCTTCGACTA |
| | TAAACTCTACAAGAATAGGAATCTTCGTATTTTTTTCCTCTGCTGAATTCCTAGTGCCCAGATTAGTGCTTGGCACATG |
| | ATTATAAGCGCCATGGCTATGGCTAGTGTTAAATTGCTTGCCGGTGTTTTAAGAAAGCCAGATGCCTGGATTGGACTC |
| | TGGGGTGTTCTCCGAGGGACACCTTCATCATACAAACTCTGTACTTCCTGGAATCGATACTTGTATTTTTCTAGTACCA |
| | AGTTACGTGCACCAAATTATAAAACACTTTTTTATAATATTTTCTCACTGAGACTCCCAGGGCTTTTACTATCTCCAGA |
| | ATGTATTTTTCCTTTTTCCGTAAGACTCAAAAGTAATATAAGGTCTACAAAATCTACTAAAAAGTCTCTGCAAAAAGT |
| | AGATGAAGAGGACTCTGATGAAGAAAGCCATCATGATGAGATGAGTGAGCAGGAAGAGGAGCTTGAGGATGATCCT |
| | ACTGTAGTCAAAAACTATAAAGACCTGGAAAAAGCAGTTCAGTCTTTTCGGTATGATGTTGTCCTGAAGACGGGGCT |
| | AGATATTGGGAGAAACAAAGTGGAAGATGCTTTCTACAAAGGTGAACTCAGGCTGAATGAGGAAAAATTATGGAAG |
| | AAAAGCAGAACGGTGAAAGTGGGAGATACATTGGATCTTCTCATTGGAGAGGATAAAGAAGCAGGAACAGAGACAG |
| | TTATGCGGATTCTCTTGAAAAAAGTGTTTGAAGAGAAGACTGAAAGTGAAAAATACAGAGTGGTGTTACGGCGGTGG |
| | AAAAGTTTAAAGTTGCCTAAGAAGAGAATGTCTAAATAAATGGATTGCTTTTTAGCAATAGAGCTGCTTTCTAGTGGT |
| | AAAGGAAGGGGTCACCTGAAAAATAGGACATTTTTATTAAAATAAAGTTCTCTTAGCGTT |

What is claimed is:

1. A composition comprising an oligonucleotide that targets MTRES1 and when administered to a subject in an effective amount decreases central nervous system (CNS) MTRES1.

2. The composition of claim 1, wherein the CNS MTRES1 decreased by about 10% or more, as compared to prior to administration.

3. A composition comprising an oligonucleotide that targets MTRES1 and when administered to a subject in an effective amount increases cognitive function or slows cognitive decline.

4. The composition of claim 3, wherein the cognitive function is increased by about 10% or more, as compared to prior to administration.

5. The composition of claim 3, wherein the cognitive decline is slowed by about 10% or more, as compared to prior to administration.

6. A composition comprising an oligonucleotide that targets MTRES1 and when administered to a subject in an effective amount decreases a marker of neurodegeneration.

7. The composition of claim 6, wherein the marker of neurodegeneration comprises a central nervous system (CNS) or cerebrospinal fluid (CSF) marker of neurodegeneration.

8. The composition of claim 6, wherein the marker of neurodegeneration comprises a measurement of central nervous system (CNS) amyloid plaques, CNS tau accumulation, cerebrospinal fluid (CSF) beta-amyloid 42, CSF tau, CSF phospho-tau, CSF or plasma neurofilament light chain (NfL), Lewy bodies, or CSF alpha-synuclein.

9. The composition of any one of claims 6-8, wherein the marker of neurodegeneration is decreased by about 10% or more, as compared to prior to administration.

10. The composition of any one of claims 1, 3, or 6, wherein the oligonucleotide comprises a modified internucleoside linkage.

11. The composition of claim 10, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

12. The composition of claim 10, wherein the modified internucleoside linkage comprises one or more phosphorothioate linkages.

13. The composition of any one of claims 1, 3, or 6, wherein the oligonucleotide comprises 1, 2, 3, 4, 5, 6, 7, 8, 9, 10, 11, 12, 13, 14, 15, 16, 17, 18, 19, or 20 modified internucleoside linkages.

14. The composition of any one of claims 1, 3, or 6, wherein the oligonucleotide comprises a modified nucleoside.

15. The composition of claim 14, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

16. The composition of claim 14, wherein the modified nucleoside comprises a LNA.

17. The composition of claim 14, wherein the modified nucleoside comprises a 2′,4′ constrained ethyl nucleic acid.

18. The composition of claim 14, wherein the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP)nucleoside, or 2′-ara-F, or a combination thereof.

19. The composition of claim 14, wherein the modified nucleoside comprises one or more 2′fluoro modified nucleosides.

20. The composition of claim 14, wherein the modified nucleoside comprises a 2′ O-alkyl modified nucleoside.

21. The composition of any one of claims 1, 3, or 6, wherein the oligonucleotide comprises 1, 2, 3, 4, 5, 6, 7, 8, 9, 10, 11, 12, 13, 14, 15, 16, 17, 18, 19, 20, or 21 modified nucleosides.

22. The composition of claim any one of claims 1, 3, or 6, wherein the oligonucleotide comprises a lipophilic moiety attached at a 3′ or 5′ terminus of the oligonucleotide.

23. The composition of claim 22, wherein the lipophilic moiety comprises cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl, palmitic acid, myristic acid, 03-(oleoyl)lithocholic acid, 03-(oleoyl)cholenic acid, ibuprofen, naproxen, dimethoxytrityl, or phenoxazine.

24. The composition of claim 22, wherein the lipophilic moiety comprises a C4-C30 hydrocarbon chain.

25. The composition of claim 22, wherein the lipophilic moiety comprises a lipid.

26. The composition of claim 25, wherein the lipid comprises myristoyl, palmitoyl, stearoyl, lithocholoyl, docosanoyl, docosahexaenoyl, myristyl, palmityl stearyl, or α-tocopherol, or a combination thereof.

27. The composition of claim any one of claims 1, 3, or 6, wherein the oligonucleotide comprises a small interfering RNA (siRNA) comprising a sense strand and an antisense strand.

28. The composition of claim 27, wherein the sense strand is 12-30 nucleosides in length.

29. The composition of claim 27, wherein the antisense strand is 12-30 nucleosides in length.

30. A composition comprising an oligonucleotide that inhibits the expression of MTRES1, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 2443.

31. The composition of claim 27, wherein any one of the following is true with regard to the sense strand:
all purines comprise 2′ fluoro modified purines, and all pyrimidines comprise a mixture of 2′ fluoro and 2′ methyl modified pyrimidines;
all purines comprise 2′ methyl modified purines, and all pyrimidines comprise a mixture of 2′ fluoro and 2′ methyl modified pyrimidines;
all purines comprise 2′ fluoro modified purines, and all pyrimidines comprise 2′ methyl modified pyrimidines;
all pyrimidines comprise 2′ fluoro modified pyrimidines, and all purines comprise a mixture of 2′ fluoro and 2′ methyl modified purines;
all pyrimidines comprise 2′ methyl modified pyrimidines, and all purines comprise a mixture of 2′ fluoro and 2′ methyl modified purines; or
all pyrimidines comprise 2′ fluoro modified pyrimidines, and all purines comprise 2′ methyl modified purines.

32. The composition of claim 27, wherein any one of the following is true with regard to the antisense strand:
all purines comprise 2′ fluoro modified purines, and all pyrimidines comprise a mixture of 2′ fluoro and 2′ methyl modified pyrimidines;
all purines comprise 2′ methyl modified purines, and all pyrimidines comprise a mixture of 2′ fluoro and 2′ methyl modified pyrimidines;
all purines comprise 2′ methyl modified purines, and all pyrimidines comprise 2′ fluoro modified pyrimidines;
all pyrimidines comprise 2′ fluoro modified pyrimidines, and all purines comprise a mixture of 2′ fluoro and 2′ methyl modified purines;
all pyrimidines comprise 2′ methyl modified pyrimidines, and all purines comprise a mixture of 2′ fluoro and 2′ methyl modified purines; or
all pyrimidines comprise 2′ methyl modified pyrimidines, and all purines comprise 2′ fluoro modified purines.

33. The composition of claim 27, wherein the oligonucleotide comprises a phosphate at the 5′ end of the antisense strand.

34. The composition of claim 27, wherein the oligonucleotide comprises a phosphate mimic at the 5′ end of the antisense strand.

35. The composition of claim 34, wherein the phosphate mimic comprises a 5′-vinyl phosphonate (VP).

36. The composition of any one of claims 1, 3, or 6, wherein the oligonucleotide comprises an antisense oligonucleotide (ASO).

37. The composition of claim 36, wherein the ASO is 12-30 nucleosides in length.

38. A composition comprising an oligonucleotide that inhibits the expression of MTRES1, wherein the oligonucleotide comprises an ASO about 12-30 nucleosides in length and a nucleoside sequence complementary to about 12-30 contiguous nucleosides of SEQ ID NO: 2443.

39. The composition of any one of claims 1, 3, 6, or 38, further comprising a pharmaceutically acceptable carrier.

40. A method of treating a subject having a neurological disorder, comprising administering an effective amount of the composition of claim 39 to the subject.

41. The method of claim 40, wherein the neurological disorder comprises dementia, Alzheimer's disease, delirium, cognitive decline, vascular dementia, or Parkinson's disease.
